(19) United States
(12) Patent Application Publication
(10) Pub. No.: US 2026/0031230 A1
(43) Pub. Date: Jan. 29, 2026

(54) METHODS OF IDENTIFYING PANCREATIC CANCER

(71) Applicant: PrognomiQ, Inc., San Mateo, CA (US)

(72) Inventors: Philip MA, San Jose, CA (US); Bruce WILCOX, Keezletown, VA (US); Chinmay BELTHANGADY, San Jose, CA (US); John BLUME, Bellingham, WA (US); Manway LIU, Burlingame, CA (US); Ehdieh KHALEDIAN, Belmont, CA (US); Preston B. WILLIAMS, San Mateo, CA (US)

(21) Appl. No.: 19/074,146

(22) Filed: Mar. 7, 2025

Related U.S. Application Data
(63) Continuation of application No. PCT/US2023/073688, filed on Sep. 7, 2023.
(60) Provisional application No. 63/375,020, filed on Sep. 8, 2022.
(60) Provisional application No. 63/485,190, filed on Feb. 15, 2023.

(51) Int. Cl. G16H 50/20; G16B 25/10; G16H 30/40

(52) U.S. Cl. CPC G16H 50/20; G16B 25/10; G16H 30/40

(57) ABSTRACT

Described herein are methods for identifying a biological state such as pancreatic cancer in a subject. For example, a method may include obtaining protein data, transcriptomic data, genomic data, lipidomic data, or metabolomic data of a subject and identifying a likelihood of the subject having pancreatic cancer. The disclosure includes methods of making and using classifiers.

### CROSS-REFERENCE

This application is a continuation of International Application No. PCT/US2023/073688, filed Sep. 7, 2023, which claims the benefit of U.S. Provisional Application No. 63/375,020, filed Sep. 8, 2022, and U.S. Provisional Application No. 63/485,190, filed Feb. 15, 2023, all of which are incorporated herein by reference.

### INCORPORATION BY REFERENCE OF SEQUENCE LISTING

The instant application contains a Sequence Listing which has been submitted electronically in XML format and is hereby incorporated by reference in its entirety. Said XML copy, created on Mar. 7, 2025, is named 59521-714_301_SL.xml and is 21,209 bytes in size.

### BACKGROUND

There is a need for accurate detection of cancers such as pancreatic cancer at an early stage. Accurately detecting cancer at an early stage can lead to effective treatments and improved prognosis for a subject having the cancer.

### SUMMARY

Disclosed herein, in some aspects, are detection methods. Some aspects include measuring, in a biofluid sample of a subject suspected of having pancreatic cancer, biomarkers comprising AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1, or a combination thereof, to obtain biomarker measurements. Some aspects include applying a classifier to the biomarker measurements to evaluate pancreatic cancer in the subject. Some aspects include identifying the biomarker measurements as indicative of the pancreatic cancer in the subject, or as identifying the biomarker measurements as indicative of a lack of the pancreatic cancer in the subject. Some aspects include administering a pancreatic cancer treatment to the subject when the biomarker measurements are identified as indicative of the pancreatic cancer, and observing or treating the subject without administering the pancreatic cancer treatment to the subject when the biomarker measurements are identified as indicative of a lack of the pancreatic cancer. Disclosed herein, in some aspects, are evaluation methods. Some aspects include obtaining a data set comprising biomarker measurements from a biofluid sample from a subject suspected of having pancreatic cancer, the biomarkers comprising AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1, or a combination thereof, and applying a classifier to the data set to evaluate pancreatic cancer in the subject. In some aspects, evaluating pancreatic cancer in the subject comprises identifying the data set as indicative of the pancreatic cancer in the subject, or as identifying the data set as indicative of a lack of the pancreatic cancer in the subject. Some aspects include administering a pancreatic cancer treatment to the subject when the data set is identified as indicative of the pancreatic cancer, and observing or treating the subject without administering the pancreatic cancer treatment to the subject when the data set is identified as indicative of a lack of the pancreatic cancer. In some aspects, the classifier comprises a performance, as determined with by a receiver operating characteristic (ROC) curve having an area under the curve (AUC) greater than 0.85, greater than 0.86, greater than 0.87, greater than 0.88, greater than 0.89, greater than 0.90, greater than 0.91, greater than 0.92, greater than 0.93, greater than 0.94, greater than 0.95, greater than 0.96, or greater than 0.97, in distinguishing between pancreatic cancer and a lack of pancreatic cancer. In some aspects, the classifier comprises a performance, as determined with by a sensitivity greater than 50%, greater than 55%, greater than 60%, greater than 65%, greater than 70%, greater than 75%, greater than 80%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99%, in identifying pancreatic cancer from a lack of pancreatic cancer. In some aspects, the classifier comprises a performance, as determined with by a specificity greater than 80%, greater than 81%, greater than 82%, greater than 83%, greater than 84%, greater than 85%, greater than 86%, greater than 87%, greater than 88%, greater than 0.89%, greater than 0.90%, greater than 0.91%, greater than 0.92%, greater than 93%, greater than 94%, greater than 95%, greater than 96%, greater than 97%, greater than 98%, or greater than 99%, in distinguishing between pancreatic cancer and a lack of pancreatic cancer. In some aspects, the biomarkers comprise A1AT. In some aspects, the biomarkers comprise A2GL. In some aspects, the biomarkers comprise AACT. In some aspects, the biomarkers comprise AMPN. In some aspects, the biomarkers comprise APOA1. In some aspects, the biomarkers comprise APOA2. In some aspects, the biomarkers comprise CO2. In some aspects, the biomarkers comprise CO5. In some aspects, the biomarkers comprise CO9. In some aspects, the biomarkers comprise CRP. In some aspects, the biomarkers comprise F13B. In some aspects, the biomarkers comprise FCG3A. In some aspects, the biomarkers comprise ICAM1. In some aspects, the biomarkers comprise ITIH3. In some aspects, the biomarkers comprise LBP. In some aspects, the biomarkers comprise NOE1. In some aspects, the biomarkers comprise PIGR. In some aspects, the biomarkers comprise RET4. In some aspects, the biomarkers comprise S10A8. In some aspects, the biomarkers comprise TETN. In some aspects, the biomarkers comprise CA19-9. In some aspects, the biomarkers comprise two or more, three or more, four or more, five or more, six or more, seven or more, eight or more, nine or more, ten or more, 12 or more, 14 or more, 16 or more, 18 or more, or 20 or more of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, APOA1, or CA19-9. In some aspects, the measurements are obtained by adding an internal standard for any of the biomarkers to the sample. In some aspects, the internal standard is labeled. In some aspects, the internal standard is isotopically labeled. In some aspects, biomarker the measurements are obtained using mass spectrometry. In some aspects, the biomarker measurements are obtained using an immunoassay. In some aspects, the biomarker measurements are obtained using molecular probes. In some aspects, the biomarker measurements are obtained using chromatography. In some aspects, the biofluid comprises pancreatic cyst fluid, blood, plasma, or serum. In some aspects, the subject is a mammal. In some aspects, the subject is a human. In some aspects, the classifier identifies a pancreatic cancer stage in the subject. In some aspects, the pancreatic cancer comprises stage I or stage II pancreatic cancer. In some aspects, the pancreatic cancer comprises stage III or IV pancreatic cancer. In some aspects, the pancreatic cancer comprises pancreatic ductal adenocarcinoma (PDAC). In some aspects, when the cancer evaluation method indicates that the subject has a probability exceeding a predetermined threshold of having pancreatic cancer, the method further comprises conducting a subsequent pancreatic cancer treatment, or advising the subject to undergo a subsequent pancreatic cancer treatment, to determine the presence of pancreatic cancer. In some aspects, the subsequent pancreatic cancer treatment comprises a biopsy. In some aspects, the subsequent pancreatic cancer treatment comprises pancreatic imaging. In some aspects, the imaging is performed using an ultrasound or computed tomography. In some aspects, when the cancer evaluation method indicates that the subject has a probability exceeding a predetermined threshold of having a pancreatic cancer, the method further comprises treating the subject with a pancreatic cancer treatment for treating the pancreatic cancer or advising the subject to undergo such pancreatic cancer treatment In some aspects, the pancreatic cancer treatment is selected from the group consisting of: surgery for pancreatic cancer, radiation therapy for pancreatic cancer, cryotherapy for pancreatic cancer, hormone therapy for pancreatic cancer, chemotherapy for pancreatic cancer, ablative treatments for pancreatic cancer, and immunotherapy for pancreatic cancer. In some aspects, the predetermined threshold is greater than 10%, greater than 20%, greater than 30%, greater than 40%, greater than 50%, greater than 60%, greater than 70%, greater than 80%, or greater than 90%. In some aspects, the classifier identifies a pancreatic cancer stage in the subject.

Disclosed herein, in some aspects, are a method of detecting pancreatic cancer in a subject, comprising: identifying a subject at risk of having pancreatic cancer; obtaining a biofluid sample from the subject; contacting the biofluid sample with particles such that the particles adsorb biomolecules comprising proteins to the particles; assaying the biomolecules adsorbed to the particles to generate proteomic data; and classifying the proteomic data as indicative of pancreatic cancer or as not indicative of pancreatic cancer. In some aspects, identifying the subject as at risk of having pancreatic cancer comprises identifying the subject as having a computed tomography (CT) scan indicative of pancreatic cancer, having a magnetic resonance imaging (MRI) scan indicative of pancreatic cancer, having a positron emission tomography (PET) scan indicative of pancreatic cancer, having an ultrasound indicative of pancreatic cancer, having a cholangiopancreatography indicative of pancreatic cancer, having an angiography indicative of pancreatic cancer, having a liver function test (LFT) indicative of pancreatic cancer, having an elevated carcinoembryonic antigen (CEA) level relative to a control or baseline measurement, having an elevated carbohydrate antigen (CA) 19-9 level relative to a control or baseline measurement, having jaundice, having abdominal pain, having gallbladder or liver enlargement, having a blood clot, or having a pancreatic cyst, or a combination thereof. Some aspects include identifying a likelihood of the subject having pancreatic cancer based on the proteomic data. In some aspects, classifying the proteomic data as indicative of pancreatic cancer or as not indicative of pancreatic cancer comprises applying a classifier to the proteomic data. In some aspects, the classifier comprises features to identify a likelihood of the subject having pancreatic cancer. In some aspects, the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. In some aspects, the proteomic data is indicative of pancreatic cancer with a sensitivity or specificity of at least about 50%, at least about 60%, at least about 70%, at least about 80%, or at least about 90%. Some aspects include recommending a pancreatic cancer treatment for the subject when the proteomic data is classified as indicative of pancreatic cancer. Some aspects include administering a pancreatic cancer treatment to the subject when the proteomic data is classified as indicative of pancreatic cancer. Some aspects include recommending or performing a biopsy when the proteomic data is classified as indicative of pancreatic cancer. Some aspects include recommending observation of the subject without administering a pancreatic cancer treatment to the subject, or recommending observation of the subject without obtaining a biopsy of the subject, when the proteomic data is not classified as indicative of pancreatic cancer. Some aspects include observing the subject without administering a pancreatic cancer treatment to the subject, or observing the subject without obtaining a biopsy of the subject, when the proteomic data is not classified as indicative of pancreatic cancer. In some aspects, the pancreatic cancer treatment comprises chemotherapy, radiation therapy, immunotherapy, targeted therapy, surgery, or surgical resection, or a combination thereof. In some aspects, the pancreatic cancer treatment comprises administration of a pharmaceutical composition comprising capecitabine, erlotinib, fluorouracil, gemcitabine, irinotecan, leucovorin, nab-paclitaxel, nanoliposomal irinotecan, oxaliplatin, olaparib, or larotrectinib, or a combination thereof. In some aspects, the particles comprise nanoparticles. In some aspects, the particles comprise lipid particles, metal particles, silica particles, or polymer particles. In some aspects, the particles comprise carboxylate particles, poly acrylic acid particles, dextran particles, polystyrene particles, dimethylamine particles, amino particles, silica particles, or N-(3-trimethoxysilylpropyl)diethylenetriamine particles. In some aspects, the particles comprise physiochemically distinct groups of nanoparticles. In some aspects, assaying the biomolecules comprises performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some aspects, assaying the biomolecules comprises performing mass spectrometry. In some aspects, assaying the biomolecules comprises measuring a readout indicative of the presence, absence or amount of the biomolecules. In some aspects, the pancreatic cancer comprises early stage pancreatic cancer. In some aspects, the pancreatic cancer comprises late stage pancreatic cancer. Some aspects include monitoring the subject and assaying biomolecules in a second biofluid sample obtained from the subject at a later time. In some aspects, the proteins comprise secreted proteins. In some aspects, the biofluid comprises blood, plasma, or serum. In some aspects, the subject has the pancreatic cancer. In some aspect, the subject does not have the pancreatic cancer. In some aspects, the subject is a mammal. In some aspects, the subject is a human.

Disclosed herein, in some aspects, are a method comprising: assaying proteins in a biofluid sample obtained from a subject identified as at risk of having pancreatic cancer to obtain protein measurements; and applying a classifier to the protein measurements, thereby identifying the protein measurements as indicative of the subject having pancreatic cancer, wherein the classifier is generated using proteomic data obtained by contacting training samples with particles such that the particles adsorb proteins in the training samples and assaying the proteins adsorbed to the particles. In some aspects, the subject is identified as at risk of having pancreatic cancer by identifying the subject as having a computed tomography (CT) scan indicative of pancreatic cancer, having a magnetic resonance imaging (MRI) scan indicative of pancreatic cancer, having a positron emission tomography (PET) scan indicative of pancreatic cancer, having an ultrasound indicative of pancreatic cancer, having a cholangiopancreatography indicative of pancreatic cancer, having an angiography indicative of pancreatic cancer, having a liver function test (LFT) indicative of pancreatic cancer, having an elevated carcinoembryonic antigen (CEA) level relative to a control or baseline measurement, having an elevated carbohydrate antigen (CA) 19-9 level relative to a control or baseline measurement, having jaundice, having abdominal pain, having gallbladder or liver enlargement, having a blood clot, or having a pancreatic cyst, or a combination thereof. Some aspects include identifying a likelihood of the subject having pancreatic cancer based on the proteomic data. In some aspects, the classifier comprises features to identify a likelihood of the subject having pancreatic cancer. In some aspects, the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. In some aspects, the proteomic data is indicative of pancreatic cancer with a sensitivity or specificity of at least about 50%, at least about 60%, at least about 70%, at least about 80%, or at least about 90%. Some aspects include recommending a pancreatic cancer treatment for the subject when the proteomic data is classified as indicative of pancreatic cancer. Some aspects include administering a pancreatic cancer treatment to the subject when the proteomic data is classified as indicative of pancreatic cancer. Some aspects include recommending or performing a biopsy when the proteomic data is classified as indicative of pancreatic cancer. Some aspects include recommending observation of the subject without administering a pancreatic cancer treatment to the subject, or recommending observation of the subject without obtaining a biopsy of the subject, when the proteomic data is not classified as indicative of pancreatic cancer. Some aspects include observing the subject without administering a pancreatic cancer treatment to the subject, or observing the subject without obtaining a biopsy of the subject, when the proteomic data is not classified as indicative of pancreatic cancer. In some aspects, the pancreatic cancer treatment comprises chemotherapy, radiation therapy, immunotherapy, targeted therapy, surgery, or surgical resection, or a combination thereof. In some aspects, the pancreatic cancer treatment comprises administration of a pharmaceutical composition comprising capecitabine, erlotinib, fluorouracil, gemcitabine, irinotecan, leucovorin, nab-paclitaxel, nanoliposomal irinotecan, oxaliplatin, olaparib, or larotrectinib, or a combination thereof. In some aspects, the particles comprise nanoparticles. In some aspects, the particles comprise lipid particles, metal particles, silica particles, or polymer particles. In some aspects, the particles comprise carboxylate particles, poly acrylic acid particles, dextran particles, polystyrene particles, dimethylamine particles, amino particles, silica particles, or N-(3-trimethoxysilylpropyl)diethylenetriamine particles. In some aspects, the particles comprise physiochemically distinct groups of nanoparticles. In some aspects, assaying the proteins comprises performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some aspects, assaying the proteins comprises performing mass spectrometry. In some aspects, assaying the proteins comprises measuring a readout indicative of the presence, absence or amount of the proteins. In some aspects, the pancreatic cancer comprises early stage pancreatic cancer. In some aspects, the pancreatic cancer comprises late stage pancreatic cancer. Some aspects include monitoring the subject and assaying proteins in a second biofluid sample obtained from the subject at a later time. In some aspects, the proteins comprise secreted proteins. In some aspects, the biofluid comprises blood, plasma, or serum. In some aspects, the subject has the pancreatic cancer. In some aspect, the subject does not have the pancreatic cancer. In some aspects, the subject is a mammal. In some aspects, the subject is a human.

Disclosed herein, in some aspects, are a method of treatment, comprising: identifying a mass in a pancreas of a subject; obtaining a biofluid sample from the subject; contacting the biofluid sample with particles such that the particles adsorb biomolecules comprising proteins to the particles; assaying the biomolecules adsorbed to the particles to generate proteomic data; and classifying the proteomic data as indicative of the mass comprising pancreatic cancer or as not indicative of the mass comprising pancreatic cancer. Some aspects include biopsying the mass when the proteomic data is classified as indicative of the mass comprising pancreatic cancer, and not biopsying the mass when the proteomic data is classified as not indicative of the mass comprising pancreatic cancer. The mass may include a pancreatic cyst. The mass may be identified by a medical imaging technique such as a CT scan or MRI. In some aspects, the subject is identified as at risk of having pancreatic cancer by identifying the subject as having a computed tomography (CT) scan indicative of pancreatic cancer, having a magnetic resonance imaging (MRI) scan indicative of pancreatic cancer, having a positron emission tomography (PET) scan indicative of pancreatic cancer, having an ultrasound indicative of pancreatic cancer, having a cholangiopancreatography indicative of pancreatic cancer, having an angiography indicative of pancreatic cancer, having a liver function test (LFT) indicative of pancreatic cancer, having an elevated carcinoembryonic antigen (CEA) level relative to a control or baseline measurement, having an elevated carbohydrate antigen (CA) 19-9 level relative to a control or baseline measurement, having jaundice, having abdominal pain, having gallbladder or liver enlargement, having a blood clot, or having a pancreatic cyst, or a combination thereof. Some aspects include identifying a likelihood of the mass being cancerous based on the proteomic data. In some aspects, classifying the proteomic data as indicative of the mass being cancerous or not comprises applying a classifier to the proteomic data. In some aspects, the classifier comprises features to identify a likelihood of the mass being cancerous. In some aspects, the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. In some aspects, the proteomic data is indicative of the mass being cancerous with a sensitivity or specificity of at least about 50%, at least about 60%, at least about 70%, at least about 80%, or at least about 90%. Some aspects include recommending a pancreatic cancer treatment for the subject when the proteomic data is classified as indicative of the mass being cancerous. Some aspects include administering a pancreatic cancer treatment to the subject when the proteomic data is classified as indicative of the mass being cancerous. Some aspects include recommending or performing a biopsy when the proteomic data is classified as indicative of the mass being cancerous. Some aspects include recommending observation of the subject without administering a pancreatic cancer treatment to the subject, or recommending observation of the subject without obtaining a biopsy of the subject, when the proteomic data is not classified as indicative of the mass being cancerous. Some aspects include observing the subject without administering a pancreatic cancer treatment to the subject, or observing the subject without obtaining a biopsy of the subject, when the proteomic data is not classified as indicative of the mass being cancerous. In some aspects, the pancreatic cancer treatment comprises chemotherapy, radiation therapy, immunotherapy, targeted therapy, surgery, or surgical resection, or a combination thereof. In some aspects, the pancreatic cancer treatment comprises administration of a pharmaceutical composition comprising capecitabine, erlotinib, fluorouracil, gemcitabine, irinotecan, leucovorin, nab-paclitaxel, nanoliposomal irinotecan, oxaliplatin, olaparib, or larotrectinib, or a combination thereof. In some aspects, the particles comprise nanoparticles. In some aspects, the particles comprise lipid particles, metal particles, silica particles, or polymer particles. In some aspects, the particles comprise carboxylate particles, poly acrylic acid particles, dextran particles, polystyrene particles, dimethylamine particles, amino particles, silica particles, or N-(3-trimethoxysilylpropyl)diethylenetriamine particles. In some aspects, the particles comprise physiochemically distinct groups of nanoparticles. In some aspects, assaying the biomolecules comprises performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some aspects, assaying the biomolecules comprises performing mass spectrometry. In some aspects, assaying the biomolecules comprises measuring a readout indicative of the presence, absence or amount of the biomolecules. In some aspects, the pancreatic cancer comprises early stage pancreatic cancer. In some aspects, the pancreatic cancer comprises late stage pancreatic cancer. Some aspects include monitoring the subject and assaying biomolecules in a second biofluid sample obtained from the subject at a later time. In some aspects, the proteins comprise secreted proteins. In some aspects, the biofluid comprises blood, plasma, or serum. In some aspects, the mass is cancerous. In some aspect, the mass is not cancerous. In some aspects, the subject is a mammal. In some aspects, the subject is a human.

Disclosed herein, in some aspects, are multi-omic cancer detection methods, comprising: obtaining multi-omic data generated from one or more biofluid samples collected from a subject, the multi-omic data comprising a first omic data and a second omic data, wherein the first omic data comprises a first omic data type comprises proteomic data, metabolomic data, transcriptomic data, or genomic data, and wherein the second omic data comprises a second omic data type different from the first omic data type and comprises proteomic data, metabolomic data, transcriptomic data, or genomic data; using a first classifier to assign a first label corresponding to a presence, absence, or likelihood of pancreatic cancer to the first omic data; using a second classifier to assign a second label corresponding to a presence, absence, or likelihood of pancreatic cancer to the second omic data; and based on a combination of the first and second labels, identifying the multi-omic data as indicative or as not indicative of pancreatic cancer, wherein the first and second classifiers are independent, and wherein the combination of the first and second labels identifies the multi-omic data as indicative or as not indicative of pancreatic cancer with greater accuracy than the first or second label alone. In some aspects, the first or second omic data type comprises proteomic data. In some aspects, the proteomic data comprises measurements of at least 1000 proteins or peptides. In some aspects, the proteomic data are generated from contacting a biofluid sample of the one or more biofluid samples with particles such that the particles adsorb biomolecules comprising proteins. In some aspects, the particles comprise a metal, polymer, or lipid. In some aspects, the particles comprise physiochemically distinct groups of nanoparticles. In some aspects, the proteomic data are generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some aspects, the genomic or the transcriptomic data are generated by sequencing, microarray analysis, hybridization, polymerase chain reaction, electrophoresis, or a combination thereof. In some aspects, the first or second omic data type comprises transcriptomic data. In some aspects, the transcriptomic data comprise mRNA or microRNA expression data. In some aspects, the first or second omic data type comprises genomic data. In some aspects, the genomic data comprise DNA sequence data or epigenetic data. In some aspects, the epigenetic data comprise DNA methylation data, DNA hydroxymethylation data, or histone modification data. In some aspects, the first or second omic data type comprises metabolomic data. Some aspects include identifying the multi-omic data as indicative or as not indicative of pancreatic cancer comprises generating or obtaining a majority voting score based on the first and second labels. In some aspects, identifying the multi-omic data as indicative or as not indicative of pancreatic cancer comprises generating or obtaining a weighted average of the first and second labels. Some aspects include assigning weights to the first and second classifiers, thereby obtaining the weighted average. In some aspects, the weights are assigned based on area under a ROC curve, area under a precision-recall curve, accuracy, precision, recall, sensitivity, F1-score, specificity, or a combination thereof. In some aspects, the first and second classifiers err independently with regard to pancreatic cancer identification. Some aspects include transmitting or outputting a report comprising information on the identification. Some aspects include transmitting or outputting a recommendation of a pancreatic cancer treatment of the subject based on the pancreatic cancer identification. In some aspects, the pancreatic cancer is labeled as indicative of pancreatic cancer with an accuracy as characterized by a receiver operating characteristic (ROC) curve having an area under the curve (AUC) greater than 0.7, greater than 0.75, greater than 0.8, greater than 0.85, greater than 0.9, greater than 0.91 or greater than 0.92.

Disclosed herein, in some aspects, are methods of evaluating a subject suspected of having pancreatic cancer, comprising: measuring biomarkers in a biofluid sample from the subject, wherein the biomarkers comprise A2GL, AKR1B1, ANPEP, ANTXR1, ANTXR2, BTK, CALR, CDH1, CDH11, CDH2, CDHR2, CILP2, CLEC3B, COL18A1, CRP, EXT1, F13A1, FAT1, FGL1, FLT4, ICAM1, IDH2, LCN2, LPP, MAPK1, MAP2K1, MYH9, NOTCH1, NOTCH2, PIGR, PPP2R1A, PRKAR1A, PXDN, RELN, RHOA, S100A8, S100A9, S100A12, SAA1, SAA2, SERPINA3, SLAIN2, SND1, SVEP1, TSP2, TUBB, TUBB1, or VCAN.

Disclosed herein, in some aspects, are methods, comprising: assaying biomolecules in a biofluid sample obtained from a subject suspected of having pancreatic cancer to obtain biomolecule measurements; and identifying the protein measurements as indicative of the subject having the pancreatic cancer or as not having the pancreatic cancer by applying a classifier to the biomolecule measurements, wherein the classifier is characterized by a receiver operating characteristic (ROC) curve having an area under the curve (AUC) greater than 0.7, greater than 0.75, greater than 0.8, greater than 0.85, greater than 0.9, greater than 0.91 or greater than 0.92, based on biomolecule measurement features. In some aspects, the AUC is no greater than 0.75, no greater than 0.8, no greater than 0.85, no greater than 0.9, no greater than 0.91, no greater than 0.92, no greater than 0.93, or no greater than 0.94. In some aspects, the biomolecules comprise proteins, lipids, and metabolites.

Disclosed herein, in some aspects of the inventive concepts, are method for generating a multi-omic classifier comprising: obtaining first omic data of a first omic data type and obtaining second omic data of a second omic data type different from the first omic data type. The first and second omic data may correspond to biomolecules present in biological samples of subjects. A first classifier of a biological state using features of the first omic data may be generated and a second classifier of the biological state using features of the second omic data may also be generated. The method may further comprise assigning feature importance scores to the features of the first and second classifiers. The method may further comprise selecting top features of the first classifier, and selecting top features of the second classifier. A combined classifier using the selected top features of the first and second classifiers may then be generated.

In some aspects, the method may comprise generating a first classifier using features of a first omic data comprises using all available features of the first omic data. In some aspects, the method may further comprise generating a second classifier using features of a second omic data comprises using all available features of the second omic data. In some aspects, the method may further comprise generating a first classifier using features of the first omic data which comprises performing machine learning with the features of the first omic data. In some aspects, the method may further comprise generating a second classifier using features of a second omic data which comprises performing machine learning with the features of the second omic data. In some aspects, generating the first classifier using features of the first omic data may comprise performing repeated cross-validation (RCV) using the features of the first omic data. In some aspects, the method may further comprise generating the second classifier using features of the second omic data comprises performing RCV using the features of the second omic data. The method may further comprise the features of the first and second omic data comprise measurements of biomolecules.

In some aspects, the method may comprise the selected top features of the first classifier wherein the first classifier comprises 1, 2, 3, 4, 5, 6, 7, 8, 9, 10, 11, 12, 13, 14, 15, 16, 17, 18, 19, or 20 features. In some aspects, the method may further comprise the selected top features of the second classifier wherein the second classifier comprises 1, 2, 3, 4, 5, 6, 7, 8, 9, 10, 11, 12, 13, 14, 15, 16, 17, 18, 19, or 20 features. In some aspects, the method may comprise the selected top features of the first classifier, and wherein the first classifier comprises the same number of features as the selected top features of the second classifier. In some aspects, the method may further comprise generating the combined classifier comprises performing RCV using the selected top features of the first classifier. In some aspects, the method may further comprise generating the combined classifier comprises performing RCV using the selected top features of the second classifier. In some aspects, the method may further comprise generating the combined classifier comprises using the features selected from each of the initial omics models from a first shuffling of the subjects into RCV repeats and folding in a second RCV shuffling of the subjects into newly grouped repeats and folds. In some aspects, the combined classifier may further comprise using another re-sampling method. The re-sampling method may be a nested cross-validation (NCV) or a leave-one-out-cross validation (LOOCV), among others. Any re-sampling method that builds error estimates for eventual generalization to a help out test or validation set. sent In some aspects, the method may further comprise generating a combined classifier comprises excluding features that fall below an importance threshold. In some aspects, the method may further comprising identifying features of the combined classifier that fall below a predetermined importance threshold, and training a final combined classifier that excludes the features that fall below the predetermined importance threshold. In some aspects, the combined classifier comprises a linear classifier. In some aspects, the first and second omic data are selected from proteomic data, metabolomic data, lipidomic data, transcriptomic data, and genomic data. In some aspects, the first omic data comprise measurements of biomolecules captured by a first particle type and the second omic data comprise measurements of biomolecules captured by a second particle. The first and second particles may be physiochemically distinct from each other. The first and second particles may comprise lipid particles, metal particles, silica particles, or polymer particles. The first and second particles may comprise nanoparticles.

In some aspects, the method may further comprise obtaining a third omic data of a third omic data type corresponding to biomolecules present in the biological samples, generating a third classifier of the biological state using features of the third omic data, assigning feature importance scores to the features of the third classifier, and selecting top features of the third classifier. Then possibly generating a combined classifier comprises using the selected top features of the first, second, and third classifiers. The method may further comprise obtaining fourth omic data of a fourth omic data type corresponding to biomolecules present in the biological samples, generating a fourth classifier of the biological state using features of the fourth omic data, assigning feature importance scores to the features of the fourth classifier, and selecting top features of the fourth classifier. Then possibly generating a combined classifier comprises using the selected top features of the first, second, third, and fourth classifiers. In some aspects, the first omic, the second omic, the third omic, and the fourth omic are independently selected from proteomic data, metabolomic data, lipidomic data, transcriptomic data, and genomic data. In some aspects, the first omic data may comprises proteomic data, the second omic data may comprises metabolomic data, the third omic data may comprises lipidomic data, and the fourth omic data may comprises transcriptomic data. In some aspects, the combined classifier identifies subjects as having the biological state and as not having the biological state with a sensitivity of at least 70%, at 99% specificity. In some aspects, the combined classifier identifies subjects as having the biological state and as not having the biological state with a performance characterized by a receiver operating characteristic curve (ROC) having an area under the curve (AUC) of at least 0.95.

In some aspects, the biological state may comprise a disease. In some aspects, the disease may comprise cancer. In some aspects, the cancer may comprise pancreatic cancer. In some aspects, the pancreatic cancer may comprise pancreatic ductal adenocarcinoma. In some aspects, the cancer may comprise stage I or stage II cancer. In some aspects, the cancer may comprise stage III or stage IV cancer. In some aspects, the cancer may comprise stage I, II, III, or IV pancreatic cancer. In some aspects, the method may use a biological samples comprising a biofluid. In some aspects, the biofluid may comprise a blood, serum, plasma, or a combination thereof. In some aspects, the biofluid may be essentially cell-free. In some aspects, a classifier generated using the methods described herein may be used to evaluate a biological state of a subject using biomolecule data obtained from a sample of the subject. This evaluation may then further comprise administering a disease treatment to the subject based on the evaluation. In some embodiments, the disease treatment may be any of the pancreatic cancer treatments disclosed herein. For example, in some embodiments, the disease treatment may include surgery, organ transplantation, pharmaceutical composition administration, radiation therapy, chemotherapy, immunotherapy, hormone therapy, monoclonal antibody treatment, stem cell transplantation, gene therapy, or chimeric antigen receptor (CAR)-T cell or transgenic T cell administration. In some embodiments, the disease treatment may comprise chemotherapy, radiation therapy, immunotherapy, targeted therapy, surgery, or surgical resection, or a combination thereof. In some embodiments, the methods disclosed herein may recommend a disease treatment comprising administration of a pharmaceutical composition comprising capecitabine, erlotinib, fluorouracil, gemcitabine, irinotecan, leucovorin, nab-paclitaxel, nanoliposomal irinotecan, oxaliplatin, olaparib, or larotrectinib, or a combination thereof.

In some aspects, the top features for a classifier may be selected from at least 500, at least 1,000, at least 2,000, at least 3,000, at least 4,000, at least 5,000, at least 7,500, at least 10,000, at least 12,500, at least 15,000, at least 20,000, at least 30,000, at least 40,000, at least 50,000, at least 75,000, or at least 100,000 features of the first omic data. In some aspects, the top features for a classifier may be selected from no more than 500, no more than 1,000, no more than 2,000, no more than 3,000, no more than 4,000, no more than 5,000, no more than 7,500, no more than 10,000, no more than 12,500, no more than 15,000, no more than 20,000, no more than 30,000, no more than 40,000, no more than 50,000, no more than 75,000, or no more than 100,000 features of the first omic data. The number of feature of an omic set before choosing the top features may be defined by a range of any of the previous values. The top features may be chosen from a different number of features for each different omic data set. The top features chosen from multiple omic types for a combined classifier may be chosen from the same number of features or a different number of features. The number of features chosen from each set of features of each of the omic data sets may be the same or different. The difference in the number of features in each set of the omic data sets, prior to selection of the top features, may be in a range of a multiple of the number of features in another omic data set. The number of features from which the top features are chosen from a second omics data set may be at least 1 times, at least 10 times, at least 100 times, at least 1,000 times, at least 10,000 times, at least 100,000 times the number of features. It may be not more than 1 times, not more than 10 times, not more than 100 times, not more than 1,000 times, not more than 10,000 times, not more than 100,000 times the number of features from which the top features were chosen for the first omics data set. The ranges of number of features in each individual omic data set may be in the range disclosed above. If there are multiple omic data sets, each may have a range of number of features drawn from the range above, independent of any other omic set. A top feature classifier may have omic data sets that have numbers of features independently selected from the ranges of above without relationships between the number of features between any individual omic data set. They may also be the same number of features.

In some aspects, the inventive concepts may comprise a classifier generated using multi-omic data obtained from a sample of an intent-to-test population. The omics groups may be selected to represent physiological, biological, genetic, or function systems or structurers within a patient. These systems or structure may be given a weight based on the usefulness of the system or structure to provide predictive data for the classifier. These omic groups may be combined with or without prior knowledge weighting. The classifier generate may have an improved ability to classification in the true intent-to-test population. The combination may be based on a combination of different variables of the data. The variables may be chosen from feature importance score, omic group weighting, cumulative relative importance, or cumulative relative classification ability. One variable may be chosen, or a combination of variable may be used. Any combination of the previous variables along with any other variable may be used.

In some aspects, assigning the feature importance score to each feature may further comprises assigning one or more biological process associated with the feature. In some aspects, the one or more biological process may be a human biological process or gene ontology-biological process or any biological process that may be involved in the diagnosis or treatment of an altered biological state. In some aspects, the selection of the top feature may further comprises calculating the total number of biological processes of the top features. This may be performed so that the combined classifier has at least a certain number of biological process represented by the top features. This may create a classifier that has a higher sensitivity or specificity in the total population than a classifier that comprises features that represent fewer biological processes.

In some aspects, assigning one or more biological processes may further comprises calculating a significance of the association. The significance of the association may be calculated based on a formal test of statistical significance. The formal test of statistical significance may comprise a log Odds Ratio (LOR) calculation. The log Odds Ratio (LOR) calculation may comprise the equation:

LOR=ln((associations of specific process in the first omic data type/total associations of all processes in the first omic type−instances of the specific process in the first omic data type)/(associations of specific process in the second omic data type/total associations of all processes in the second omic type-instances of the specific process in the second omic data type))

and a Fisher's test for significance of proportionality differences and a Bonferroni correction of the raw p-value is used. A positive LOR may indicate significance for the first omic data type and a negative LOR may indicate significance for the second omic data type. In some aspects, only with an LOR of greater than 0.5 or less than −0.5 at a p value of <0.05 may an association with a feature of the omic data set with which the process has significance be made.

In some aspects, the subjects may comprise two separate groups, a first set of training subjects and a second set of training subjects. Generating the first classifier and the second classifier may use omic data corresponding to biomolecules present in biological samples of the first set of training subjects. In some aspects, the generating a combined classifier may further comprises using omic data corresponding to biomolecules present in biological samples of the second set of training subjects.

In some aspects, the training data used to generate the classifiers may be separated into two groups, a first set of training data and a second set of training data. The first omics-type-specific, all-features-in models may be trained using only the first set of training data. The first omic-type-specific, all-features-in models may be used for the purpose of important feature selection. The second set of training data may be used to generate a second, final top-feature-combined model.

Disclosed herein, in some aspects, are methods for detecting pancreatic cancer, comprising: (a) obtaining biomarkers from a biofluid sample of a subject; and (b) applying a classifier to the biomarkers to evaluate the pancreatic cancer, wherein the classifier distinguishes between biofluid samples of subjects with and without pancreatic cancer with a performance characterized by a receiver operating characteristic (ROC) curve having an average or median area under the curve (AUC) of at least 0.9; and wherein the biomarkers comprises any of the following peptides GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), DNC(UniMod:4)PHLPNSGQEDFDK (SEQ ID NO. 4), GLVLGAGWAEGYLR (SEQ ID NO. 5), LVFNPDQEDLDGDGRGDIC(UniMod:4)K (SEQ ID NO. 6), AFDLYFVLDK (SEQ ID NO. 7), VFLVGNVEIR (SEQ ID NO. 8), RVSPVGETYIHEGLK (SEQ ID NO. 9), ASEQIYYENR (SEQ ID NO. 10), VLPGGDTYMHEGFER (SEQ ID NO. 11), AVDIPHMDIEALK (SEQ ID NO. 12), AMGIMNSFVNDIFER (SEQ ID NO. 13), MPEQEYEFPEPR (SEQ ID NO. 14), SGVISDTELQQALSNGTWTPFNPVTVR (SEQ ID NO. 15), M(UniMod:35)EDVNSNVNADQEVR (SEQ ID NO. 16), VGHDYQWIGLNDK (SEQ ID NO. 17), HAEC(UniMod:4)IYLGHFSDPMYK (SEQ ID NO. 18), or NGIFWGTWPGVSEAHPGGYK (SEQ ID NO. 19); any of the following RNAs ENST00000483727.5, ENST00000531734.6, ENST00000437154.6, ENST00000531997.1, ENST00000424185.7, ENST00000652176.1, ENST00000392593.9, ENST00000532853.5, ENST00000429947.1, ENST00000580914.1, ENST00000368205.7, ENST00000531709.6, ENST00000524817.5, ENST00000651281.1, ENST00000499685.2, ENST00000311921.8, ENST00000472111.5, ENST00000585172.2, ENST00000287713.7, or ENST00000547687.2; any of the following lipids NEG_PC(18:2_20:5)+AcO, POS_DAG(18:1_20:0)+NH4, NEG_PE(O-16:0_22:6)−H, NEG_PC(18:2_20:3)+AcO, POS_CER(d18:1/18:0)+H, POS_CE(22:0)+NH4, NEG_PE(14:0_22:5)−H, NEG_PC(20:5_20:5)+AcO, POS_PE(P-18:0_18:3)+H, NEG_PE(O-16:0_20:3)−H, POS_CE(18:3)+NH4, NEG_PE(O-18:0_22:5)−H, NEG_PE(O-18:0_20:5)−H, POS_PE(P-20:0_20:3)+H, NEG_PE(O-16:0_20:2)−H, POS_CER(d18:1/24:0)+H, NEG_PA(20:1_20:3)−H, NEG_PA(20:0_20:5)−H, POS_CE(20:0)+NH4, or NEG_PC(16:1_20:3)+AcO; or any of the following metabolites NEG_AICAR POS_Cystine, NEG_CMP, NEG_Gentisate, POS_Creatine, POS_Imidazoleacetic acid, POS_Inosine, NEG_n-Isovalerylglycine, NEG_Glucose-6-Phosphate, POS_Metanephrine, NEG_N-Acetylglutamate, NEG_5-Thymidilic acid (dTMP), POS_UMP, NEG_Fructose-6-Phosphate, NEG_Cystine, POS_Ubiquinol, POS_Guanine, NEG_Shikimic Acid, POS_1-Methylimidazole acetate, or POS_Flavone 2. In some aspects, the biomarkers comprise two or more of at least one peptide, at least one RNA, at least one lipid, and at least one metabolite. In some aspects, the biomarkers comprise three or more of at least one peptide, at least one RNA, at least one lipid, and at least one metabolite. In some aspects, the biomarkers comprise at least one peptide, at least one RNA, at least one lipid, and at least one metabolite. In some aspects, the biomarkers comprise any of the following peptides GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), DNC(UniMod:4)PHLPNSGQEDFDK (SEQ ID NO. 4), GLVLGAGWAEGYLR (SEQ ID NO. 5), LVFNPDQEDLDGDGRGDIC(UniMod:4)K (SEQ ID NO. 6), AFDLYFVLDK (SEQ ID NO. 7), VFLVGNVEIR (SEQ ID NO. 8), RVSPVGETYIHEGLK (SEQ ID NO. 9), ASEQIYYENR (SEQ ID NO. 10), VLPGGDTYMHEGFER (SEQ ID NO. 11), AVDIPHMDIEALK (SEQ ID NO. 12), AMGIMNSFVNDIFER (SEQ ID NO. 13), MPEQEYEFPEPR (SEQ ID NO. 14), SGVISDTELQQALSNGTWTPFNPVTVR (SEQ ID NO. 15), M(UniMod:35)EDVNSNVNADQEVR (SEQ ID NO. 16), VGHDYQWIGLNDK (SEQ ID NO. 17), HAEC(UniMod:4)IYLGHFSDPMYK (SEQ ID NO. 18), or NGIFWGTWPGVSEAHPGGYK (SEQ ID NO. 19). In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more of the peptides. In some aspects, the biomarkers comprise any of the following RNAs ENST00000483727.5, ENST00000531734.6, ENST00000437154.6, ENST00000531997.1, ENST00000424185.7, ENST00000652176.1, ENST00000392593.9, ENST00000532853.5, ENST00000429947.1, ENST00000580914.1, ENST00000368205.7, ENST00000531709.6, ENST00000524817.5, ENST00000651281.1, ENST00000499685.2, ENST00000311921.8, ENST00000472111.5, ENST00000585172.2, ENST00000287713.7, or ENST00000547687.2. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the RNAs. In some aspects, the biomarkers comprise any of the following lipids NEG_PC(18:2_20:5)+AcO, POS_DAG(18:1_20:0)+NH4, NEG_PE(O-16:0_22:6)−H, NEG_PC(18:2_20:3)+AcO, POS_CER(d18:1/18:0)+H, POS_CE(22:0)+NH4, NEG_PE(14:0_22:5)−H, NEG_PC(20:5_20:5)+AcO, POS_PE(P-18:0_18:3)+H, NEG_PE(0-16:0_20:3)−H, POS_CE(18:3)+NH4, NEG_PE(0-18:0_22:5)−H, NEG_PE(0-18:0_20:5)−H, POS_PE(P-20:0_20:3)+H, NEG_PE(0-16:0_20:2)−H, POS_CER(d18:1/24:0)+H, NEG_PA(20:1_20:3)−H, NEG_PA(20:0_20:5)−H, POS_CE(20:0)+NH4, or NEG_PC(16:1_20:3)+AcO. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the lipids. In some aspects, the biomarkers comprise any of the following metabolites NEG_AICAR, POS_Cystine, NEG_CMP, NEG_Gentisate, POS_Creatine, POS_Imidazoleacetic acid, POS_Inosine, NEG_n-Isovalerylglycine, NEG_Glucose-6-Phosphate, POS_Metanephrine, NEG_N-Acetylglutamate, NEG_5-Thymidilic acid (dTMP), POS_UMP, NEG_Fructose-6-Phosphate, NEG_Cystine, POS_Ubiquinol, POS_Guanine, NEG_Shikimic Acid, POS_1-Methylimidazole acetate, or POS_Flavone 2. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the metabolites. In some aspects, the classifier comprises a performance characterized by a receiver operating characteristic (ROC) curve having an average or median area under the curve (AUC) of at least 0.90. In some aspects, the subject is suspected of having the pancreatic cancer. In some aspects, the method further comprises administering a pancreatic cancer treatment to the subject when the subject has pancreatic cancer. In some aspects, the method further comprises monitoring the subject when the subject does not have the pancreatic cancer.

Disclosed herein, in some aspects, are methods for treating pancreatic cancer, the method comprising: administering to a subject having the pancreatic cancer a pancreatic cancer treatment, wherein the pancreatic cancer is evaluated by a method comprising: (a) obtaining biomarkers from a biofluid sample of the subject; and (b) applying a classifier to the biomarkers to evaluate the pancreatic cancer, wherein the classifier distinguishes between biofluid samples of subjects with and without pancreatic cancer with a performance characterized by a receiver operating characteristic (ROC) curve having an average or median area under the curve (AUC) of at least 0.9; and wherein the biomarkers comprises any of the following peptides GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), DNC(UniMod:4)PHLPNSGQEDFDK (SEQ ID NO. 4), GLVLGAGWAEGYLR (SEQ ID NO. 5), LVFNPDQEDLDGDGRGDIC(UniMod:4)K (SEQ ID NO. 6), AFDLYFVLDK (SEQ ID NO. 7), VFLVGNVEIR (SEQ ID NO. 8), RVSPVGETYIHEGLK (SEQ ID NO. 9), ASEQIYYENR (SEQ ID NO. 10), VLPGGDTYMHEGFER (SEQ ID NO. 11), AVDIPHMDIEALK (SEQ ID NO. 12), AMGIMNSFVNDIFER (SEQ ID NO. 13), MPEQEYEFPEPR (SEQ ID NO. 14), SGVISDTELQQALSNGTWTPFNPVTVR (SEQ ID NO. 15), M(UniMod:35)EDVNSNVNADQEVR (SEQ ID NO. 16), VGHDYQWIGLNDK (SEQ ID NO. 17), HAEC(UniMod:4)IYLGHFSDPMYK (SEQ ID NO. 18), or NGIFWGTWPGVSEAHPGGYK (SEQ ID NO. 19); any of the following RNAs ENST00000483727.5, ENST00000531734.6, ENST00000437154.6, ENST00000531997.1, ENST00000424185.7, ENST00000652176.1, ENST00000392593.9, ENST00000532853.5, ENST00000429947.1, ENST00000580914.1, ENST00000368205.7, ENST00000531709.6, ENST00000524817.5, ENST00000651281.1, ENST00000499685.2, ENST00000311921.8, ENST00000472111.5, ENST00000585172.2, ENST00000287713.7, or ENST00000547687.2; any of the following lipids NEG_PC(18:2_20:5)+AcO, POS_DAG(18:1_20:0)+NH4, NEG_PE(O-16:0_22:6)−H, NEG_PC(18:2_20:3)+AcO, POS_CER(d18:1/18:0)+H, POS_CE(22:0)+NH4, NEG_PE(14:0_22:5)−H, NEG_PC(20:5_20:5)+AcO, POS_PE(P-18:0_18:3)+H, NEG_PE(O-16:0_20:3)−H, POS_CE(18:3)+NH4, NEG_PE(O-18:0_22:5)−H, NEG_PE(O-18:0_20:5)−H, POS_PE(P-20:0_20:3)+H, NEG_PE(O-16:0_20:2)−H, POS_CER(d18:1/24:0)+H, NEG_PA(20:1_20:3)−H, NEG_PA(20:0_20:5)−H, POS_CE(20:0)+NH4, or NEG_PC(16:1_20:3)+AcO; or any of the following metabolites NEG_AICAR POS_Cystine, NEG_CMP, NEG_Gentisate, POS_Creatine, POS_Imidazoleacetic acid, POS_Inosine, NEG_n-Isovalerylglycine, NEG_Glucose-6-Phosphate, POS_Metanephrine, NEG_N-Acetylglutamate, NEG_5-Thymidilic acid (dTMP), POS_UMP, NEG_Fructose-6-Phosphate, NEG_Cystine, POS_Ubiquinol, POS_Guanine, NEG_Shikimic Acid, POS_1-Methylimidazole acetate, or POS_Flavone 2. In some aspects, the biomarkers comprise two or more of at least one peptide, at least one RNA, at least one lipid, and at least one metabolite. In some aspects, the biomarkers comprise three or more of at least one peptide, at least one RNA, at least one lipid, and at least one metabolite. In some aspects, the biomarkers comprise at least one peptide, at least one RNA, at least one lipid, and at least one metabolite. In some aspects, the biomarkers comprise any of the following peptides GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), DNC(UniMod:4)PHLPNSGQEDFDK (SEQ ID NO. 4), GLVLGAGWAEGYLR (SEQ ID NO. 5), LVFNPDQEDLDGDGRGDIC(UniMod:4)K (SEQ ID NO. 6), AFDLYFVLDK (SEQ ID NO. 7), VFLVGNVEIR (SEQ ID NO. 8), RVSPVGETYIHEGLK (SEQ ID NO. 9), ASEQIYYENR (SEQ ID NO. 10), VLPGGDTYMHEGFER (SEQ ID NO. 11), AVDIPHMDIEALK (SEQ ID NO. 12), AMGIMNSFVNDIFER (SEQ ID NO. 13), MPEQEYEFPEPR (SEQ ID NO. 14), SGVISDTELQQALSNGTWTPFNPVTVR (SEQ ID NO. 15), M(UniMod:35)EDVNSNVNADQEVR (SEQ ID NO. 16), VGHDYQWIGLNDK (SEQ ID NO. 17), HAEC(UniMod:4)IYLGHFSDPMYK (SEQ ID NO. 18), or NGIFWGTWPGVSEAHPGGYK (SEQ ID NO. 19). In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more of the peptides. In some aspects, the biomarkers comprise any of the following RNAs ENST00000483727.5, ENST00000531734.6, ENST00000437154.6, ENST00000531997.1, ENST00000424185.7, ENST00000652176.1, ENST00000392593.9, ENST00000532853.5, ENST00000429947.1, ENST00000580914.1, ENST00000368205.7, ENST00000531709.6, ENST00000524817.5, ENST00000651281.1, ENST00000499685.2, ENST00000311921.8, ENST00000472111.5, ENST00000585172.2, ENST00000287713.7, or ENST00000547687.2. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the RNAs. In some aspects, the biomarkers comprise any of the following lipids NEG_PC(18:2_20:5)+AcO, POS_DAG(18:1_20:0)+NH4, NEG_PE(O-16:0_22:6)−H, NEG_PC(18:2_20:3)+AcO, POS_CER(d18:1/18:0)+H, POS_CE(22:0)+NH4, NEG_PE(14:0_22:5)−H, NEG_PC(20:5_20:5)+AcO, POS_PE(P-18:0_18:3)+H, NEG_PE(O-16:0_20:3)−H, POS_CE(18:3)+NH4, NEG_PE(O-18:0_22:5)−H, NEG_PE(O-18:0_20:5)−H, POS_PE(P-20:0_20:3)+H, NEG_PE(O-16:0_20:2)−H, POS_CER(d18:1/24:0)+H, NEG_PA(20:1_20:3)−H, NEG_PA(20:0_20:5)−H, POS_CE(20:0)+NH4, or NEG_PC(16:1_20:3)+AcO. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the lipids. In some aspects, the biomarkers comprise any of the following metabolites NEG_AICAR, POS_Cystine, NEG_CMP, NEG_Gentisate, POS_Creatine, POS_Imidazoleacetic acid, POS_Inosine, NEG_n-Isovalerylglycine, NEG_Glucose-6-Phosphate, POS_Metanephrine, NEG_N-Acetylglutamate, NEG_5-Thymidilic acid (dTMP), POS_UMP, NEG_Fructose-6-Phosphate, NEG_Cystine, POS_Ubiquinol, POS_Guanine, NEG_Shikimic Acid, POS_1-Methylimidazole acetate, or POS_Flavone 2. In some aspects, the biomarkers comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more of the metabolites.

### BRIEF DESCRIPTION OF THE DRAWINGS

FIG. 1 shows exemplary methods for generating and applying the classifiers described herein.

FIG. 2 shows examples of stages in pancreatic cancer patient screening and treatment.

FIG. 3 shows a non-limiting example of a computing device; in this case, a device with one or more processors, memory, storage, and a network interface.

FIG. 4 shows a diagram of classifier and feature information, in accordance with some aspects described herein.

FIG. 5A shows results of a Wilcox test for age comparisons and Fisher's exact test for gender proportionality.

FIG. 5B shows results of a Wilcox test for age comparisons and Fisher's exact test for gender proportionality.

FIG. 6A shows numbers of proteins detected across subject samples in an analysis of biofluid samples from control and cancer patients.

FIG. 6B shows numbers of proteins detected across subject samples in an analysis of biofluid samples from control and cancer patients.

FIG. 6C shows reproducibility of platform indicates ability to detect biological signal. Analysis Groups: C=Control; S=Sample. Left panel: only proteins with n>1 detections/analysis group were retained. 2 features with CV>300% out of 2,089 were removed for clarity. Right panel: only proteins with n>1 detections/analysis group were retained. 48 features with CV>300% out of 7,672 were removed for clarity.

FIG. 6D shows detection of more than 5,000 proteins in feasibility study of 212 subjects. A median of 4 peptides per protein was detected for proteins present in >25% of the samples with search parameters: 0.1% peptide/protein FDR, default timsTOF parameters with complete UniProt human proteome database with contaminants (50% reversed decoys).

FIG. 6E shows large numbers of proteins are reproducibly detected across samples. Individual nanoparticles yielded both complementary and common protein identifications. Unique protein groups were shown for each sample/particle+panel with grouping by sample and collection site.

FIG. 6F shows enhanced proteome coverage detecting known cancer related proteins. All detected, matching proteins from samples plotted on HPPP curve. GeneCards data used score reported from matching gene id and search term “cancer”. Detected HPPP1 proteins covered 8 orders of magnitude difference: highest concentration: P00450—Ceruloplasmin; 830,000 ng/mL; and lowest concentration: Q7Z627—E3 ubiquitin-protein ligase HUWE1; 0.0034 ng/mL.

FIG. 6G shows deep and efficient plasma proteomics at scale.

FIG. 6H shows quantitative performance of Proteograph suitable for large scale studies.

FIG. 6I shows reproducibility of protein enrichment by Proteograph at scale. Reproducibility of Proteograph enrichment ideally suited for biomarker discovery. Data collected across 191 enrichments of identical sample. Scope of collection includes 3 instruments; 3 cohort studies; 5 operators; 8 months of run time; 121 plates; and 1500+ subject samples.

FIG. 6J shows reproducibility of the platform over time (months) and instruments. Median MS1 peak areas for iRT peptides were all below 15% with majority below 10%.

FIG. 6K shows Application of platform to pancreatic cancer biomarker discovery.

FIG. 7A shows a plot of some top proteins differentially detected in biofluid samples from cancer patients relative to biofluid samples from control patients.

FIG. 7B is a plot showing a distribution of OpenTargets (OT) scores. OT scores (from 0 to 0.8) are on the x-axis includes, while the y-axis includes density (0 to 15).

FIG. 8A includes plots showing comparisons of gross signal medians by sample, analyte-type and class.

FIG. 8B shows box and whisker plots of most significantly different analytes per omics workflow ((i): lipid; (ii): metabolite; and (iii): Protein).

FIG. 8C shows an exemplary multimers classifier performance combining proteomics, lipidomics, and metabolomics measurements.

FIG. 9A includes a volcano plot of intensity differences and P-values for proteins adsorbed to nanoparticles and detected in biofluid samples from cancer patients, relative to biofluid samples from control patients. The volcano plot displays magnitude of difference on the x-axis, and significance on the y-axis, with most significant analytes highlighted.

FIG. 9B includes data for top protein P35442 after a particle-based measurement method.

FIG. 9C includes a volcano plot of intensity differences and P-values for proteins detected in biofluid samples from cancer patients, relative to biofluid samples from control patients. The volcano plot displays magnitude of difference on the x-axis, and significance on the y-axis, with the most significant analyte highlighted.

FIG. 9D includes data for top protein P01011 after a proteomic measurement.

FIG. 10A includes a volcano plot of intensity differences and P-values for lipids detected in biofluid samples from cancer patients, relative to biofluid samples from control patients. The volcano plot displays magnitude of difference on the x-axis, and significance on the y-axis, with the most significant analyte highlighted.

FIG. 10B includes data for top lipid CER(d18:1_18:0) after a lipidomic measurement.

FIG. 11A includes a volcano plot of intensity differences and P-values for metabolites detected in biofluid samples from cancer patients, relative to biofluid samples from control patients. The volcano plot displays magnitude of difference on the x-axis, and significance on the y-axis, with the most significant analyte highlighted.

FIG. 11B includes data for top metabolite AICAR after a metabolomic measurement.

FIG. 12A depicts cancer and healthy sample classification by UMAP projection, based on combined data.

FIG. 12B depicts cancer and healthy sample classification by PCA projection, based on combined data.

FIG. 12C depicts cancer and healthy sample classification by UMAP projection, based on Proteograph data.

FIG. 12D depicts cancer and healthy sample classification by PCA projection, based on Proteograph data.

FIG. 12E depicts cancer and healthy sample classification by UMAP projection, based on PiQuant data.

FIG. 12F depicts cancer and healthy sample classification by PCA projection, based on PiQuant data.

FIG. 12G depicts cancer and healthy sample classification by UMAP projection, based on lipid data.

FIG. 12H depicts cancer and healthy sample classification by PCA projection, based on lipid data.

FIG. 12I depicts cancer and healthy sample classification by UMAP projection, based on metabolite data.

FIG. 12J depicts cancer and healthy sample classification by PCA projection, based on metabolite data.

FIG. 13 protein, lipid, and metabolite features included in a classifier.

FIG. 14 shows classifier performance in a multi-omic study and includes receiver operating characteristic (ROC) curves for disease state classification. Area under the curve (AUC) values are also included in the figure with 90% confidence intervals in parentheses.

FIG. 15A shows performance of a classifier trained with data from genomics assays and includes a ROC curve for disease state classification. The AUC value at the bottom of the figure is shown with ±values based on 90% confidence.

FIG. 15B shows performance of a classifier trained with data from genomics assays (“Genomics”), a classifier trained with data from mass spectrometry assays (“Mass-spec”), and a classifier trained with data from genomics and mass spectrometry assays (“Combined”). The data shown in the figure include ROC curves for disease state classification. The AUC values include ±values based on 90% confidence.

FIG. 16A shows volcano plot showing the intensity difference between pancreatic cancer samples and healthy samples.

FIG. 16B shows study comparison group (H: healthy; PC: Pancreatic cancer). 124 of 3,381 detected proteins were statistically significant.

FIG. 17A shows a volcano plot showing differential abundance of lipid species between pancreatic cancer samples and healthy samples.

FIG. 17B illustrates a graph showing top hit lipids based on the volcano plot in FIG. 17A.

FIG. 17C shows a volcano plot showing differential abundance of lipid species between pancreatic cancer samples and healthy samples.

FIG. 17D illustrates a graph showing top hit metabolites based on the volcano plot in FIG. 17C.

FIG. 18A shows quantitative performance of Proteograph suitable for large scale studies (e.g., study in Example 7).

FIG. 18B shows reproducibility of protein enrichment by Proteograph at scale. Reproducibility of Proteograph enrichment ideally suited for biomarker discovery. System provides high throughput, reproducible and deep proteome coverage for novel discoveries. Quantitative, deep, untargeted proteomics biomarker studies were enabled by Proteograph reproducibility. Protein enrichment by Proteograph at scale was highly reproducible (NP1=0; NP2=0; NP3=2; NP4=0; and NP5=2).

FIG. 19A shows evaluation of K562 precursor detection with SWATH vs. Zeno SWATH DIA. Minimum increase of 26% in precursor identifications was detected utilizing Zeno SWATH DIA. All data was generated from pr and pg matrix (all quantified precursors and proteins called were identified) from DIA-NN output. All data searched in DIA-NN with “robust LC” and SCIEX K562 spectral library.

FIG. 19B shows evaluation of K562 precursor detection with SWATH vs. Zeno SWATH DIA. Minimum increase of 13% in protein group identifications was detected utilizing Zeno SWATH DIA. All data was generated from pr and pg matrix (all quantified precursors and proteins called were identified) from DIA-NN output. All data searched in DIA-NN with “robust LC” and SCIEX K562 spectral library.

FIG. 20 shows improved sensitivity increasing number of low abundant peptides species detected. Detection of low abundant peptides was improved with Zenon SWATH DI compared to SWATH.

FIG. 21 shows graphs generated from all qualified precursors. Data was searched in DIA-NN with “robust LV” and SCIEX K562 spectral library.

FIG. 22 shows quantitative sensitivity increases with mass on SWATH and Zeno SWATH DIA. Zeno SWATH DIA MS1 peak areas (K562) were distributed to lower abundance peptides.

FIG. 23A shows Zeno SWATCH DIA acquisition resulted in higher K562 MS2-based precursor quantity compared to SWATH acquisition alone across different peptide injection masses based on all qualified precursors. Data was searched in DIA-NN with “robust LC” and SCIEX K562 spectral library.

FIG. 23B shows Zeno SWATH DIA acquisition resulted in lower CV(5) for K562 precursor-level quantities compared to SWATCH acquisition alone across different peptide injection massed based on all quantified precursors. Data was searched in DIA-NN with “robust LC” and SCIEX K562 spectral library.

FIG. 24 shows Zeno Swatch DIA MS/MS acquisition resulted in 53-85% more peptide identifications from Proteograph generated from pooled control samples when compared to SWATH MS/MS DIA acquisition.

FIG. 25 shows 2,357 protein groups across all five nanoparticles in the representative subject cohort. The 1077 protein groups were identified in at least 25% of the patient samples.

FIG. 26A shows large numbers of proteins that were reproducibly detected across samples. Individual nanoparticles yielded both complementary and common protein identifications.

FIG. 26B shows improved sensitivity equates to detection of more low abundant peptides in Proteograph peptide detection.

FIG. 27 illustrates a machine-learning analysis scheme.

FIG. 28A illustrates collection sites and dates of subject enrollment for the 184 subjects.

FIG. 28B illustrates age and gender comparisons between pancreatic ductal adenocarcinoma (PDAC) and control study groups.

FIG. 29A illustrates distribution of SIS-normalized protein values by sample.

FIG. 29B illustrates distribution of SIS-normalized protein values sample medians by group.

FIG. 30 illustrates an outlier rejection analysis using MARLE.

FIG. 31 illustrates a volcano plot of Wilcoxon test values.

FIG. 32 illustrates individual protein expression quantities for study subjects.

FIG. 33 illustrates a PCA multi-variate analysis of study group separability.

FIG. 34 illustrates unsupervised hierarchical clustering of the protein data with two forced groups.

FIG. 35 illustrates a comparison of subject training and validation groups in a pancreatic cancer study.

FIG. 36 illustrates race tube ANOVA results for XGBoost RCV-based model parameter evaluation.

FIG. 37 illustrates combined ROC plots for 10×10 XGBoost RCV with optimal hyperparameters.

FIG. 38 illustrates an evaluation of GLMnet hyperparameter combinations in 10×10 RCV.

FIG. 39A illustrates GLMnet top feature RCV ROC plots with best hyperparameters.

FIG. 39B illustrates GLMnet top feature final model coefficients.

FIG. 40 illustrates validation of a ROC plot for a final top feature GLMnet model.

FIG. 41A illustrates CA19-9 levels in PDAC versus control groups.

FIG. 41B illustrates CA19-9 levels in PDAC stages versus controls at separate cancer stages.

FIG. 42 illustrates a CA19-9 model performance in a validation subject group.

FIG. 43A illustrates GLMnet combined final model coefficients.

FIG. 43B illustrates GLMnet combined RCV ROC plots with best hyperparameters.

FIG. 44 illustrates some validation details of a combined feature GLMnet-based classifier.

FIG. 45 illustrates a comparison of top feature OpenTargets scores to a database.

FIG. 46 includes a ROC plot illustrating classifier performance in a stagewise analysis for biofluid samples from subjects with pancreatic cancer.

FIG. 47 depicts sample and analysis details used in multi-omics experiments for pancreatic cancer.

FIG. 48 includes volcano plots in an analysis of biofluid samples from subjects with pancreatic cancer.

FIG. 49 includes a heat map of biomarkers and results from an analysis of biofluid samples from subjects with pancreatic cancer.

FIG. 50A depicts variance decomposition results for all samples in an analysis of biofluid samples from subjects with pancreatic cancer.

FIG. 50B depicts variance decomposition results for samples from subjects with cancer in a pancreatic cancer analysis.

FIG. 51 is a Venn diagram showing overlap of biomarkers in an analysis of biofluid samples from subjects with pancreatic cancer.

FIG. 52A-52C include plots that illustrate that multi-omics readouts can also be statistically combined to improve the interpretation of biological processes, and include characteristics related to some biomarkers.

FIG. 53 includes a plot showing trend analysis showed where groups of biomarker types changed similarly and correlated by degree based on cancer stage.

FIG. 54A-54D show PCA using all features and samples that were measured for each omics type. The ellipses show 95% confidence intervals for grouping of PDAC and control subjects, and the shapes represent the specific PDAC stages for that group. FIG. 54A: Protein, FIG. 54B: RNA, FIG. 54C: lipids, and FIG. 54D: metabolites.

FIG. 55 displays combined top feature, multi-omics GLMnet regression model coefficients. The resulting coefficients were plotted in order of magnitude decreasing from left to right. The features were annotated for omics type for protein, RNA, lipids, and metabolites. This graph demonstrated that no single omics type dominated the coefficients with all 4 classes represented in the top 7 features for this experimental data.

FIG. 56 displays CA19-9 levels measured in PDAC and control subjects.

FIG. 57A-D display plasma levels of the 20 features from the multi-omics model measured in validation subjects. FIG. 57A: Proteins, FIG. 57B: RNA, FIG. 57C: lipids, and FIG. 57D: metabolites.

FIG. 58A-58D show volcano plots of blood analyte features for each omics type measured in subjects. FIG. 58A: Peptide-nanoparticle features from a Proteograph protein analysis, FIG. 58B: RNA features as mapped to ENSTs from the RNAseq data, FIG. 58C: lipid from the targeted MS data, and FIG. 58D: metabolite features from the targeted MS data.

FIG. 59A-59D display the feature importance scores for individual omics models. The top 20 features from each individual omics final XGBoost model were ranked by arbitrary feature importance units. FIG. 59A: Peptide features with the specific Proteograph nanoparticle-modified sequence pair. FIG. 59B: RNA transcripts as mapped to ENSTs. FIG. 59C: Lipids with the MS data acquisition ionization mode, positive (POS) or negative (NEG) annotated. FIG. 59D: metabolites with the MS data acquisition ionization mode, positive (POS) or negative (NEG) annotated.

FIG. 60A-60B display classification performance of individual omics models for distinguishing PDAC from non-cancer controls in the validation cohort. The omics types used were protein, RNA, lipid, and metabolite. FIG. 60A: PDAC all-stage validation result comprising 26 PDAC and 46 control subjects and FIG. 60B: PDAC early-stage (I/II) validation result (subset of all-stage result) comprising 7 PDAC and 46 control subjects.

FIG. 61 displays a comparison of individual omics models' predicted class probabilities.

FIG. 62 displays combined top feature, multi-omics model classification performance for distinguishing PDAC (all stages) from non-cancer samples compared to CA19-9 performance in the validation cohort. For the multi-omics and CA19-9 models, performance in the validation subjects was plotted as a ROC curve. ROC AUCs with 95% confidence intervals were annotated.

FIG. 63 displays the counts for proteins and RNAs at various detection frequencies across the PDAC study subjects. There were 3,215 unique Uniprot entries and 131,059 unique Ensembl ENST entries detected in at least 25% of the 146 subjects in the study.

FIG. 64 displays the overlap of enumerated GOBP terms between the protein and RNA omics types from features of the PDAC test. All but 66 of the 6,040 terms associated with at least one protein feature are represented in the RNA omics type, and there are 5,966 out of 11,940 terms that are unique to RNA.

FIG. 65 displays the distribution of ln OddsRatio (LOR) values for GOBP terms in proteins vs RNA demonstrating the enrichment of a given GOBP term in one omics type or the other from the PDAC study.

FIG. 66 shows significance vs magnitude for GOBP LOR comparing protein to RNA. The raw p-value for the Fisher test, [−log 10(p-value)], was plotted against the magnitude of the enrichment calculated as LOR. 40 features, 23 for proteins and 17 for RNA, were significantly different after Bonferroni correction (light gray points). The GOBP names for the 20 most significant features are annotated.

FIG. 67 displays the normalized expression levels of four different protein features in normal versus PDAC blood.

FIG. 68 displays the normalized expression levels of different metabolite features in normal versus PDAC blood.

FIG. 69A-69B show that different molecular assays captured analytes from distinct biological processes. FIG. 69A illustrates biological processes captured by RNA-seq. FIG. 69B illustrates biological processes captured by untargeted proteomics.

### DETAILED DESCRIPTION

This disclosure provides non-invasive methods for detecting presence of a cancer such as pancreatic cancer, or risk of developing the cancer in a subject. Identifying cancer in a subject at an early stage can save the subject from further development of the cancer if treatment is provided early. Non-invasive tests can also be used to rule out the presence of a cancer, thereby saving subjects from having to undergo invasive testing such as a biopsy, which can be painful and stressful, or may risk damaging the subject.

Some insights from the study examples disclosed herein are that univariate analysis of individual 'omics has revealed multiple molecular markers that are statistically significantly different between cancer and non-cancer samples, unsupervised clustering on significantly associated cancer biomarkers has shown separation by disease status, decomposition of the variance into joint and individual components has shown that there may be some shared biological signals across omic types but also biology that is unique to each individual omic type, gene-set enrichment analysis has shown that a multi-omic approach can reveal unique associations with disease biology, and a trend analysis has shown multiple molecular markers across omic types that correlated with cancer stages. Included herein is a classifier that may distinguish pancreatic cancer stages based on a biofluid sample from a subject.

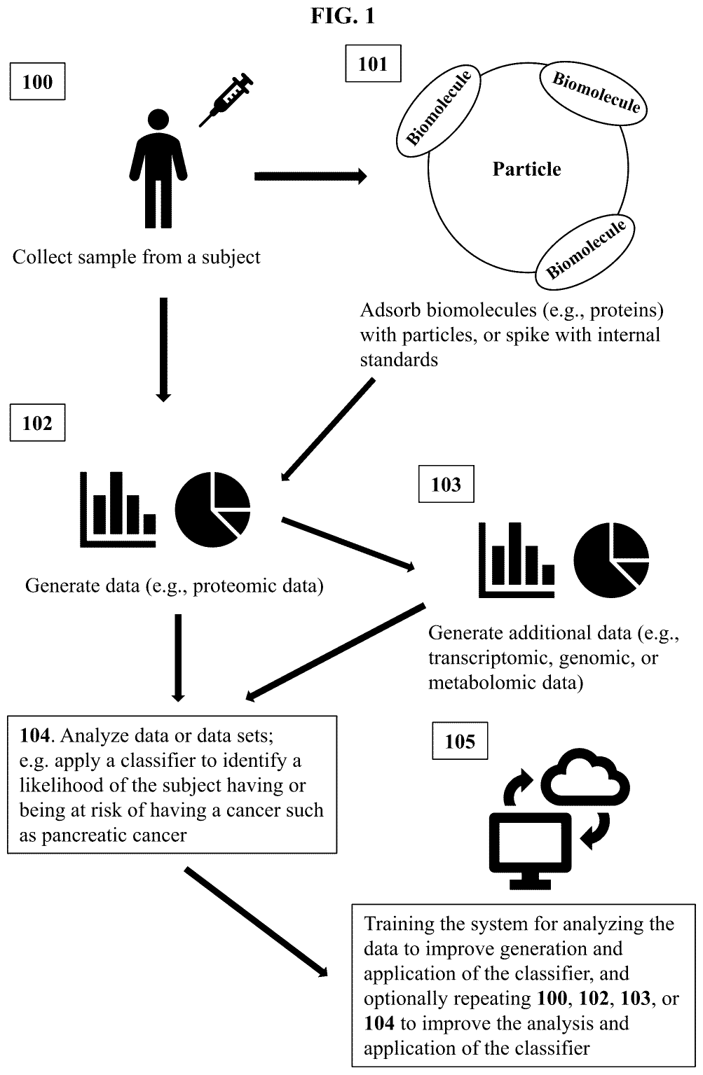

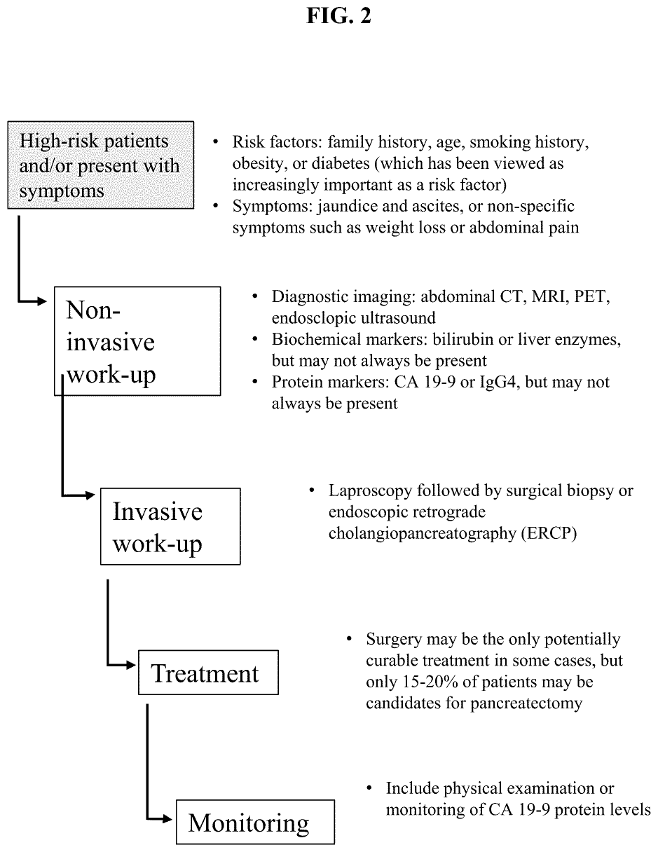

FIG. 1 illustrates a non-limiting example of methods for predicting whether a subject has or is at risk of developing a cancer such as pancreatic cancer based on assaying and analyzing a biofluid sample obtained from the subject (100). The biofluid sample can be any one of or any combination of the biofluids described herein. The sample can be either: directly analyzed to generate data (102) such as proteomic data; or contacted with particle described herein to obtain adsorbed biomolecules (103) prior to the analysis of 102. After obtaining the data from the analysis of 102, additional analysis (103) can be performed from the sample obtained from 100 or 101 to obtain additional data sets such as transcriptomic data, genomic data, metabolomic data, or a combination thereof. The data or data sets obtained from the analysis of 102 or 103 can then be used to generate a classifier, where the classifier can be applied to identify a likelihood of the subject having or at risk of having the cancer (105). The generation and the application of the classifier can be further repeated and refined to improve the analysis and application of the classifier. Furthermore, the analysis as illustrated in FIG. 1 can be applied before or during a procedure included in FIG. 2, for example early on in the process before an invasive workup. With the current pancreatic cancer patient journey, an opportunity lies in screening high-risk patients before biopsy or pancreatoscopy. For example, a primary opportunity for using the methods described herein includes screening high risk patients for early detection with improved accuracy and convenience. Another opportunity may lie in improved decision making for an imaging or biopsy procedure.

In some aspects, the cancer to be detected by the methods described herein can be pancreatic cancer. The pancreatic cancer may be early-stage pancreatic cancer. In other aspects, the pancreatic cancer may be late-stage pancreatic cancer. Non-invasively obtained samples can be used for cancer diagnosis by generating data and identifying patterns in the data that associate with the cancer such as pancreatic cancer. Diagnosis of cancer may be improved by obtaining proteomic data. Diagnosis of cancer may be improved by combining multiple types of data (e.g., multiple data sets) into the analysis. For example, combining multiple data types comprising proteomic, transcriptomic, genomic, metabolomic, or a combination thereof may improve the accuracy of prediction of whether a subject has the cancer. In some aspects, the methods described herein include generating or obtaining data and using the data to predict whether a subject has or does not have a cancer. Various ways of combining or analyzing the data are described, and the uses of the data for cancer assessment are further elaborated.

In certain aspects, the method of detecting a cancer may comprise additional screening or diagnosing methods such as a computed tomography (CT) scan indicative of pancreatic cancer, a magnetic resonance imaging (MRI) scan indicative of pancreatic cancer, a positron emission tomography (PET) scan indicative of pancreatic cancer, an ultrasound indicative of pancreatic cancer, a cholangiopancreatography indicative of pancreatic cancer, an angiography indicative of pancreatic cancer, a liver function test (LFT) indicative of pancreatic cancer, an elevated carcinoembryonic antigen (CEA) level relative to a control or baseline measurement, an elevated carbohydrate antigen (CA) 19-9 level relative to a control or baseline measurement, or a combination thereof. In some aspects, the method of detecting pancreatic cancer may comprise identifying a symptom of a subject such as jaundice, abdominal pain, gallbladder or liver enlargement, a blood clot, digestion problems, or depression, or a combination thereof.

A classification method may include any biomarker such as AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. In some embodiments, the biomarkers comprises two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or each of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. Any of said biomarkers may be useful in a classifier for identifying the presence of pancreatic cancer, ruling out pancreatic cancer, or for distinguishing between pancreatic cancer and a lack of pancreatic cancer in a biofluid sample from a subject suspected of having pancreatic cancer.

### Subjects and Samples

The methods described herein may be used to identify a subject as likely or at risk to have a cancer such as pancreatic cancer. The cancer may include adenocarcinoma, for example pancreatic adenocarcinoma. The subject may be a vertebrate. The subject may be a mammal. The subject may be a human. The subject may be male or female. The subject may have the cancer. The subject may not have the cancer. The subject may have the pancreatic cancer. The subject may not have the pancreatic cancer. The subject may be at risk of having pancreatic cancer. For example, the subject may have a mass (e.g., nodule or cyst) in the pancreas.

A sample may be obtained from the subject for purposes of identifying a cancer in the subject. The subject may be suspected of having the cancer or as not having the cancer. The method may be used to confirm or refute the suspected cancer.

The subject may be subjected of having pancreatic cancer. The subject may have pancreatic cancer. The cancer may include pancreatic cancer. The pancreatic cancer may include early-stage pancreatic cancer. The pancreatic cancer may include late-stage pancreatic cancer. The pancreatic cancer may be stage 1 pancreatic cancer. The pancreatic cancer may be stage 2 pancreatic cancer. The pancreatic cancer may be stage 1 or 2. The pancreatic cancer may be stage 3 pancreatic cancer. The pancreatic cancer may be stage 4 pancreatic cancer. The pancreatic cancer may be stage 3 or 4. The pancreatic cancer may be stage 1, 2, 3, or 4. The pancreatic cancer may include pancreatic ductal adenocarcinoma (PDAC).

Data described herein may be generated from a sample of a subject. The sample may be a biofluid sample or a mass sample (e.g., an abnormal growth biopsied from the subject). Examples of biofluids include blood, serum, or plasma. The sample may include a blood sample. The sample may include a serum sample. The sample may include a plasma sample. Other examples of biofluids include urine, tears, semen, milk, vaginal fluid, mucus, saliva, or sweat.

A biofluid sample may be obtained from a subject. For example, a blood, serum, or plasma sample may be obtained from a subject by a blood draw. Other ways of obtaining biofluid samples include aspiration or swabbing.

The biofluid sample may be cell-free or substantially cell-free. To obtain a cell-free or substantially cell-free biofluid sample, a biofluid may undergo a sample preparation method such as centrifugation and pellet removal.

A non-biofluid sample may be obtained from a patient. A sample may include a tissue sample. The tissue sample may include a pancreatic tissue sample. For example, the sample may include a mass taken from the pancreas of a subject, which is suspected of being cancerous. The mass may include a pancreatic cyst. The cyst may be identified by a physician as a high or low-risk cyst prior to performing the methods described herein. The mass may be examined under a microscope. The sample may include a cell sample. The sample may include a homogenate of a cell or tissue. The sample may include a supernatant of a centrifuged homogenate of a cell or tissue.

The sample (e.g., biofluid or tissue sample) can be obtained from the subject during any phase of the screening procedures for diagnosing for the cancer. For example, the biofluid sample can be obtained before, during, or after any one of the procedures described here in FIG. 2. The biofluid sample can be obtained before or during a stage where the subject is a candidate for biopsy or pancreatoscopy, for early detection of the cancer. In other aspects, the biofluid sample can be obtained before or during a non-invasive work-up, an invasive work-up, treatment, a monitoring stage.

### Data Generation

### Proteomic Data

The data described herein may include protein data or proteomic data. The methods disclosed herein may include obtaining data generated from one or more samples such as a biofluid sample collected from the subject. The data may include biomolecule measurements such as protein measurements, transcript measurements, genetic material measurements, or metabolite measurements. Data may include any of the following types of omic data: proteomic data, genomic data, transcriptomic data, or metabolomic data. This section includes some ways of generating each of these types of omic data. Methods of generating or analyzing omic data may also be applied to methods of generating or analyzing individual biomolecules or sets of biomolecules. Other types of data may also be generated. The data may be labeled or identified as indicative of pancreatic cancer or as not indicative of pancreatic cancer.

Proteomic data may involve data about proteins, peptides, or proteoforms. Proteomic data may include just peptides or proteins, or a combination of both. An example of a peptide is an amino acid chain. An example of a protein is a peptide or a combination of peptides. For example, a protein may include one, two or more peptides bound together. A protein may also include any post-translational modifications. A protein may be a secreted protein. Proteomic data may include data about various proteoforms. Proteoforms can include different forms of a protein produced from a genome with any variety of sequence variations, splice isoforms, or post-translational modifications.

Proteomic data may include information on the presence, absence, or amount of various proteins, peptides. For example, proteomic data may include amounts of proteins. A protein amount may be indicated as a concentration or quantity of proteins, for example a concentration of a protein in a biofluid. A protein amount may be relative to another protein or to another biomolecule. Proteomic data may include information on the presence of proteins or peptides. Proteomic data may include information on the absence of proteins or peptides. Proteomic data may be distinguished by subtype, where each subtype includes a different type of protein, peptide, or proteoform.

Proteomic data generally includes data on a number of proteins or peptides. For example, proteomic data may include information on the presence, absence, or amount of 1000 or more proteins or peptides. In some cases, proteomic data may include information on the presence, absence, or amount of 5000, 10,000, 20,000, or more peptides, proteins, or proteoforms. Proteomic data may even include up to about 1 million proteoforms. Proteomic data may include a range of proteins, peptides, or proteoforms defined by any of the aforementioned numbers of proteins, peptides, or proteoforms.

Proteomic data may be generated by any of a variety of methods. Generating proteomic data may include using a detection reagent that binds to a peptide or protein and yields a detectable signal. After use of a detection reagent that binds to a peptide or protein and yields a detectable signal, a readout may be obtained that is indicative of the presence, absence or amount of the protein or peptide. Generating proteomic data may include concentrating, filtering, or centrifuging a sample.

Proteomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. Some examples of methods for generating proteomic data include using mass spectrometry, a protein chip, or a reverse-phased protein microarray. Proteomic data may also be generated using an immunoassay such as an enzyme-linked immunosorbent assay, western blot, dot blot, or immunohistochemistry assay. Generating proteomic data may involve use of an immunoassay panel.

One way of obtaining proteomic data includes use of mass spectrometry. An example of a mass spectrometry method includes use of high resolution, two-dimensional electrophoresis to separate proteins from different samples in parallel, followed by selection or staining of differentially expressed proteins to be identified by mass spectrometry. Another method uses stable isotope tags to differentially label proteins from two different complex mixtures. The proteins within a complex mixture may be labeled isotopically and then digested to yield labeled peptides. Then the labeled mixtures may be combined, and the peptides may be separated by multidimensional liquid chromatography and analyzed by tandem mass spectrometry. A mass spectrometry method may include use of liquid chromatography-mass spectrometry (LC-MS), a technique that may combine physical separation capabilities of liquid chromatography (e.g., HPLC) with mass spectrometry.

In addition to any of the above methods, generating proteomic data may include contacting a sample with particles such that the particles adsorb biomolecules comprising proteins. The adsorbed proteins may be part of a biomolecule corona. The adsorbed proteins may be measured or identified in generating the proteomic data.

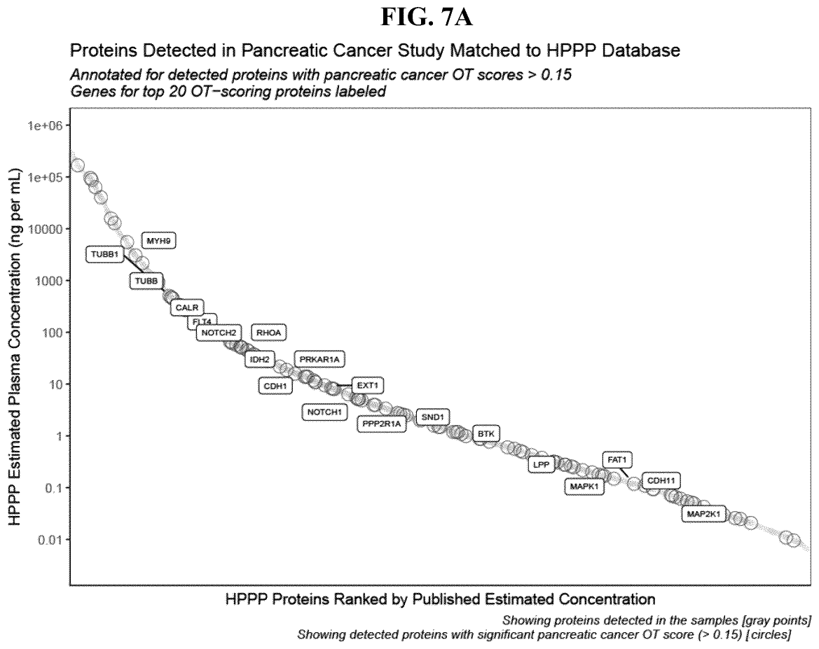

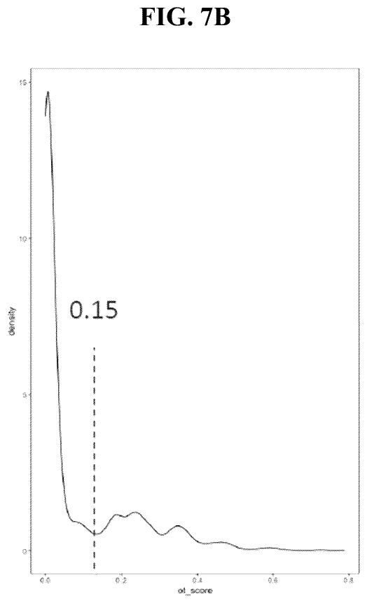

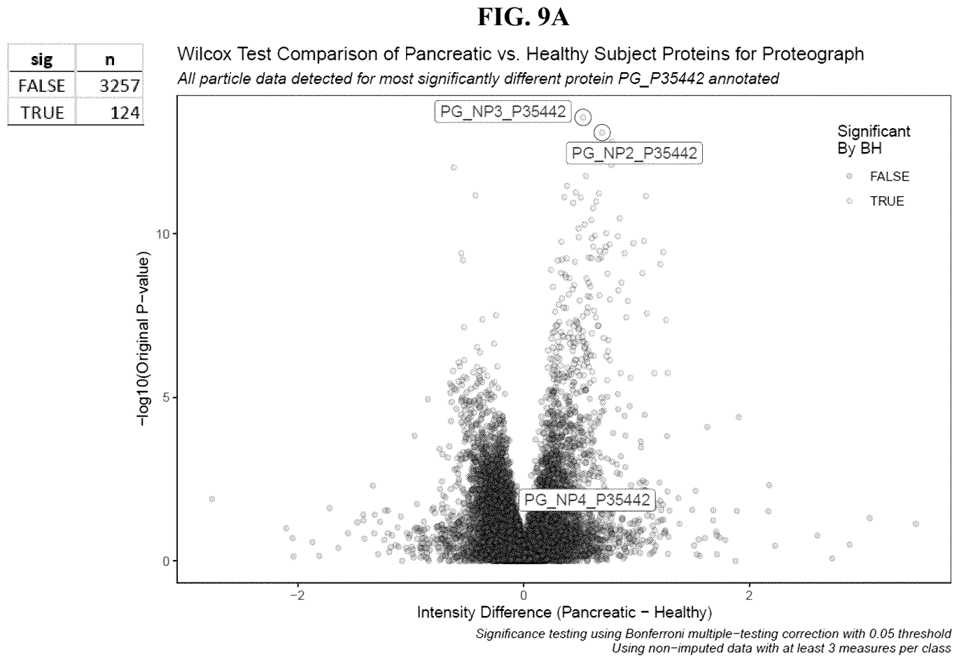

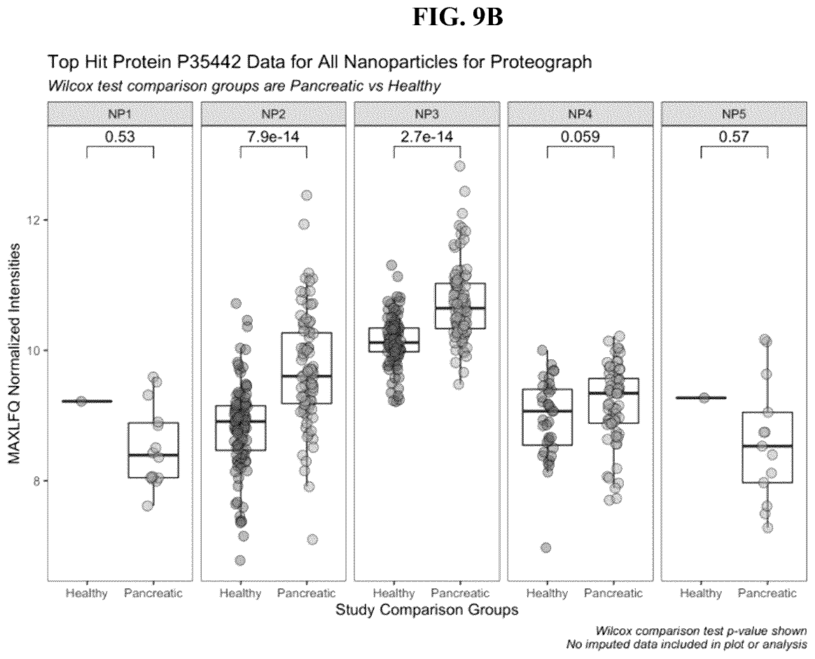

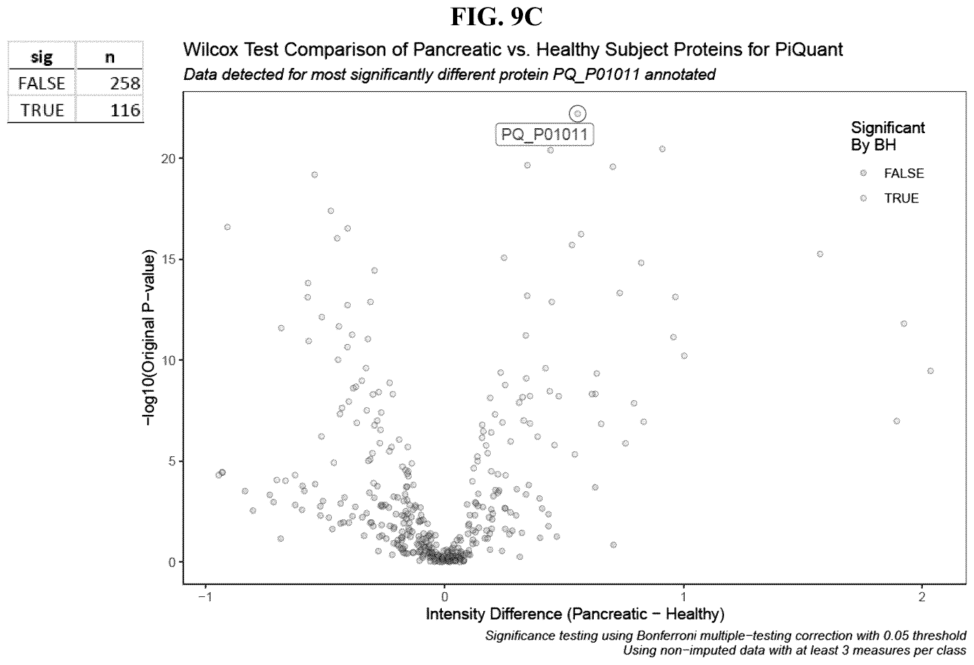

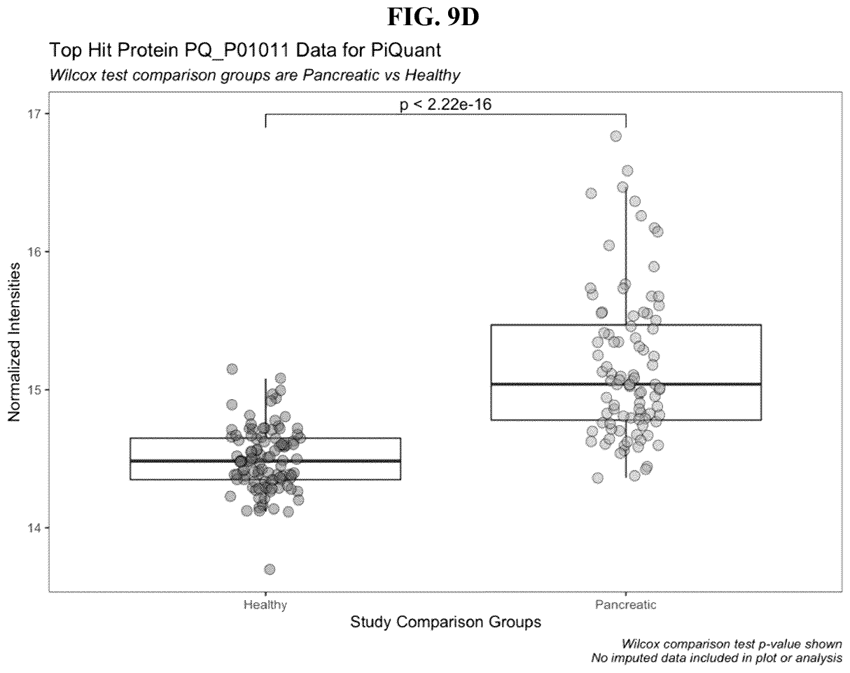

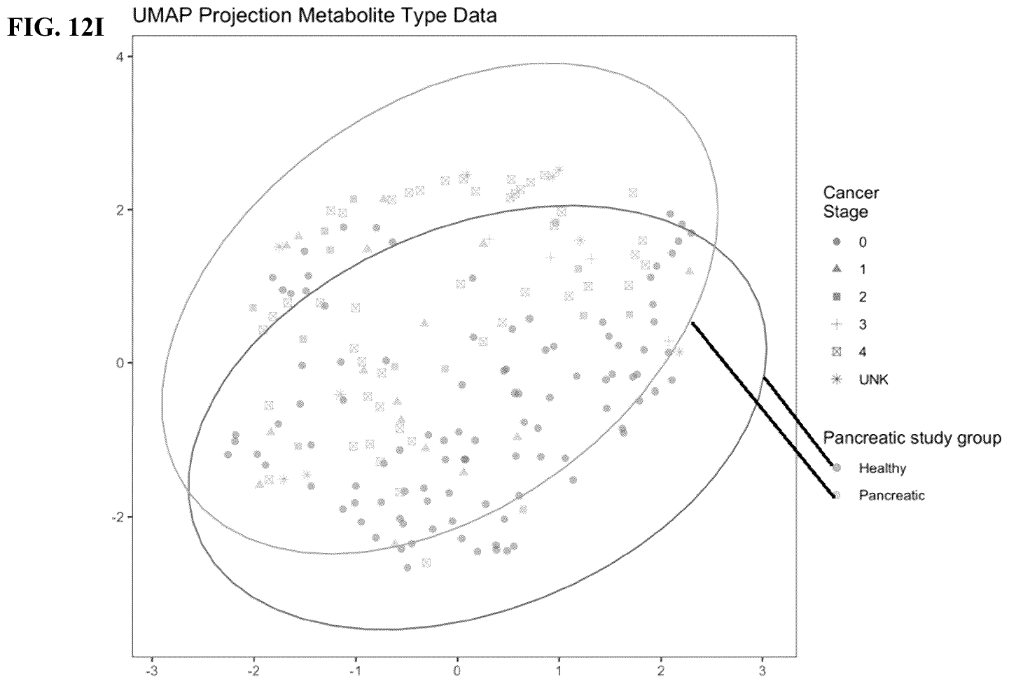

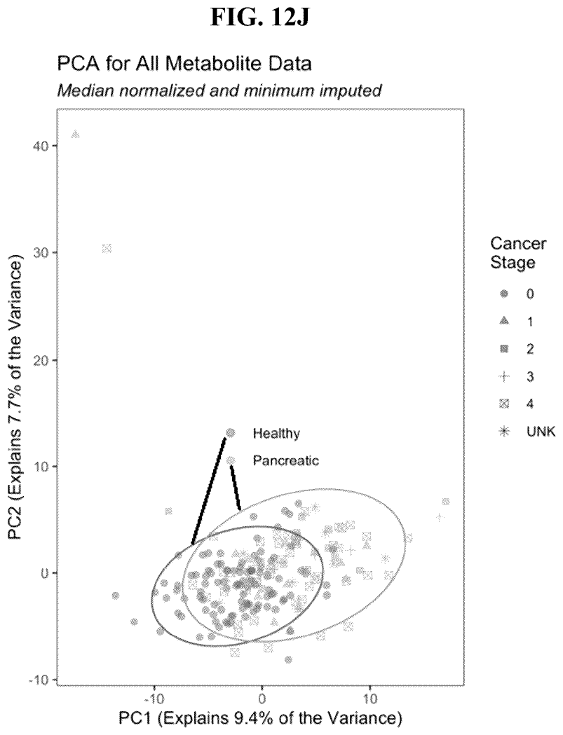

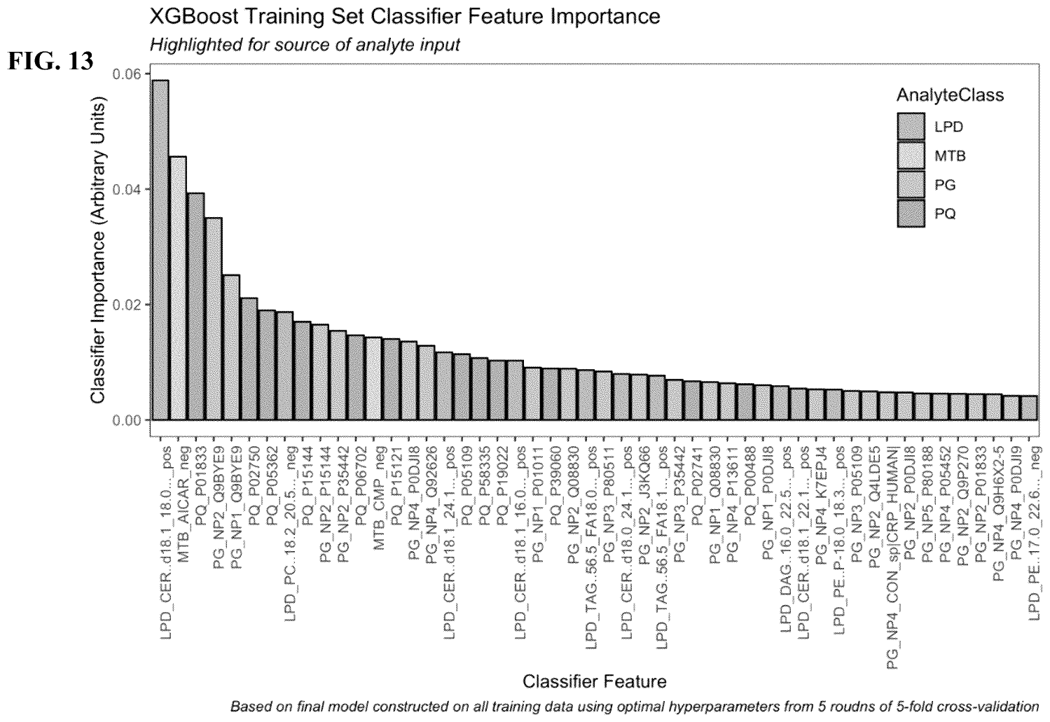

Some examples of proteins are shown in FIG. 7A. Proteins that may be detected in a method described herein include Myosin-9 (MYH9), Tubulin beta-1 chain (TUBB1), Tubulin beta chain (TUBB), Calreticulin (CALR), Vascular endothelial growth factor receptor 3 (FLT4), Neurogenic locus notch homolog protein 2 (NOTCH2), Transforming protein RhoA (RHOA), Isocitrate dehydrogenase [NADP], mitochondrial (IDH2), Cadherin-1 (CDH1), cAMP-dependent protein kinase type I-alpha regulatory subunit (PRKAR1A), Neurogenic locus notch homolog protein 1 (NOTCH1), Exostosin-1 (EXT1), Serine/threonine-protein phosphatase 2A 65 kDa regulatory subunit A alpha isoform (PPP2R1A), Staphylococcal nuclease domain-containing protein 1 (SND1), Tyrosine-protein kinase BTK (BTK), Lipoma-preferred partner (LPP), Mitogen-activated protein kinase (MAPK1), Fat1 protein (FAT1), Cadherin-11 (CDH11), or Dual specificity mitogen-activated protein kinase 1 (MAP2K1). Another example of a protein is shown in FIG. 9A-9B. A protein to be detected in a method described herein may include Thrombospondin-2 (TSP2 or P35442). Another example of a protein is shown in FIG. 9C-9D. A protein to be detected in a method described herein may include P01011. Some examples of proteins are shown in FIG. 13. A protein to be detected in a method described herein may include Polymeric immunoglobulin receptor (PIGR, UniProt P01833), Cadherin-related family member 2 (CDHR2, UniProt Q9BYE9), Leucine-rich alpha-2-glycoprotein (LRG1 or A2GL, UniProt P02750), Intercellular adhesion molecule 1 (ICAM1, UniProt P05362), Aminopeptidase N (AMPN or ANPEP, UniProt P15144), Thrombospondin-2 (TSP2, UniProt P35442), Protein S100-A9 (S10A9 or S100A9, UniProt P06702), Aldo-keto reductase family 1 member B1 (ALDR or AKR1B1, UniProt P15121), Serum amyloid A-1 protein (SAA1, UniProt P0DJI8), Peroxidasin homolog (PXDN, UniProt Q92626), Protein S100-A8 (S10A8 or S100A8, UniProt P05109), Anthrax toxin receptor 2 (ANTR2 or ANTXR2, UniProt P58335), Cadherin-2 (CADH2 or CDH2, UniProt P19022), Alpha-1-antichymotrypsin (AACT or SERPINA3, UniProt P01011), Collagen alpha-1(XVIII) chain (COIA1 or COL18A1, UniProt P39060), Fibrinogen-like protein 1 (FGL1, UniProt Q08830), Protein S100-A12 (S10AC or S100A12, UniProt P80511), Reelin (RELN, UniProt J3KQ66), C-reactive protein (CRP, UniProt P02741), Versican core protein (CSPG2 or VCAN, UniProt P13611), Coagulation factor XIII A chain (F13A or F13A1, UniProt P00488), Cartilage intermediate layer protein 2 (CILP2, UniProt K7EPJ4), Sushi, von Willebrand factor type A, EGF and pentraxin domain-containing protein 1 (SVEP1, UniProt Q4LDE5), Neutrophil gelatinase-associated lipocalin (NGAL or LCN2, UniProt P80188), Tetranectin (TETN or CLEC3B, UniProt P05452), SLAIN motif-containing protein 2 (SLAI2 or SLAIN2, UniProt Q9P270), Anthrax toxin receptor 1 (ANTR1 or ANTXR1, UniProt Q9H6X2, e.g. isoform 5 [UniProt Q9H6X2-5]), or Serum amyloid A-2 protein (SAA2, UniProt P0DJI9). Any number of the aforementioned proteins may be used. Any of the proteins may be used in a classifier.

A method may include measuring biomarkers in a biofluid sample, wherein the biomarkers comprise A2GL, AKR1B1, ANPEP, ANTXR1, ANTXR2, BTK, CALR, CDH1, CDH11, CDH2, CDHR2, CILP2, CLEC3B, COL18A1, CRP, EXT1, F13A1, FAT1, FGL1, FLT4, ICAM1, IDH2, LCN2, LPP, MAPK1, MAP2K1, MYH9, NOTCH1, NOTCH2, PIGR, PPP2R1A, PRKAR1A, PXDN, RELN, RHOA, S100A8, S100A9, S100A12, SAA1, SAA2, SERPINA3, SLAIN2, SND1, SVEP1, TSP2, TUBB, TUBB1, or VCAN. In some aspects, the biomarkers comprise 1, 2, 3, 4, 5, 6, 7, 8, 9, 10, 11, 12, 13, 14, 15, 20, 25, 30, 35, 40, 45, or 48 of the aforementioned biomarkers, or a range of biomarkers defined by any two of the aforementioned integers.

Any biomarker in Table 5 may be used in a method described herein, for example in a pancreatic cancer evaluation method. Some such examples may include: Alpha-1-antichymotrypsin, Leucine-rich alpha-2-glycoprotein, Alpha-1-antitrypsin, Aminopeptidase N, Lipopolysaccharide-binding protein, Intercellular adhesion molecule 1, Polymeric immunoglobulin receptor, Protein S100-A8, Complement C2, Complement C5, Complement C9, Inter-alpha-trypsin inhibitor heavy chain H3, Retinol-binding protein 4, Low affinity immunoglobulin gamma Fc region receptor III-A, C-reactive protein, Tetranectin, Noelin, Coagulation factor XIII B chain, Apolipoprotein A-II, or Apolipoprotein A-I. A biomarker may include Alpha-1-antitrypsin. A biomarker may include Alpha-1-antichymotrypsin. A biomarker may include Polymeric immunoglobulin receptor. A biomarker may include C-reactive protein. A biomarker may include Leucine-rich alpha-2-glycoprotein. A biomarker may include Complement C2. A biomarker may include Serum amyloid A-1 protein. A biomarker may include Serum amyloid A-2 protein. A biomarker may include Inter-alpha-trypsin inhibitor heavy chain H3. A biomarker may include Peptidase inhibitor 16. Any number or combination of these biomarkers may be used. For example, 1, 2, 3, 4, 5, 6, 7, 8, 9, 10, 11, 12, 13, 14, 15, 16, 17, 18, 19, or 20, or a range defined by any two of the aforementioned integers, may be included as biomarkers in methods described herein such as in a cancer evaluation method. Any of these biomarkers may be used as features in a classifier, such as for classifying a biofluid sample as indicative of a cancer such as pancreatic cancer or not, or for ruling out the presence of the cancer. Any of these biomarkers may be measured in conjunction with an internal reference standard such as a labeled version of the biomarker. Any of these biomarkers may be combined with other biomarker or biomarkers such as any described herein.

In some cases, a cancer evaluation method includes the use of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19, of the following biomarkers from Table 5: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. In some cases, the biomarkers comprises two or more, three or more, four or more, five or more, six or more, seven or more, eight or more, nine or more, 10 or more, 11 or more, 12 or more, 13 or more, 14 or more, 15 or more, 16 or more, 17 or more, 18 or more, or 19 or each of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. In some cases, all of the following biomarkers are included: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. In some cases, a cancer evaluation method includes the use of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, or no more than 20, of the following biomarkers: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. Some methods use a subgroup of said biomarkers. For example, the subgroup may exclude A1AT. The subgroup may exclude A2G. The subgroup may exclude AACT. The subgroup may exclude AMPN. The subgroup may exclude APOA1. The subgroup may exclude APOA2. The subgroup may exclude CO2. The subgroup may exclude CO5. The subgroup may exclude CO9. The subgroup may exclude CRP. The subgroup may exclude F13B. The subgroup may exclude FCG3A. The subgroup may exclude ICAM1. The subgroup may exclude ITIH3. The subgroup may exclude LBP. The subgroup may exclude NOE1. The subgroup may exclude PIGR. The subgroup may exclude RET4. The subgroup may exclude S10A8. The subgroup may exclude TETN. Any number or combination of the following biomarkers may be used: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. Any number or combination of the following biomarkers may be used: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1. In some cases, all of the following biomarkers are included: AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1.

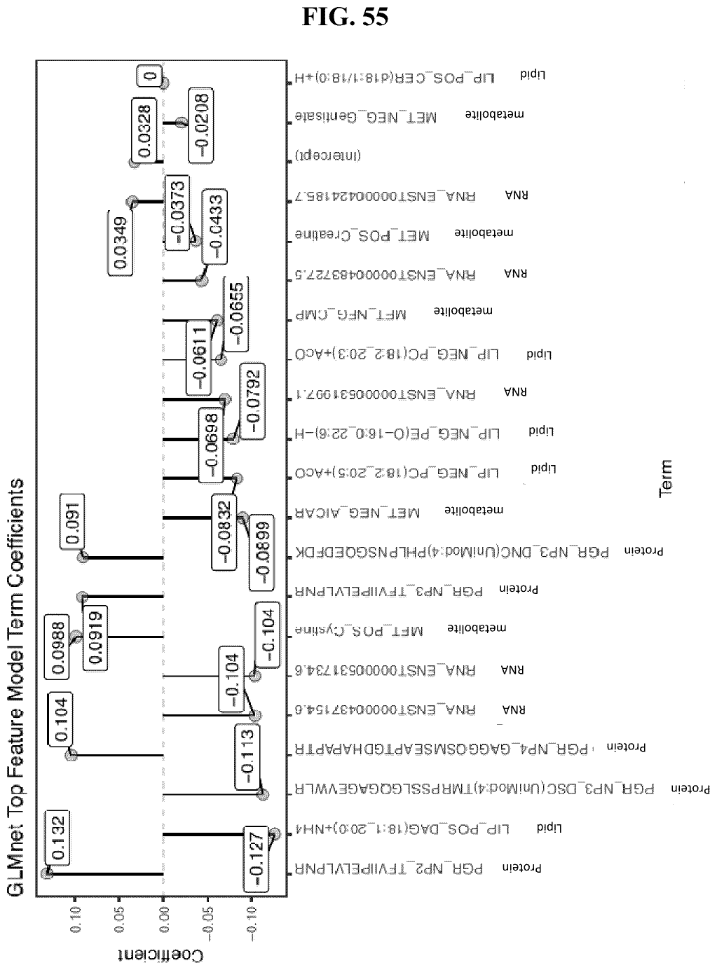

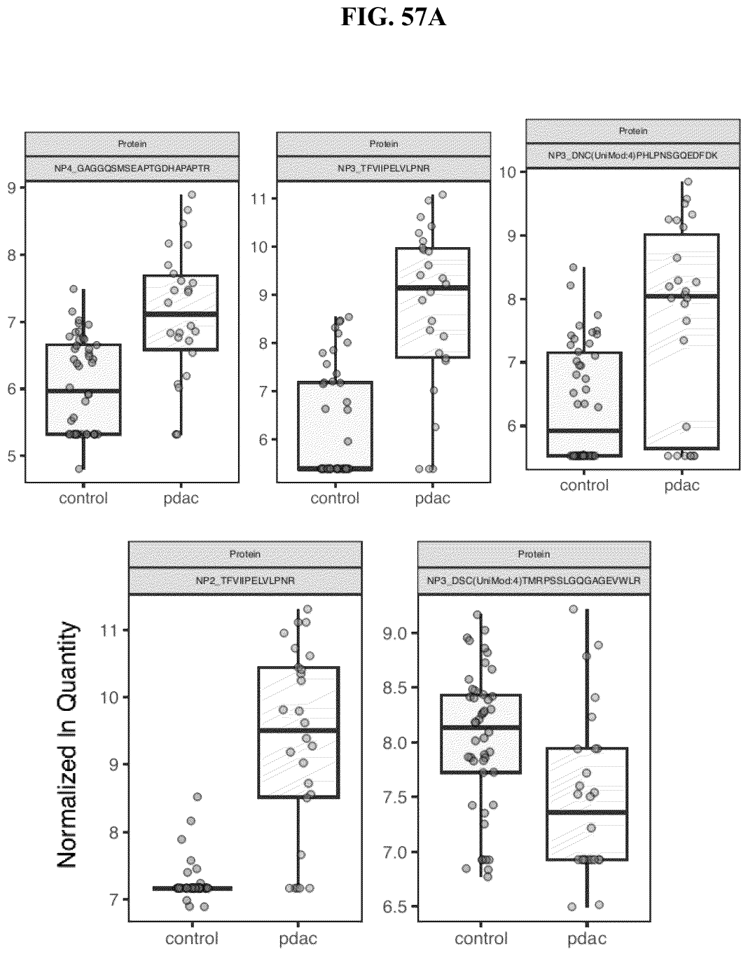

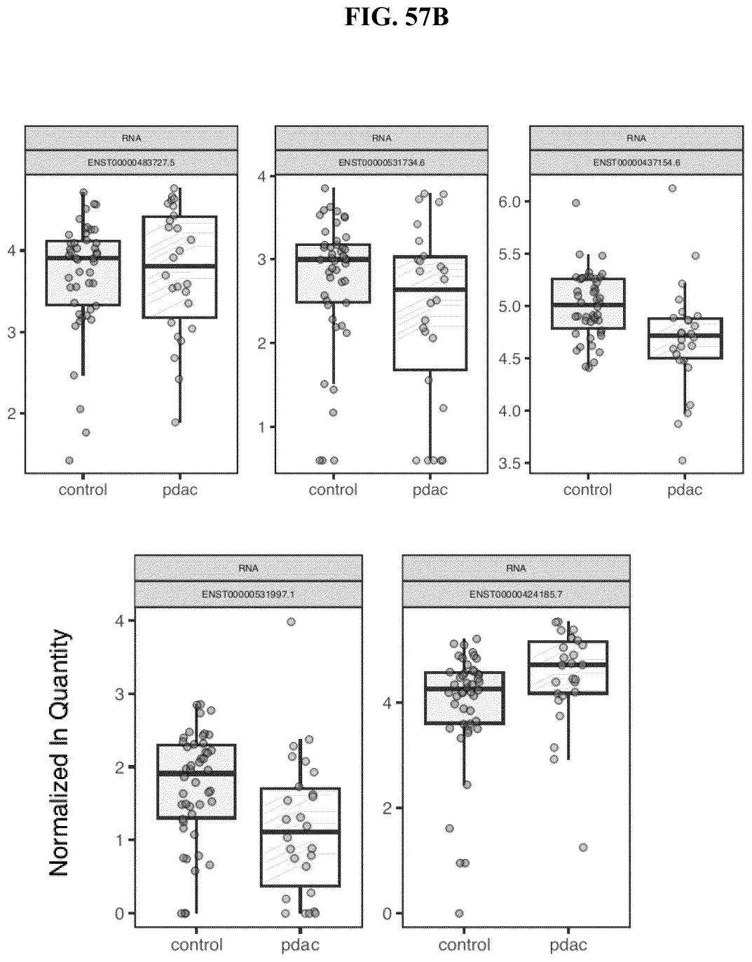

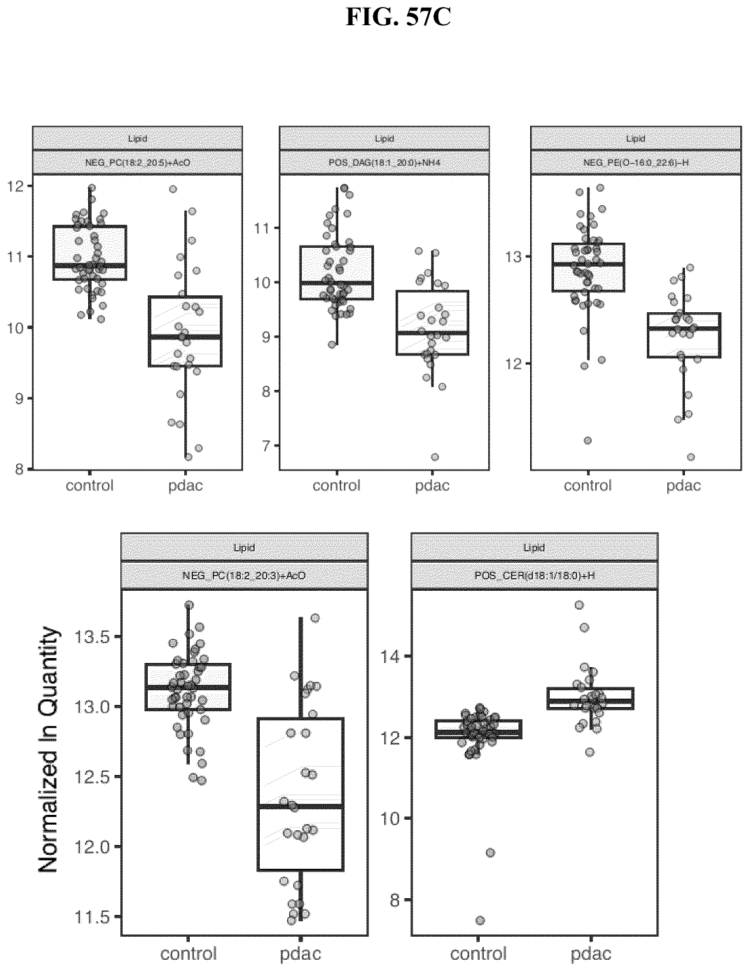

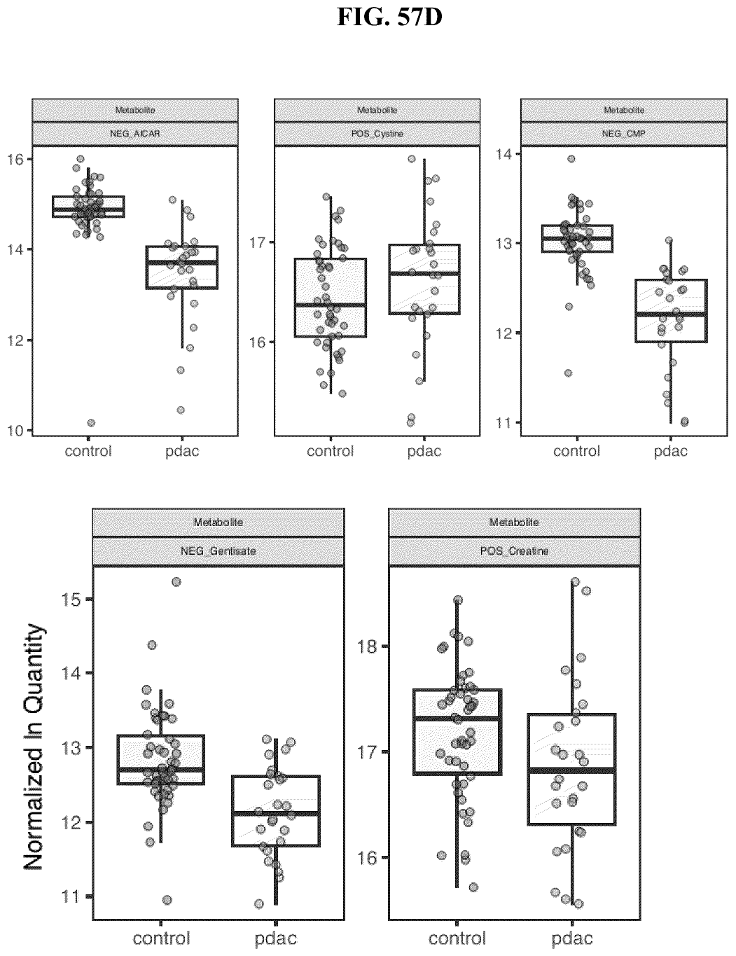

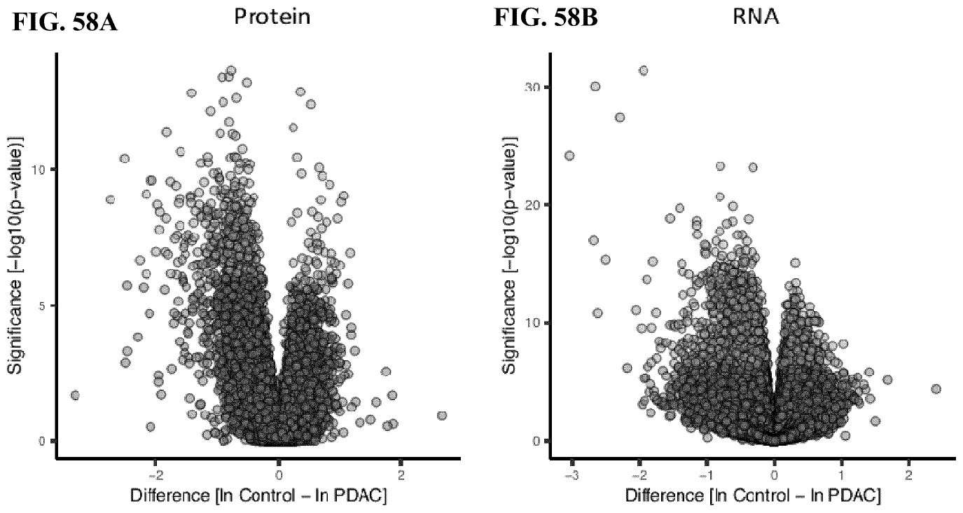

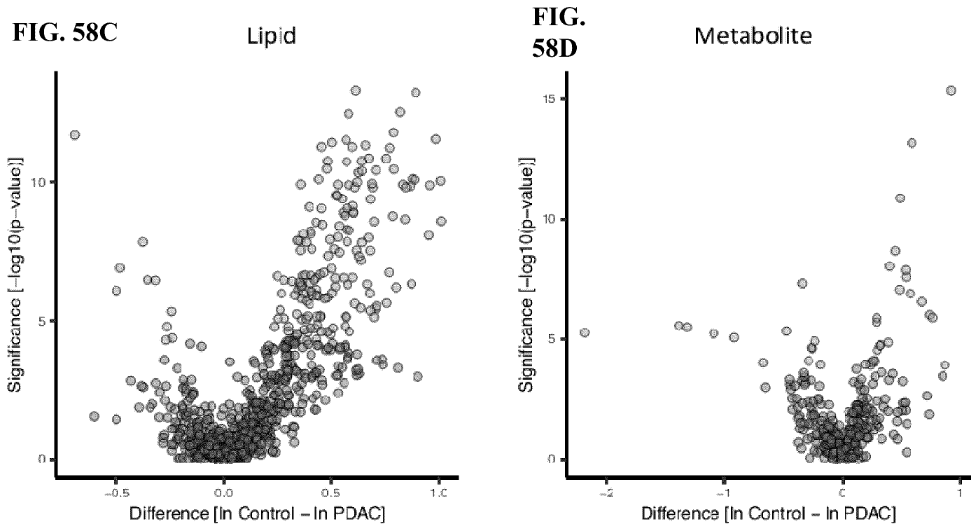

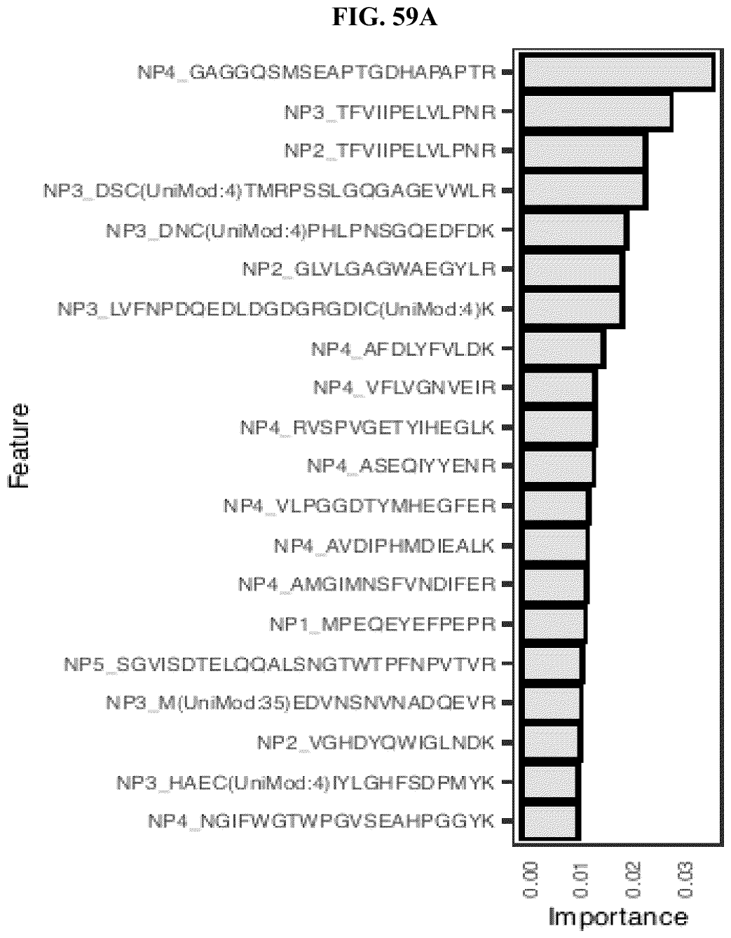

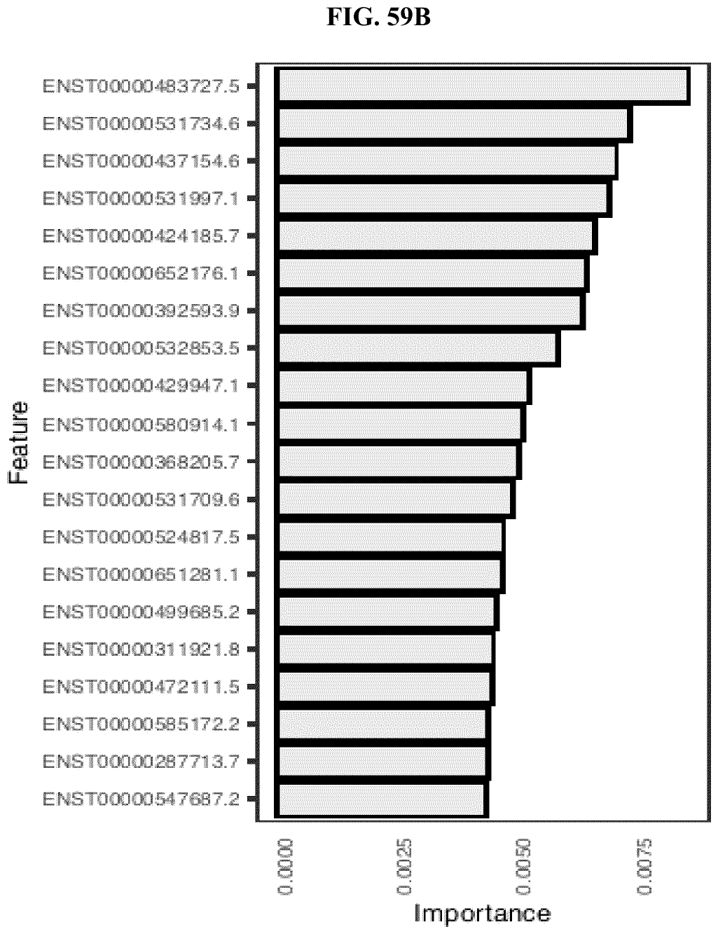

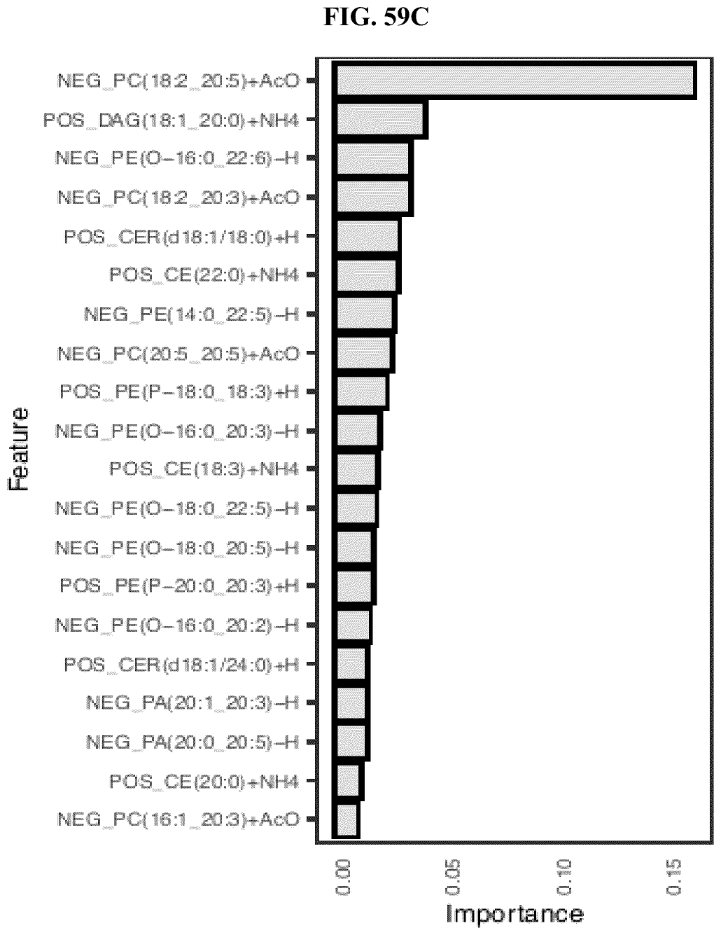

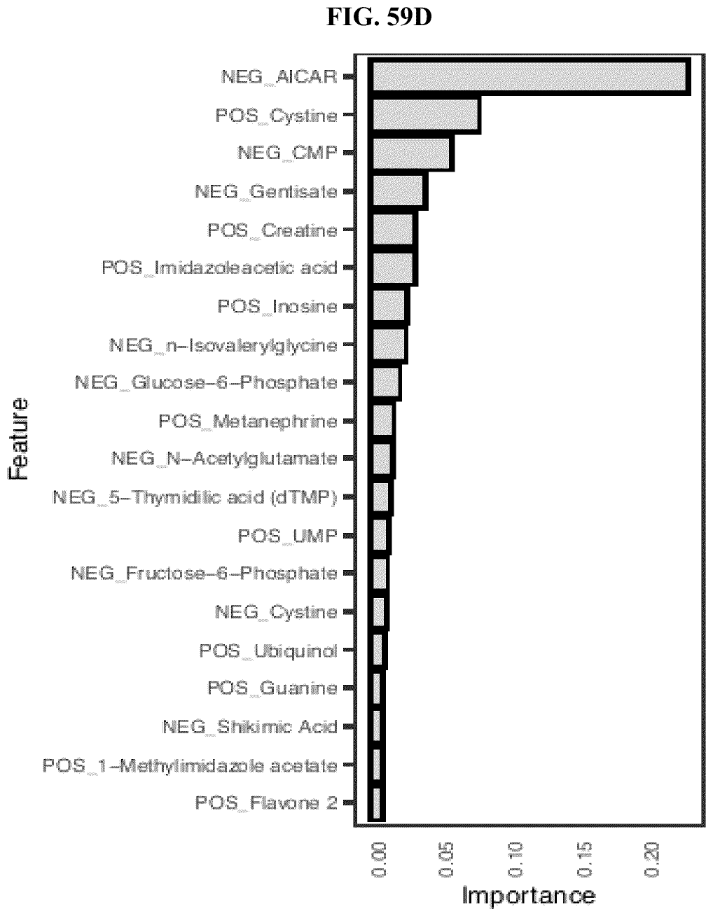

Examples of protein or peptide biomarkers that may be useful in the methods described herein may include a biomarker in FIG. 55. Examples of protein or peptide biomarkers that may be useful in the methods described herein may include a biomarker in FIG. 57A. Examples of protein or peptide biomarkers that may be useful in the methods described herein may include a biomarker in FIG. 59A. Any combination or number of the biomarkers in FIG. 55, FIG. 57A, or FIG. 59A may be useful. For example, any of the biomarkers in FIG. 55, FIG. 57A, or FIG. 59A may be useful in distinguishing between biofluid samples of subjects with and without a cancer such as pancreatic cancer. In some cases, a cancer evaluation method includes the use of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 of the biomarkers from Table 19. In some cases, any of the biomarkers may be chosen from GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), DNC(UniMod:4)PHLPNSGQEDFDK (SEQ ID NO. 4), GLVLGAGWAEGYLR (SEQ ID NO. 5), LVFNPDQEDLDGDGRGDIC(UniMod:4)K (SEQ ID NO. 6), AFDLYFVLDK (SEQ ID NO. 7), VFLVGNVEIR (SEQ ID NO. 8), RVSPVGETYIHEGLK (SEQ ID NO. 9), ASEQIYYENR (SEQ ID NO. 10), VLPGGDTYMHEGFER (SEQ ID NO. 11), AVDIPHMDIEALK (SEQ ID NO. 12), AMGIMNSFVNDIFER (SEQ ID NO. 13), MPEQEYEFPEPR (SEQ ID NO. 14), SGVISDTELQQALSNGTWTPFNPVTVR (SEQ ID NO. 15), M(UniMod:35)EDVNSNVNADQEVR (SEQ ID NO. 16), VGHDYQWIGLNDK (SEQ ID NO. 17), HAEC(UniMod:4)IYLGHFSDPMYK (SEQ ID NO. 18), or NGIFWGTWPGVSEAHPGGYK (SEQ ID NO. 19). UniMod:4 represents an amino acid modified with an iodoacetamide derivative while UniMod:35 represents an amino acid modified with a methionine sulfoxide. Some methods use a subgroup of said biomarkers. In some cases, the biomarkers may contain a first TFVIIPELVLPNR (SEQ ID NO. 2) and a second TFVIIPELVLPNR (SEQ ID NO. 2), each of which is detected by means of a different particle. For example, in some cases, the first TFVIIPELVLPNR (SEQ ID NO. 2) may be detected on a first nanoparticle (NP1) and the second TFVIIPELVLPNR (SEQ ID NO. 2) may be detected on a second nanoparticle (NP2). In some cases, the biomarkers may include GAGGQSMSEAPTGDHAPAPTR (SEQ ID NO. 1), TFVIIPELVLPNR (SEQ ID NO. 2), TFVIIPELVLPNR (SEQ ID NO. 2), DSC(UniMod:4)TMRPSSLGQGAGEVWLR (SEQ ID NO. 3), or DNC(UniMod:4)PHLPNSGQEDFDK (SEQ ID NO. 4) or a combination thereof. In some cases, any of the biomarkers may be chosen from LTBP2, CSHR2, FGFBP2, or THBS2. In some cases, a biomarker may include LTBP2. In some cases, a biomarker may include CSHR2. In some cases, a biomarker may include FGFBP2. In some cases, a biomarker may include THBS2. In some cases, any of the biomarkers may be chosen from Q14767-LTBP2, Q9BYE9-CDHR2, Q9BYJ0-FGFBP2, or P35442-TSP2. In some cases, a biomarker may include Q14767-LTBP2. In some cases, a biomarker may include Q9BYE9-CDHR2. In some cases, a biomarker may include Q9BYJ0-FGFBP2. In some cases, a biomarker may include P35442-TSP2. In some cases, the biomarkers may contain GAGGQSMSEAPTGDHAPAPTR (SEQ ID NO. 1). In some cases, the biomarkers may be an increase in GAGGQSMSEAPTGDHAPAPTR (SEQ ID NO. 1). In some cases, the biomarkers may contain TFVIIPELVLPNR (SEQ ID NO. 2). In some cases, the biomarkers may be an increase in TFVIIPELVLPNR (SEQ ID NO. 2). In some cases, the biomarkers may contain a second TFVIIPELVLPNR (SEQ ID NO. 2). The second TFVIIPELVLPNR (SEQ ID NO. 2) may detected by means of a different particle. In some cases, the biomarkers may contain DSCTMRPSSLGQGAGEVWLR (SEQ ID NO. 20). In some cases, the DSCTMRPSSLGQGAGEVWLR (SEQ ID NO. 20) may be modified with an iodoacetamide derivative. The iodoacetamide derivative may be attached to the cysteine. In some cases, the biomarkers may be a decrease in DSCTMRPSSLGQGAGEVWLR (SEQ ID NO. 20). In some cases, the biomarkers may contain DNCPHLPNSGQEDFDK (SEQ ID NO. 21). In some cases, the biomarkers may be an increase DNCPHLPNSGQEDFDK (SEQ ID NO. 21). The DNCPHLPNSGQEDFDK (SEQ ID NO. 21) may be modified with an iodoacetamide derivative. The iodoacetamide derivative may be attached to the cysteine.

Any combination or number of the protein or peptide biomarkers in this section or described herein may be useful. For example, some of the biomarkers may be useful in distinguishing between biofluid samples of subjects with and without a cancer.

### Transcriptomic Data

The data described herein may include transcript data or transcriptomic data. Transcriptomic data may involve data about nucleotide transcripts such as RNA. Examples of RNA include messenger RNA (mRNA), ribosomal RNA (rRNA), signal recognition particle (SRP) RNA, transfer RNA (tRNA), small nuclear RNA (snRNA), small nucleoar RNA (snoRNA), long noncoding RNA (lncRNA), microRNA (miRNA), noncoding RNA (ncRNA), or piwi-interacting RNA (piRNA). The RNA may include mRNA. The RNA may include miRNA. Transcriptomic data may be distinguished by subtype, where each subtype includes a different type of RNA or transcript. For example, mRNA data may be included in one subtype, and miRNA data may be included in another subtype.

Transcriptomic data may include information on the presence, absence, or amount of various RNAs. For example, transcriptomic data may include amounts of RNAs. An RNA amount may be indicated as a concentration or number or RNA molecules, for example a concentration of an RNA in a biofluid. An RNA amount may be relative to another RNA or to another biomolecule. Transcriptomic data may include information on the presence of RNAs. Transcriptomic data may include information on the absence of RNA.

Transcriptomic data generally includes data on a number of RNAs. For example, transcriptomic data may include information on the presence, absence, or amount of 1000 or more RNAs. In some cases, transcriptomic data may include information on the presence, absence, or amount of 5000, 10,000, 20,000, or more RNAs. Transcriptomic data may even include up to about 200,000 transcripts. Transcriptomic data may include a range of transcripts defined by any of the aforementioned numbers of RNAs or transcripts.

Transcriptomic data may be generated by any of a variety of methods. Generating transcriptomic data may include using a detection reagent that binds to an RNA and yields a detectable signal. After use of a detection reagent that binds to an RNA and yields a detectable signal, a readout may be obtained that is indicative of the presence, absence or amount of the RNA. Generating transcriptomic data may include concentrating, filtering, or centrifuging a sample.

Transcriptomic data may include RNA sequence data. Some examples of methods for generating RNA sequence data include use of sequencing, microarray analysis, hybridization, polymerase chain reaction (PCR), or electrophoresis, or a combination thereof. A microarray may be used for generating transcriptomic data. PCR may be used for generating transcriptomic data. PCR may include quantitative PCR (qPCR). Such methods may include use of a detectable probe (e.g. a fluorescent probe) that intercalates with double-stranded nucleotides, or that binds to a target nucleotide sequence. PCR may include reverse transcriptase quantitative PCR (RT-qPCR). Generating transcriptomic data may involve use of a PCR panel.

RNA sequence data may be generated by sequencing a subject's RNA or by converting the subject's RNA into DNA (e.g., complementary DNA (cDNA)) first and sequencing the DNA. Sequencing may include massive parallel sequencing. Examples of massive parallel sequencing techniques include pyrosequencing, sequencing by reversible terminator chemistry, sequencing-by-ligation mediated by ligase enzymes, or phospholinked fluorescent nucleotides or real-time sequencing. Generating transcriptomic data may include preparing a sample or template for sequencing. A reverse transcriptase may be used to convert RNA into cDNA. Some template preparation methods include use of amplified templates originating from single RNA or cDNA molecules, or single RNA or cDNA molecule templates. Examples of amplification methods include emulsion PCR, rolling circle, or solid-phase amplification

In addition to any of the above methods, generating transcriptomic data may include contacting a sample with particles such that the particles adsorb biomolecules comprising RNA. The adsorbed RNA may be part of a biomolecule corona. The adsorbed RNA may be measured or identified in generating the transcriptomic data.

In some methods the RNA biomarkers that may be useful in the methods described herein may include a biomarker in FIG. 55. Examples of RNA biomarkers that may be useful in the methods described herein may include a biomarker in FIG. 57B. Examples of RNA biomarkers that may be useful in the methods described herein may include a biomarker in FIG. 59B. Any combination or number of the biomarkers in FIG. 55, FIG. 57A, or FIG. 59A may be useful. For example, any of the biomarkers in FIG. 55, FIG. 57A, or FIG. 59A may be useful in distinguishing between biofluid samples of subjects with and without a cancer such as pancreatic cancer. In some cases, a cancer evaluation method includes the use of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 biomarkers from Table 20. In some cases, any of the biomarkers may be chosen from ENST00000483727.5, ENST00000531734.6, ENST00000437154.6, ENST00000531997.1, ENST00000424185.7, ENST00000652176.1, ENST00000392593.9, ENST00000532853.5, ENST00000429947.1, ENST00000580914.1, ENST00000368205.7, ENST00000531709.6, ENST00000524817.5, ENST00000651281.1, ENST00000499685.2, ENST00000311921.8, ENST00000472111.5, ENST00000585172.2, ENST00000287713.7, or ENST00000547687.2. In some cases the RNA may encode itchy E3 ubiquitin protein ligase, adenosine monophosphate deaminase 2, ADAM metallopeptidase domain 28, glycine N-acyltransferase-like protein 1 (GLYATL1), NAD(P)HX dehydratase, BICD cargo adaptor 1, phospholipase D family member 4, solute carrier family 27 member 3, long intergenic non-protein coding RNA 1237, nucleolar protein 11, protein tyrosine phosphatase receptor type K, nuclear RNA export factor 1, switching B cell complex subunit SWAP70, ERCC excision repair 5 endonuclease, BTG1 divergent transcript, zinc finger protein 507, galactose-1-phosphate uridylyltransferase, a novel pseudogene, nicotinamide nucleotide adenylyltransferase 2, or charged multivesicular body protein 1A. Some methods use a subgroup of said biomarkers. In some cases, the biomarkers may include ENST00000483727.5, ENST00000531734.6, ENST00000437154.6, ENST00000531997.1 or ENST00000424185.7 or a combination thereof. In some cases, the biomarkers may include RNA encoded by a gene or protein such as AMPD2, ENSG00000078747, ADAM28, GLYATL1 pseudogene, or NAXD. In some cases, a biomarker may include AMPD2. In some cases, a biomarker may include ENSG00000078747. In some cases, a biomarker may include ADAM28. In some cases, a biomarker may include GLYATL1 pseudogene. In some cases, a biomarker may include NAXD. In some cases, the biomarkers may include RNA encoding a protein such as Q01433-AMPD2, Q9UKQ2-ADA28 or Q8IW45-NNRD. In some cases, a biomarker may include Q01433-AMPD2. In some cases, a biomarker may include Q9UKQ2-ADA28. In some cases, a biomarker may include Q8IW45-NNRD. In some cases, a biomarker may include ENST00000483727.5, ENST00000531734.6, ENST00000437154.6, ENST00000531997.1 or ENST00000424185.7 or a combination thereof. In some cases, the biomarkers may contain ENST00000483727.5. In some cases, the biomarkers may be a decrease in ENST00000483727.5. In some cases, the biomarkers may contain ENST00000531734.6. In some cases, the biomarkers may be a decrease in ENST00000531734.6. In some cases, the biomarkers may contain ENST00000437154.6. In some cases, the biomarkers may be a decrease in ENST00000437154.6. In some cases, the biomarkers may contain ENST00000531997.1. In some cases, the biomarkers may be a decrease in ENST00000531997.1. In some cases, the biomarkers may contain ENST00000424185.7. In some cases, the biomarkers may be an increased in ENST00000424185.7.

Any combination or number of the transcriptomic biomarkers in this section or described herein may be useful. For example, some of the biomarkers may be useful in distinguishing between biofluid samples of subjects with and without a cancer.

### Genomic Data

The data described herein may include data related to genetic material or genomic data. Genomic data may include data about genetic material such as nucleic acids or histones. The nucleic acids may include DNA. Genomic data may include information on the presence, absence, or amount of the genetic material. An amount of genetic material may be indicated as a concentration, absolute number, or may be relative.

Genomic data may include DNA sequence data. The sequence data may include gene sequences. For example, the genomic data may include sequence data for up to about 20,000 genes. The genomic data may also include sequence data for non-coding DNA regions. DNA sequence data may include information on the presence, absence, or amount of DNA sequences. The DNA sequence data may include information on the presence or absence of a mutation such as a single nucleotide polymorphism. The DNA sequence data may include DNA measurement of an amount of mutated DNA, for example a measurement of mutated DNA from cancer cells.

Genomic data may include epigenetic data. Examples of epigenetic data include DNA methylation data, DNA hydroxymethylation data, or histone modification data. Epigenetic data may include DNA methylation or hydroxymethylation. DNA methylation or hydroxymethylation may be measured in whole or at regions within the DNA. Methylated DNA may include methylated cytosine (e.g., 5-methylcytosine). Cytosine is often methylated at CpG sites and may be indicative of gene activation.

Epigenetic data may include histone modification data. Histone modification data may include the presence, absence, or amount of a histone modification. Examples of histone modifications include serotonylation, methylation, citrullination, acetylation, or phosphorylation. Some specific examples of histone modifications may include lysine methylation, glutamine serotonylation, arginine methylation, arginine citrullination, lysine acetylation, serine phosphorylation, threonine phosphorylation, or tyrosine phosphorylation. Histone modifications may be indicative of gene activation.

Genomic data may be distinguished by subtype, where each subtype includes a different type of genomic data. For example, DNA sequence data may be included in another subtype, and epigenetic data may be included in one subtype, or different types of epigenetic data may be included in different subtypes.

Genomic data may be generated by any of a variety of methods. Generating genomic data may include using a detection reagent that binds to a genetic material such as DNA or histones and yields a detectable signal. After use of a detection reagent that binds to genetic material and yields a detectable signal, a readout may be obtained that is indicative of the presence, absence or amount of the genetic material. Generating genomic data may include concentrating, filtering, or centrifuging a sample.

Some examples of methods for generating DNA sequence data include use of sequencing, microarray analysis (e.g., a SNP microarray), hybridization, polymerase chain reaction, or electrophoresis, or a combination thereof. DNA sequence data may be generated by sequencing a subject's DNA. Sequencing may include massive parallel sequencing. Examples of massive parallel sequencing techniques include pyrosequencing, sequencing by reversible terminator chemistry, sequencing-by-ligation mediated by ligase enzymes, or phospholinked fluorescent nucleotides or real-time sequencing. Generating genomic data may include preparing a sample or template for sequencing. Some template preparation methods include use of amplified templates originating from single DNA molecules, or single DNA molecule templates. Examples of amplification methods include emulsion PCR, rolling circle, or solid-phase amplification

DNA methylation can be detected by use of mass spectrometry, methylation-specific PCR, bisulfite sequencing, a HpaII tiny fragment enrichment by ligation-mediated PCR assay, a Glal hydrolysis and ligation adapter dependent PCR assay, a chromatin immunoprecipitation (ChIP) assay combined with a DNA microarray (a ChIP-on-chip assay), restriction landmark genomic scanning, methylated DNA immunoprecipitation, pyrosequencing of bisulfite treated DNA, a molecular break light assay for DNA adenine methyltransferase activity, methyl sensitive Southern blotting, methylCpG binding proteins, high resolution melt analysis, a methylation sensitive single nucleotide primer extension assay, another methylation assay, or a combination thereof.

Histone modifications may be detected by using mass spectrometry or an immunoassay, an enzyme-linked immunosorbent assay, a western blot, a dot blot, or immunostaining, or a combination thereof.

In addition to any of the above methods, generating genomic data may include contacting a sample with particles such that the particles adsorb biomolecules comprising genetic material. The adsorbed genetic material may be part of a biomolecule corona. The adsorbed genetic material may be measured or identified in generating the genomic data.

### Lipidomic Data

The data such as multi-omic data described herein may include lipid data or lipidomic data. Lipidomic data may include information on the presence, absence, or amount of various lipids. For example, lipidomic data may include amounts of lipids. A lipid amount may be indicated as a concentration or quantity of lipids, for example a concentration of a lipid in a biofluid. A lipid amount may be relative to another lipid or to another biomolecule. Lipidomic data may include information on the presence of lipids. Lipidomic data may include information on the absence of lipids.

Many organisms contain complex arrays of lipids (for example, humans express over 600 types of lipids), whose relative expression can serve as a powerful marker for biological state and health determinations. Lipids are a diverse class of biomolecules which include fatty acids (e.g., long carbohydrates with carboxylate tail groups), di-, tri-, and poly-glycerides, phospholipids, prenols, sterols (e.g., cholesterol), and ladderanes, among many other types. While lipids are primarily found in membranes, free, protein-complexed, and nucleic acid-complexed lipids are typically present in a range of biofluids, and in some cases may be differentially fractionated from membrane bound lipids. For example, lipid-binding proteins (e.g., albumin) may be collected from a sample by immunohistochemical precipitation, and then chemically induced to release bound lipids for subsequent collection and detection.

Lipids may be an integral component in the development of diseases such as cancer. For example, lipids may be key players in cancer biology, as they may affect or be involved in feeding membrane and cell proliferation, lipotoxicity (where lipid content balance may aid in protection from lipotoxicity), empowering cellular processes, membrane biophysics, oncogenic signaling and metastasis, protection from oxidative stress, signaling in the microenvironment, or immune-modulation. Some lipid classes may be relevant to cancers, such as glycerophospholipids in hepatocellular carcinomas, glycerophospholipids and acylcarnitines, choline containing lipids and phospholipids increase during metastasis, or sphingolipid regulation of cancer cell survival and death.

Lipid data may be generated from a sample after the sample has been treated to isolate or enrich lipids in the sample. Generating lipid data may include concentrating, filtering, or centrifuging a sample. Lipid analysis can comprise lipid fractionation. In many cases, lipids may be readily separated from other biomolecule types for lipid-specific analysis. As many lipids are strongly hydrophobic, organic solvent extractions and gradient chromatography methods can cleanly separate lipids from other biomolecule-types present within a sample. Lipid data may be generated using mass spectrometry. Lipid analysis may then distinguish lipids by class (e.g., distinguish sphingolipids from chlorolipids) or by individual type.

Lipidomic data may be generated by any of a variety of methods. Generating lipidomic data may include using a detection reagent that binds to a lipid and yields a detectable signal. After use of a detection reagent that binds to a lipid and yields a detectable signal, a readout may be obtained that is indicative of the presence, absence or amount of the lipid. Generating lipidomic data may include concentrating, filtering, or centrifuging a sample.

Lipidomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. An example of a method for generating lipidomic data includes using mass spectrometry. Mass spectrometry may include a separation method step such as liquid chromatography (e.g., HPLC). Mass spectrometry may include an ionization method such as electron ionization, atmospheric-pressure chemical ionization, electrospray ionization, or secondary electrospray ionization. Mass spectrometry may include surface-based mass spectrometry or secondary ion mass spectrometry. Another example of a method for generating lipidomic data includes nuclear magnetic resonance (NMR). Other examples of methods for generating lipidomic data include Fourier-transform ion cyclotron resonance, ion-mobility spectrometry, electrochemical detection (e.g., coupled to HPLC), or Raman spectroscopy and radiolabel (e.g., when combined with thin-layer chromatography). Some mass spectrometry methods described for generating lipidomic data may be used for generating proteomic data, or vice versa. Lipidomic data may also be generated using a immunoassays such as enzyme-linked immunosorbent assays, western blots, dot blots, or immunohistochemistry. Generating lipidomic data may involve use of a lipid panel.

In addition to any of the above methods, generating lipidomic data may include contacting a sample with particles such that the particles adsorb biomolecules comprising lipids. The adsorbed lipids may be part of a biomolecule corona. The adsorbed lipids may be measured or identified in generating the lipidomic data.

Lipids may have associations with biology of a disease such as cancer. Lipids may include phospholipids. Examples of phospholipids include phosphatidylethanolamine (PE), phosphatidylcholine (PC), phosphatidylinositol (PI), or phosphatidylglycerol (PG). Some phospholipids are components of cellular membrane and may play roles in cells such as chemical-energy storage, cellular signaling, cell membrane, or cellular interactions within tissue. A lipid may include a ceramide (CER). Ceramides may act as tumor suppressors and may be a therapeutic pathway to target. For example, the efficacy of some chemotherapeutics and targeted therapies may be dictated by ceramide levels. A lipid may include a diacylglyceride (DAG). A lipid may include a triacylglyceride (TAG). A lipid may include a fatty acid (FA).

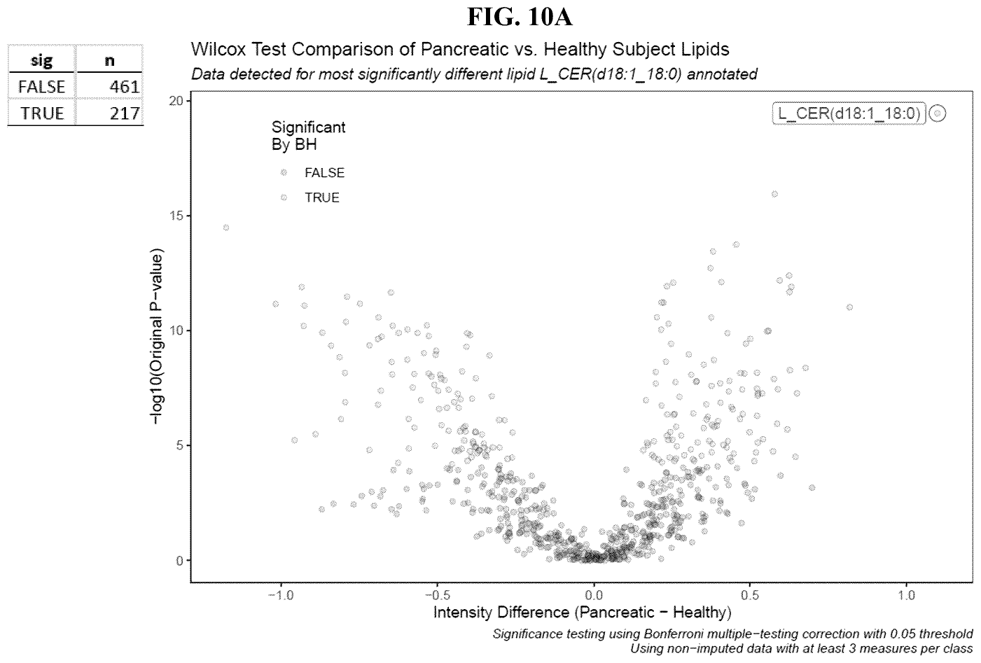

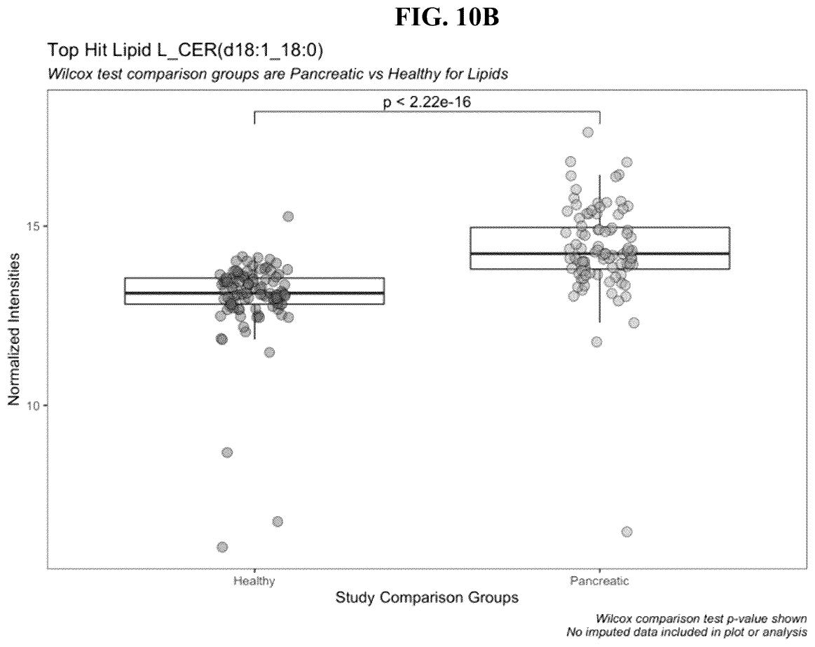

An example of a lipid is shown in FIG. 10A-10B. A lipid to be detected in a method described herein may include CER(d18:1_10:0). Some examples of lipids are shown in FIG. 13. A lipid to be detected in a method described herein may include CER(d18.1_18.0), PC(18.2_20.5), CER(d18.1_24.1), CER(d18.1_16.0), TAG(56.5_FA18.0), CER(d18.0_24.1), TAG(56.5_FA18.1), DAG(16.0_22.5), CER(d18.1_22.1), PE(P-18.0_18.3), or PE(17.0_22.6). Any number of the aforementioned lipids may be used. Any of the lipids may be used in a classifier.

Examples of lipid biomarkers that may be useful in the methods described herein may include a biomarker in FIG. 55. Examples of lipid biomarkers that may be useful in the methods described herein may include a biomarker in FIG. 57C. Examples of lipid biomarkers that may be useful in the methods described herein may include a biomarker in FIG. 59C. Any combination or number of the lipid biomarkers in FIG. 55, FIG. 57A, or FIG. 59A may be useful. For example, any of the biomarkers in FIG. 55, FIG. 57A, or FIG. 59A may be useful in distinguishing between biofluid samples of subjects with and without a cancer such as pancreatic cancer. In some cases, a cancer evaluation method includes the use of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 of the biomarkers from Table 21. In some cases, any of the biomarkers may be chosen from PC(18:2_20:5)+AcO, DAG(18:1_20:0)+NH4, PE(O-16:0_22:6)−H, PC(18:2_20:3)+AcO, CER(d18:1/18:0)+H, CE(22:0)+NH4, PE(14:0_22:5)−H, PC(20:5_20:5)+AcO, PE(P-18:0_18:3)+H, PE(O-16:0_20:3)−H, CE(18:3)+NH4, PE(O-18:0_22:5)−H, PE(O-18:0_20:5)−H, PE(P-20:0_20:3)+H, PE(O-16:0_20:2)−H, CER(d18:1/24:0)+H, PA(20:1_20:3)−H, PA(20:0_20:5)−H, CE(20:0)+NH4, or PC(16:1_20:3)+AcO. Some biomarkers may be used or included based on a presences of lipid species. Some biomarkers may be used or included based on the absence of lipid species. Some methods use a subgroup of said biomarkers. In some cases, the biomarkers may include PC(18:2_20:5)+AcO, DAG(18:1_20:0)+NH4, PE(O-16:0_22:6)−H, PC(18:2_20:3)+AcO, CER(d18:1/18:0)+H or a combination thereof, in presence or absence. In some cases, the biomarkers may contain PC(18:2_20:5)+AcO. In some cases, the biomarkers may be a decrease in PC(18:2_20:5)+AcO. In some cases, the biomarkers may contain DAG(18:1_20:0)+NH4. In some cases, the biomarkers may be a decrease in DAG(18:1_20:0)+NH4. In some cases, the biomarkers may contain PE(O-16:0_22:6)−H. In some cases, the biomarkers may be a decrease in PE(O-16:0_22:6)−H. In some cases, the biomarkers may contain PC(18:2_20:3)+AcO. In some cases, the biomarkers may be a decrease in PC(18:2_20:3)+AcO. In some cases, the biomarkers may contain CER(d18:1/18:0)+H. In some cases, the biomarkers may be an increase in CER(d18:1/18:0)+H.

Any combination or number of the lipid biomarkers in this section or described herein may be useful. For example, some of the biomarkers may be useful in distinguishing between biofluid samples of subjects with and without a cancer.

### Metabolomic Data

The data described herein may include metabolite data or metabolomic data. Metabolomic data may include information on small-molecule (e.g., less than 1.5 kDa) metabolites (such as metabolic intermediates, hormones or other signaling molecules, or secondary metabolites). Metabolomic data may involve data about metabolites. Metabolites may include are substrates, intermediates or products of metabolism. A metabolite may be any molecule less than 1.5 kDa in size. Examples of metabolites may include sugars, lipids, amino acids, fatty acids, phenolic compounds, or alkaloids. Metabolomic data may be distinguished by subtype, where each subtype includes a different type of metabolite. Metabolomic data may include some lipid data.

Metabolomic data may include information on the presence, absence, or amount of various metabolites. For example, metabolomic data may include amounts of metabolites. A metabolite amount may be indicated as a concentration or quantity of metabolites, for example a concentration of a metabolite in a biofluid. A metabolite amount may be relative to another metabolite or to another biomolecule. Metabolomic data may include information on the presence of metabolites. Metabolomic data may include information on the absence of metabolites.

Metabolomic data generally includes data on a number of metabolites. For example, metabolomic data may include information on the presence, absence, or amount of 1000 or more metabolites. In some cases, metabolomic data may include information on the presence, absence, or amount of 5000, 10,000, 20,000, 50,000, 100,000, 500,000, 1 million, 1.5 million, 2 million, or more metabolites, or a range of metabolites defined by any two of the aforementioned numbers of metabolites.

Metabolomic data may be generated by any of a variety of methods. Generating metabolomic data may include using a detection reagent that binds to a metabolite and yields a detectable signal. After use of a detection reagent that binds to a metabolite and yields a detectable signal, a readout may be obtained that is indicative of the presence, absence or amount of the metabolite. Generating metabolomic data may include concentrating, filtering, or centrifuging a sample.

Metabolomic data may be generated using mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. An example of a method for generating metabolomic data includes using mass spectrometry. Mass spectrometry may include a separation method step such as liquid chromatography (e.g., HPLC). Mass spectrometry may include an ionization method such as electron ionization, atmospheric-pressure chemical ionization, electrospray ionization, or secondary electrospray ionization. Mass spectrometry may include surface-based mass spectrometry or secondary ion mass spectrometry. Another example of a method for generating metabolomic data includes nuclear magnetic resonance (NMR). Other examples of methods for generating metabolomic data include Fourier-transform ion cyclotron resonance, ion-mobility spectrometry, electrochemical detection (e.g., coupled to HPLC), or Raman spectroscopy and radiolabel (e.g., when combined with thin-layer chromatography). Some mass spectrometry methods described for generating metabolomic data may be used for generating proteomic data, or vice versa. Metabolomic data may also be generated using a immunoassays such as enzyme-linked immunosorbent assays, western blots, dot blots, or immunohistochemistry. Generating metabolomic data may involve use of a lipid panel.

In addition to any of the above methods, generating metabolomic data may include contacting a sample with particles such that the particles adsorb biomolecules comprising metabolites. The adsorbed metabolites may be part of a biomolecule corona. The adsorbed metabolites may be measured or identified in generating the metabolomic data.

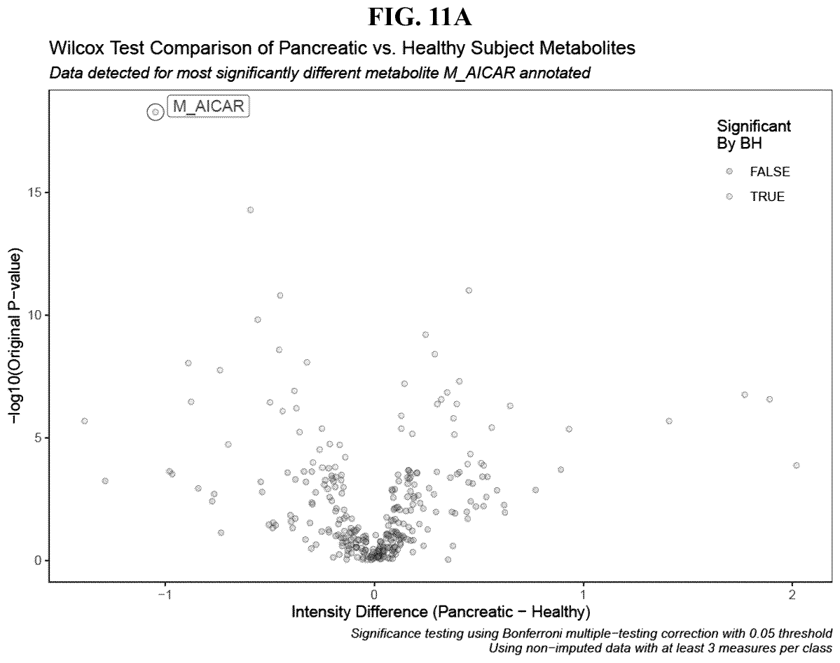

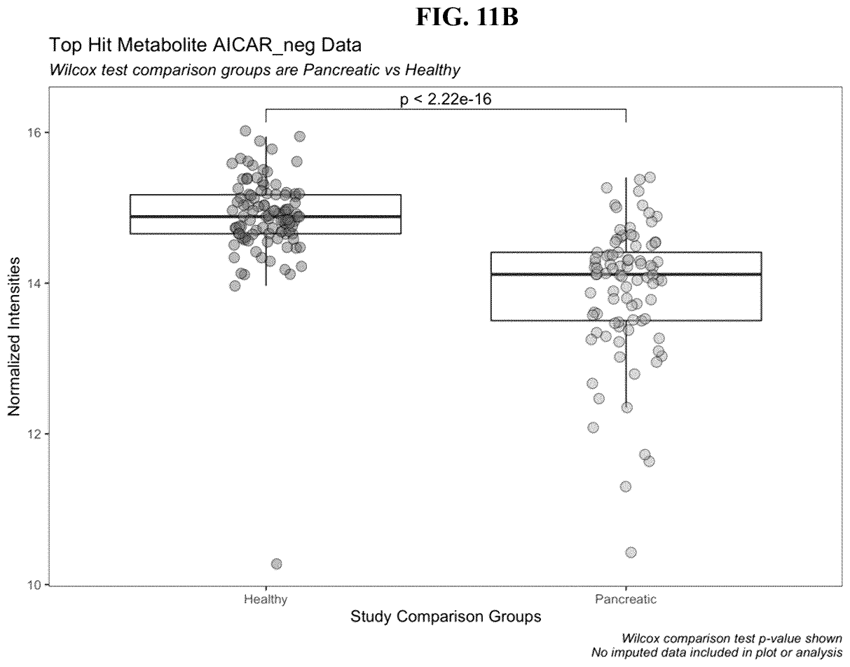

An example of a metabolite is shown in FIG. 11A-11B. A metabolite to be detected in a method described herein may include 5-Aminoimidazole-4-carboxamide ribonucleotide (AICAR). The metabolite may include a nucleotide such as a monophosphate nucleotide. Some examples of metabolites are shown in FIG. 13. A metabolite to be detected in a method described herein may include cytidine monophosphate (CMP). The metabolite may include AICAR or CMP. Metabolites to be detected may include AICAR and CMP. Any number of the aforementioned metabolites may be used. Any of the metabolites may be used in a classifier.

CA19-9 may be used as a biomarker. CA19-9 may be used alone or in combination with any other biomarker or group of biomarkers.

Examples of metabolite biomarkers that may be useful in the methods described herein may include a biomarker in FIG. 55. Examples of metabolite biomarkers that may be useful in the methods described herein may include a biomarker in FIG. 57D. Examples of metabolite biomarkers that may be useful in the methods described herein may include a biomarker in FIG. 59D. Any combination or number of the metabolite biomarkers in FIG. 55, FIG. 57A, or FIG. 59A may be useful. For example, any of the biomarkers in FIG. 55, FIG. 57A, or FIG. 59A may be useful in distinguishing between biofluid samples of subjects with and without a cancer such as pancreatic cancer. In some cases, a cancer evaluation method includes the use of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 of the biomarkers from Table 22. In some cases, any of the biomarkers may be chosen from AICAR, cystine, CMP, gentisate, creatine, imidazoleacetic acid, inosine, N-isovalerylglycine, glucose-6-phosphate, metanephrine, N-acetylglutamate, 5-thymidilic acid (dTMP), UMP, fructose-6-phosphate, cystine, ubiquinol, guanine, shikimic acid, 1-methylimidazole acetate or flavone 2. Some biomarkers may be based on a presence of a metabolite. Some biomarkers may be based on the absence of a metabolite. Some methods use a subgroup of said biomarkers. In some cases, the biomarkers may include AICAR, cystine, CMP, gentisate, creatine, or a combination thereof, in presence or absence. In some cases, the biomarkers may include AICAR. In some cases, the biomarker may be a decrease in AICAR. In some cases, the biomarkers may include cystine. In some cases, the biomarker may be an increase in cystine. In some cases, the biomarkers may include CMP. In some cases, the biomarker may be a decrease CMIP. In some cases, the biomarkers may include gentisate. In some cases, the biomarker may be a decrease in gentisate. In some cases, the biomarkers may include creatine. In some cases, the biomarker may be a decrease in creatine.

Any combination or number of the metabolite biomarkers in this section or described herein may be useful. For example, some of the biomarkers may be useful in distinguishing between biofluid samples of subjects with and without a cancer.

### Use of Reference Biomolecules

In some aspects, obtaining proteomic data can include the use of a reference biomolecule, which may be labeled. Samples may be contacted with a reference biomolecule, for example prior to generating data. The data described herein may generated using reference biomolecules. For example, a method may include contacting a sample with reference biomolecules that comprise labeled versions of each biomolecule such as each protein. The reference biomolecule may comprise an internal standard. For example, the reference biomolecule may be added at a predetermined amount to the biological sample to serve as an internal standard, and to aid in identification of similar biomolecules that are endogenous to the sample. For example, isotopically labeled reference proteins may be spiked into a sample, measured along with endogenous proteins using mass spectrometry, used to identify the endogenous proteins on mass spectra, and also used to help determine an accurate amount of the endogenous proteins. An internal standard may include a biomolecule that is added in a constant or known amount to the biological sample. Internal standards may comprise a non-endogenous labeled version of the endogenous biomolecules. Some examples refer to the use of internal labeled standards as “PiQuant.”

Of the labeled and endogenous biomolecules, individual labeled biomolecules may correspond to the individual endogenous biomolecules. For example, the biomolecules may comprise proteins, and the endogenous proteins may comprise 100-1500 different proteins and the labeled biomolecules may comprise the same 100-1500 proteins but each labeled biomolecule may comprise a label.

The reference biomolecules may include at least 5, at least 10, at least 50, at least 100, at least 250, at least 500, at least 750, at least 1000, at least 1500, at least 2000, at least 2500, at least 5000, at least 7500, at least 10,000, at least 15,000, at least 20,000, or at least 25,000 individual or distinct biomolecules. In some instances, the reference biomolecules include less than 5, less than 10, less than 50, less than 100, less than 250, less than 500, less than 750, less than 1000, less than 1500, less than 2000, less than 2500, less than 5000, less than 7500, less than 10,000, less than 15,000, less than 20,000, or less than 25,000 individual or distinct biomolecules.

As an example, a sample comprises endogenous protein A, endogenous protein B, and endogenous protein C. Endogenous proteins A, B and C are difficult to measure because of their low abundance. Upon spiking predetermined amounts of isotopically labeled versions of proteins A, B and C into the sample, endogenous proteins A, B, and C, and the isotopically labeled versions of proteins A, B and C are analyzed together using mass spectrometry. Because the isotopically labeled versions are heavier, their mass spectra are shifted, and are distinguishable from mass spectra for the endogenous proteins. The isotopically labeled versions are more readily identifiable on a mass spectrometry readout thereby facilitating the identification of mass spectra for endogenous proteins A, B and C on the mass spectrometry readout. Because a predetermined amount of isotopically labeled proteins A, B, and C was added to spiked into the sample, their concentration is known, and the mass spectra for isotopically labeled proteins A, B, and C are used to accurately measure the amounts of endogenous proteins A, B, and C from the mass spectrometry readout. The accurate measurements of the endogenous proteins A, B, and C may be obtained by comparing the relative intensities of the mass spectrometry readouts for endogenous proteins A, B, and C relative to the intensities of the mass spectrometry readouts for the known concentrations or amounts of isotopically labeled proteins A, B, and C.

### Use of Particles

Samples may be contacted with particles, for example prior to generating data. The data described herein may generated using particles. For example, a method may include contacting a sample with particles such that the particles adsorb biomolecules such as proteins, transcripts, genetic materials, or metabolites. The particles may attract different sets of biomolecules than may normally be measured accurately by obtaining a measurement directly on a sample. For example, a dominant biomolecule may make up a large percentage of certain type of biomolecules in a sample. For example, one protein may make up a large portion of proteins in circulation that is collected by blood sampling. By adhering biomolecules to particles prior to analyzing the biomolecules, a subset of biomolecules may be obtained that does not include the dominant biomolecule. Removing dominant biomolecules in this way may increase the accuracy of biomolecule measurements and sensitivity of an analysis using those measurements.

The biomolecules that may be adsorbed to particles may include proteins. The adsorbed biomolecules may make up a biomolecule corona around the particle. The adsorbed biomolecules may be measured or identified in generating the data (e.g., proteomic data).

Particles can be made from various materials. Such materials may include metals, magnetic materials, polymers, or lipids. A particle may be made from a combination of materials. A particle may comprise layers of different materials. The different materials may have different properties. A particle may include a core comprising one material and be coated with another material. The core and the coating may have different properties.

A particle may include a metal. For example, a particle may include gold, silver, copper, nickel, cobalt, palladium, platinum, iridium, osmium, rhodium, ruthenium, rhenium, vanadium, chromium, manganese, niobium, molybdenum, tungsten, tantalum, iron, or cadmium, or a combination thereof.

A particle may be magnetic (e.g., ferromagnetic or ferrimagnetic). A particle comprising iron oxide may be magnetic. A particle may be a superparamagnetic iron oxide nanoparticle (SPION).

A particle may include a polymer. Examples of the polymer may include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). A particle may be made from a combination of polymers.

A particle may include a lipid. Examples of the lipid include dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, or cholesterol. A particle may be made from a combination of lipids.

Further examples of materials include silica, carbon, carboxylate, polyacrylic acid, carbohydrates, dextran, polystyrene, dimethylamine, amines, or silanes. Some examples of particles include a carboxylate SPION, a phenol-formaldehyde coated SPION, a silica-coated SPION, a polystyrene coated SPION, a carboxylated Poly(styrene-co-methacrylic acid), P(St-co-MAA) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(vinylbenzyltrimethylammonium chloride) (PVBTMAC) coated SPION, caboxylate coated with peracetic acid, a poly(oligo(ethylene glycol) methyl ether methacrylate) (POEGMA)-coated SPION, a polystyrene carboxyl functionalized particle, a carboxylic acid particle, a particle with an amino surface, a silica amino functionalized particle, a particle with a Jeffamine surface, or a silica silanol coated particle.

Particles of various sizes may be used. The particles may include nanoparticles. Nanoparticles may be from about 10 nm to about 1000 nm in diameter. For example, the nanoparticles can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. Some examples include diameters of about 50 nm, about 130 nm, about 150 nm, 400-600 nm, or 100-390 nm.

The particles may include microparticles. A microparticle may be a particle that is from about 1 μm to about 1000 μm in diameter. For example, the microparticles can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. A microparticle may be less than 1000 μm in diameter. Some examples include diameters of 2.0-2.9 μm.

The particles may include physiochemically distinct sets of particles (for example, 2 or more sets of physiochemically particles where 1 set of particles is physiochemically distinct from another set of particles). Examples of physiochemical properties include charge (e.g., positive, negative, or neutral) or hydrophobicity (e.g., hydrophobic or hydrophilic). The particles may include 2, 3, 4, 5, 6, 7, 8, 9, 10, 11, 12, 13, 14, 15, 16, 17, 18, 19, 20, or more sets of particles, or a range of sets of particles including any of said numbers of sets of particles.

### Computer Systems

Certain aspects of the methods described herein may be carried out using a computer system. For example, data analysis may be carried out using a computer system. Likewise, multiple data sets may be obtained through the use of a computer system. A readout indicative of the presence, absence or amount of a biomolecule (e.g., a protein, transcript, genetic material, or metabolite) may be obtained at least in part using a computer system. The computer system may be used to carry out a method of using a classifier to assign a label corresponding to a presence, absence, or likelihood of a cancer to data, or to identify multiple data sets as indicative or as not indicative of the cancer. In certain aspects, the cancer is pancreatic cancer. The pancreatic cancer can be early-stage pancreatic cancer or late-stage pancreatic cancer. The computer system may generate a report identifying a likelihood of the subject having a cancer. The computer system may transmit the report. For example, a diagnostic laboratory may transmit a report regarding the cancer identification to a medical practitioner. A computer system may receive a report.

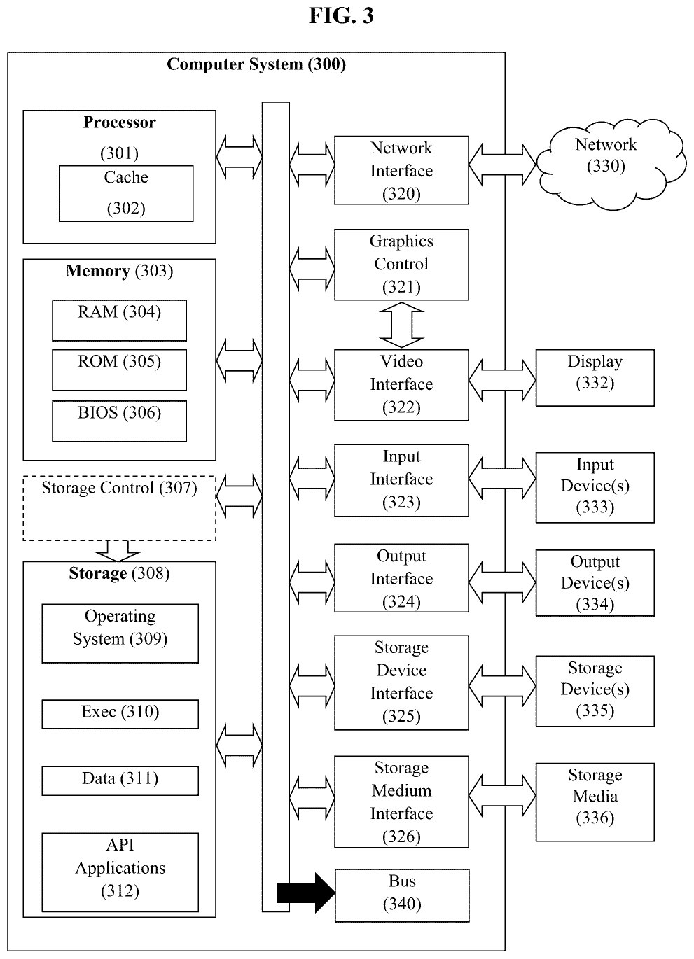

A computer system that carries out a method described herein may include some or all of the components shown in FIG. 3. Referring to FIG. 3, a block diagram is shown depicting an exemplary machine that includes a computer system 300 (e.g., a processing or computing system) within which a set of instructions can execute for causing a device to perform or execute any one or more of the aspects and/or methodologies for static code scheduling of the present disclosure. The components in FIG. 3 are examples only and do not limit the scope of use or functionality of any hardware, software, embedded logic component, or a combination of two or more such components implementing particular embodiments.

Computer system 300 may include one or more processors 301, a memory 303, and a storage 108 that communicate with each other, and with other components, via a bus 340. The bus 340 may also link a display 332, one or more input devices 333 (which may, for example, include a keypad, a keyboard, a mouse, a stylus, etc.), one or more output devices 334, one or more storage devices 335, and various tangible storage media 336. All of these elements may interface directly or via one or more interfaces or adaptors to the bus 340. For instance, the various tangible storage media 336 can interface with the bus 340 via storage medium interface 326. Computer system 300 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 300 includes one or more processor(s) 301 (e.g., central processing units (CPUs) or general-purpose graphics processing units (GPGPUs)) that carry out functions. Processor(s) 301 optionally contains a cache memory unit 302 for temporary local storage of instructions, data, or computer addresses. Processor(s) 301 are configured to assist in execution of computer readable instructions. Computer system 300 may provide functionality for the components depicted in FIG. 3 as a result of the processor(s) 301 executing non-transitory, processor-executable instructions embodied in one or more tangible computer-readable storage media, such as memory 303, storage 308, storage devices 335, and/or storage medium 336. The computer-readable media may store software that implements particular embodiments, and processor(s) 301 may execute the software. Memory 303 may read the software from one or more other computer-readable media (such as mass storage device(s) 335, 336) or from one or more other sources through a suitable interface, such as network interface 320. The software may cause processor(s) 301 to carry out one or more processes or one or more steps of one or more processes described or illustrated herein. Carrying out such processes or steps may include defining data structures stored in memory 303 and modifying the data structures as directed by the software.

The memory 303 may include various components (e.g., machine readable media) including, but not limited to, a random access memory component (e.g., RAM 304) (e.g., static RAM (SRAM), dynamic RAM (DRAM), ferroelectric random access memory (FRAM), phase-change random access memory (PRAM), etc.), a read-only memory component (e.g., ROM 305), and any combinations thereof. ROM 305 may act to communicate data and instructions unidirectionally to processor(s) 301, and RAM 304 may act to communicate data and instructions bidirectionally with processor(s) 301. ROM 305 and RAM 304 may include any suitable tangible computer-readable media described below. In one example, a basic input/output system 306 (BIOS), including basic routines that help to transfer information between elements within computer system 300, such as during start-up, may be stored in the memory 303.

Fixed storage 308 is connected bidirectionally to processor(s) 301, optionally through storage control unit 307. Fixed storage 308 provides additional data storage capacity and may also include any suitable tangible computer-readable media described herein. Storage 308 may be used to store operating system 309, executable(s) 310, data 311, applications 312 (application programs), and the like. Storage 308 can also include an optical disk drive, a solid-state memory device (e.g., flash-based systems), or a combination of any of the above. Information in storage 308 may, in appropriate cases, be incorporated as virtual memory in memory 303.

In one example, storage device(s) 335 may be removably interfaced with computer system 300 (e.g., via an external port connector (not shown)) via a storage device interface 325. Particularly, storage device(s) 335 and an associated machine-readable medium may provide non-volatile and/or volatile storage of machine-readable instructions, data structures, program modules, and/or other data for the computer system 300. In one example, software may reside, completely or partially, within a machine-readable medium on storage device(s) 335. In another example, software may reside, completely or partially, within processor(s) 301.

Bus 340 connects a wide variety of subsystems. Herein, reference to a bus may encompass one or more digital signal lines serving a common function, where appropriate. Bus 340 may be any of several types of bus structures including, but not limited to, a memory bus, a memory controller, a peripheral bus, a local bus, and any combinations thereof, using any of a variety of bus architectures. As an example and not by way of limitation, such architectures may include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, or any combination thereof.

Computer system 300 may also include an input device 333. In one example, a user of computer system 300 may enter commands and/or other information into computer system 300 via input device(s) 333. Examples of an input device(s) 333 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), or any combinations thereof. In some aspects, the input device is a Kinect, Leap Motion, or the like. Input device(s) 333 may be interfaced to bus 340 via any of a variety of input interfaces 323 (e.g., input interface 323) including, but not limited to, serial, parallel, game port, USB, FIREWIRE, THUNDERBOLT, or any combination of the above.

In particular embodiments, when computer system 300 is connected to network 330, computer system 300 may communicate with other devices, specifically mobile devices and enterprise systems, distributed computing systems, cloud storage systems, cloud computing systems, and the like, connected to network 330. Communications to and from computer system 300 may be sent through network interface 320. For example, network interface 320 may receive incoming communications (such as requests or responses from other devices) in the form of one or more packets (such as Internet Protocol (IP) packets) from network 330, and computer system 300 may store the incoming communications in memory 303 for processing. Computer system 300 may similarly store outgoing communications (such as requests or responses to other devices) in the form of one or more packets in memory 303 and communicated to network 330 from network interface 320. Processor(s) 301 may access these communication packets stored in memory 303 for processing.

Examples of the network interface 320 include, but are not limited to, a network interface card, a modem, or any combination thereof. Examples of a network 330 or network segment 330 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, or any combinations thereof. A network, such as network 330, may employ a wired and/or a wireless mode of communication. In general, any network topology may be used.

Information and data can be displayed through a display 332. Examples of a display 332 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, or any combinations thereof. The display 332 can interface to the processor(s) 301, memory 303, and fixed storage 308, as well as other devices, such as input device(s) 333, via the bus 340. The display 332 is linked to the bus 340 via a video interface 322, and transport of data between the display 332 and the bus 340 can be controlled via the graphics control 321. In some aspects, the display is a video projector. In some aspects, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 332, computer system 300 may include one or more other peripheral output devices 334 including, but not limited to, an audio speaker, a printer, a storage device, or any combinations thereof. Such peripheral output devices may be connected to the bus 340 via an output interface 324. Examples of an output interface 324 include, but are not limited to, a serial port, a parallel connection, a USB port, a FIREWIRE port, a THUNDERBOLT port, or any combinations thereof.

In addition or as an alternative, computer system 300 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices may include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

The computing device may include an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some aspects, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some cases, the platforms, systems, media, or methods disclosed herein include one or more non-transitory computer readable storage media encoded with a program including instructions executable by an operating system of a computer system. The computer system may be networked. A computer readable storage medium may be a tangible component of a computing device. A computer readable storage medium may be removable from a computing device. A computer readable storage medium may include any of, by way of non-limiting examples, CD-ROMs, DVDs, flash memory devices, solid state memory, magnetic disk drives, magnetic tape drives, optical disk drives, distributed computing systems including cloud computing systems and services, or the like. In some cases, the program and instructions are permanently, substantially permanently, semi-permanently, or non-transitorily encoded on the media.

### Data Integration and Analysis

The methods described herein, when analyzing data described herein such as proteomic data, transcriptomic data, genomic data, or metabolomic data, can include generating or using a classifier for indicating the subject of having or at risk of having pancreatic cancer with a certain sensitivity or specificity. In some aspects, a method described herein generates or uses a classifier from the data for indicating the subject of having or at risk of having pancreatic cancer with a sensitivity of at least about 50%, at least about 60%, at least about 70%, at least about 80%, or at least about 90%. In some aspects, a method described herein generates or uses a classifier from the data for indicating the subject of having or at risk of having pancreatic cancer with a specificity of at least about 50%, at least about 60%, at least about 70%, at least about 80%, or at least about 90%. In some aspects, a method described herein generates or uses a classifier from the data for indicating the subject of having or at risk of having pancreatic cancer with a sensitivity or specificity no greater than about 50%, no greater than about 60%, no greater than about 70%, no greater than about 80%, no greater than about 90%, or no greater than about 95%.

Separate data sets may be integrated into an analysis for more accurate prediction or identification of a cancer than individual data sets may provide for. For example, a method may include using more than one classifier to identify pancreatic cancer in a subject, where each classifier is used to analyze a separate data set and each classifier is independent of the other. When the classifiers err independently from each other, the combined analysis may be more accurate than an analysis using one classifier corresponding to only one data set. Alternatively, separate data sets may be combined into one data set or analyzed by a single classifier.

A method involving multiple classifiers may include using a first classifier to generate or assign a first label corresponding to a presence, absence, or likelihood of a cancer to a first data set. The method may further include using a second classifier to generate or assign a second label corresponding to a presence, absence, or likelihood of a cancer to a second data set. The method may further include using a third classifier to generate or assign a third label corresponding to a presence, absence, or likelihood of a cancer to a third data set. The method may further include using a fourth classifier to generate or assign a fourth label corresponding to a presence, absence, or likelihood of a cancer to a fourth data set. Additional classifiers may be used to generate or assign labels to further data sets. Each classifier may be trained using data or combined data from samples of subjects with cancer and from samples of control subjects. Further, each classifier may include a stand-alone machine learning model or an ensemble of machine-learning models trained on the same input features.

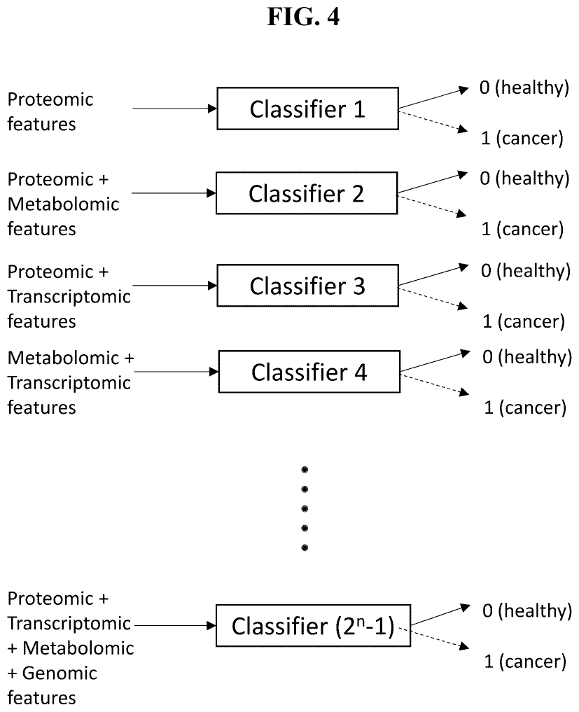

Some classifiers may analyze a combined data set, whereas other classifiers may analyze only one data set. For example, an additional classifier may generate or assign a label corresponding to a presence, absence, or likelihood of a cancer to a combined data set. The combined data set may include any combination of two or more types or subtypes of data. For example, data types may include proteomic data, transcriptomic data, genomic data, or metabolomic data. Each classifier may make a determination of the cancer as shown in FIG. 4.

The labels generated or assigned by each classifier may be used to identify the data as indicative or as not indicative of the cancer. This may entail picking a label assigned by any one or more of the classifiers or may entail generating or obtaining a majority voting score based on the first and second labels.

Identifying the multiple data sets as indicative or as not indicative of the cancer may include majority voting across of some or all of the classifier-generated labels. For example, the final determination of whether the subject is likely to have the cancer or not may be identified based on whether more classifiers assigned labels corresponding to the presence of the cancer or whether more classifiers assigned labels corresponding to the absence of the cancer. Identifying the data as indicative or as not indicative of the cancer may include generating or using a weighted average of some or all of the classifier-generated labels.

Identifying the data as indicative or as not indicative of the cancer may include obtaining or generating a weighted average of the labels generated or assigned by some or all of the classifiers. Weights of the weighted average may be based on one or more of: area under a ROC curve, area under a precision-recall curve, accuracy, precision, recall, sensitivity, F1-score, or specificity.

A method involving multiple classifiers may include identifying data as indicative or as not indicative of a cancer. This may be done based on choosing a label assigned by an individual classifier, or by combining the labels assigned by multiple classifiers. The method may include identifying data as indicative or as not indicative of the cancer based on a combination of a first label and a second label, each assigned by separate classifiers. The data may be identified as indicative of the cancer based further on a third label, a fourth label, or one or more additional labels. The data may be identified as indicative of the cancer based on a first and third label, or based on a first and fourth label, where, for example, one or more of the labels are not included in the final determination.

Some aspects include identifying a likelihood of pancreatic cancer using a classifier. The classifier may be characterized by a receiver operating characteristic (ROC) curve having an area under the curve (AUC) greater than 0.7, greater than 0.75, greater than 0.8, greater than 0.85, greater than 0.9, greater than 0.91, greater than 0.92, greater than 0.93, greater than 0.94, greater than 0.95, greater than 0.96, greater than 0.97, greater than 0.98 or greater than 0.99 based on biomolecule measurement features. In some aspects, the AUC may be no greater than 0.75, no greater than 0.8, no greater than 0.85, no greater than 0.9, no greater than 0.91, no greater than 0.92, no greater than 0.93, no greater than 0.94, no greater than 0.95, no greater than 0.96, no greater than 0.97, no greater than 0.98, or no greater than 0.99.

### Feature Selection and Simplified Classifier

The methods described herein, when creating classifiers related to a biological state, can include a method of selecting only certain features of the data sets corresponding to the different types of biological data that may be collected. In some aspects, the method may include creating a classifier for each different data set separately. Each separate classifier may be then used to assign a feature importance rating to each of the individual data points within each of the data sets. This importance score is a reflection of the usefulness of each individual feature within the set in creating the classifier. Each feature of the individual data sets may be assigned a score. The biological state may be a cancer, for example pancreatic cancer.

In some aspects, individuals feature of each data set may be combined to create a simplified feature list. The selection may be based upon an importance score. The selection may be based upon interaction between the feature and other features observed in the data set model. Each feature of each different data set may be selected. The total number of features selected to comprise the simplified feature list may be 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, or 100 or more. The total number of features selected may be 20. The number of data sets from which the features are selected may be 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, or 100 or more. The number of data sets from which features are selected may be 4. The features may be selected from among data sets comprising proteomic data, transcriptome data, genomic data, lipidomic data, or metabolomic data. The number of features selected from each individual data set may be the same or it may be different. The number of features selected from a single data set may be 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, or 100 or more. The number of features selected a single data set may be 5.

In some aspects, the simplified feature list may then be used to create a simplified model. The simplified model may be used to generate a simplified classifier. The simplified classifier may have the same predictive power as a classifier created using all of the features. The simplified classifier may be characterized by a receiver operating characteristic (ROC) curve having an area under the curve (AUC) greater than 0.7, greater than 0.75, greater than 0.8, greater than 0.85, greater than 0.9, greater than 0.91, greater than 0.92, greater than 0.93, greater than 0.94, greater than 0.95, greater than 0.96, greater than 0.97, greater than 0.98 or greater than 0.99 based on biomolecule measurement features. In some aspects, the AUC may be no greater than 0.75, no greater than 0.8, no greater than 0.85, no greater than 0.9, no greater than 0.91, no greater than 0.92, no greater than 0.93, no greater than 0.94, no greater than 0.95, no greater than 0.96, no greater than 0.97, no greater than 0.98, or no greater than 0.99.

In some aspects, the creation of the simplified classifier may be based upon separating subjects into two training groups. The first training group may be used to generate the feature selection model. This model may be created by creating models for each individual data set. Then a classifier may be generated for each data set to create separate classifiers for each individual data set. The contribution of each feature to the total classifier may then be calculated. This model may be used to select features that have high predictive power. The features selected from this first model may then be used to create a new list of features for data collection. The data for the selected features may then be collected from the second training group. This simplified data set may then be sued to with a simplified second predictive model to create a simplified second classifier.

In some aspects, the creation of the simplified classifier may be based upon a single training group. The training group may be used to generate the feature selection model. This model may be created by creating models for each individual data set. Then a classifier may be generated for each data set to create separate classifiers for each individual data set. The contribution of each feature to the total classifier may then be calculated. This model may be used to select features that have high predictive power. The features selected from this first model may then be used to create a new list of features for data collection. The data for the selected features may then be isolated from the training group. This simplified data set may then be sued to with a simplified second predictive model to create a simplified second classifier. This may require that model overfitting from the shared data set be employed so that proper confidence in the classifier can be calculated.

A number of approaches to mitigate and evaluate the risk of overfitting may implemented. First, a conservative split of the total subject population may be selected, with approximately 95%, 90%, 85%, 80%, 75%, 70%, 65%, or 60% in the training set and 40%, 35%, 30%, 25%, 20%, 15%, 10%, or 5%, in the validation set. By increasing the size of the validation set, the ability to detect overfitting, if it were to occur, is increased, even if the ability to identify important classifier components is reduced. Second, the study design may incorporate an intentional differentiation in date of enrollment and site of enrollment for test and control groups. These steps mitigate the risk of systematic bias between the groups being carried over from training to validation sets. Third, an extensive cross-validation design when may be employed when optimizing model engine parameters and important feature selection. Ten rounds of 10-fold cross-validation, while computationally intensive for many input features, is a robust approach to avoid overfitting. 2, 3, 4, 5, 6, 7, 8, 9, 10, 11, 12, 13, 14, 15, 16, 17, 18, 19, 20, 21, 22, 23, 24, or 25 rounds of 10-fold cross-validation may be used. Finally, randomly permutated training subject data groups (e.g., test vs control) may determine if the 2-stage process described here produces a final, multi-omics model with similar performance in the validation set as was observed in the individual omics models.

The simplified classifier may present advantages over the complex classifier based on all data features without the loss of predictive power. The simplified classifier may be faster to process subject data. It may process an individual data set in 1 second (s), 10 s, 20 s, 30 s, 40 s, 50 s, 60 s, 2 minutes (min), 3 min, 4 min, 5 min, 6 min, 7 min, 8 min, 9 min, or 10 min. It may also allow for point of care processing. It may allow processing on less expensive or complex computer systems. It may allow for processing to be done over a web application or on a smart phone. It may allow for cloud based or remote processing. The simplified classifier may allow for easier transformation into a clinical diagnostic. This may be facilitated by reducing the number of features that must be tested to enable the classifier to operate. This may be facilitated by increasing the confidence of end users. This may be facilitated by decreasing the complexity of the validation processes required by regulatory agencies for the development of clinical diagnostic. The simplified classifier may be easier to modify than a complex classifier. It may allow for greater manipulation in order to optimize the performance of the classifier. It may be based on a simpler model. The model may be a linear regression model. The classifier may be more easily understood. It may allow for easier explanation to individuals without training in development of computer models and classifiers.

The selected the features may be the most important within their individual data set models. They may be the most important when compared across all of the data sets used to construct the simplified feature list. However, in some aspects, the features selected may also not be the most overall important feature among all of the features. Selecting features from among a greater number of data sets instead of from a the individually most important favors may be preferred. This type of feature selection may provide greater predictive accuracy relative to the total population. This may allow for classifiers with high predictive accuracy to be generated using small training groups. The training group may include 1,000, 900, 800, 700, 600, 500, 400, 300, 250, 200, 150, 100, 75, 50, or 25 people, or a range defined by any two of the aforementioned numbers of people. The training group may include less than 1,000 people, less than 900 people, less than 800 people, less than 700 people, less than 600 people, less than 500 people, less than 400 people, less than 300 people, less than 250 people, less than 200 people, less than 150 people, less than 100 people, less than 75 people, less than 50 people, or less than 25 people. The training group may include at least 1,000 people, at least 900 people, at least 800 people, at least 700 people, at least 600 people, at least 500 people, at least 400 people, at least 300 people, at least 250 people, at least 200 people, at least 150 people, at least 100 people, at least 75 people, at least 50 people, or at least 25 people.

### Biological Process Coverage

In some aspects of the inventive concepts, features may be chosen to expand the coverage of different biological processes. Biological processes may be any functionality that a living creature undergoes. The process may be present under normal function, or it may be present when the biological state of the organism has been interrupted or interfered with. The cause of the interruption or interference may be endogenous or exogenous. It may be a disease. The disease may be cancer. The cancer may be pancreatic cancer. The feature may be upregulated when the biological process is affected. The feature may be downregulated when the biological process is affected.

In some aspects, coverage means that a feature is involved with, related to, affected by, or otherwise has some relationship to the biological process. This relation may be known, or it may be determined after selection. A biological process may be covered by a single feature or multiple features. A feature may provide coverage for one biological process or multiple biological processes. Coverage may be further defined by level or direction of change that the feature experiences when different biological states are tested. Coverage may be relative to the impact of other omic data sets or features of different omic types. This difference may be tested for significance.

The number of biological processes that are covered by the features of a reduced feature classifier may be at least 1, at least 100, at least 500, at least 1000, at least 2000, at least 3000, at least 4000, at least 5000, at least 6000, at least 7000, at least 8000, at least 9000, at least 10,000, at least 15,000, at least 30,000, at least 45,000, at least 60,000, at least 75,000, or at least 100,000. The number of biological processes that are covered by the features of a reduced feature classifier may be greater than 1, greater than 100, greater than 500, greater than 1000, greater than 2000, greater than 3000, greater than 4000, greater than 5000, greater than 6000, greater than 7000, greater than 8000, greater than 9000, greater than 10,000, greater than 15,000, greater than 30,000, greater than 45,000, greater than 60,000, greater than 75,000, or greater than 100,000.

In some aspects, the biological processes may be gene ontology biological processes. The biological processes relationship to a feature may be determined by comparing the feature to a database. The database may be a Uniprot database. The relationship may be determined by laboratory testing. The relationship may be determined by theoretical biological interactions. The relationship may be hypothetical. The relationship may result from the statistical analysis of analytical testing.

In some aspects, the reduced feature classifier may be characterized by coverage of biological processes. The features of the classifier may be chosen to maximize this value. In some aspects, that means that individual features may be chosen to be part of the classifier above other features that may have higher selective ability. The higher coverage of biological processes may allow the reduced feature classifier to maintain selective power when used to classify a population different than the sample population upon which it was trained.

In some aspects, each different type of omic data may allow for the coverage of different biological processes. Some omic data sets may provide overlapping coverage of biological processes. The overlapping coverage of the omic data sets may interrogate different aspects of the same biological process. Features from different omic data sets but related to the same biological process may provide different coverage. In some aspects, a classifier with multi-omic data features may have greater sensitivity, specificity, or accuracy due to the multi-omic feature coverage of biological processes compared to a single omic classifier.

The relationship between a biological process and a feature may further comprise calculating a statistical significance for the relationship between the pair. The significance may be formal test of statistical significance. The p value for the relationship may be less than 0.15, less than 0.10, less than 0.05, less than 0.005, less than 0.001, or less in order to conclude a relationship exists. The significance may further comprise the use of a log Odds Ratio (LOR). The LOR may compare the relationship of the biological process to two or more omic groups. The LOR may indicate to which omics group the biological process is more related. It may indicate which feature represent a stronger relationship to a biological process. It may indicate a feature or omic group is better at detecting a change in or associated with a biological process. The LOR may be used in the calculation of coverage by a top feature classifier.

### Subject Monitoring and Treatment

In some cases, the subject is monitored. For example, information about a likelihood of the subject having a biological state such as cancer may be used to determine to monitor a subject without administering a treatment to the subject. In other circumstances, the subject may be monitored while receiving treatment to see if a cancer in the subject improves. In some aspects, the cancer described herein is pancreatic cancer. The methods described herein may include recommending or administering a pancreatic cancer treatment for the subject when the proteomic data is classified as indicative of pancreatic cancer. In certain aspects, the method recommends administering a pancreatic cancer treatment to the subject when the proteomic data is classified as indicative of pancreatic cancer. In certain aspects, the method recommends performing a biopsy or pancreatoscopy when the proteomic data is classified as indicative of pancreatic cancer. In certain aspects, the method recommends observation of the subject without administering a pancreatic cancer treatment to the subject. In certain aspects, the method recommends observation of the subject without obtaining a biopsy or pancreatoscopy of the subject, when the proteomic data is not classified as indicative of pancreatic cancer. In certain aspects, the method recommends observing the subject without administering a pancreatic cancer treatment to the subject. In certain aspects, the method recommends observing the subject without obtaining a biopsy or pancreatoscopy of the subject, when the proteomic data is not classified as indicative of pancreatic cancer. The decision to treat the subject, or to obtain a biopsy or not, may be based on whether the proteomic data is indicative of a mass in the subject's pancreas (e.g., a pancreatic cyst) being cancerous or not. For example, a physician may find a pancreatic cyst by CT scanning, and then order a blood test that involves a method described herein.

When the subject is identified as not having the cancer, the subject may avoid an otherwise unfavorable cancer treatment (and associated side effects of the cancer treatment), or is able to avoid having to be biopsied or tested invasively for the cancer. When the subject is identified as not having the cancer, the subject may be monitored without receiving a treatment. When the subject is identified as not having the cancer, the subject may be monitored without receiving a biopsy. In some cases, the subject identified as not having the cancer may be treated with palliative care such as a pharmaceutical composition for pain. In some cases, the subject is identified as having another disease different from the initially suspected cancer and is provided treatment for the other disease.

When the subject is identified as having the cancer, the subject may be provided a treatment for the cancer. For example, if the cancer is pancreatic cancer, the subject may be provided a pancreatic cancer treatment. Examples of treatments include surgery, organ transplantation, pharmaceutical composition administration, radiation therapy, chemotherapy, immunotherapy, hormone therapy, monoclonal antibody treatment, stem cell transplantation, gene therapy, or chimeric antigen receptor (CAR)-T cell or transgenic T cell administration. In certain aspects, the cancer is pancreatic cancer, and the pancreatic cancer treatment comprises chemotherapy, radiation therapy, immunotherapy, targeted therapy, surgery, or surgical resection, or a combination thereof. In certain aspects, the method recommends pancreatic cancer treatment comprising administration of a pharmaceutical composition comprising capecitabine, erlotinib, fluorouracil, gemcitabine, irinotecan, leucovorin, nab-paclitaxel, nanoliposomal irinotecan, oxaliplatin, olaparib, or larotrectinib, or a combination thereof.

When the subject is identified as having the cancer, the subject may be further evaluated for the cancer. For example, a subject suspected of having the cancer may be subjected to a biopsy after a method disclosed herein indicates that he or she may have the cancer.

Some cases include recommending a treatment or monitoring of the subject. For example, a medical practitioner may receive a report generated by a method described herein. The report may indicate a likelihood of the subject having a cancer. The medical practitioner may then provide or recommend the treatment or monitoring to the subject or to another medical practitioner. Some cases include recommending a treatment for the subject. Some cases include recommending monitoring of the subject.

In some aspects, when the cancer evaluation method indicates that the subject has a probability exceeding a predetermined threshold of having pancreatic cancer, the method further comprises conducting a subsequent pancreatic cancer treatment, or advising the subject to undergo a subsequent pancreatic cancer treatment, to determine the presence of pancreatic cancer. In some aspects, the subsequent pancreatic cancer treatment comprises a biopsy. In some aspects, the subsequent pancreatic cancer treatment comprises pancreatic imaging. In some aspects, the imaging is performed using an ultrasound or computed tomography. In some aspects, when the cancer evaluation method indicates that the subject has a probability exceeding a predetermined threshold of having a pancreatic cancer, the method further comprises treating the subject with a pancreatic cancer treatment for treating the pancreatic cancer or advising the subject to undergo such pancreatic cancer treatment In some aspects, the therapy is selected from the group consisting of: surgery for pancreatic cancer, radiation therapy for pancreatic cancer, cryotherapy for pancreatic cancer, hormone therapy for pancreatic cancer, chemotherapy for pancreatic cancer, ablative treatments for pancreatic cancer, and immunotherapy for pancreatic cancer. In some aspects, the predetermined threshold is greater than 10%, 20%, 30%, 40%, 50%, 60%, 70%, 80%, or 90%.

### EXAMPLES

The following illustrative examples are representative of embodiments of the stimulation, systems, and methods described herein and are not meant to be limiting in any way.

### Example 1. Identifying a Likelihood of Pancreatic Cancer in a Subject

A subject comes into a doctor's office having jaundice and abdominal pain. The doctor determines that the subject may be at risk of having cancer, and performs a non-invasive work-up, including a CT scan but nothing of note is detected. A plasma sample is obtained from the patient to be analyzed by the methods described herein. The lab measures the presence and abundance of several proteins. The lab then applies a classifier to generate an output report to the physician for determining whether the subject has pancreatic cancer. The report indicates that the patient likely has pancreatic cancer. It's possible that the pancreatic cancer is small and developing at an early stage, which explains why the scan did not detect the pancreatic cancer. The physician asks the patient to return for a regular check-up once every 6 months in order to continue monitoring the pancreatic cancer. During one of the subsequent check-ups, the analysis of the biofluid sample obtained from the subject indicates that the pancreatic cancer has progressed. The physician then prescribes or administers a pancreatic cancer treatment regimen.

### Example 2. Deep, Unbiased Multi-Omics Approach for Identification of Pancreatic Cancer Biomarkers from Blood

Pancreatic cancer is the seventh leading cause of cancer related death worldwide and the third leading cause of cancer related death in the USA. The low survival rate of pancreatic cancer is often due to challenges relating to the early detection of the disease, highlighting the need for early diagnostic test development. While cancer signatures are less challenging to identify at the localized pancreatic tumor via biopsy, cancer signals found in the blood stream due to cellular leakage, metastasis, signaling, or innate immune response may also be useful due to reduced invasive sampling.

Challenges encountered in liquid biopsy cancer biomarker discovery studies have included analyte degradation and dilution in a complex biological matrix, which limit high specificity and sensitivity measurements. To overcome these challenges, a comprehensive multi-omics platform was developed that facilitates uncovering previously untapped information to gain a more holistic biological perspective at unprecedented depths and integrate molecular signatures across complex levels of biology. Implementation of this approach has led to the discovery of new pancreatic cancer specific biomarkers and a deeper understanding of the integrated pathways of pancreatic cancer.

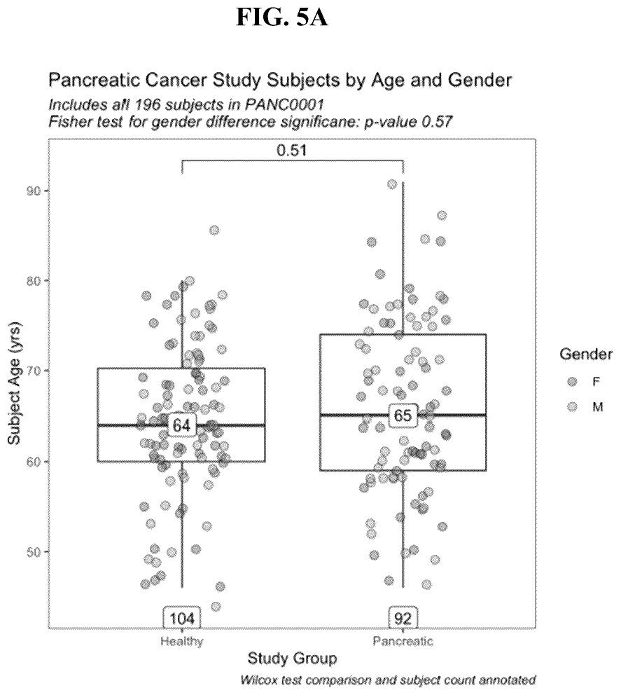

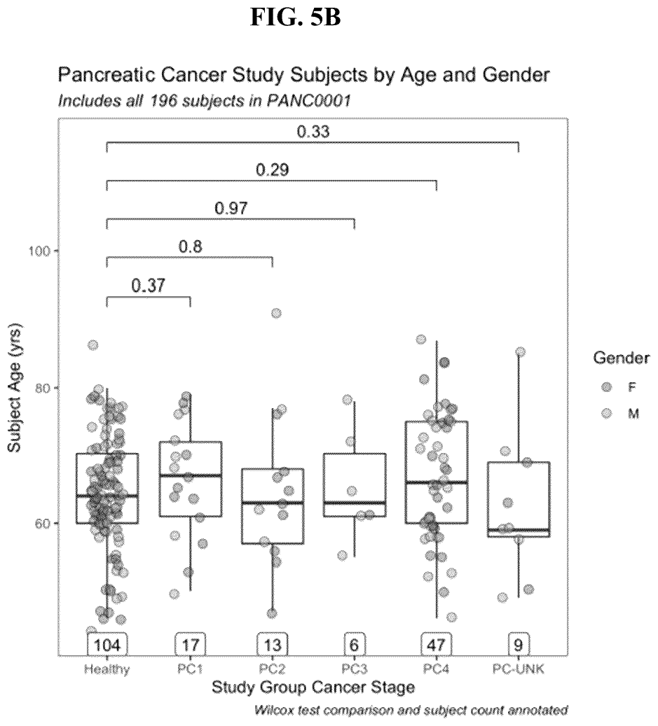

In this case-control study, plasma proteomic, metabolomic, and lipidomic data were collected from 196 human plasma samples. The samples included plasma from 92 patients with pancreatic cancer (“cancer samples” or “PC”), and plasma from 104 healthy subjects without cancer (“healthy controls”). Specifically, the pancreatic cancer included pancreatic adenocarcinoma. The cancer patients were age- and gender-matched with the healthy subjects (Table 1, FIGS. 5A-5B). In some tables and figures herein, samples from healthy subjects without cancer are referred to as “healthy,” and samples from subjects with pancreatic cancer are referred to as “pancreatic.” The cancer samples were from patients with a variety of stages of pancreatic cancer and included 9 samples from subjects with an undefined cancer stage (“unknown”). No bias was observed based on age or gender comparisons between classes.

TABLE 1
196 subjects

| Gender | Healthy | Pancreatic |
|---|---|---|
| F | 53 | 43 |
| M | 51 | 49 |

The data were obtained using liquid chromatography-mass spectrometry (LC-MS). Samples from the subjects with cancer were collected after diagnosis and before treatment of the pancreatic cancer. Data from the cancer samples were compared to the healthy controls. Sample collection and handling was observed to be the same for all samples.

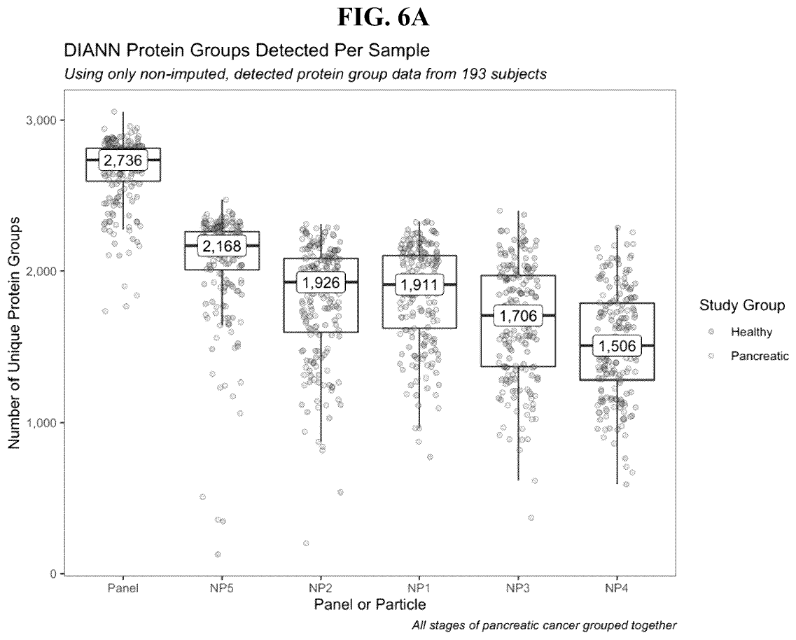

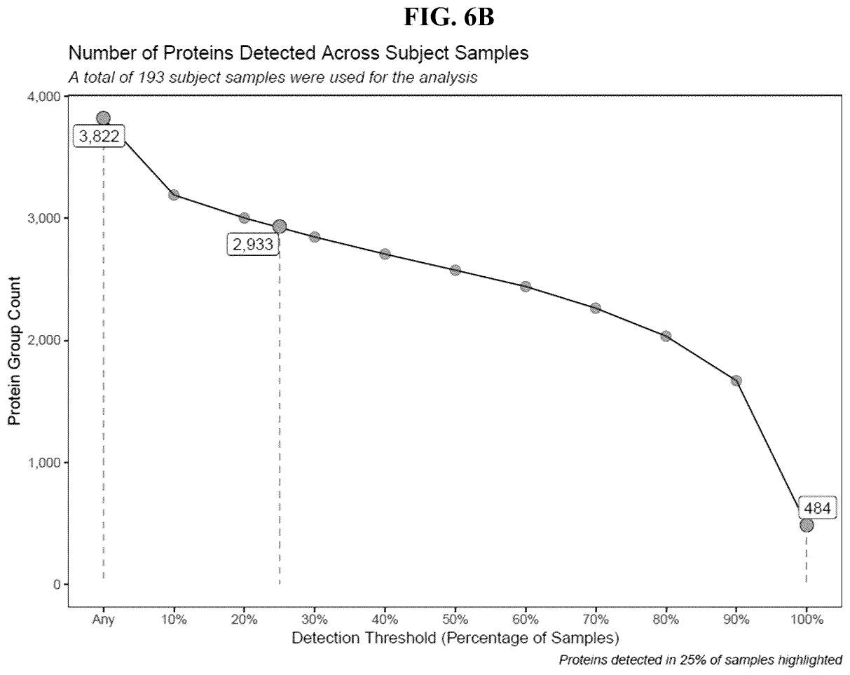

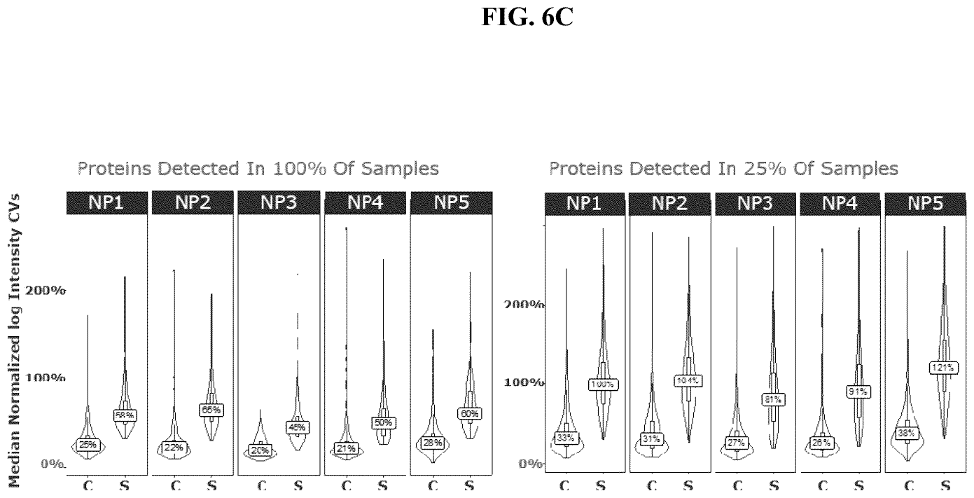

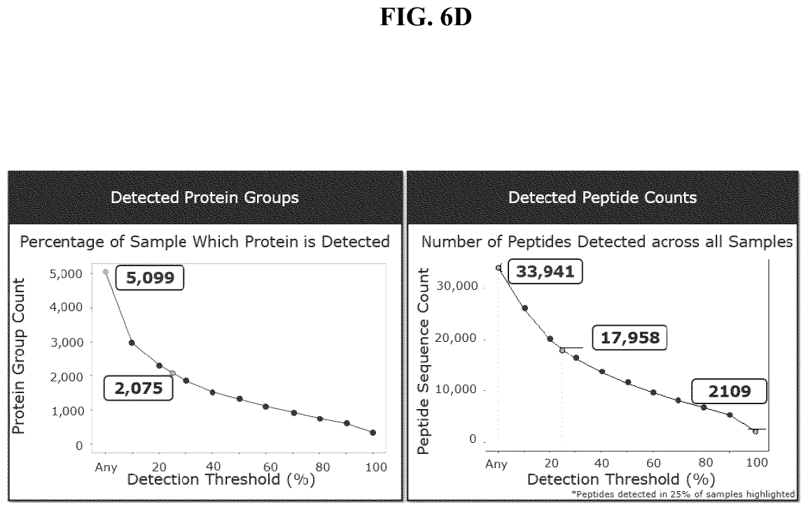

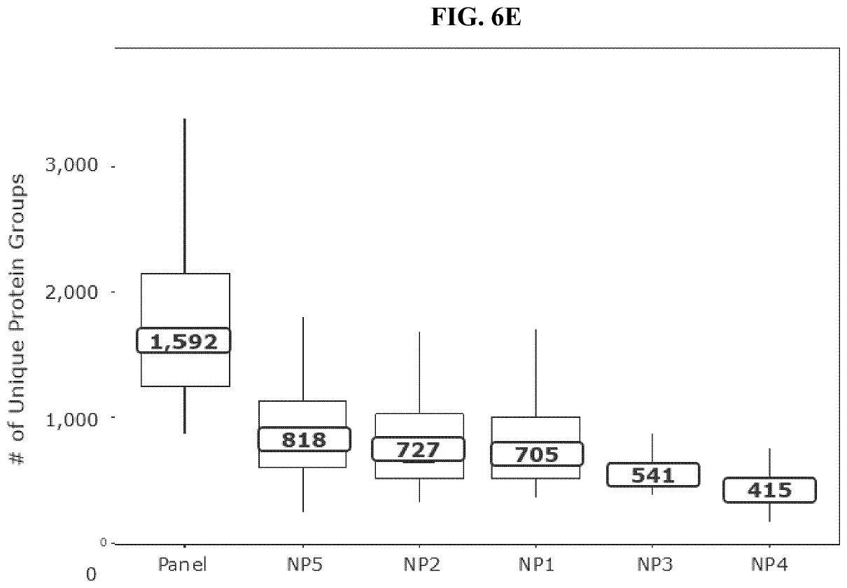

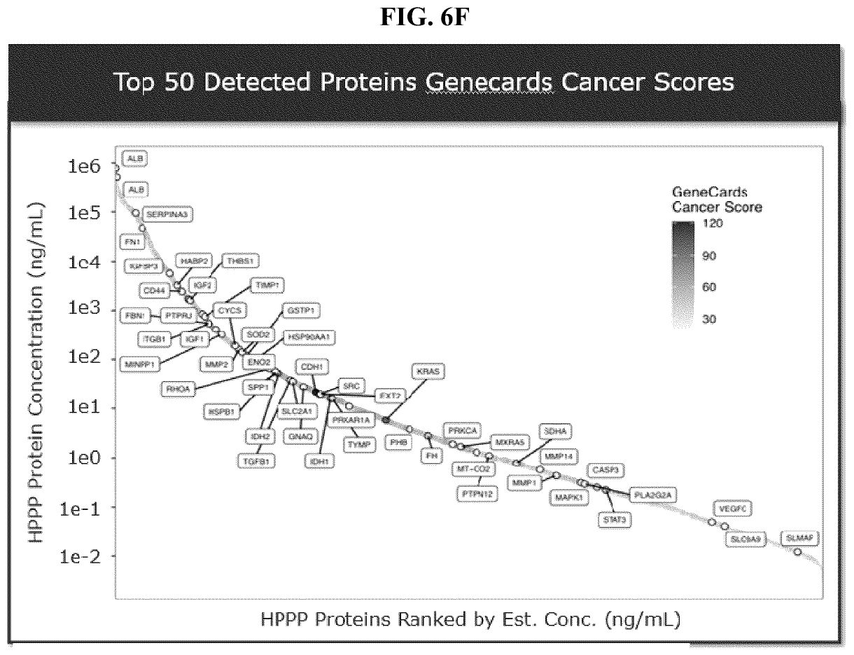

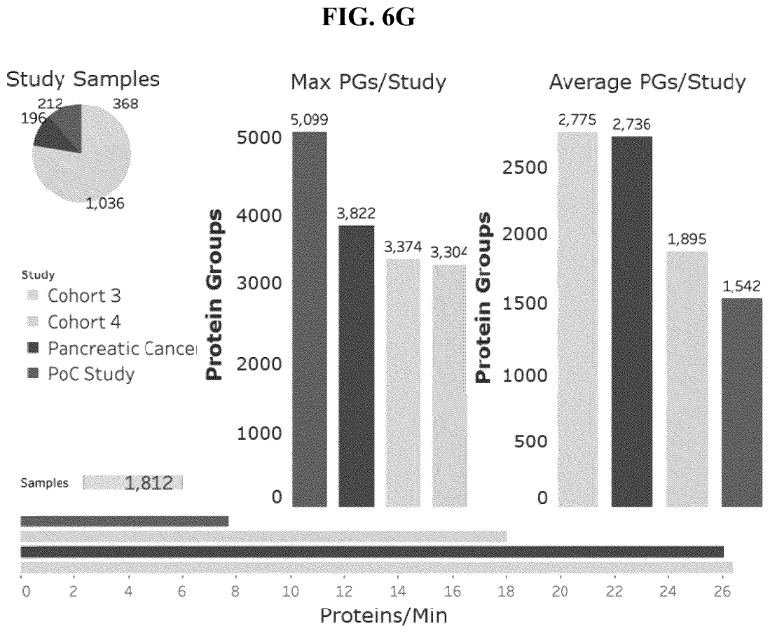

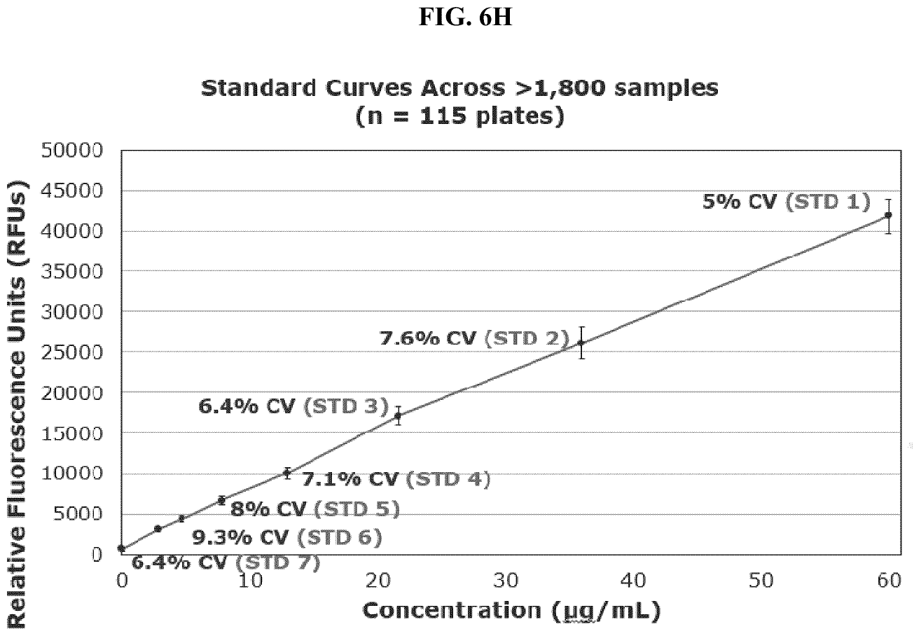

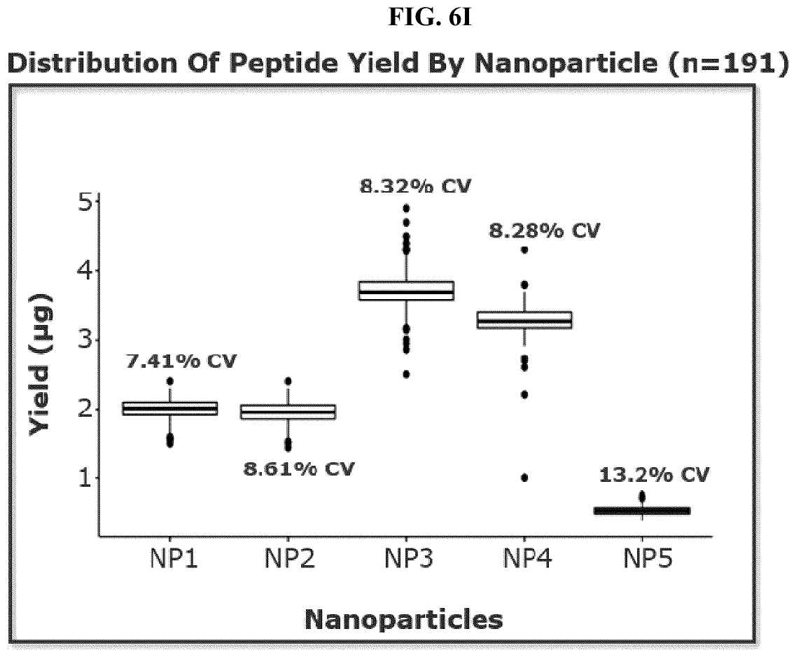

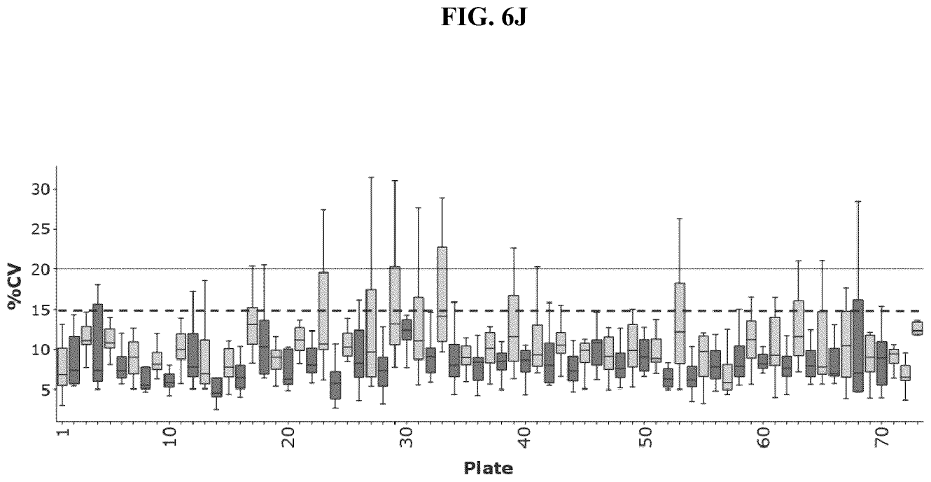

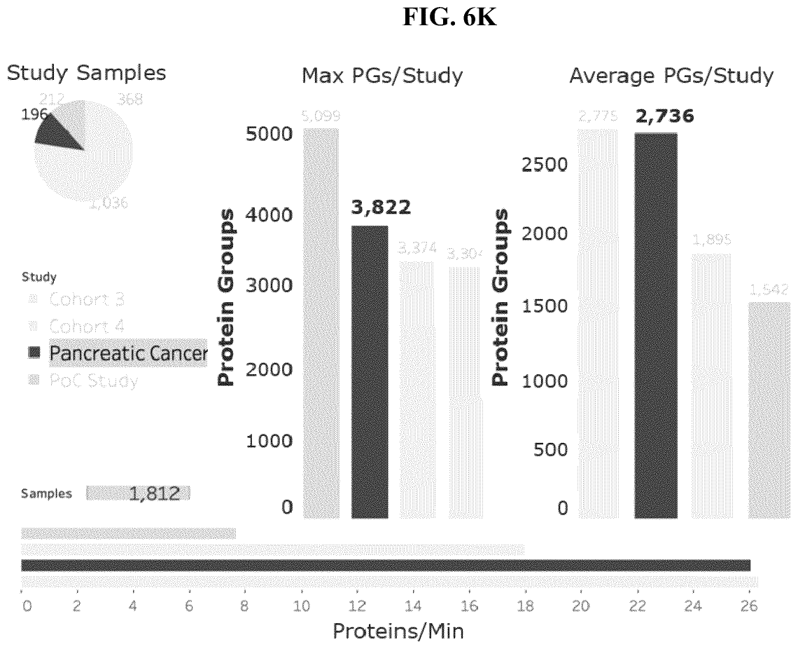

Proteins were measured separately by two methods. One protein measurement method (referred to herein as “Proteograph”) included the use of particles, where plasma samples were contacted individually with particles to adsorb proteins from the plasma onto a corona around each particle. Proteins adsorbed to the particles were then assessed by liquid chromatography-mass spectrometry (LC-MS). Proteomic data were obtained from the use of 5 physiochemically distinct particle types (designated “NP1,” “NP2,” “NP3,” “NP4,” and “NP5”). Data from the nanoparticles were analyzed separately, as well as in a combined panel. These particles were purchased commercially from Seer, Inc. where they were identified as 5-003, 5-006, 5-007, P-039, and P-073, respectively. FIGS. 6A-6B show total numbers of proteins observed by Proteograph per sample. Here, MAXLFQ processing of DIANN report data was used.

The second protein measurement method included the use of known amounts of isotopically labeled, internal reference proteins (referred to herein as “PiQuant”). The internal reference proteins were spiked into each plasma sample, then used to identify mass spectra of individual endogenous proteins, and further used as standards for determining amounts of the individual endogenous proteins.

In the analysis, 3,381 proteins were detected in all samples (where a protein was detected in a minimum of 3 samples). Using a Bonferroni correction (FDR=0.05), 124 proteins were measured at statistically significant levels in the cancer samples compared to the healthy controls. The data also included ˜200 lipids out of 678 total lipids and 49 of 299 metabolites present in all samples (minimum of 3 samples per class) that were determined to be at statistically significant differential levels (using a Bonferroni correction; FDR=0.05). The detected analytes (proteins, lipids and metabolites) included analytes that were previously unassociated with pancreatic cancer. Additional analyses will be performed to further integrate the multi-omics datasets and determine multivariate statistical performance to detect pancreatic cancer.

Proteins were detected through a full range of a plasma proteome, including a significant number of high OpenTargets (OT)-scoring proteins for pancreatic carcinoma. Table 2 shows some aspects of 2,933 proteins total, where about 50% mapped to HPPP. Table 3 shows aspects of 10 proteins (out of 213 that had an OT score of 0.15 or greater) that had the highest OT scores. FIG. 7A shows some data that included mapping to 3,486 proteins in the HPPP database, and includes estimated ng per mL concentrations. The proteins in FIG. 7A include MYH9, TUBB1, TUBB, CALR, FLT4, NOTCH2, RHOA, IDH2, CDH1, PRKAR1A, NOTCH1, EXT1, PPP2R1A, SND1, BTK, LPP, MAPK1, FAT1, CDH11, and MAP2K1. FIG. 7B shows a pancreatic carcinoma OT score distribution, where an arbitrary threshold (0.15) for significance is included and was based on inspection of distribution.

TABLE 2

| N | High OT | HPPP |
|---|---|---|
| 1436 | FALSE | FALSE |
| 1337 | FALSE | TRUE |
| 50 | TRUE | FALSE |
| 110 | TRUE | TRUE |

TABLE 3

| Gene ID | OT Score |
|---|---|
| GNAS | 0.67 |
| EGFR | 0.65 |
| TUBB4B | 0.61 |
| RRM1 | 0.60 |
| TUBB1 | 0.58 |
| TUBB6 | 0.58 |
| TUBB8 | 0.58 |
| TUBB | 0.58 |
| SMAD3 | 0.55 |
| MAPK1 | 0.52 |

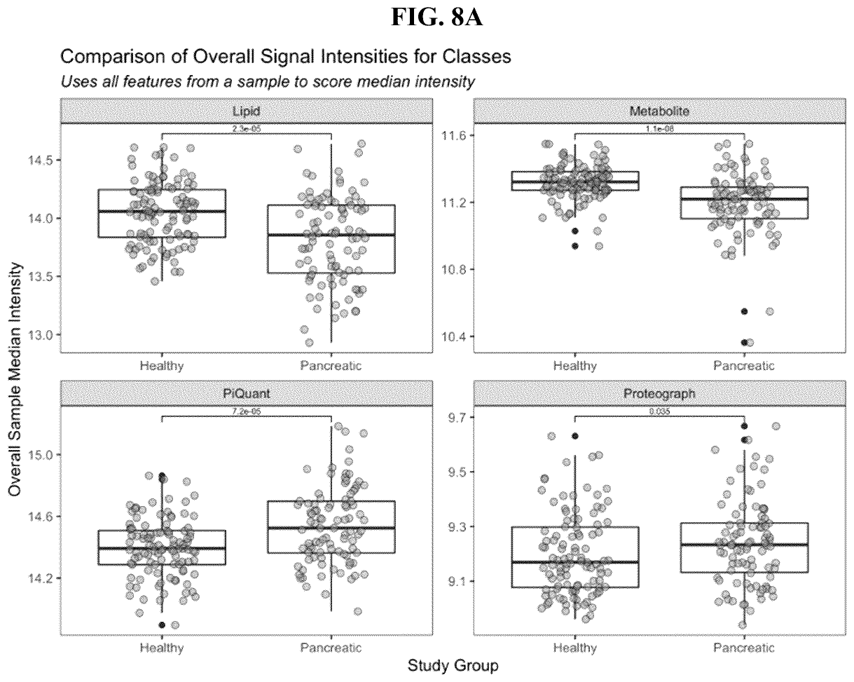

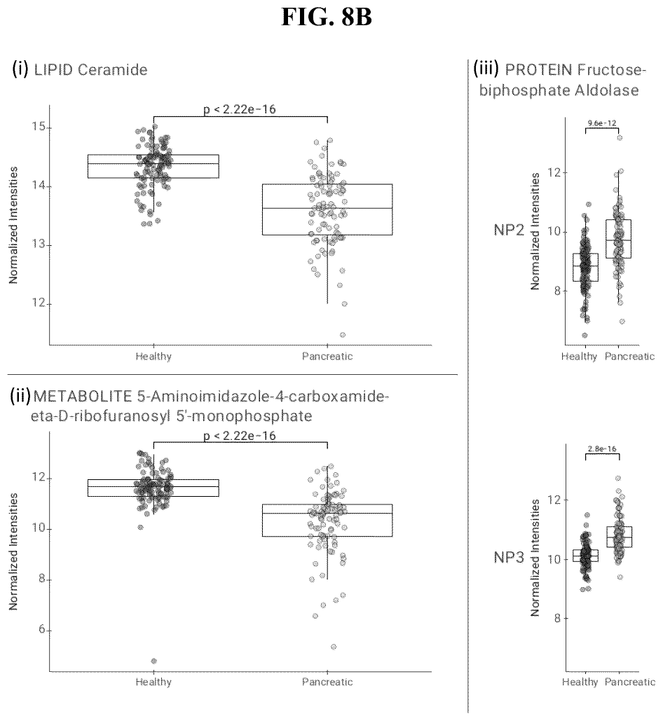

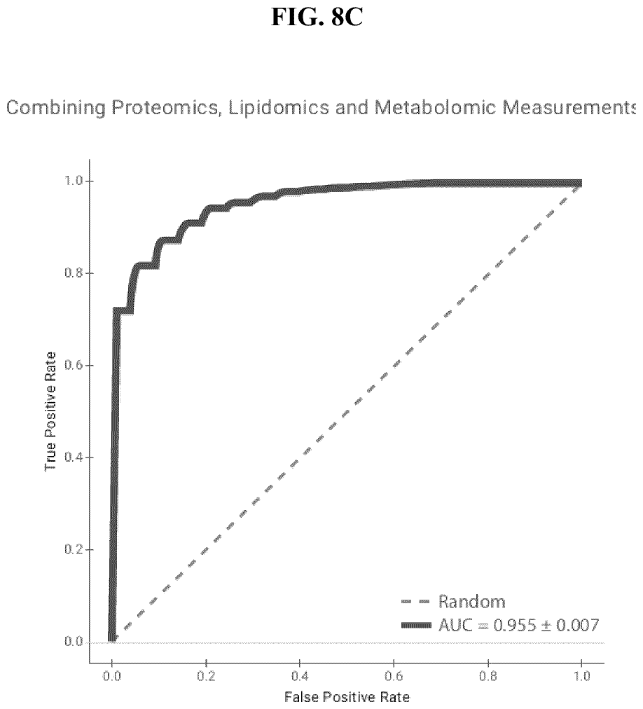

FIG. 8A shows a comparison of gross signal medians by sample, analyte-type, and class, where large-scale differences may be observed with targeted methods.

FIG. 8B shows box and whisker plots of most significantly different analytes per omics workflow ((i): lipid; (ii): metabolite; and (iii): Protein). Box and whisker plots of the most significantly different analytes in each of the omic classes were investigated. The most significantly different lipid was ceramide. The most significantly different metabolite was 5-aminoimidazole-4-carboxamide-1-beta-D-ribofuranosyl 5′-monophosphate (AICAR). The most significantly different protein, fructose-biphosphate aldolase, was significantly different in two of the five nanoparticle (NP) samples. This highlights the power of the Proteograph assay, which utilized five unique individual NP chemistries that provides complementary protein identifications.

FIG. 8C shows an exemplary multimers classifier performance combining proteomics, lipidomics, and metabolomics measurements. The model was trained with all available samples where cancer stage was known. Then, performance was assessed on each individual or groups of stages. Five-fold cross validation was performed and repeated 30 times. The average AUC was computed across 150 runs. Random forest algorithm was used for proteomics data, and logistic regression was used for metabolomics and lipidomics data.

FIGS. 9A and 9B include results from non-parametric (Wilcox) study group univariate comparisons (EDA) for Proteograph data, using any analyte present in >2 samples per class, and with Bonferroni multiple-testing correction. FIGS. 9C and 9D include results from non-parametric (Wilcox) study group univariate comparisons (EDA) for PiQuant data, using any analyte present in >2 samples per class, and with Bonferroni multiple-testing correction. FIGS. 10A and 10B include results from non-parametric (Wilcox) study group univariate comparisons (EDA) for lipid data, using any analyte present in >2 samples per class, and with Bonferroni multiple-testing correction. FIGS. 11A and 11B include results from non-parametric (Wilcox) study group univariate comparisons (EDA) for metabolite data, using any analyte present in >2 samples per class, and with Bonferroni multiple-testing correction.

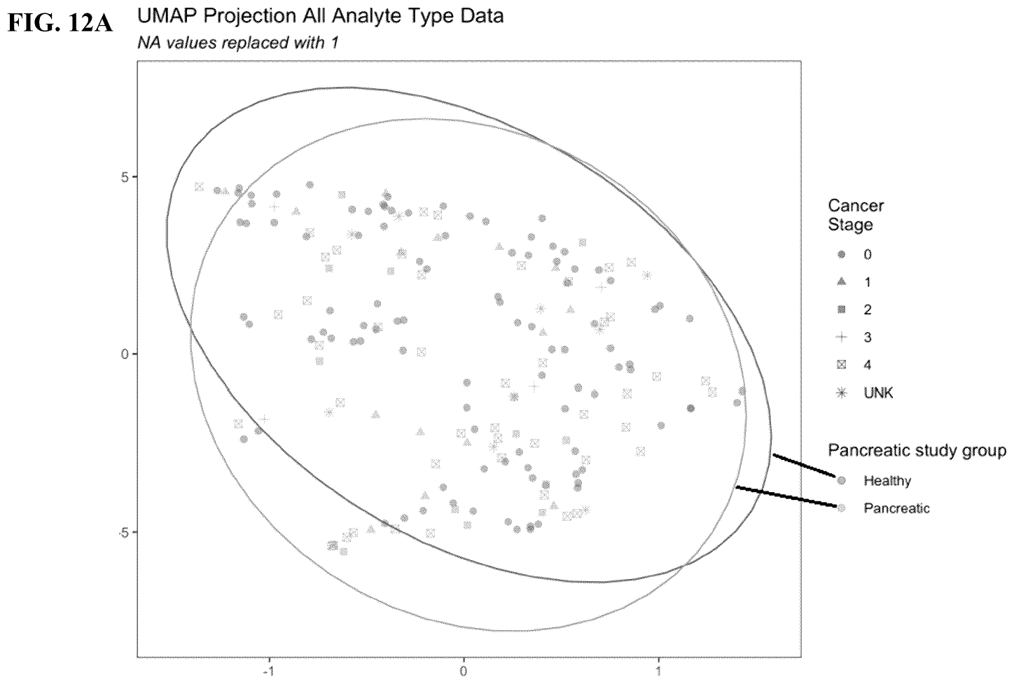

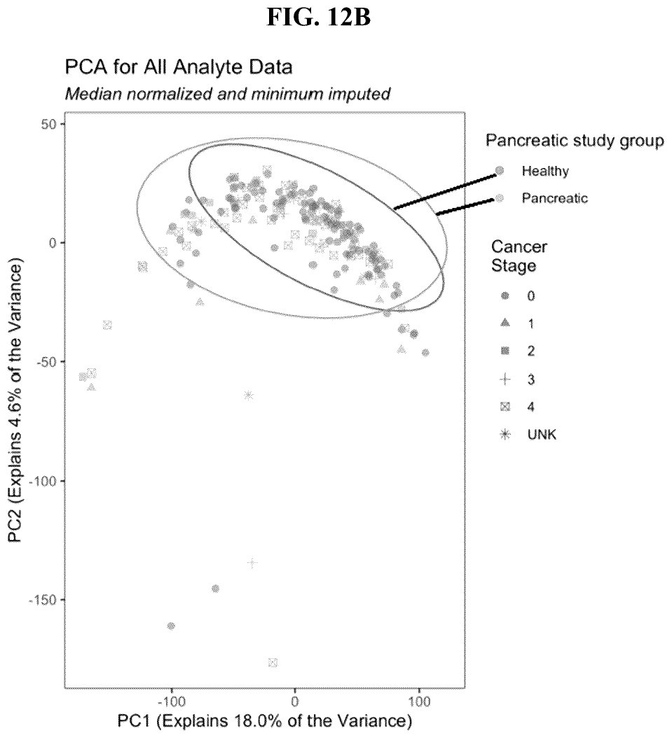

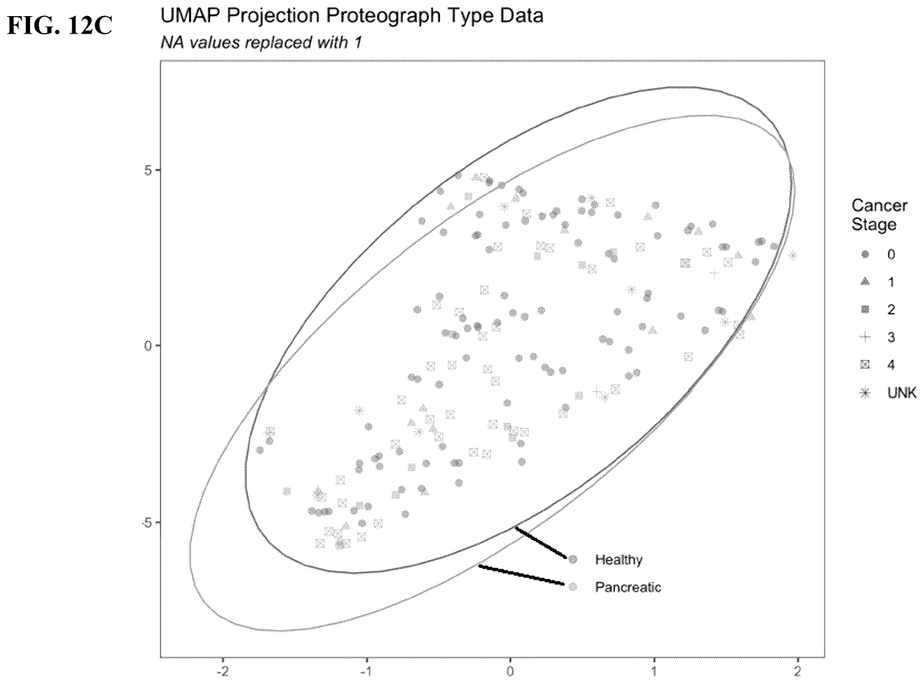

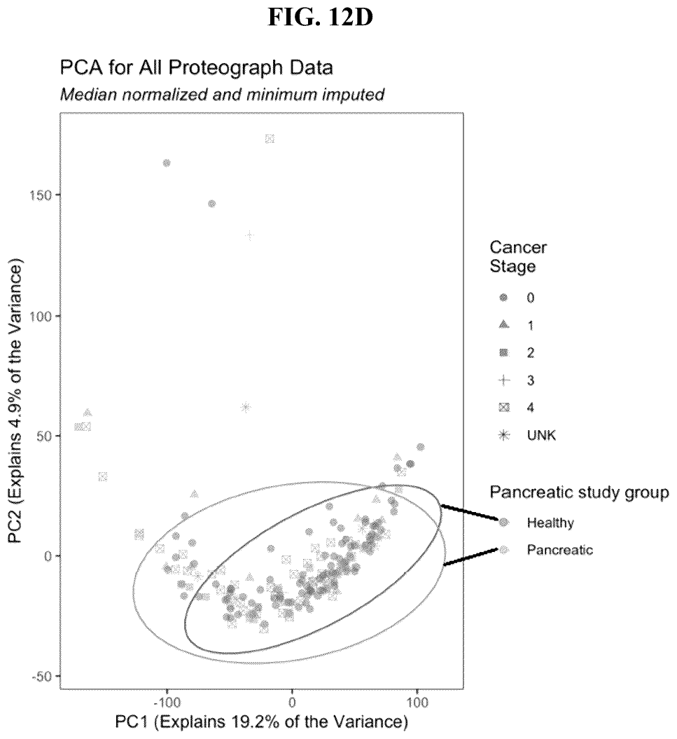

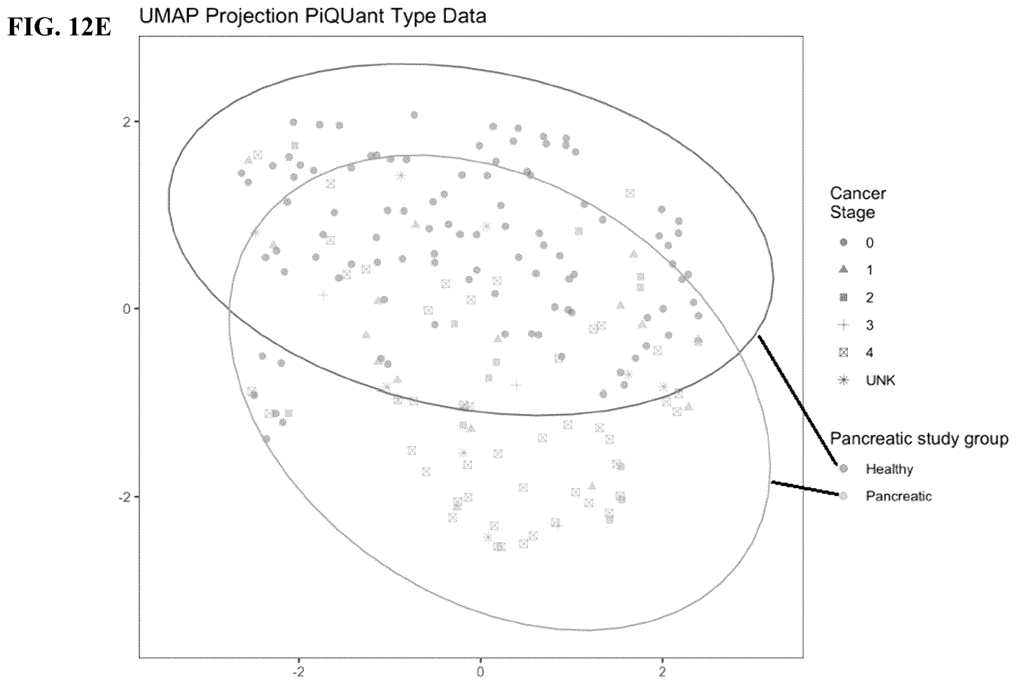

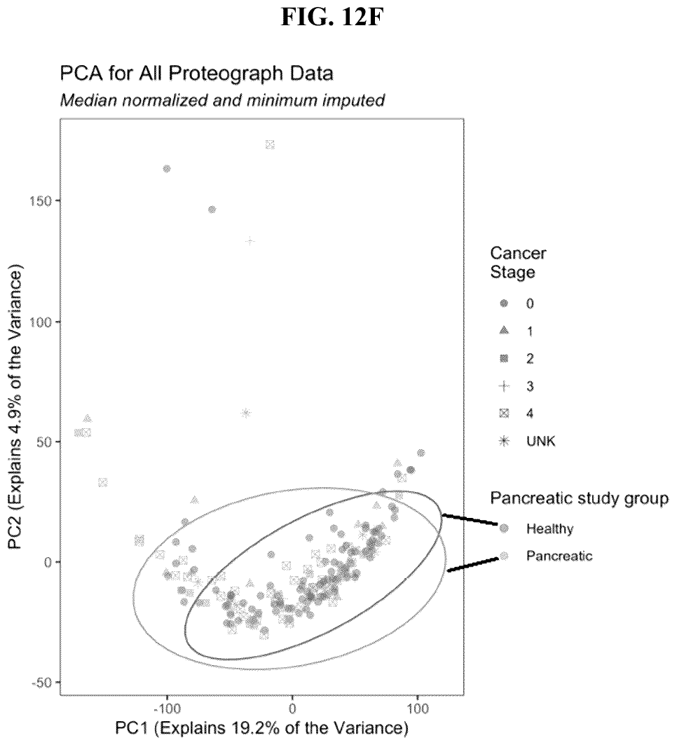

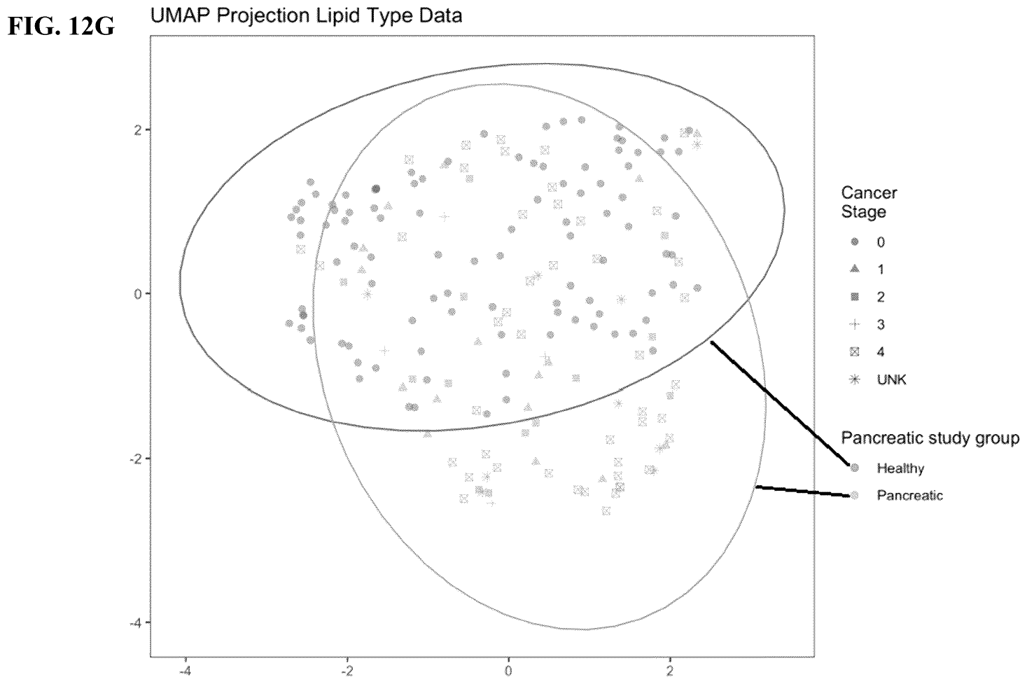

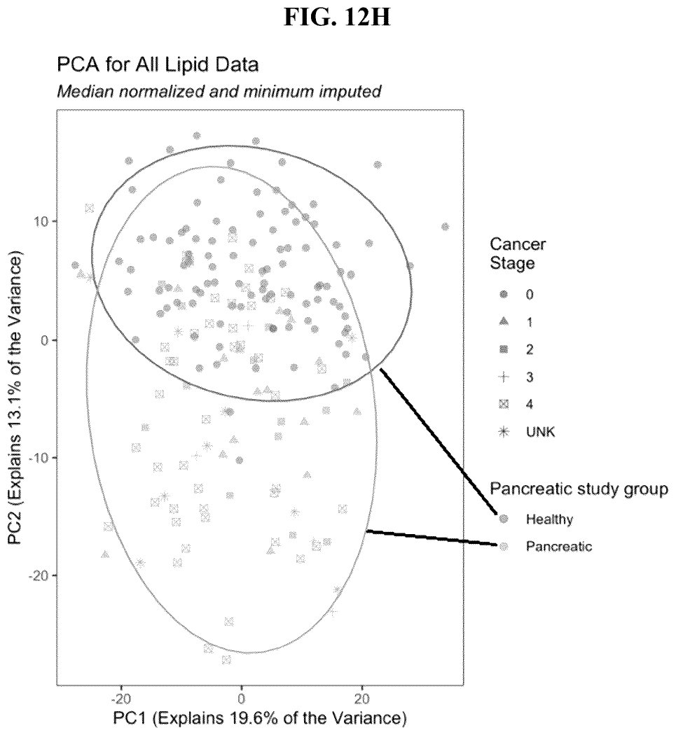

Initial multi-variate class separations were performed using analyte-complete samples, based on parametric (PCA) and non-parametric (UMAP) projections. Separation data are shown in FIGS. 12A-12J. In particular, FIGS. 12A-12B are based on combined data (Proteograph, PiQuant, lipid, and metabolite data), FIGS. 12C-12D are based on Proteograph data, FIGS. 12E-12F are based on PiQuant data, FIGS. 12G-12H are based on lipid data, and FIGS. 121-12J are based on metabolite data. In FIGS. 12C-12D, missing values were replaced with an arbitrary minimum value.

The intent of this study was to detect a biological signal for pancreatic cancer in non-invasively collected liquid samples. This analysis indicates that there are significant differences between classes in the samples as collected, and that they may be useful in detecting pancreatic cancer. Further experiments will combine additional features within and across analyte classes to further improve cancer detection. For example, additional proteomic and transcriptomic data will be included in this analysis, including methylation, mRNA, and miRNA data.

### Example 3. Multi-Variate Machine Learning Using Gradient Boosted Trees

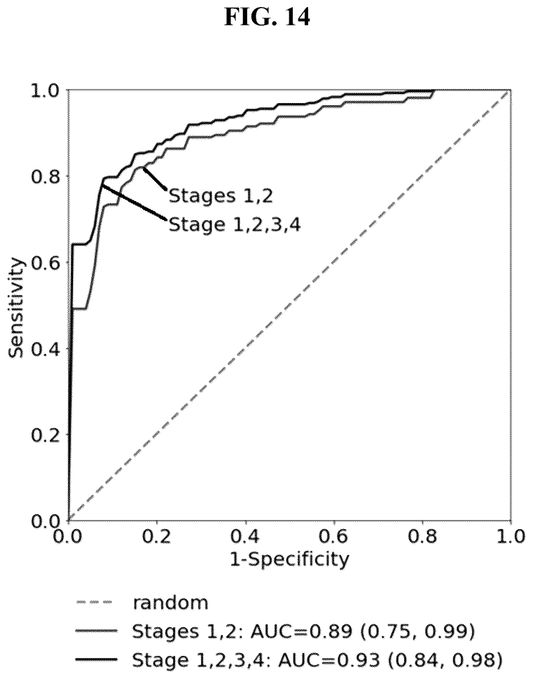

A training subset of the study was used in initial cross-validation analyses using XGBoost. In-transformation and median normalization of all intensity data was performed for 189 feature-complete cases from the proteomic, lipidomic, and metabolomic data generated in Example 2. The proteomic data included Proteograph and PiQuant data. Analytes were filtered to those present in at least 25% of the study samples. The 189 complete subjects were split into a training set (n=141) and a held-out validation set (n=48). The training set was used to select hyperparameters for XGBoost-modeling via five rounds of 5-fold cross-validation, with 112-114 for training and 29-27 for testing in each fold. FIG. 13 shows some top features in the training set, where “LPD” refers to a lipid, “MTB” indicates a metabolite, “PQ” refers to protein as assessed by PiQuant methodology, and “PG” refers to protein as assessed by Proteograph methodology. The PQ and PG proteins are included as UniProt reference numbers. Receiver operating characteristic (ROC) curves were generated, and results showed that the combined classifier had an area under the curve (AUC) of 0.924±0.012 (std. err., n=25) when differentiating pancreatic cancer at any stage from non-cancer, or an AUC of 0.89 for identifying early-stage pancreatic cancer (here, stage 1 or 2) (FIG. 14). An additional model can be built on the training data with selected parameters and validated on the n=48 validation set.

In this example, a combined classifier was trained on data from mass spectrometry-based assays, including protein, metabolite, and lipid data. The combined classifier may be used to detect pancreatic cancer. Similar classifiers may be trained from samples of subjects having other diseases or cancers and used to detect the other diseases or cancers.

### Example 4. Analyses of Multiple Blood-Based Genomic Assays in Pancreatic Cancer

Pancreatic cancer is the third leading cause of cancer-related deaths in the United States. While the 5-year survival rate across all stages is only 10%, in early stages when the disease is localized, the survival rate may reach 40%. Detecting early pancreatic cancer thus helps to reduce mortality; however, most diagnoses are made at stage IV, after onset of clinically detectable symptoms. Hence, there is a need to prioritize between individuals for further testing using minimally invasive procedures, such as liquid biopsies.

A case-control, proof-of-concept study was conducted using 69 subjects: 36 pathology confirmed, treatment naïve cases (5 stage I, 5 stage II, 2 stage III, 22 stage IV, and 2 unknown stages of pancreatic cancer) and 33 demographically matched controls without any pancreatic disease.

For each subject, up to 50 mL of blood was collected in assay-specific tubes. Cell-free DNA as well as mRNA and miRNA from white blood cells were isolated from these samples and assayed following standard NGS protocols. Measurements on CpG methylations, mRNA, and miRNA transcript abundances were then collected. These measurements together may be collectively referred to as genomics assays. Univariate differential analyses of cases versus controls were performed.

The genomic measurements were collected, including CpG methylations and mRNA, and miRNA transcript for cancer and non-cancer subjects. The methylation percentage on CpG sites that covered at least 11 reads was considered. Also, log-transformed counts on canonical mRNA transcripts and miRNA transcripts were used. Then data was split into a training set and a hold-out set. Next, a model on each dataset (omic) was built to differentiate between cancer and non-cancer subjects by training an ensemble classifier on the training data. Each classifier was trained using 30 repeats of 5-fold nested cross-validation with hyperparameter tuning. The domain of the hyperparameters for the classifier was divided into a discrete grid. Then, every combination of the grid values was tried, calculating the performance metrics in the nested cross-validation, and average performance across all runs for each dataset was reported. Eventually, a final performance for all three omics was reported by averaging the predictions of each one. The hyperparameters selected during the search were then used to configure a final model, and the final model was fitted on the entire training dataset for each omic. Then, each model was used to make predictions on the hold-out dataset. A final prediction on the hold-out dataset was computed by averaging the predictions on the hold-out dataset across all omics.

Generally, the final classifier included a random-forest-based classifier trained on the CpG methylations, mRNA, and miRNA data to differentiate between pancreatic cancer cases and noncancer controls. This classifier may be referred to as a genomics classifier.

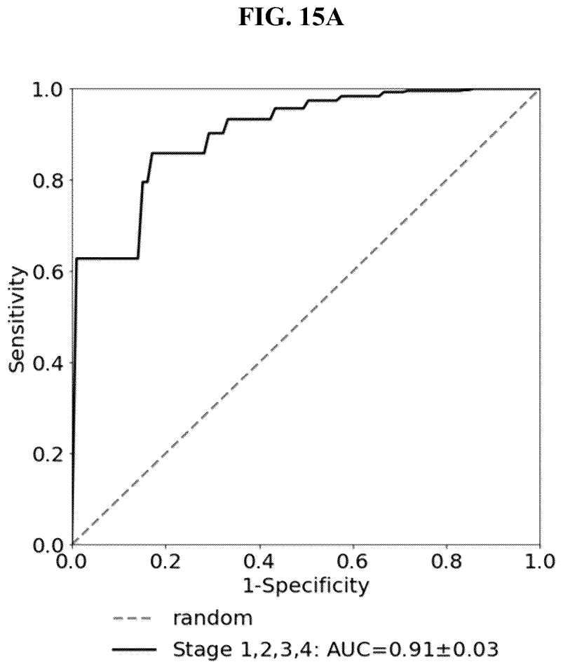

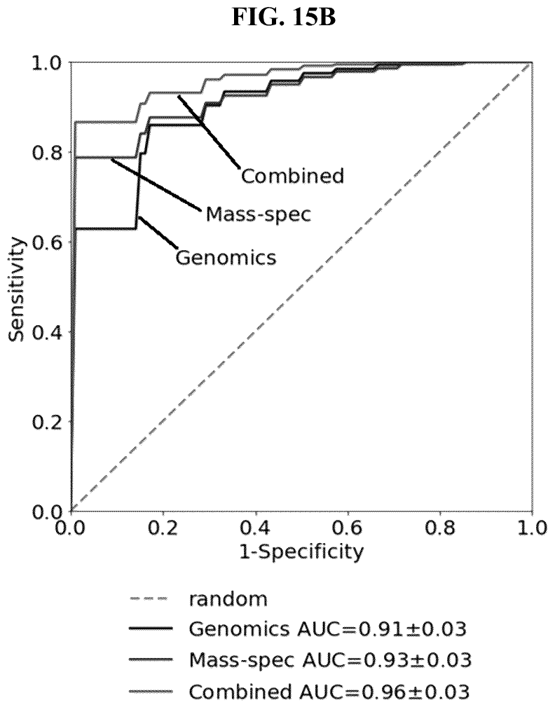

Overall, log-transformed counts on 18045 canonical mRNA transcripts and 1035 miRNA transcripts, as well as percentage methylation on 9290 CpG sites (filtered by adequate read coverage) were used. Univariate analyses identified 8769 mRNAs, 204 miRNAs, and 3128 CpG sites that were significantly differentially expressed (or methylated) at a Benjamini-Hochberg FDR <0.05, including both novel and known biomarkers associated with pancreatic cancer. A majority of these mRNAs were less abundant in cases compared to controls while the opposite was true of the miRNAs. CpG site methylations were generally more balanced but were nonetheless more likely unmethylated in cases compared to controls. The random-forest-based genomics classifier was trained using 30 repeats of 5-fold nested cross-validation with hyperparameter tuning. Across all repeats, mean sensitivities of 46% (95% CI, 20%-72%) were observed for stage 1, 2, 3, 72% (95% CI, 59%-85%) for stage 4, and 64% (95% CI, 52%-76%) for all stages at a specificity of 92%. Data for the genomics classifier are shown in FIG. 15A.

In this initial study on pancreatic cancer using multi-omics readouts from a liquid biopsy, substantial numbers of dysregulated mRNA and miRNA transcripts were observed, which may reflect cancer-associated changes to the immune system. The most discriminative transcripts included novel biomarkers as well as genes under investigation as therapeutic targets in multiple cancers. Machine learning modeling additionally yielded a classifier whose cross-validation performance highlights the potential of multi-omics towards both disease diagnosis as well as novel target discovery.

### Example 5. Analyses of Multiple Blood-Based Mass Spectrometry and Genomic Assays in Pancreatic Cancer

Plasma samples from the subjects described in Example 4 were also analyzed using mass spectrometry-based omics assays, including protein (Proteograph and PiQuant), lipid, and metabolite assays. A classifier was trained using these mass spectrometry-based omics assays, which may be referred to as a mass spec classifier. A combined classifier was trained using both the mass spectrometry-based omics assays in this example, and the genomics assays in Example 4. The mass spec and combined classifiers were trained and tested similarly to the genomics classifier of Example 4 but using the different or additional data types (including mass spectrometry assays).

Performance of the mass spec classifier of this example, the genomics classifier of Example 4, and the combined classifier of this example, were all compared. Data are shown in FIG. 15B. Based on classifier performance, the mass spec assays and genomics assays appear to provide complementary information such that the performance of the combined classifier was better than those of the component ones.

### Example 6. Unbiased Multi-Omics Approach for the Detection of Pancreatic Cancer Biomarkers Utilizing Ion-Mobility Mass Spectrometry and Nanoparticle Based Proteograph Technology

Pancreatic cancer is the seventh leading cause of cancer-related death worldwide and the third leading cause of cancer-related death in the USA. Challenges in early detection have led to poor survival rates, highlighting the need for early diagnostic test development. Biomarkers measured in liquid biopsies offer a less invasive and accessible strategy for early cancer detection. Analyte degradation and dilution in complex biological matrix limit high specificity and sensitivity measurements, making biomarker discovery from blood a formidable challenge.

A comprehensive multi-omics platform has been developed that integrates multiple analyte measurements, cutting-edge analytical instrumentation, and novel data-analysis approaches. To demonstrate this platform's power, an unbiased multi-omics study of a pancreatic cancer cohort of 196 subjects was conducted, resulting in the detection of novel biological signals. The study included the same samples and protein data as were used in Example 2. However, this study utilized a different approach for generating lipid data.

The study cohort comprised 196 human subjects. Out of the 196 subjects, 92 had pancreatic cancer and 104 were healthy. Subject samples were collected post-diagnosis, but pre-treatment for cancer subjects versus healthy controls. Plasma samples were processed for proteomics on the nanoparticle based Proteograph platform (Seer Inc.). Resulting peptides were analyzed by LC-MS/MS on an Evosep One (60 samples per day) interfacing with a Bruker timsTOF Pro2 mass spectrometer. MS data were acquired in DIA-PASEF mode and analyzed using DIA-NN. Plasma samples were also processed for total lipids utilizing an extraction mixture of 1:1 v/v butanol:methanol. Clean extract from each subject was analyzed by LC-MS/MS on a Bruker timsTOF Pro2 in positive ionization mode utilizing DDA-PASEF. Data was analyzed utilizing Metaboscape to detect, deconvolute, and annotate lipids.

In the initial analysis, 3,381 proteins were detected in all samples (minimum of 3 samples per class). Of these, over 100 proteins were differentially measured with statistical significance in pancreatic cancer subjects following a Bonferroni correction (5% false discovery rate). The initial analysis also annotated >260 lipids in positive ion mode from ˜8,000 features following a conservative rules-based annotation approach that incorporated the high resolution, high mass accuracy, ion mobility CCS values, and MS2 spectra of the DDA PASEF data collection. Example lipid classes that were detected included phospholipids, triglycerides, sphingolipids, and cholesteryl esters. Protein and lipid classes measured in the study have previously reported associations with pancreatic cancer, thereby adding confidence to the initial proteomic and lipidomic measurements. The data also comprised protein and lipid classes with no currently known association with pancreatic cancer. Ongoing analysis of the detected proteins and lipids could enable discovery of previously unknown biology and expand the realm of biomarker analytes for early detection of pancreatic cancer.

Preliminary analysis of the cohort study indicated that biological signatures of pancreatic cancer can be inferred using the multi-omics approach evidenced by significant differences between pancreatic cancer and healthy subject across analyte classes. Further analysis of this cohort study will determine if feature integration within and across analyte classes could improve biomarker detection. This is a case-control study, not an intent to test study. This study indicated the detection of pancreatic cancer across a multitude of analyte classes.

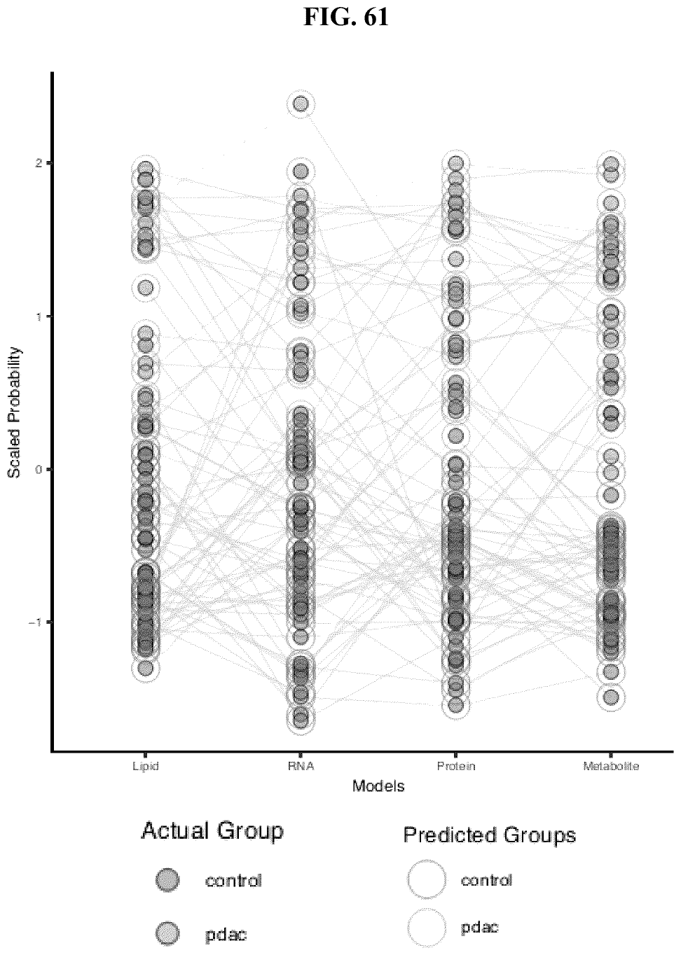

FIG. 6A and FIG. 6B illustrate the results of proteins detected across the samples obtained from the 193 subjects. FIG. 6A illustrates a median of 2,736 protein groups across five nanoparticles (NP1-NP5) for the 193 subjects in this study, where an average of 1664 proteins were detected. FIG. 6B illustrates 3,822 protein groups detected across five nanoparticles for the 193 pancreatic cancer and healthy subject samples using DIA-NN. 2,933 protein groups were identified in 25% of cohort, and 484 proteins were consistently identified in 100% of the cohort. FIG. 6C shows reproducibility of the platform indicates the ability to detect biological signal. Analysis Groups: C=Control; S=Sample. Left panel: only proteins with n>1 detections/analysis group were retained. 2 features with CV>300% out of 2,089 were removed for clarity. Right panel: only proteins with n>1 detections/analysis group were retained. 48 features with CV>300% out of 7,672 were removed for clarity. Proteins detected in 25% of all samples were utilized for classifier development. Increased sample variability was expected in a case control study. Reproducibility across 15 plates and 2 months demonstrated reproducibility of controls highlights platform performance and ability to detect biological signal. Increased variability of samples indicates biological signal was captured. FIG. 6D shows detection of more than 5,000 proteins in feasibility study of 212 subjects. A median of 4 peptides per protein was detected for proteins present in >25% of the samples with search parameters: 0.1% peptide/protein FDR, default timsTOF parameters with complete UniProt human proteome database with contaminants (50% reversed decoys). FIG. 6E shows large numbers of proteins are reproducibly detected across samples. Individual nanoparticles yielded both complementary and common protein identifications. Unique protein groups were shown for each sample/particle+panel with grouping by sample and collection site. FIG. 6F shows enhanced proteome coverage detecting known cancer related proteins. All detected, matching proteins from samples plotted on HPPP curve. GeneCards data used score reported from matching gene id and search term “cancer”. Detected HPPP1 proteins covered 8 orders of magnitude difference: highest concentration: P00450—Ceruloplasmin; 830,000 ng/mL; and lowest concentration: Q7Z627—E3 ubiquitin-protein ligase HUWE1; 0.0034 ng/mL. FIG. 6G shows deep and efficient plasma proteomics at scale. FIG. 6H shows quantitative performance of Proteograph suitable for large scale studies. FIG. 61 shows reproducibility of protein enrichment by Proteograph at scale. Reproducibility of Proteograph enrichment ideally suited for biomarker discovery. Data collected across 191 enrichments of identical sample. Scope of collection includes 3 instruments; 3 cohort studies; 5 operators; 8 months of run time; 121 plates; and 1500+ subject samples. FIG. 6J shows reproducibility of the platform overtime (months) and instruments. Median MS1 peak areas for iRT peptides were all below 15% with majority below 10%. FIG. 6K shows Application of platform to pancreatic cancer biomarker discovery.

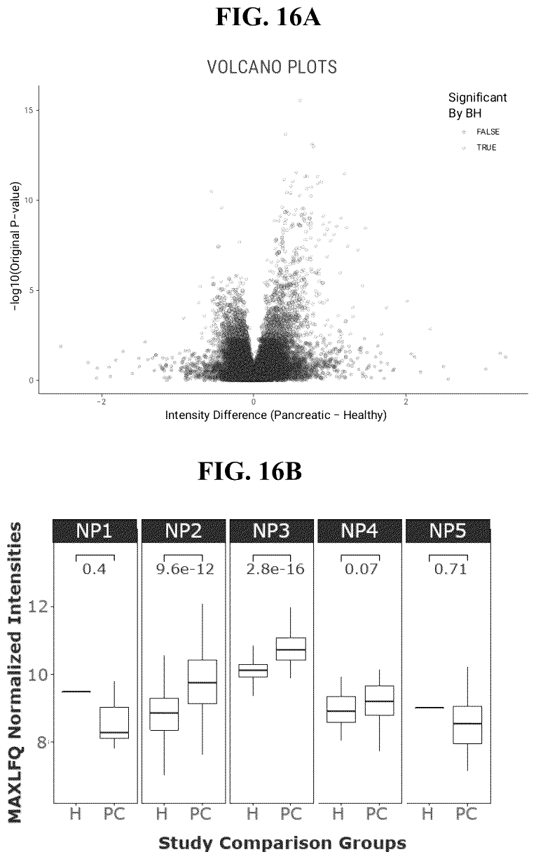

FIG. 7A illustrates the 3,822 detected protein groups mapped to the HPPP database. Identified proteins had concentrations ranging eight orders of magnitude. All identified proteins were displayed with proteins having significant pancreatic cancer OT scores<0.15 highlighted. FIG. 16A illustrates volcano plot showing intensity difference between pancreatic cancer samples and healthy samples. The volcano plot indicates 124 the of a total of 3,822 protein groups were statistically significantly different between healthy and pancreatic cancer subjects calculated based on Wilcox test with Benjamini-Hochberg correction (p=0.05). Significance testing using multiple-testing correction with 0.05 threshold using non-imputed data with at least three measures per class were used in this analysis. FIG. 16B shows study comparison group (H: healthy; PC: Pancreatic cancer). 124 of 3,381 detected proteins were statistically significant.

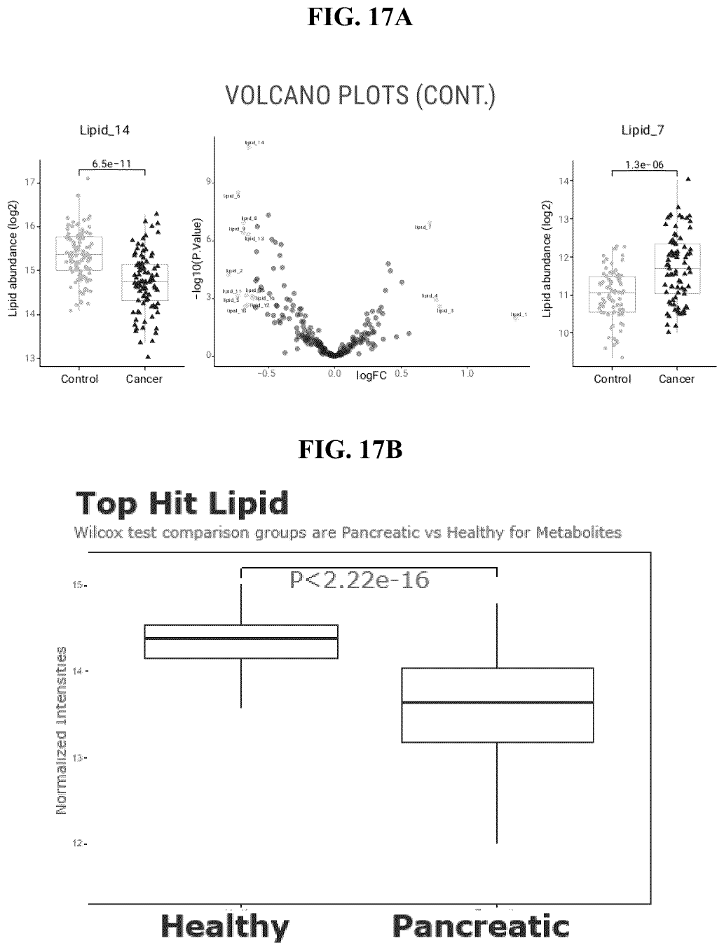

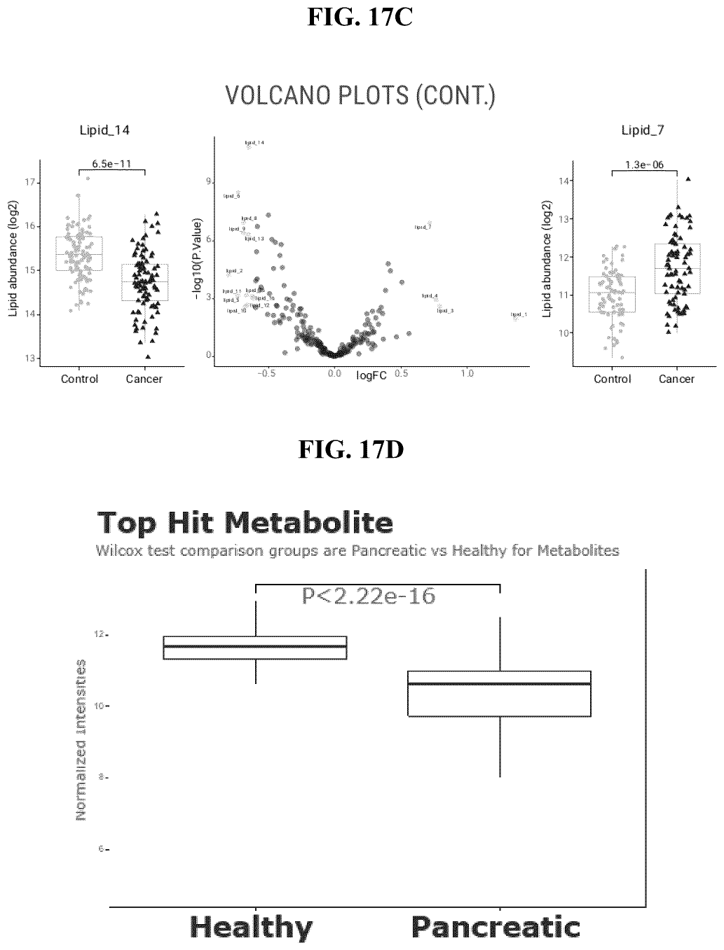

FIG. 17 illustrates volcano plots showing differential abundance of lipid species between pancreatic cancer samples and healthy samples. FIG. 17A illustrates volcano plot showing differential abundance of lipid species between pancreatic cancer samples and healthy samples calculated based on t-test and Benjamini-Hochberg correction. 16 of 259 lipids species were significantly different (adjusted p-value<0.05) between healthy and pancreatic cancer subjects. Representative boxplots for two of the lipid species depicted the differential abundance between healthy and cancer subjects. FIG. 17B illustrates an exemplary graph showing top hit lipids based on FIGS. 17A. and 17C illustrates volcano plot showing differential abundance of lipid species between pancreatic cancer samples (stages 1 and 2) and healthy samples calculated based on t-test and Benjamini-Hochberg correction. 5 of 259 lipids species were significantly different (adjusted p-value<0.05) between healthy and pancreatic cancer subjects (stage 1 and 2). Representative boxplots for two of the lipid species depicted the differential abundance between healthy and early-stage cancer subjects. FIG. 17D illustrates an exemplary graph showing top hit metabolites based on FIG. 17C.

The multiple-omics platform described in Example 6 had shown to facilitate the evaluation of the global proteome and lipidome of the pancreatic cancer cohort and identified multiple putative biomarker candidates across analyte classes for early disease (e.g., early stage of pancreatic cancer) detection. Untargeted DIA proteomics data yielded 124 statistically significant proteins out of a total of 3,822. Untargeted lipidomics data showed 16 of 259 lipids species that were significantly different between healthy and pancreatic cancer subjects. 5 out of 259 lipid species between healthy and stage 1 and 2 cancer subjects to be statistically significantly different which highlighted the detection of a biological signal related to early stages of pancreatic cancer.

This unbiased multi-omics platform leveraging 4D-mass spectrometry can integrate molecular signatures of cancer across multiple analytes to facilitate early biomarker discovery. Also, this platform can be integrated with other analytes from genomics, transcriptomics, metabolomics, DNA methylomics, and glycomics.

### Example 7. Combining Proteograph Technology with Zeno SWATH Acquisition Further Improves Deep, Unbiased Discovery of Biomarkers in Blood

Recent proteomic advancements have enabled large-scale studies to investigate biomarkers relevant to disease diagnosis and prognosis, while giving insight into the pathogenesis of complex diseases such as cancer. Liquid biopsies have been increasingly investigated for large-scale biomarker studies due to the non-invasive nature of sample collection, compared to invasive techniques such as tissue biopsies, potentially enabling improved prognosis and survival. Despite the challenges of achieving deep proteome coverage in complex biological matrices, innovative sample preparation and liquid chromatography mass spectrometry (LC-MS) technology have facilitated identification and quantification of cancer-specific biomarkers in wide ranges of concentrations in liquid biopsies. This study addresses the unmet need for deep, reproducible identification from the human plasma proteome utilizing advanced sample preparation and LC-MS technology.

From the large multi-omics oncology discovery study, comprised of >1,750 subjects across three different cancers, a retrospective case-control sub-study was performed to survey the plasma proteome profiles of 104 normal and 92 pancreatic cancer subjects (the same plasma samples as in Example 2). The samples were processed utilizing the nanoparticle based Proteograph technology from Seer. The samples were then subjected to data acquisition using a Waters ACQUITY M-class system (LC) with capillary flow rates (5 μL/min) synchronized to the ZenoToF 7600 system from SCIEX (MS). Duplicate injections were made into the mass spectrometer with and without enabling prototype Zeno SWATH acquisition in data independent acquisition (DIA) mode. The data processing and downstream analysis was performed using DIANN.

In this study, the nanoparticle based Proteograph technology was implemented along with prototype Zeno SWATH acquisition methods to yield highly reproducible proteome data while increasing the depth of coverage of low abundant proteins.

An average of >1,500 protein groups and >13,000 peptides were annotated per plasma sample due to the increased sensitivity of Zeno SWATH acquisition methods combined with the additional proteome depth provided by the Proteograph technology. A sub-study of ˜200 biological samples and process controls generated robust plasma protein measurements across ˜1,000 injections, demonstrating the robustness and reproducibility advantages of a capillary LC combined with Zeno SWATH acquisition. In addition, large differences were observed in reproducible protein identification using Zeno SWATH acquisition versus SWATH acquisition using the same experimental and analytical parameters. These results further demonstrated the feasibility of running larger cohort studies with thousands of clinical samples that address historical technical challenges related to translating proteomics to the clinic.

Furthermore, this study indicated that the Proteograph or Zeno SWATH acquisition workflow may be used to facilitate identifying and quantifying thousands of proteins from human plasma without compromising throughput or reproducibility, creating a unique opportunity to detect robust protein biomarkers that translate into viable clinical tests for complex diseases. Quantification of thousands of plasma proteins was enabled at least in part by combining nanoparticle-assisted sample preparation with reproducible and sensitive MS measurements.

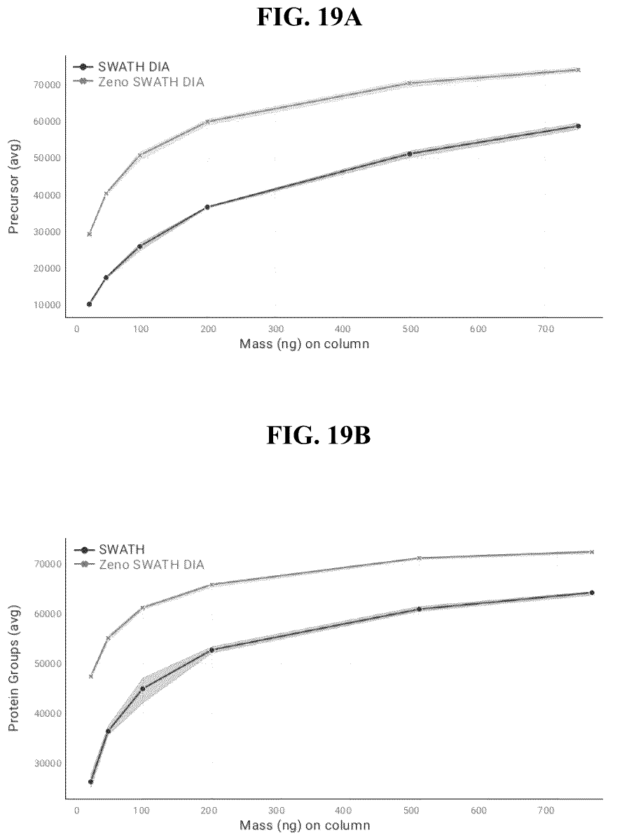

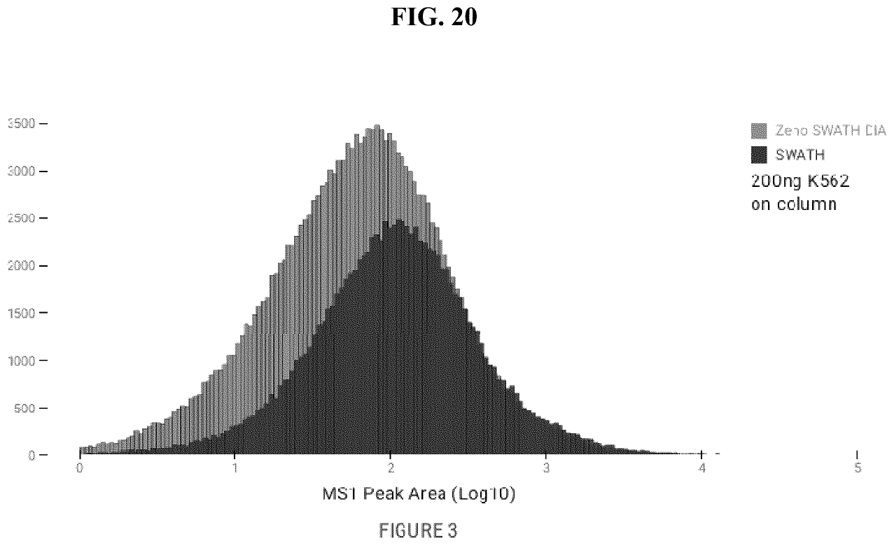

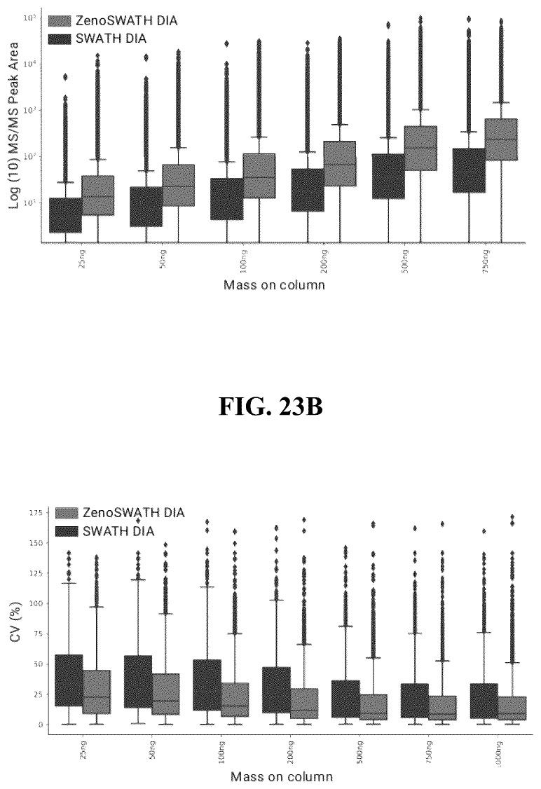

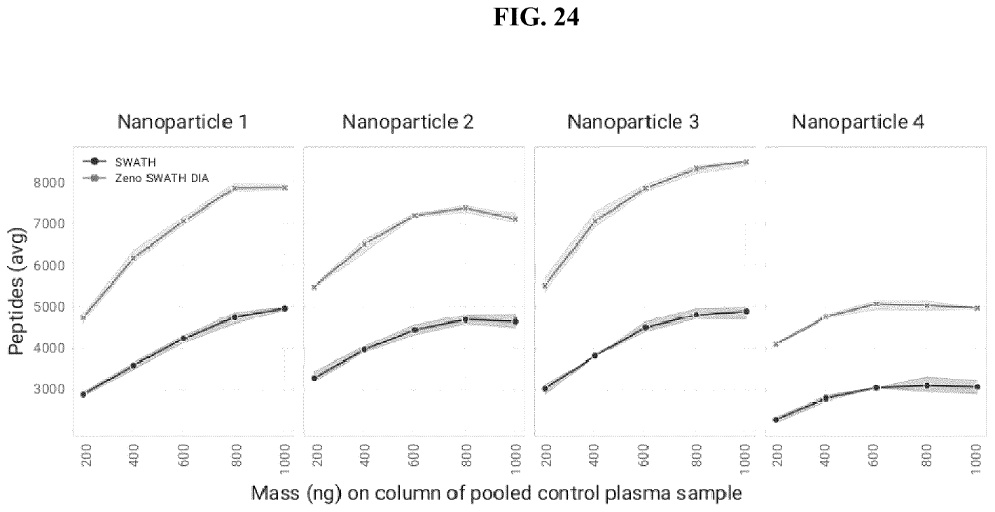

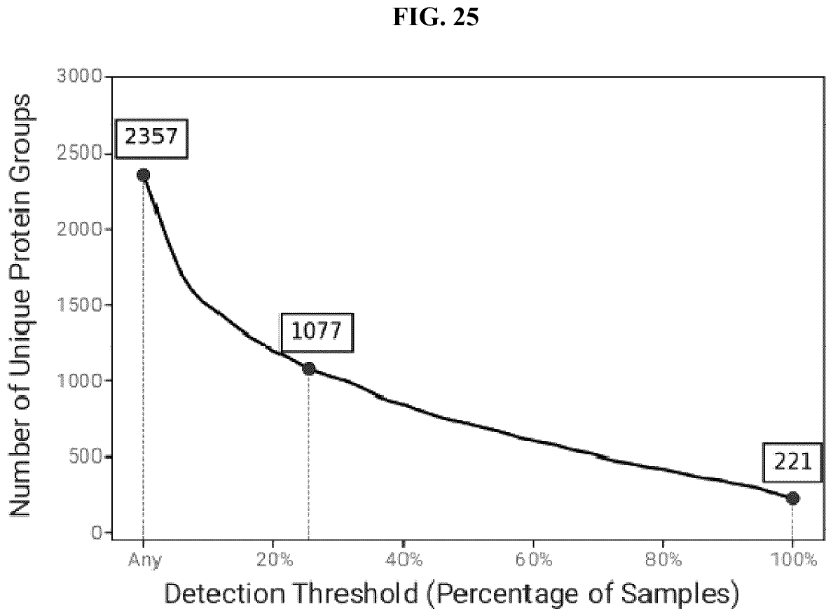

It was found that Zeno SWATCH DIA acquisition on K562 standard cell lysate led to a minimum increase of 26% in the total number of precursors and an increase of 13% to 83% of identified protein groups compared to traditional SWATCH acquisitions (FIG. 19A and FIG. 19B). Use of Zeno SWATCH DIA technology demonstrated slight increases in overall MS peak area and substantially increased MS/MS peak area for low abundant species leading to improved identifications in both the peptide and proteins levels (FIGS. 20-22). Zeno SWATCH DIA has improved reproducibility to a wider degree when compared to a SWTCH, even when minimal load mass was introduced to the instrument. A 4% to 13% decrease in CV (%) of precursor-level intensities with Zeno SWTCH DIA was observed when compared to SWATH acquisition (FIG. 23). The nanoparticle based Proteograph processing of both subject and pooled control plasma samples were combined with Zeno SWATH DIA acquisition. Increase depth of protein identification was observed. In nanoparticle-derived samples from pooled control samples, a 53 to 85% increase in peptide identification with Zeno SWATH DIA acquisition was observed when compared to SWATCH (FIG. 24). The analysis of a subset (55) of 196 control and pancreatic cohort showed that an average of 2,357 protein group were found in at least one sample, and 1,077 protein groups were found in at least 25% of all subject samples (FIG. 25).

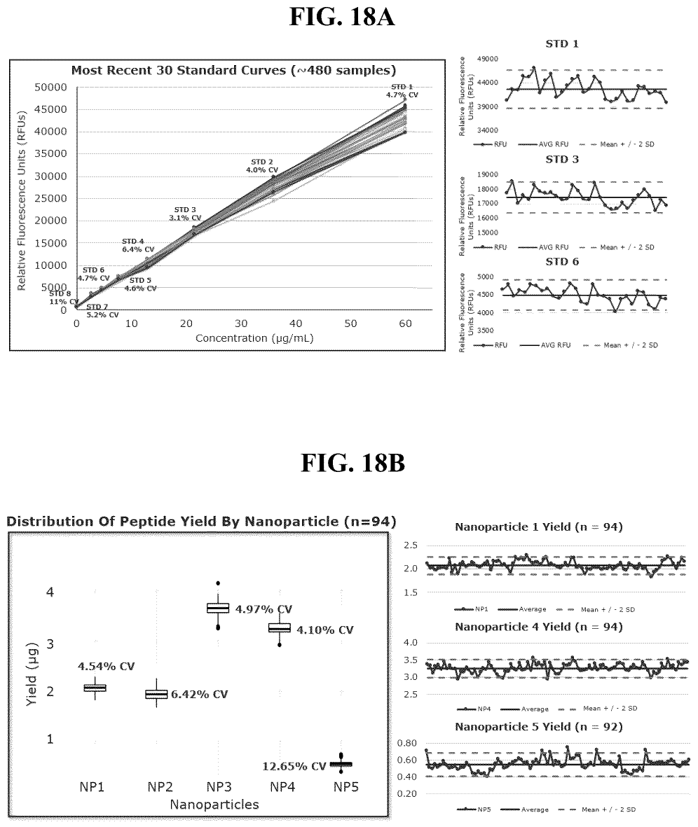

FIG. 18A shows quantitative performance of Proteograph suitable for large scale studies (e.g., study in Example 7). FIG. 18B shows reproducibility of protein enrichment by Proteograph at scale. Reproducibility of Proteograph enrichment ideally suited for biomarker discovery. System provides high throughput, reproducible and deep proteome coverage for novel discoveries. Quantitative, deep, untargeted proteomics biomarker studies were enabled by Proteograph reproducibility. Protein enrichment by Proteograph at scale was highly reproducible (NP1=0; NP2=0; NP3=2; NP4=0; and NP5=2). FIG. 19A shows evaluation of K562 precursor detection with SWATH vs. Zeno SWATH DIA. Minimum increase of 26% in precursor identifications was detected utilizing Zeno SWATH DIA. All data was generated from pr and pg matrix (all quantified precursors and proteins called were identified) from DIA-NN output. All data searched in DIA-NN with “robust LC” and SCIEX K562 spectral library. FIG. 19B shows evaluation of K562 precursor detection with SWATH vs. Zeno SWATH DIA. Minimum increase of 13% in protein group identifications was detected utilizing Zeno SWATH DIA. All data was generated from pr and pg matrix (all quantified precursors and proteins called were identified) from DIA-NN output. All data searched in DIA-NN with “robust LC” and SCIEX K562 spectral library.

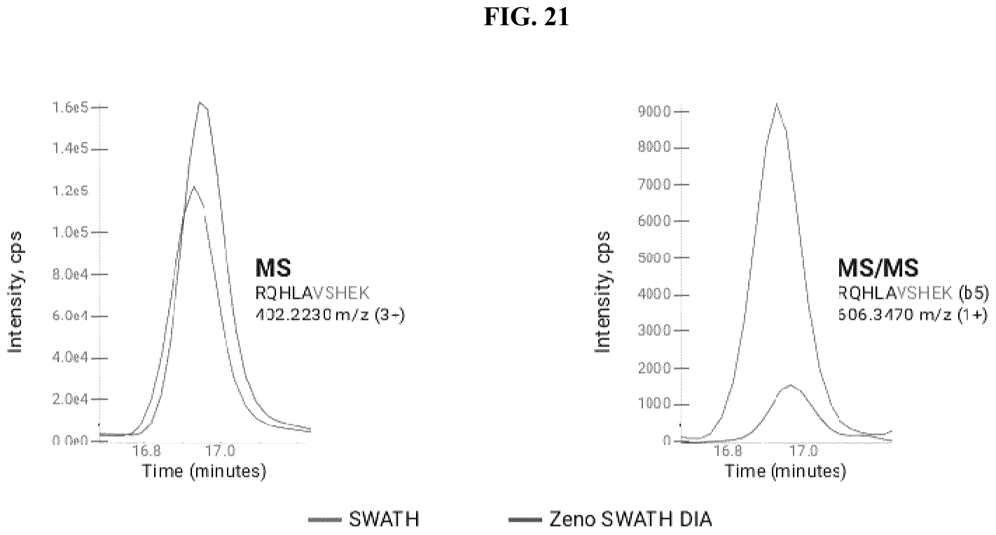

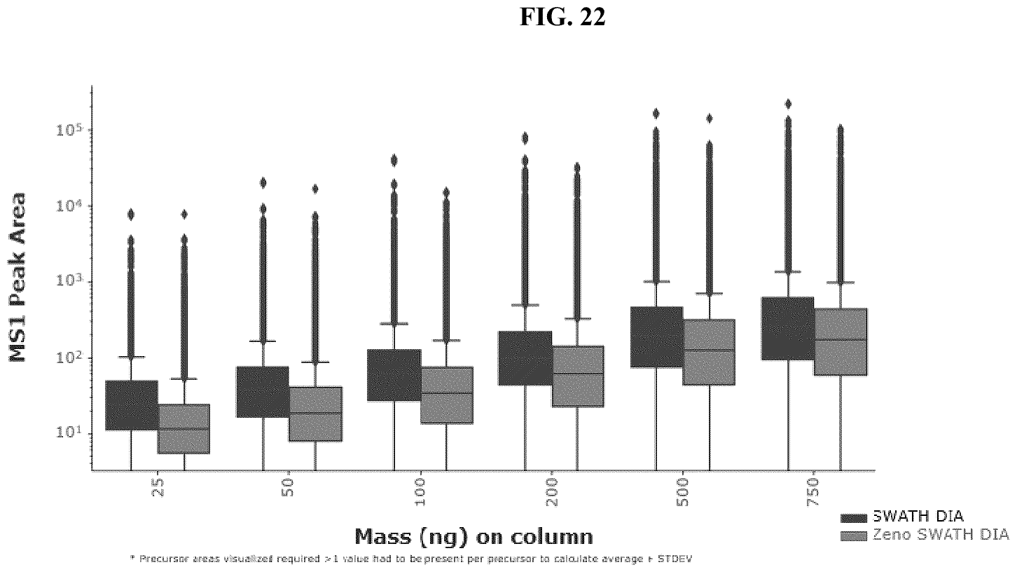

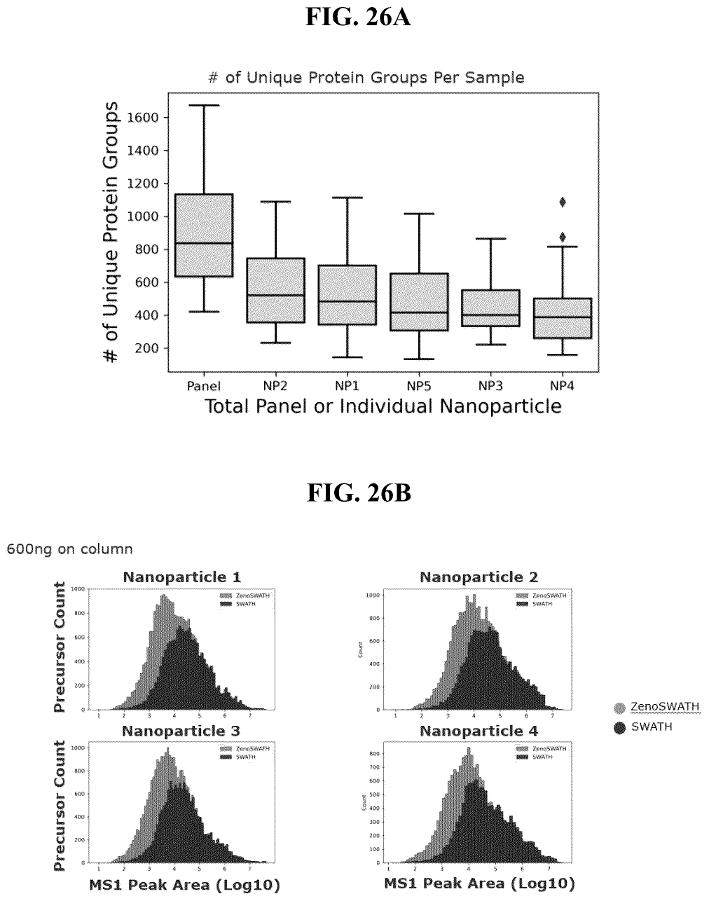

FIG. 20 shows improved sensitivity increasing number of low abundant peptides species detected. Detection of low abundant peptides was improved with Zenon SWATH DI compared to SWATH. FIG. 21 shows graphs generated from all qualified precursors. Data was searched in DIA-NN with “robust LV” and SCIEX K562 spectral library. FIG. 22 shows quantitative sensitivity increases with mass on SWATH and Zeno SWATH DIA. Zeno SWATH DIA MS1 peak areas (K562) were distributed to lower abundance peptides. FIG. 23A shows Zeno SWATCH DIA acquisition resulted in higher K562 MS2-based precursor quantity compared to SWATH acquisition alone across different peptide injection masses based on all qualified precursors. Data was searched in DIA-NN with “robust LC” and SCIEX K562 spectral library. FIG. 23B shows Zeno SWATH DIA acquisition resulted in lower CV(5) for K562 precursor-level quantities compared to SWATCH acquisition alone across different peptide injection massed based on all quantified precursors. Data was searched in DIA-NN with “robust LC” and SCIEX K562 spectral library. FIG. 24 shows Zeno Swatch DIA MS/MS acquisition resulted in 53-85% more peptide identifications from Proteograph generated from pooled control samples when compared to SWATH MS/MS DIA acquisition. FIG. 25 shows 2,357 protein groups across all five nanoparticles in the representative subject cohort. The 1077 protein groups were identified in at least 25% of the patient samples. FIG. 26A shows large numbers of proteins that were reproducibly detected across samples. Individual nanoparticles yielded both complementary and common protein identifications. FIG. 26B shows improved sensitivity equates to detection of more low abundant peptides in Proteograph peptide detection.

### Example 8. Validated Pancreatic Ductal Adeno Carcinoma (PDAC) Classifier Based on a Panel of Proteins Measured in Plasma with Targeted Mass Spectrometry Assay in a Case-Control Study of 182 Subjects

Early detection of a pancreatic cancer such as pancreatic ductal adenocarcinoma (PDAC) may be beneficial for avoiding negative outcomes of late-stage detection in which the five-year survival rate for distant, metastatic cancer is only 3%. It may be useful to provide a simple, blood-based test for PDAC with adequate sensitivity and specificity to be efficiently and efficaciously deployed in either a naïve, general screening population, or an at-risk population such as patients with pancreatitis, chronic or acute, or with newly diagnosed adult-onset diabetes. One cancer biomarker, CA19-9, is sometimes used in PDAC and other cancers, especially for recurrence testing, but a lack of performance, particularly specificity, may make it clinically unacceptable in the intent-to-test populations described above. In addition, 5-10% of the general population is Lewis negative and cannot produce the CA19-9 cancer antigen at all.

Advances in methods for detecting large-numbers of proteins and other analytes from subject samples as well as advances in machine-learning based methods for classification using the data collected with those methods may be useful to improve upon tests such as CA19-9. A plurality of signal inputs may be useful to detect as well as distinguish complex pathologies such as cancer. Toward that goal, a large, unbiased, targeted protein mass-spectrometry (MS) panel was used in a case-control study of PDAC subjects versus age- and gender-matched non-cancer controls to construct and validate a multi-protein panel with performance characteristics superior to CA19-9.

The cases and controls for this IRB-approved observational, sample-collection study were collected from 17 different sites across a period of more than two years, with PDAC subjects enrolled from 15 of the sites and non-cancerous controls enrolled from 5 of the sites. The major inclusion/exclusion criteria were based on newly diagnosed, biopsy-confirmed PDAC in the absence of any other cancer or cancer history for at least the preceding five years. The PDAC subjects were diagnosis-aware but treatment-naïve, and samples were generally collected within several weeks of confirmed pathology.

In the initial data analysis (EDA), a cohort of 184 age- and gender-matched subjects from collection were evaluated by univariate, non-parametric Wilcoxon tests as well as by multi-variate, principal component analysis (PCA) and hierarchical clustering. Of the 447 proteins out of the original 554 in the targeted mass spectrometry (MS) panel that were present in at least 50% of at least one of the classes, 113 showed a significant difference in the Wilcoxon test with a Bonferroni-adjusted p-value less than or equal to 0.05. Most (e.g., 94 out of 113) of the significant differences were elevated in PDAC. Performance of multi-variate analysis by PCA and hierarchical clustering further indicated the usefulness of efficacious combinatorial-marker class separation by regression-based and decision-tree based classification methods.

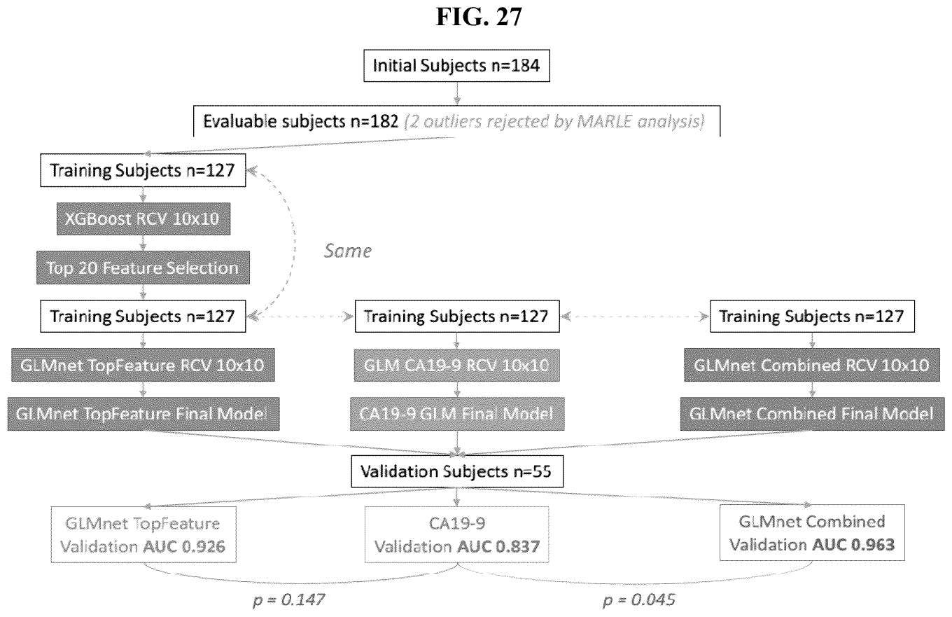

For a machine-learning based classification model analysis (summarized with results in FIG. 27), a two-stage approach was selected. First, cohort was randomly divided into training (n=127) and validation groups (n=55) with cancer status-stage stratification to retain proportionality. Two-stages of repeated cross-validation (ten repeats of 10-fold RCV) were then used in the training group, first with XGBoost, a gradient boosted-ensemble decision tree method, for the selection of the top 20 most important features, and then, after a new, random assignment of the same subjects into repeats and folds, a second round of RCV using GLMnet, a regularized logistic regression method, using only the selected features. In addition to this analysis with the targeted MS protein panel, CA19-9 was directly measured from these subjects using a specific clinical assay and evaluated that marker both individually and with the top protein 20 features in a combined model. Using the final GLMnet model, created using all the training subjects and the top 20 proteins, a performance of 0.926 AUC was demonstrated in the validation group. In comparison, using the stand-alone CA19-9 model a validation performance of 0.838 AUC was demonstrated. In a combined approach, which used the top 20 proteins as well as CA19-9 for a final GLMnet model, a validation performance of 0.963 AUC was achieved. This classifier's performance was statistically better than CA19-9 alone (p=0.045). The combined model's performance at 99%, 98%, and 95% specificities was 77%, 77%, and 82% sensitivity, respectively. The combined model's high performance extended across all PDAC stages with seven of the nine (78%) stage 1/2 cancers in the validation group correctly assigned. Although 20 as the number of features were chosen to move forward from initial XGBoost RCV feature selection to final GLMnet RCV model construction, smaller numbers of features from the top 20 may give similar performance. As such, a pancreatic cancer classifier including any of the top 20 features in this model, or any combination thereof, may be useful in detecting pancreatic cancer at an early stage.

Using a PDAC-association annotation listing for 4,886 genes downloaded from OpenTargets, the prior association of the top 20 proteins was examined. The feature Uniprot IDs were mapped to gene names and IDs, and then mapped to the OpenTargets annotations. Four of the top 20 proteins had no PDAC OT score, indicating that there is little to no evidence in that database for prior association to PDAC. The remaining 16 proteins had non-zero scores, but the highest score was still lower than approximately 80% of all of the 4,886 proteins in the database. This indicates that the selected panel of 20 top protein features is likely a novel combination of proteins for identifying a pancreatic cancer, and also that individual top protein features use herein may be useful in a classifier for pancreatic cancer either alone or in combination with others.

### Study Subject Analysis

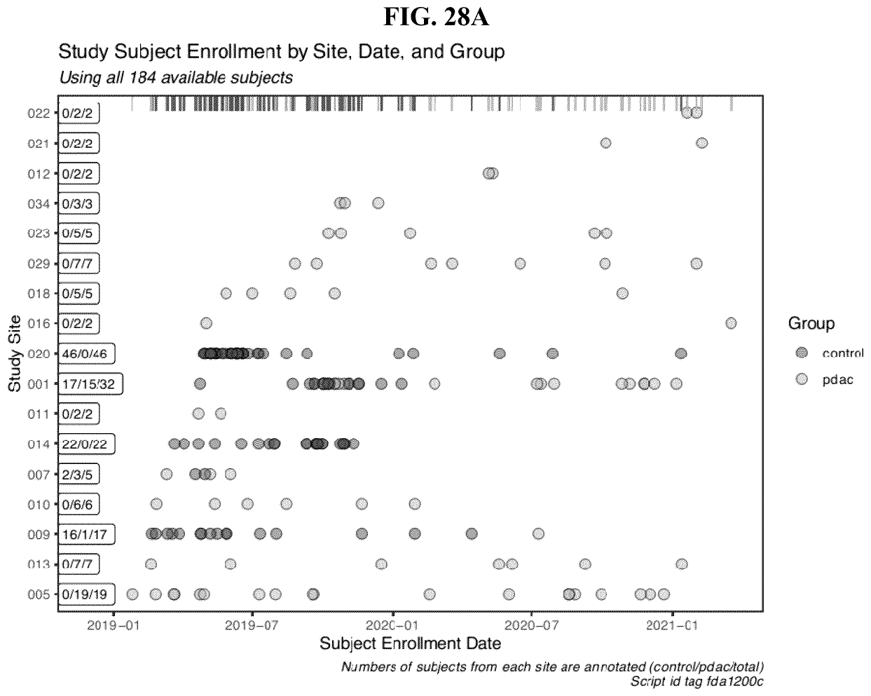

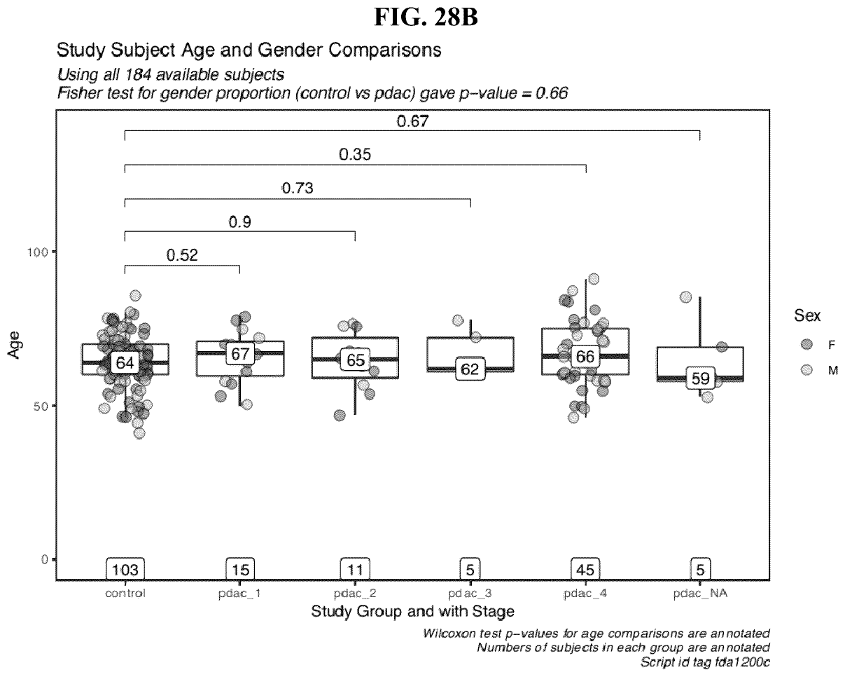

As described above, the study subjects were derived from an IRB-approved observational study that collected samples from individuals with biopsy-confirmed PDAC immediately after diagnosis but before any treatment. As such these individuals were diagnosis-aware but treatment-naïve. Multiple sample types were collected in this study to enable a wide variety of omics studies, but this exampled focused on the use of plasma samples for measurement via targeted MS of a wide variety of proteins, selected without any known bias towards PDAC-associated proteins. To avoid introducing any confounding bias in the study that aligns with the groups, samples were collected from many sites and across a large span of time, the distribution of which are shown in FIG. 28A. Although collection site and enrollment date were not completely random with respect to class, PDAC or control, the large number of sites and the large windows in time mitigated any meaningful bias between the study groups. The controls were age- and gender-matched individuals who met the study-specified inclusion/criteria that excluded any history of cancer within the preceding five years. FIG. 28B demonstrates the lack of age or gender bias between the groups broken down by stage of cancer. Age was compared by Wilcoxon test and gender proportionality was confirmed by Fisher's test. These 184 subjects, with data for both the unbiased, targeted protein panel MS assay and the analyte-specific CA19-9 ELISA assay, were used as input to the subsequent EDA and machine learning-based classifier analysis.

### PiQuant Protein Assay Data Preparation

A panel of stable-isotope-standard (SIS)-peptides was used to assay 554 proteins by targeted MS on a ThermoFisher OrbiTrap MS. In this example, this method is referred to as “PiQuant.” The use of SIS-peptides as an internal calibrant enabled highly accurate and precise determination of peptide and protein levels in each sample. For 401 of the 554 proteins, a single peptide was used per protein. For the remaining proteins, 2, 3, or 4 peptides were used. MS data including the MS2 fragments, or transitions, for each peptide were initially processed by Biognosys' Spectrodive analytical software.

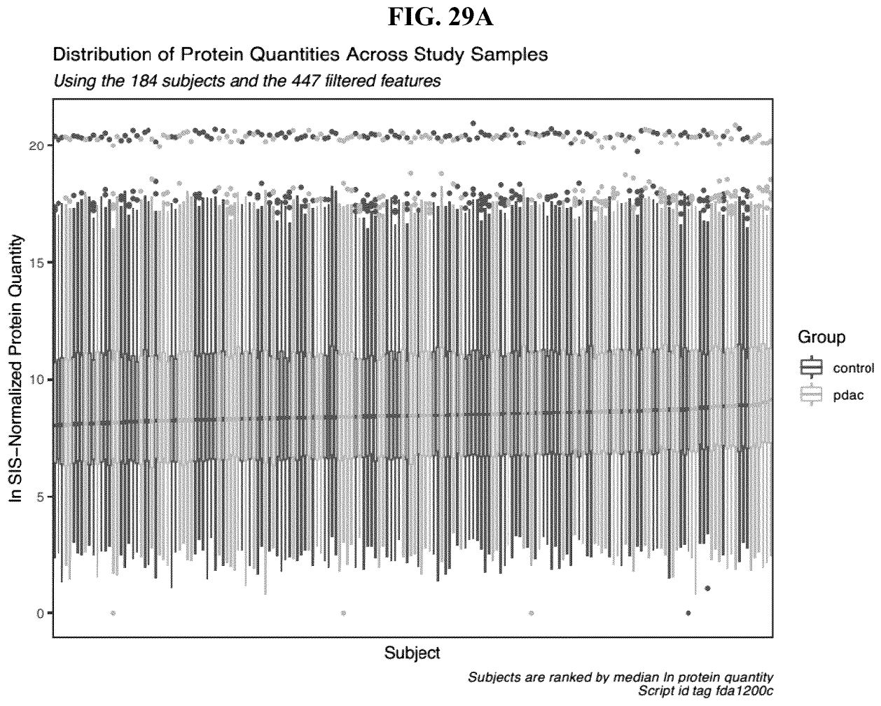

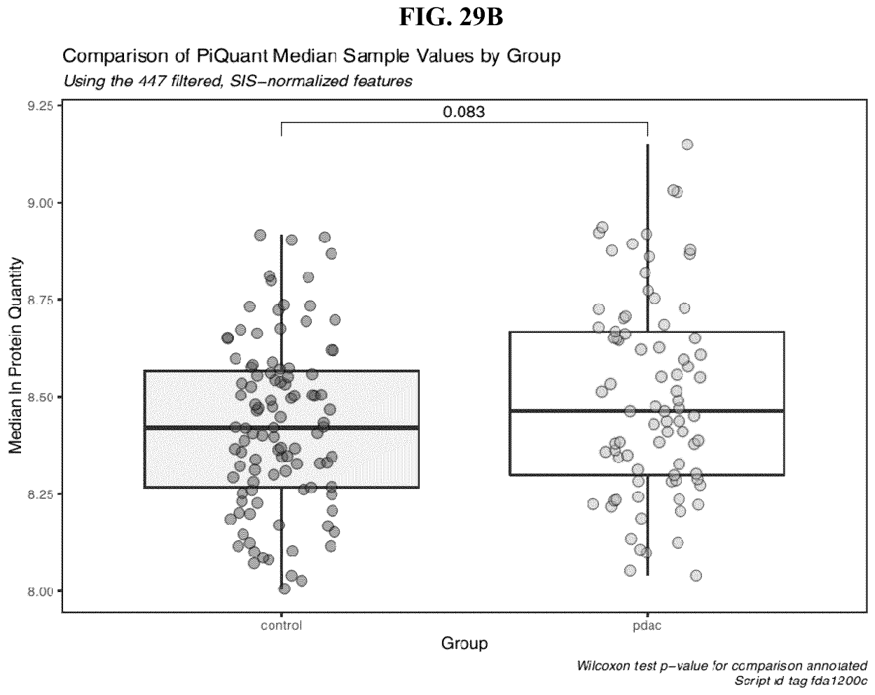

To calculate a protein-level value for each member of the panel in each sample, the median value for each the SIS peptides transitions was calculated as measured across all subject samples and then applied a sample specific correction factor derived from these data to each samples' endogenous peptide transitions. Each transition used in the calculation of a peptide value had to have a signal-to-noise ratio greater than 3, and the three transitions with the highest median values across the samples were summed for each samples' peptide level determinations. Values were log transformed to improve normality. As additional filtering, prior to subject-level analysis, the proteins needed to be detected in at least 50% of at least one of the classes of the study, PDAC and/or control. 447 proteins out of 554 met that criterion. FIG. 29A shows the distribution of the protein-level values for each sample, highlighting how the SIS-based quantification had effectively normalized the data between subject samples without bias between the groups. Although it appeared by inspection that there may be a bias for higher median values for PDAC subjects, the Wilcoxon test p-value comparing protein-level medians by group was 0.083 (FIG. 29B). Subjects were randomized across plates as part of the PiQuant assay.

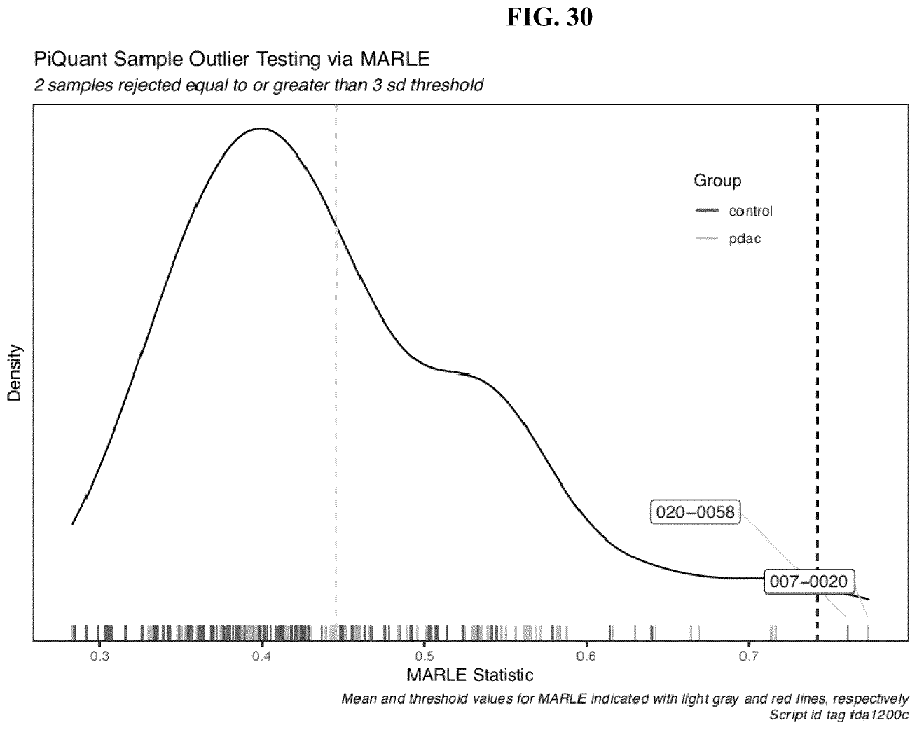

Given the sensitive nature of modern classification methods via machine learning, it is critical to avoid or remove as much unintended bias between comparison groups as possible. In addition, it is also critical to remove noisy samples from the analysis that appear to be statistical outliers. If such samples are outliers from the rest of the data set for reasons unrelated to the class distinction problem at hand, the variance that they may add to the analysis may outweigh any signal detection power that might come from the retention of the samples. One set of methods for adjudicating outliers that is particularly applicable for high-dimensional, many-measures-per-sample assay, is based on evaluating variations of median absolute deviation (MAD). In microarray genetic analyses, summing or otherwise summarizing the absolute values for the residuals for all features of an array as measured against the central tendencies of those features across all subjects in an analysis is a common metric for relative performance within a group. In the analysis, the mean absolute relative log expression or MARLE was calculated for each sample and then identified and removed outlier samples from further analysis after confirming that no class bias exists in the rejected samples. FIG. 30 shows the distribution of MARLE values for the 184 subjects in the PDAC study and highlighted two samples whose MARLE values were greater than 3 standard deviations from the mean of all subject values. Since each group was equally represented in the potential outlier rejection, no class bias (e.g., no potential class-distinguishing signal) was removed by exclusion of these subject sample data. After removing these samples, 182 subjects including 80 PDAC and 102 control remained for EDA and machine-learning based classification.

### Individual Protein Comparisons by Wilcoxon Test

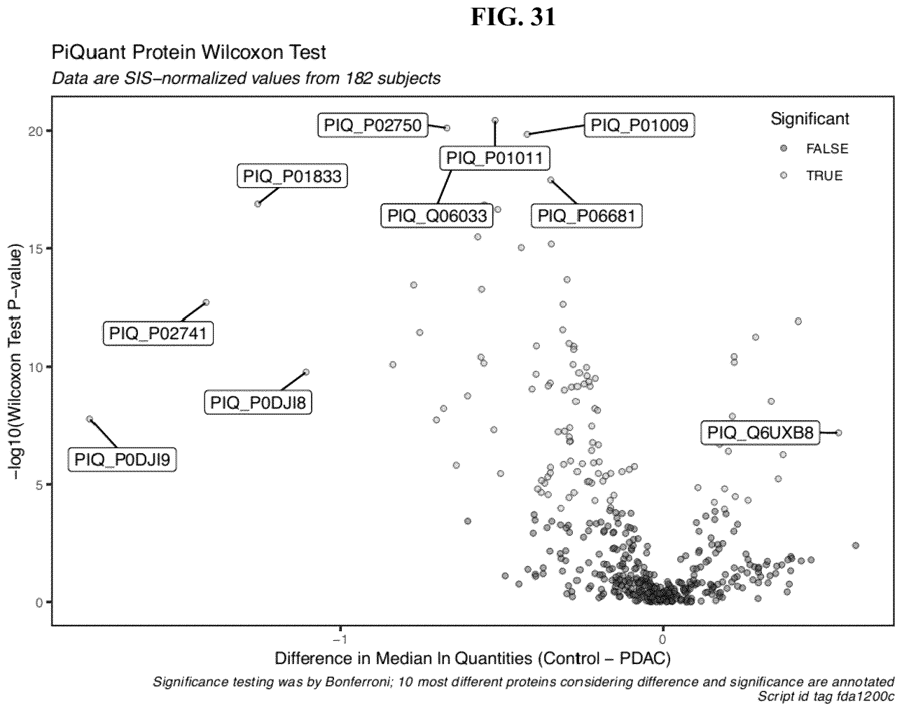

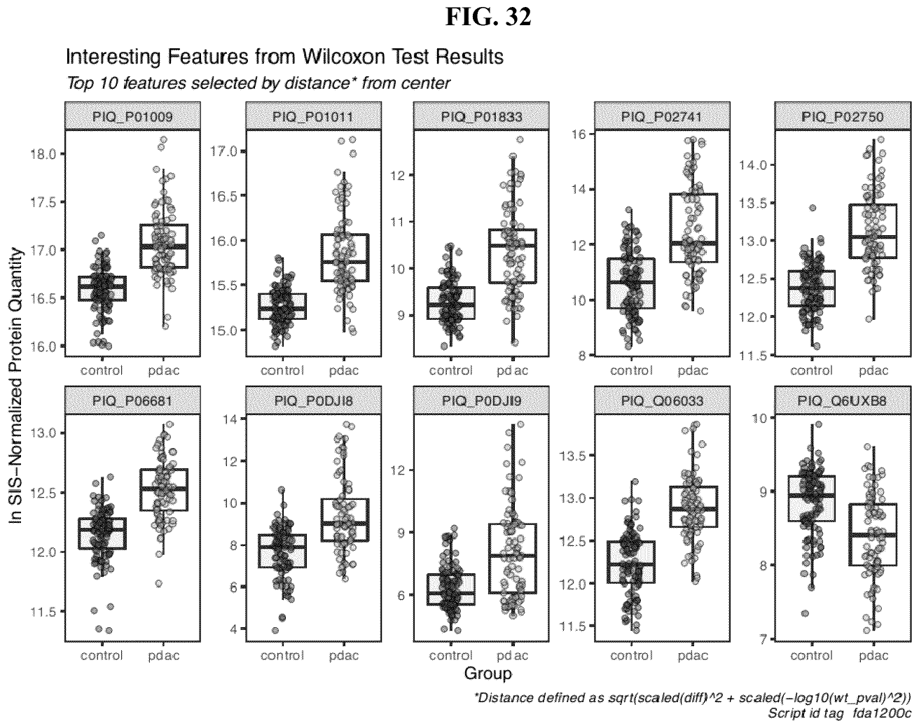

After SIS-based individual subject-protein normalization, 50% class presence study-protein filtering, and MARLE-based study-subject outlier rejection, the 447 remaining proteins in the 182 remaining subjects were evaluated for expression level differences between the study groups by non-parametric, Wilcoxon test. Given the moderately large number of tests per sample, Bonferroni correction was used to adjust the Wilcoxon p-values with p=0.05 (adjusted) set as the new significance threshold. Differences were scored as median Control—median PDAC meaning that negative differences represent proteins that are present at higher levels in PDAC subjects. FIG. 31 shows the volcano plot of −log 10 (Wilcoxon test p-value) vs the difference. As shown in the plot, a large proportion of the proteins are significantly different (e.g., 113 out of 447, with 94 being detected at higher levels in PDAC). The plot highlighted ten significantly different proteins that were selected for individual subject value inspection. The individual data points from the subjects for these ten proteins were shown in FIG. 32. As seen in the plot, although all ten proteins were indeed significantly different, no single protein completely distinguished the groups. This indicates that multi-variate analysis, both in EDA as a feasibility study, and in machine-learning based classifier construction, was useful to achieve clinically useful performance.

### Multivariate Group Comparison by PCA and Hierarchical Clustering

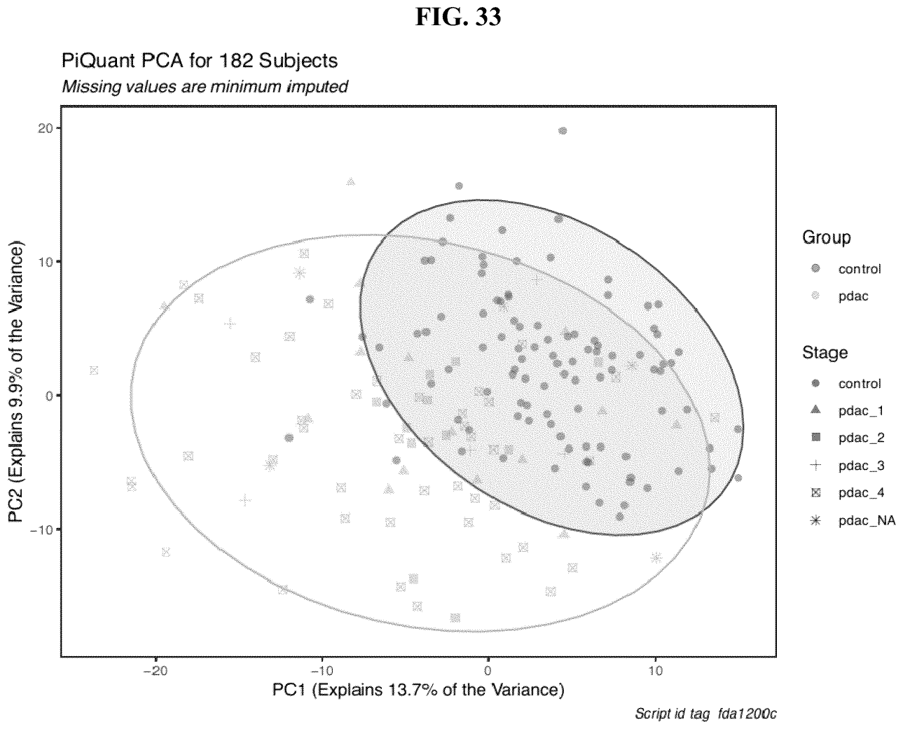

Given that individual analytes did not significantly differentiate the study groups, two multi-variate EDA methods were employed to understand the feasibility of multi-component classifiers for machine-learning. In the first method, PCA, all 447 PiQuant-measured proteins were used in the analysis, with any missing values from subjects imputed with the minimum value for that analyte across all 182 of the subjects. This implicitly assumed missing-by-detection level rather than missing-by-random-chance. In FIG. 33, modest multivariate-based separation of the groups was evident with perhaps more separation observed for the stage 4 PDAC subjects. Only the first two principal components were plotted which accounted for only 23.6% of the total variance.

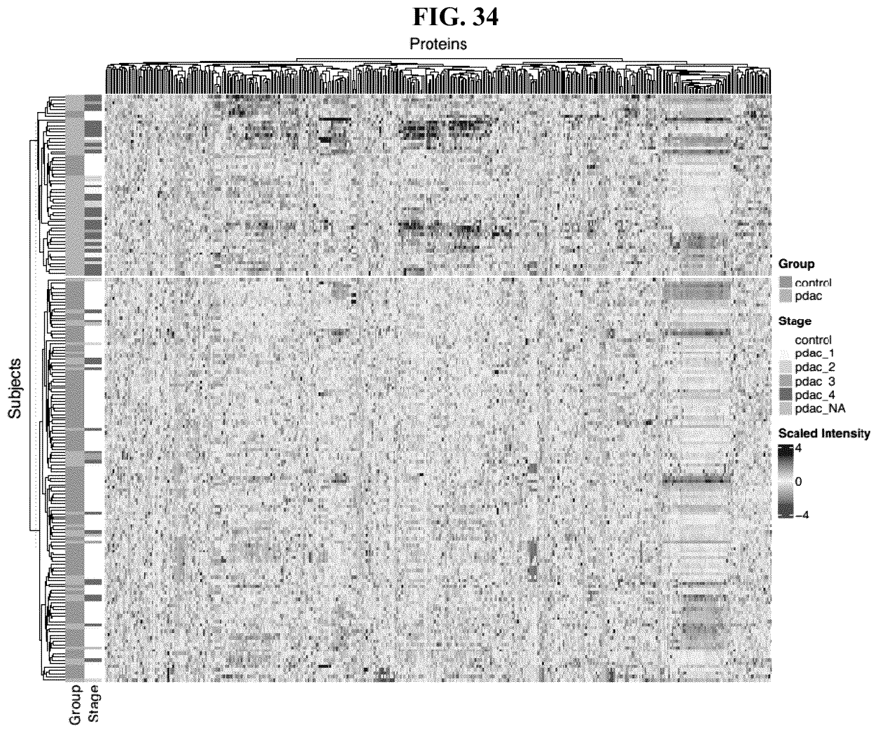

For further exploration of the potential for multivariate classification, unsupervised hierarchical clustering using all of the analytes with a complete approach using Euclidean distance measures was deployed. After clustering, the subject dendrogram was cut to produce two groups to visualize the potential for the data to separate the PDAC and controls. As seen in FIG. 34, there was significant separation of the groups with the forced split. The PDAC subjects in each branch appeared to comprise all stages of PDAC. It appeared that there was a significant amount of correlation within the protein analytes which was an important factor to consider in machine-learning based classification. To summarize the EDA, it was apparent that many protein analytes were significantly differentially expressed between the groups. Multivariate EDA, particularly the unsupervised hierarchical clustering, indicating that combining proteins into a panel of analytes may be an improved approach to developing a group classifier.

### Machine-Learning-Based Classification

Given the large number of protein analyte features (447), and the number of study subjects (182), the study was randomly split into training and validation groups, with 70/30 proportionality, and then use a two-stage approach within the training group to build a final model for validation in the held-out group (See FIG. 27). In the training group, a first round of ten repeats of 10-fold was repeated cross-validation (RCV) for most-important feature selection, and then, after randomly re-shuffling the samples into new repeat-fold groupings to minimize overfitting, a second 10×10 RCV with the important features for final model parameter selection was performed and then final training set model construction. Given the nature of the data observed during EDA, XGBoost, a gradient boosted-ensemble tree method for the first RCV and regularized, logistic regression with GLMnet was selected for the second RCV.

### Training Validation Subject Split

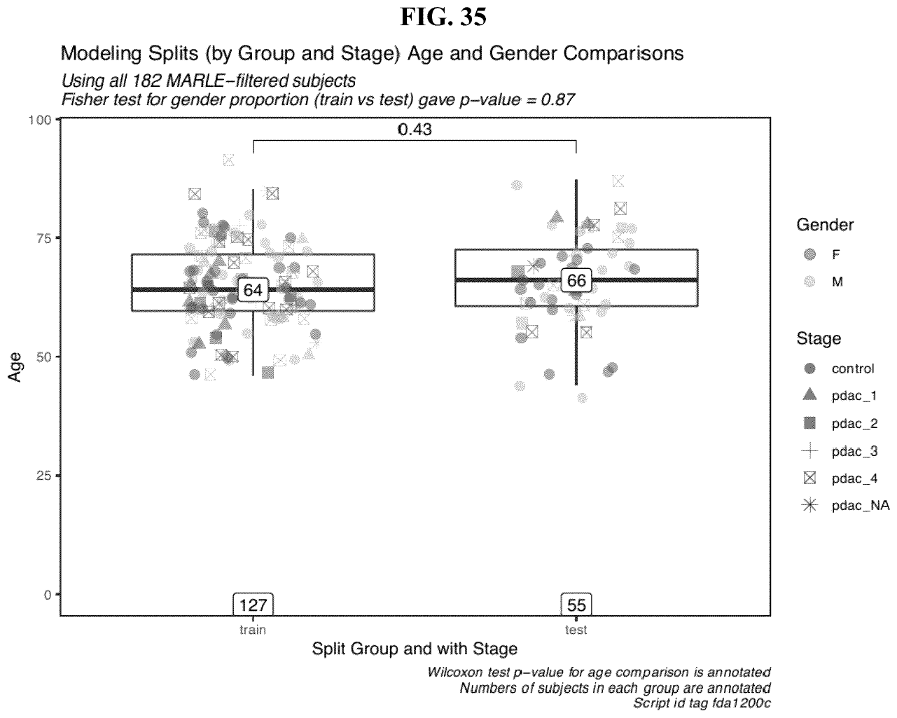

The first step in the classification process was to split the evaluable study subjects into training and validation groups with the validation set to be held-out until final testing. The subjects were randomly split for 70/30 maintaining the proportionality of cancer status-stage-status between the groups. FIG. 35 shows the splits and indicates that there were no significant differences between the groups by either age or gender.

Feature Selection by RCV with XGBoost

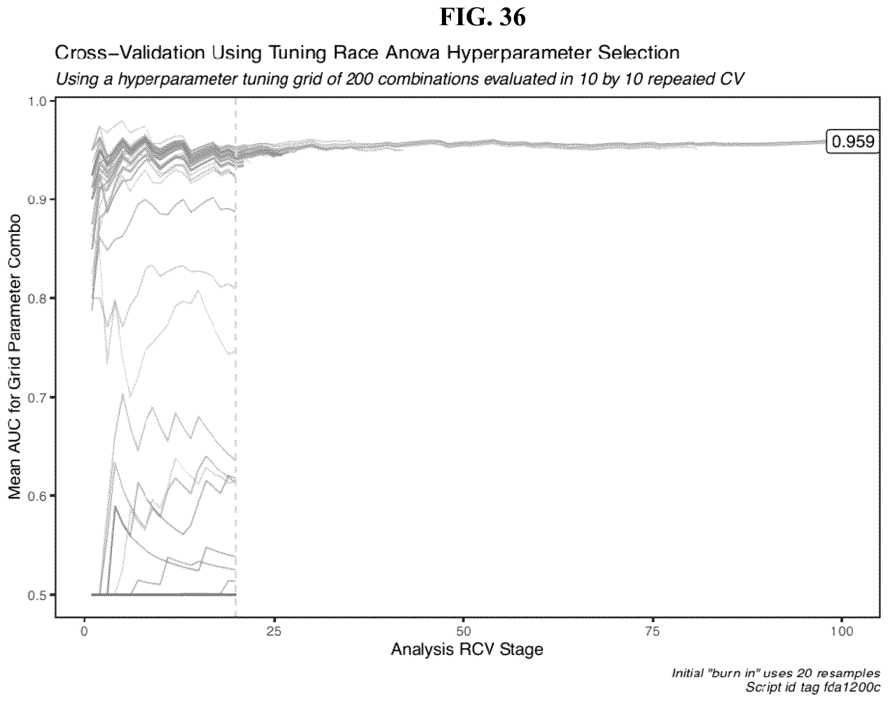

The training split (n=127) was randomly re-split into ten repeats of 10 folds, maintaining the proportionality of cancer-status, cancer-stage within the folds. Although the numbers vary slightly, a typical fold within a repeat has between 113 and 116 subjects for model creation and 14 to 11 subjects for model testing. Using these RCV splits, a large grid (n=200) of potential combinations for the seven hyperparameters for XGBoost modeling was constructed using optimized Latin-hypercube sampling. Race-tuning ANOVA was used within RCV execution to optimize the compute time necessary to complete model hyperparameter evaluation. In this approach, an initial burn-in of a random sampling of folds are evaluated across all parameter combinations and compared via ANOVA. Those models which were statistical inferior to the best current model were dropped, and then this process was repeated until the final number of repeats-folds are evaluated. FIG. 36 shows the race tuning for the XGBoost RCV deployed here. XGBoost models may be very sensitive to model parameters (and therefore sensitive to overfitting), and this was evident in the pruning rate for parameter combination in FIG. 36.

The first evaluation after the initial 20 repeat-fold burn-in removed a significant number of poorly performing models. At the completion of the evaluation, the best model combination of a parameters (shown in Table 4) achieves a mean AUC of 0.959 across all models (ten repeats of 10-fold evaluation).

TABLE 4
Optimized XGBoost model hyperparameters
selected in 10 × 10 RCV

| Parameter | Value |
|---|---|
| mtry | 70 |
| trees | 1342 |
| min_n | 5 |
| tree_depth | 7 |
| learn_rate | 0.000225973465953212 |
| loss_reduction | 4.0019445946305e−05 |
| sample_size | 0.935536098000128 |

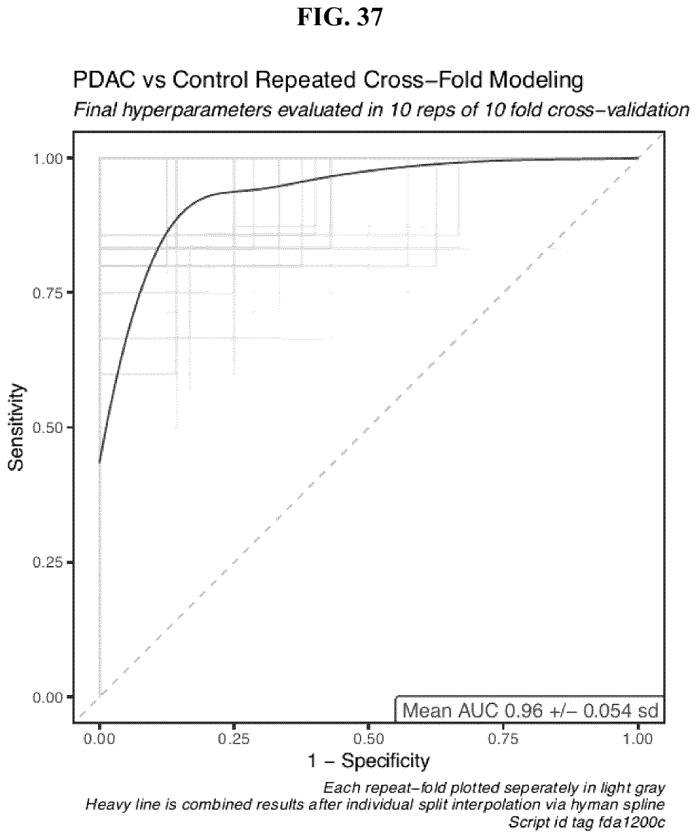

A combined ROC plot for the 10×10 RCV with the optimal parameter combination is shown in FIG. 37. The highlighted curve represents the interpolated, mean-summarized values for sensitivity and specificity for each of the comprising repeat-folds with 11-14 subjects. The light gray plots are the individual repeat-folds plots themselves.

Although the predicted performance of an XGBoost-based classifier using all 447 protein features as input predictor values is excellent in-of-itself (e.g., mean AUC 0.96), this first stage for feature-selection was used to demonstrate the potential for commercially viable, clinically useful classifiers. Top 20 features from this stage were selected to move forward into the second stage RCV, although it was possible that fewer features may be sufficient to achieve sufficient performance. To identify the top 20 features, the feature importance was selected from each of the 10×10 repeat-fold models as summarized primarily by median rank with worst and best ranks used to break ties. The protein and gene names of the top 20 protein biomarkers in this example are also listed in the Table 5. The selected important features are enumerated in Table 6.

TABLE 5
Examples of biomarkers for evaluating pancreatic cancer

| UniProt | | | |
|---|---|---|---|
| Entry | Entry Name | Protein Names | Gene Name(s) |
| P01011 | AACT_HUMAN | Alpha-1-antichymotrypsin, ACT (Cell | SERPINA3 |
| | | growth-inhibiting gene 24/25 protein) | AACT GIG24 |
| | | (Serpin A3) [Cleaved into: Alpha-1- | GIG25 |
| | | antichymotrypsin His-Pro-less] | |
| P01009 | A1AT_HUMAN | Alpha-1-antitrypsin (Alpha-1 protease | SERPINA1 |
| | | inhibitor) (Alpha-1-antiproteinase) | AAT PI |
| | | (Serpin A1) [Cleaved into: Short peptide | PRO0684 |
| | | from AAT, SPAAT] | PRO2209 |
| P02750 | A2GL_HUMAN | Leucine-rich alpha-2-glycoprotein, LRG | |
| | | LRG1 LRG | |
| P15144 | AMPN_HUMAN | Aminopeptidase N, AP-N, hAPN, EC | ANPEP APN |
| | | 3.4.11.2 (Alanyl aminopeptidase) | CD13 PEPN |
| | | (Aminopeptidase M, AP-M) (Microsomal | |
| | | aminopeptidase) (Myeloid plasma | |
| | | membrane glycoprotein CD13) (gp150) | |
| | | (CD antigen CD13) | |
| P18428 | LBP_HUMAN | Lipopolysaccharide-binding protein, LBP | LBP |
| P05362 | ICAM1_HUMAN | Intercellular adhesion molecule 1, ICAM- | ICAM1 |
| | | 1 (Major group rhinovirus receptor) (CD | |
| | | antigen CD54) | |
| P01833 | PIGR_HUMAN | Polymeric immunoglobulin receptor, | PIGR |
| | | PIgR, Poly-Ig receptor (Hepatocellular | |
| | | carcinoma-associated protein TB6) | |
| | | [Cleaved into: Secretory component] | |
| P01031 | CO5_HUMAN | Complement C5 (C3 and PZP-like alpha- | C5 CPAMD4 |
| | | 2-macroglobulin domain-containing | |
| | | protein 4) [Cleaved into: Complement C5 | |
| | | beta chain; Complement C5 alpha chain; | |
| | | C5a anaphylatoxin; Complement C5 | |
| | | alpha′ chain] | |
| P05109 | S10A8_HUMAN | Protein S100-A8 (Calgranulin-A) | S100A8 CAGA |
| | | (Calprotectin L1L subunit) (Cystic | CFAG MRP8 |
| | | fibrosis antigen, CFAG) (Leukocyte L1 | |
| | | complex light chain) (Migration inhibitory | |
| | | factor-related protein 8, MRP-8, p8) | |
| | | (S100 calcium-binding protein A8) | |
| | | (Urinary stone protein band A) | |
| P06681 | CO2_HUMAN | Complement C2, EC 3.4.21.43 (C3/C5 | C2 |
| | | convertase) [Cleaved into: Complement | |
| | | C2b fragment; Complement C2a fragment] | |
| P02748 | CO9_HUMAN | Complement component C9 [Cleaved | C9 |
| | | into: Complement component C9a; | |
| | | Complement component C9b] | |
| Q06033 | ITIH3_HUMAN | Inter-alpha-trypsin inhibitor heavy chain | ITIH3 |
| | | H3, ITI heavy chain H3, ITI-HC3, Inter- | |
| | | alpha-inhibitor heavy chain 3 (Serum- | |
| | | derived hyaluronan-associated protein, | |
| | | SHAP) | |
| P02753 | RET4_HUMAN | Retinol-binding protein 4 (Plasma retinol- | RBP4 PRO2222 |
| | | binding protein, PRBP, RBP) [Cleaved | |
| | | into: Plasma retinol-binding protein(1- | |
| | | 182); Plasma retinol-binding protein(1- | |
| | | 181); Plasma retinol-binding protein(1- | |
| | | 179); Plasma retinol-binding protein(1- | |
| | | 176)] | |
| P08637 | FCG3A_HUMAN | Low affinity immunoglobulin gamma Fc | FCGR3A |
| | | region receptor III-A, IgG Fc receptor III- | CD16A FCG3 |
| | | A (CD16-II) (CD16a antigen) (Fc-gamma | FCGR3 IGFR3 |
| | | RIII-alpha, Fc-gamma RIII, Fc-gamma | |
| | | RIIIa, FcRIII, FcRIIIa, FcgammaRIIIA) | |
| | | (FcR-10) (IgG Fc receptor III-2) (CD | |
| | | antigen CD16a) | |
| P05452 | TETN_HUMAN | Tetranectin, TN (C-type lectin domain | CLEC3B TNA |
| | | family 3 member B) (Plasminogen kringle | |
| | | 4-binding protein) | |
| P02741 | CRP_HUMAN | C-reactive protein [Cleaved into: C- | CRP PTX1 |
| | | reactive protein(1-205)] | |
| Q99784 | NOE1_HUMAN | Noelin (Neuronal olfactomedin-related | OLFM1 NOE1 |
| | | ER localized protein) (Olfactomedin-1) | NOEL1 |
| P05160 | F13B_HUMAN | Coagulation factor XIII B chain (Fibrin- | F13B |
| | | stabilizing factor B subunit) (Protein- | |
| | | glutamine gamma-glutamyltransferase B | |
| | | chain) (Transglutaminase B chain) | |
| P02652 | APOA2_HUMAN | Apolipoprotein A-II, Apo-AII, ApoA-II | APOA2 |
| | | (Apolipoprotein A2) [Cleaved into: | |
| | | Proapolipoprotein A-II, ProapoA-II; | |
| | | Truncated apolipoprotein A-II | |
| | | (Apolipoprotein A-II(1-76))] | |
| P02647 | APOA1_HUMAN | Apolipoprotein A-I, Apo-AI, ApoA-I | APOA1 |
| | | (Apolipoprotein A1) [Cleaved into: | |
| | | Proapolipoprotein A-I, ProapoA-I; | |
| | | Truncated apolipoprotein A-I | |
| | | (Apolipoprotein A-I(1-242))] | |

TABLE 6
Ranked top 20 protein features selected
from XGBoost 10 × 10 RCV

| Variable | num_in_top | median_rank | worst_rank | best_rank |
|---|---|---|---|---|
| P01011 | 100 | 1 | 2 | 1 |
| P02750 | 100 | 2 | 5 | 1 |
| P01009 | 100 | 3 | 6 | 1 |
| P15144 | 100 | 4 | 9 | 2 |
| P18428 | 100 | 5 | 11 | 2 |
| P05362 | 100 | 7 | 13 | 4 |
| P01833 | 100 | 7.5 | 12 | 3 |
| P05109 | 100 | 9 | 18 | 4 |
| P06681 | 100 | 9 | 13 | 5 |
| P01031 | 100 | 10 | 16 | 5 |
| P02748 | 100 | 10 | 16 | 5 |
| Q06033 | 100 | 11 | 16 | 4 |
| P02753 | 100 | 14 | 28 | 8 |
| P08637 | 100 | 16.5 | 37 | 8 |
| P02741 | 100 | 17.5 | 35 | 9 |
| P05452 | 100 | 18 | 40 | 11 |
| Q99784 | 100 | 18.5 | 41 | 11 |
| P05160 | 100 | 19 | 37 | 14 |
| P02647 | 100 | 20 | 40 | 13 |
| P02652 | 100 | 21 | 37 | 11 |

### Optimal Final Model Parameter Selection by GLMnet RCV

Using the top 20 features selected from the XGBoost RCV, the same subjects (n=127) were used but in a newly randomized collection of repeat-fold splits for a second stage of 10×10 RCV with GLMnet-based logistic regression. Although several modeling engines could be used here, GLMnet was selected as an exemplar to obtain individual subject class probabilities for additional comparisons to other models (e.g., to CA19-9 model performance) and created model terms (e.g., feature coefficients). This modeling engine can also be used for feature selection/reduction.

In the same manner as for the XGBoost RCV described above, a hyperparameter training grid of 200 possible combinations was created. Given the smaller number of input predictor features (20 vs 447) a complete RCV was selected, rather than using race tuning ANOVA for futility analysis, since most models were expected to perform very well, and the race selection process might introduce parameter selection variability due to very small differences in model performance.

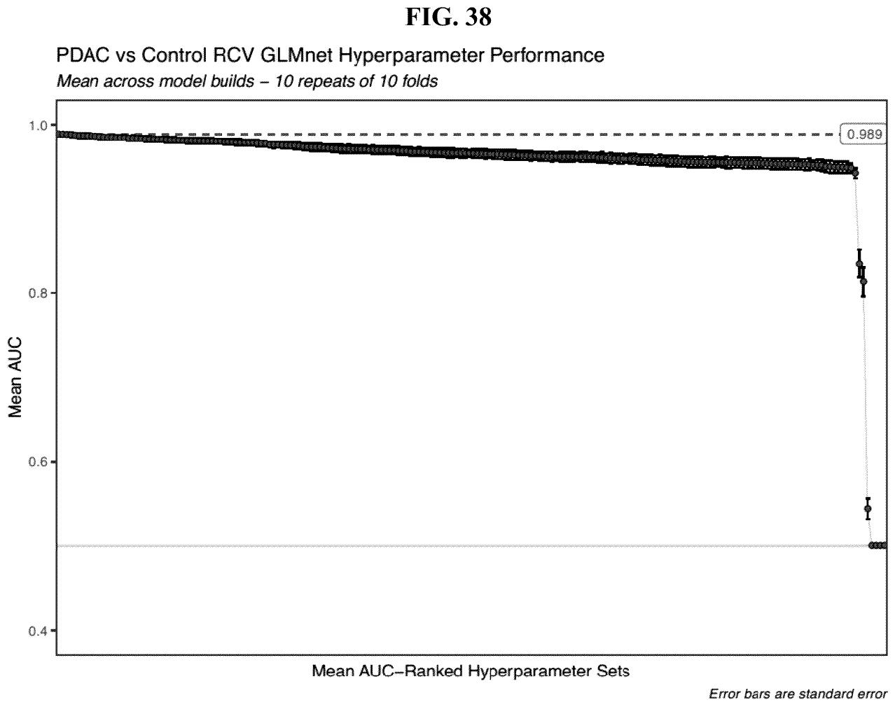

FIG. 38 shows the results of the 10×10 GLMnet RCV with respect to hyperparameter evaluation. Most of the hyperparameter combinations worked well with mean AUC's above 0.9. The best parameters are shown in Table 7 with a mean RCV AUC of 0.989.

TABLE 7
Optimized GLMnet top feature model hyperparameters
selected in 10 × 10 RCV

| Parameter | Value |
|---|---|
| penalty | 1.28037306940815e−09 |
| mixture | 0.00569066538009793 |

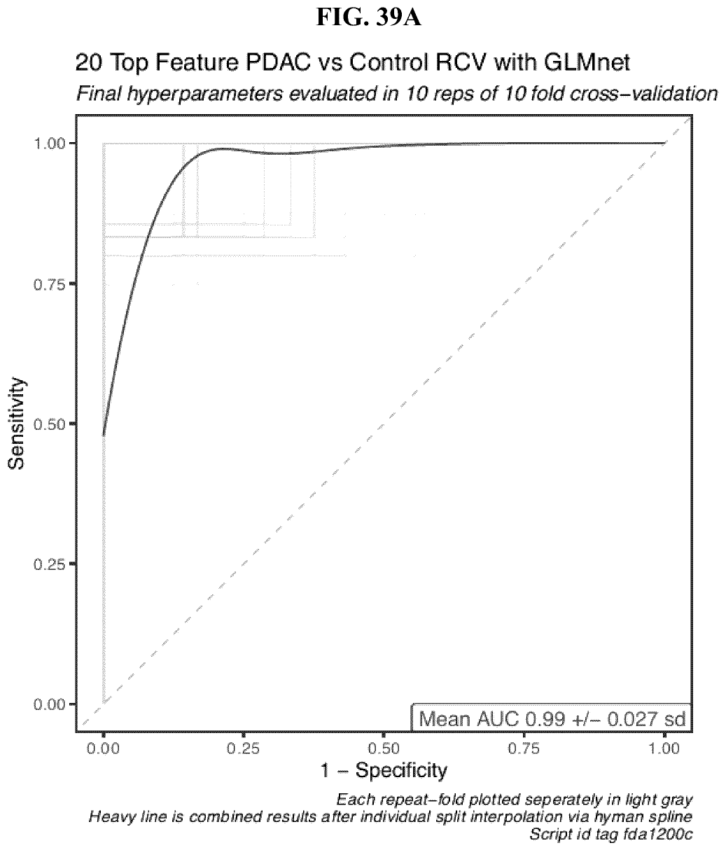

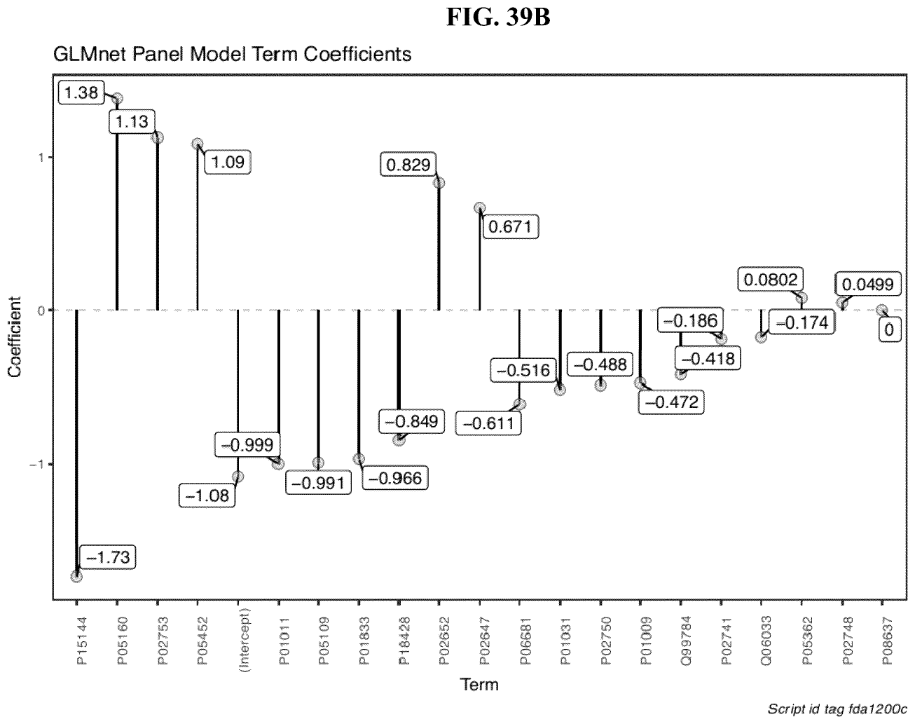

Using the selected hyperparameters, the combined ROC plots for those 100 models with the 10×10 RCV are shown in FIG. 39A. As before (see FIG. 37), the interpolated, combined results of the 10×10 RCV are shown in the highlighted line and the individual plots for the 11-14 subjects in the testing split of each repeat-fold are shown in light gray. The GLMnet model 10×10 RCV results based on the XGBoost top indicate that a final classifier constructed with the selected GLMnet parameters using all of the training data may have a useful degree of performance (e.g. with clinically useful sensitivity and specificity comprising a practicable number of features). A final GLMnet model was built using all the training data (n=127) and the optimized penalty and mixture parameters. As expected, this final model had excellent performance (AUC 0.995) when evaluated back on those training data used to construct the model. The coefficients for the logistic regression model are shown in FIG. 39B. As is evident in the figure, the coefficients are a mixture of positive and negative values, many with similar magnitude, confirming that a multivariate classifier may be more useful to achieve optimal performance than any single feature. The plot of the coefficients also indicates that subsets of the top 20 features might also achieve significant performance given the shrinkage towards zero across the panel.

### Validation of Final Top-Feature-Based GLMnet Model in Validation

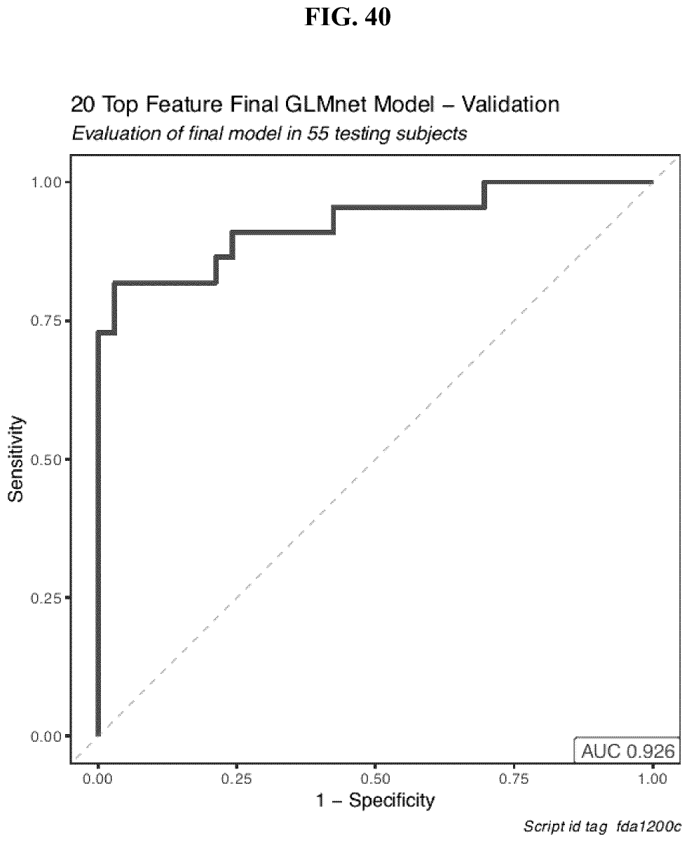

Using the final GLMnet model, the predicted classes and probabilities for the held-out validation group of subjects (n=55) was obtained. FIG. 40 shows the validation ROC plot and annotates the calculated AUC of 0.926 (0.8479-1 95% CI by DeLong). Using 2,000 stratified bootstrap re-samplings, the sensitivity of this model on the validation data at the indicated specificities was calculated and shown in Table 8.

TABLE 8
Sensitivity and specificity values for final
top feature GLMnet model in validation

| | Sensitivity | | High |
|---|---|---|---|
| Specificity | Low (95% CI) | Median | (95% CI) |
| 0.99 | 0.5455 | 0.7727 | 0.9545 |
| 0.98 | 0.5455 | 0.7727 | 0.9545 |
| 0.95 | 0.5909 | 0.8182 | 0.9545 |

A validated model performance which achieved 77% sensitivity at 99% specificity demonstrating the feasibility of this model for potential clinically useful performance for PDAC detection.

### Comparison to CA19-9 PDAC Detection

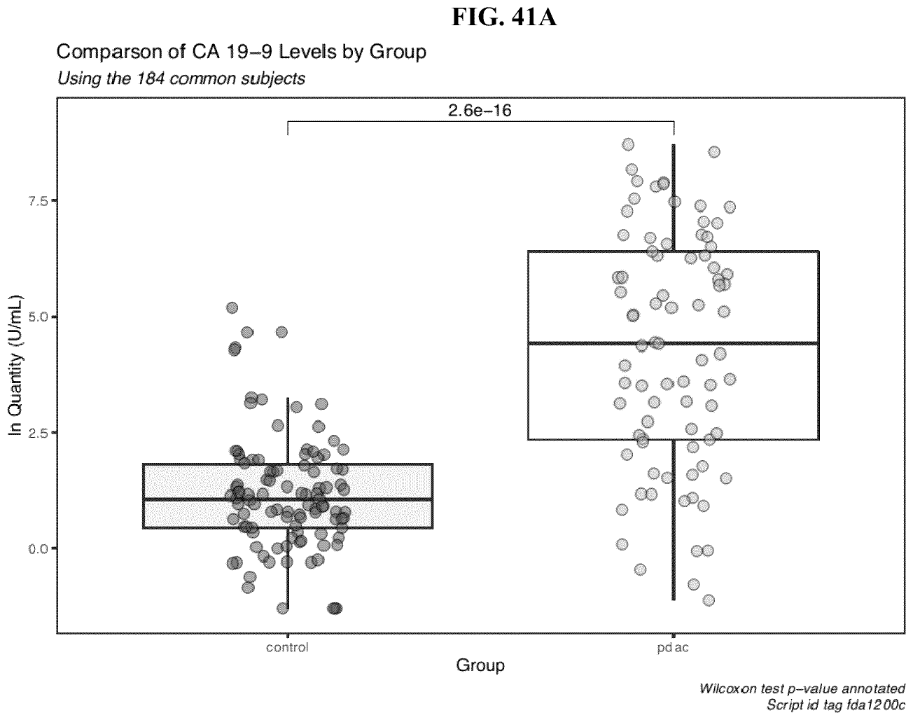

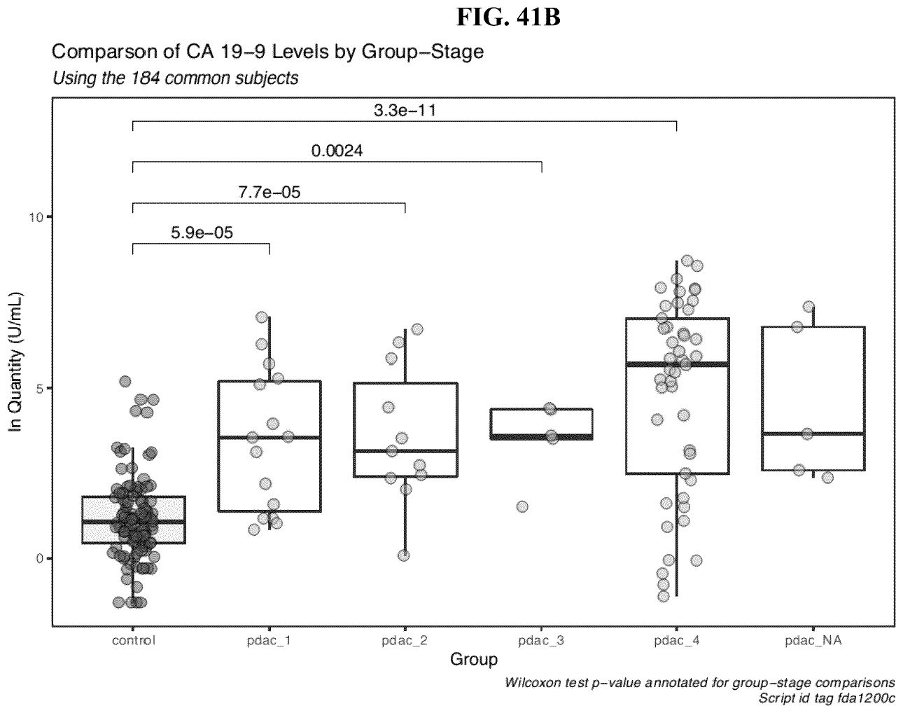

The cancer antigen CA19-9 is frequently used a marker for pancreatic cancer, usually as a recurrence test given its lack of specificity in a naïve screening population. In order to compare the classifier's performance to this marker, CA19-9 levels were measured in the subjects using an analyte-specific, clinical-grade assay. FIG. 41A and FIG. 41B below show CA19-9 levels for the cancer groups and cancer stages compared to controls, respectively. The data show that CA19-9 was significantly elevated in the PDAC subjects versus non-cancerous controls. There also appeared to be a significant increase in stage 4 levels versus earlier stages, although the comparison to stage 3 may be further validated with additional subjects.

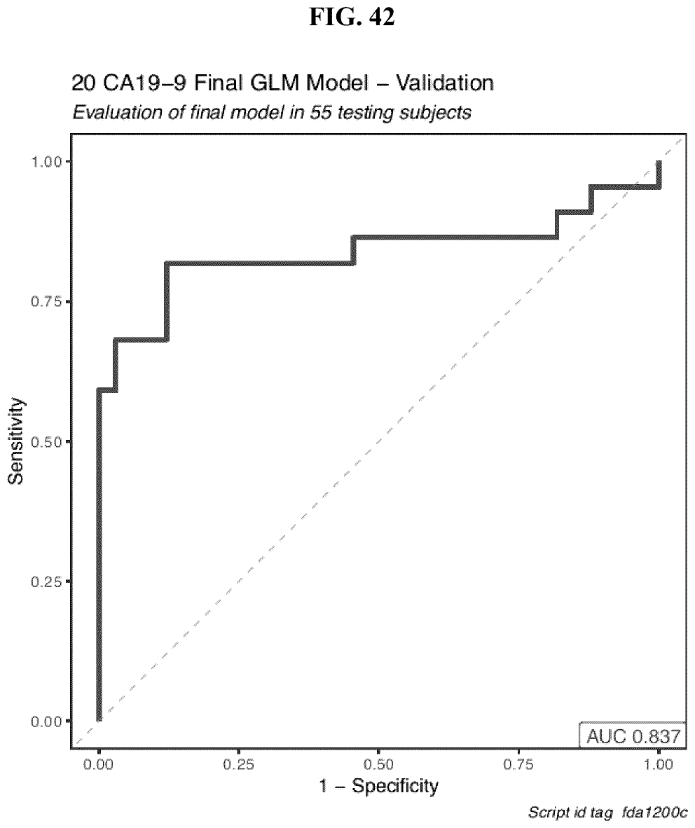

Using these measured CA19-9 levels, a simple logistic regression engine, GLM, was used to convert clinical assay values to model probabilities, first constructing the model in the full n=127 training data, and then evaluating the model in the n=55 validation set. Conversion of assay values to model probabilities enables subsequent direct comparisons of models' performances. FIG. 42 shows the performance of CA19-9 as a classifier in the validation set. As the plot indicates, an AUC of 0.8375 (0.7021-0.9729 95% CI) was achieved. The model's performance at the same specificity points as highlighted above is shown in Table 9.

TABLE 9
Sensitivity and specificity values for
final CA19-9 GLM model in validation

| | Sensitivity | | |
|---|---|---|---|
| Specificity | Low | Median | High |
| 0.99 | 0.4091 | 0.6364 | 0.8636 |
| 0.98 | 0.4091 | 0.6364 | 0.8636 |
| 0.95 | 0.4545 | 0.6818 | 0.9091 |

Comparison of the sensitivity of 64% at 99% specificity to the top feature model above (e.g., 77% sensitivity at the same specificity) indicates that the panel represents a significant improvement over existing tests such as stand-alone CA19-9. A comparison of the ROC plots via paired bootstrap resampling (n=50,000) gave a p-value of 0.147, where the differences of the two AUCs and the standard deviation of the bootstrap differences (e.g., D=(AUC1−AUC2)/s) were compared to the normal distribution.

### Combined Performance of Top Features and CA19-9

Although CA19-9 has not achieved clinical acceptance for widespread testing for cancer in naïve populations, it is possible that, when combined with other potential marker components, it may add significantly to overall performance. To evaluate this possibility, a multivariate classifier was developed using the top 20 XGBoost RCV features as selected above in combination with CA19-9 using the same GLMnet-based approach for logistic regression as described above. Using the same training data (n=127) as well as the same repeat-folds as for the top feature GLMnet classifier, a new round of 10×10 RCV was performed. Optimal hyperparameters are shown in Table 10, and a final model based on all the training data was constructed.

TABLE 10
Optimized GLMnet combined model hyperparameters
selected in 10 × 10 RCV

| Parameter | Value |
|---|---|
| penalty | 0.008859443307578 |
| mixture | 0.283842075379798 |

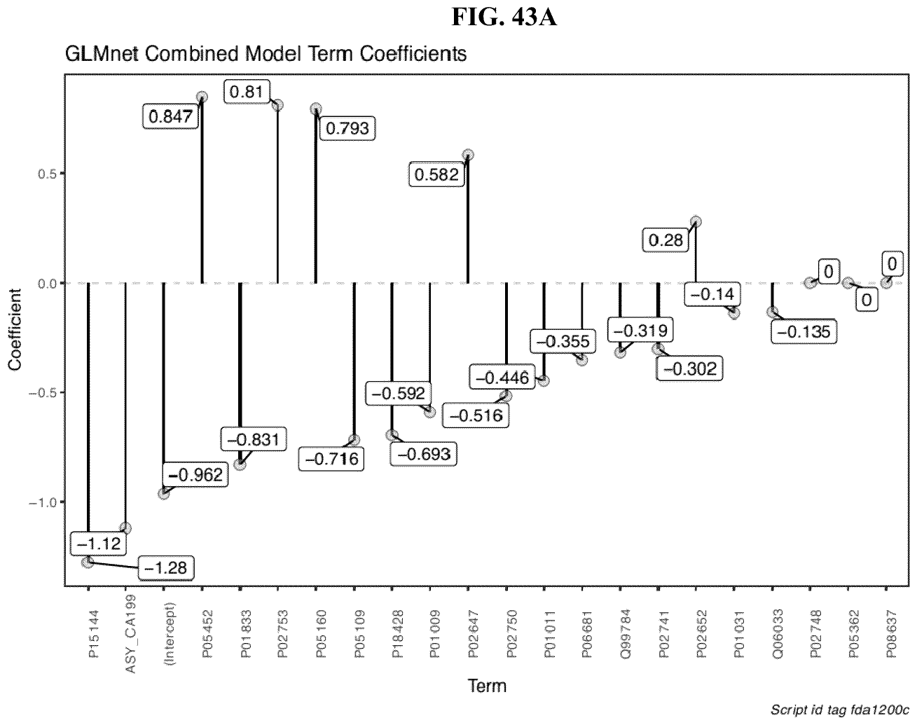

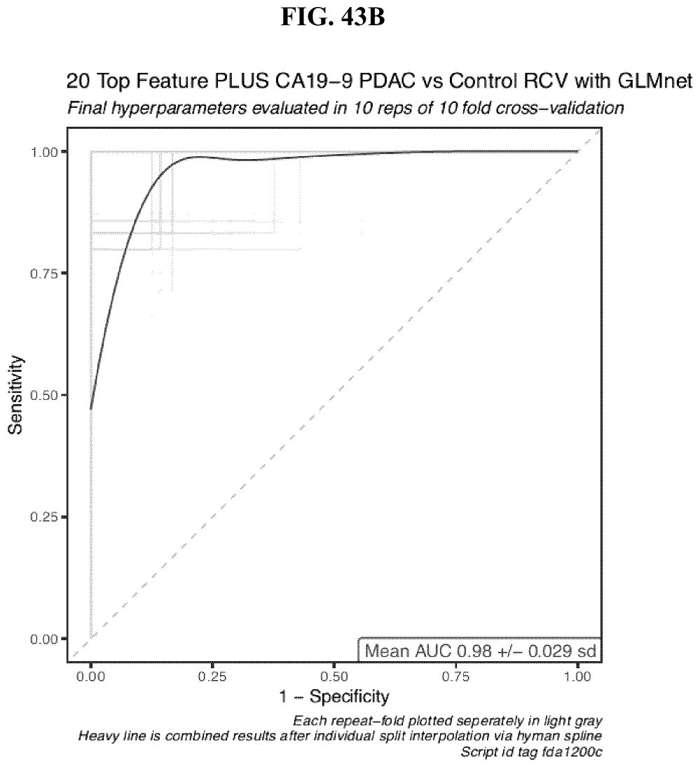

The coefficients for the final model based on all of the training data are shown in FIG. 43A, and the performance of those best parameters over the 100 models of the 10×10 RCV are shown in FIG. 43B with a mean AUC of 0.98. Although CA19-9 was ranked highly in this combined classifier with the second highest absolute value regression coefficient for its regression term, there was one other feature with a higher value (e.g., P15144) and several others with close values. As such, the features added from the XGBoost RCV selection were useful factors in this combined model.

### Final Validation of Combined (Top Features Plus CA19-9) Model

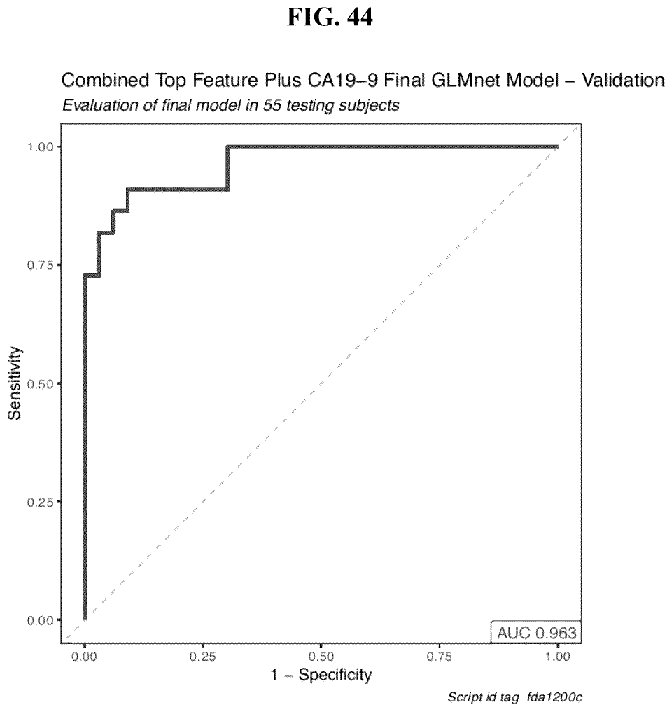

Using the final model for the combined features on the held-out, validation set of subjects (n=55), the class predictions and probabilities were obtained, and the performance was visualized in the ROC plot shown in FIG. 44. The AUC was 0.9628 (0.9193-1 95% CI)

The calculated sensitivities at various specificities, calculated as described above, are shown in Table 11 (and using 0.5 as the class probability threshold). The estimated performance of 77% sensitivity at 99% specificity is a marked improvement over the 64% sensitivity at 99% specificity described for CA19-9 alone. Comparison of the ROC curves via bootstrap resampling confirmed the statistical significance of the combined vs CA19-9 curves with a p-value=0.045. Using a class probability of 0.5 as for the table above, the prediction confusion matrix is shown in Table 12. Effectively, there is good balance between the classes for model accuracy.

TABLE 11
Sensitivity and specificity values for combined GLMnet model

| | Sensitivity | | |
|---|---|---|---|
| Specificity | Low | Median | High |
| 0.99 | 0.5455 | 0.7727 | 0.9545 |
| 0.98 | 0.5455 | 0.7727 | 0.9545 |
| 0.95 | 0.5909 | 0.8182 | 1.0000 |

TABLE 12
Confusion matrix for combined feature GLMnet-based classifier

| | Truth | |
|---|---|---|
| Prediction | PDAC | Control |
| PDAC | 19 | 2 |
| Control | 3 | 31 |

### Combined Model Performance Across PDAC Classes

Since earlier detection of PDAC is an important goal for this study and for eventual clinical utility, the classification performance of the final combined model classifier across the cancer stages represented in the validation set was evaluated. The scores in Table 13 show that 8 out of 10 (80%) stage 1-3 subjects were correctly classified, and 78% of stage 1-2 subjects were correctly classified. The data indicate that the classifier's performance extends significantly across all PDAC stages.

TABLE 13
Accuracy of final validated combined model
classification across PDAC stages

| Group-Stage | Incorrect | Correct | Accuracy |
|---|---|---|---|
| Control | 2 | 31 | 0.939 |
| pdac_1 | 1 | 4 | 0.8 |
| pdac_2 | 1 | 3 | 0.75 |
| pdac_3 | 0 | 1 | 1 |
| pdac_4 | 0 | 10 | 1 |
| pdac_NA | 1 | 1 | 0.5 |

### Novelty of Top Features

Although the MS-based assay for the 447 proteins evaluated in this PDAC versus non-cancerous control study were “targeted” from a technical point of view with respect to MS data acquisition, the proteins in that panel were not specifically biased towards PDAC or for cancer per se. These proteins were not a truly random sample of all possible plasma-detectable proteins, but given their relative unbiased nature, they were useful in finding novel combinations of known and unknown players in PDAC detection.

One way to assess novelty of classifier components is to observe their importance or interest as defined as a disease-related association score in an aggregated database such as OpenTargets. PDAC overall association scores for 4,886 genes and associated proteins are listed in a table annotated as EFO0002517 from the OpenTargets database. By mapping the Uniprot identifiers from the PiQuant-base protein features, the relative ranking of the selected proteins to those in the database can be visualized. The OT rankings include many components of interest (e.g., developed-drug, publications, genetic associations, etc.) and not just plasma-detectability, so simply selecting the genes or proteins with the highest OpenTargets scores in-of-itself may be insufficient to develop a blood-based test for the detection of any given disease.

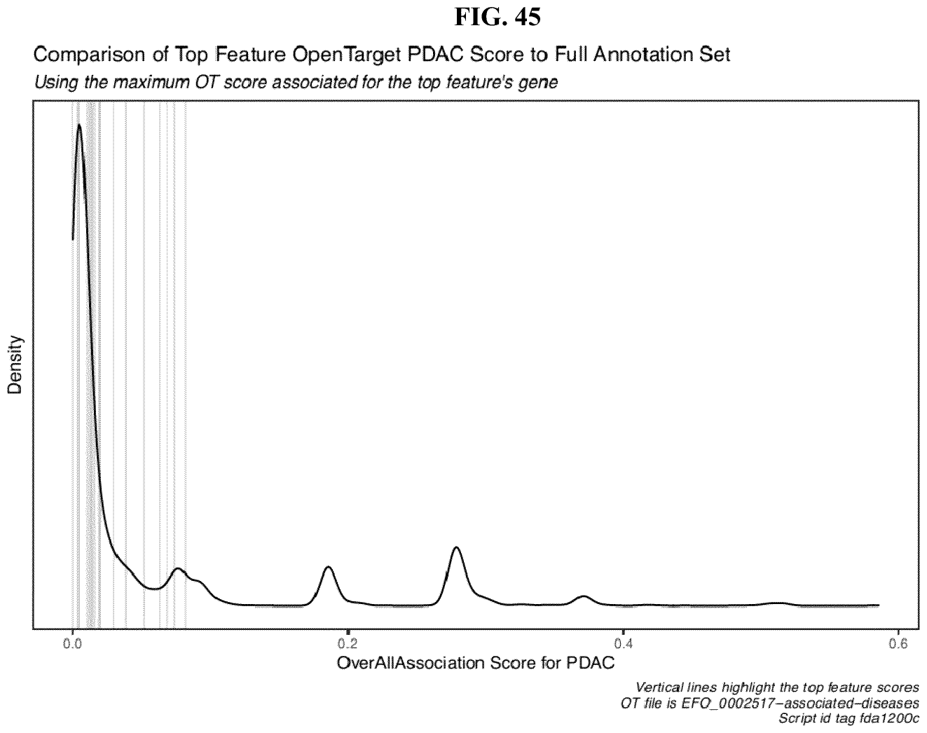

In FIG. 45, the overall distribution of the OpenTargets PDAC-related “overallAssociationScores” (n=4,886) was plotted and annotated the distribution for the 16 out of 20 combined panel proteins which have non-zero scores. As can be seen in the plot, although these 16 proteins do have scores greater than zero, they may not necessarily have been prioritized for a targeted plasma-based detection effort based strictly on score ranking. 81% of the database's features had higher scores than the maximum value for the panel proteins (0.0820 on a scale from 0 to 1). The four proteins with no PDAC scores may not have been selected at all using these annotations as a criterion.

### Example 9. Multi-Omics Data Shows Further Improves Potential for Early-Stage Pancreatic Cancer Detection

A classifier was trained on 112 plasma samples that had metabolite, lipid, proteins, methylation, and mRNA data. The samples included samples from 9 subjects with stage 1 pancreatic cancer, 9 subjects with stage II pancreatic cancer, 2 subjects with stage III pancreatic cancer, 27 subjects with stage IV pancreatic cancer, 4 subjects with pancreatic cancer of an unknown stage, and 61 subjects without cancer. At least some of these samples overlapped with samples of other examples described herein.

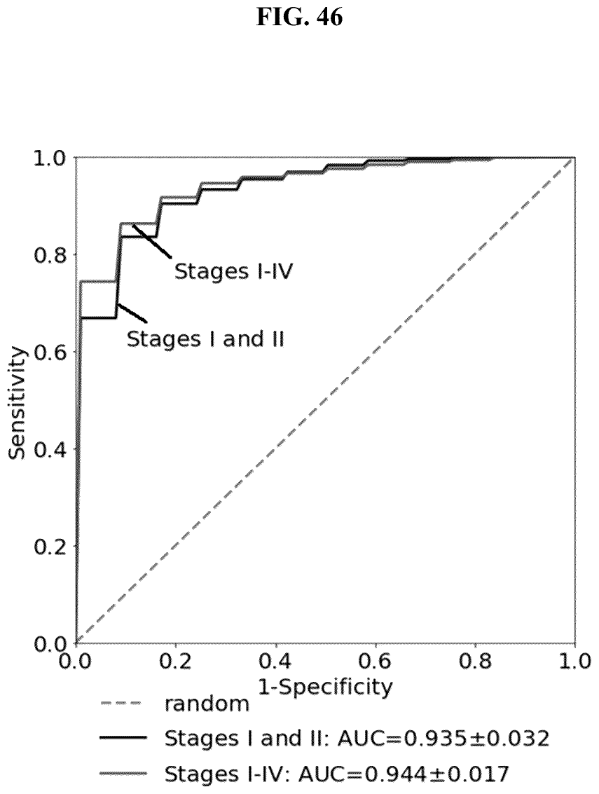

A stagewise analysis showed performance on Stages I and II (ROC AUC of 0.935) that almost as good as the performance across all stages (ROC AUC of 0.944) (FIG. 46). Sensitivity at 98% specificity ranges between 64-73% for the two sample sets analyzed to date.

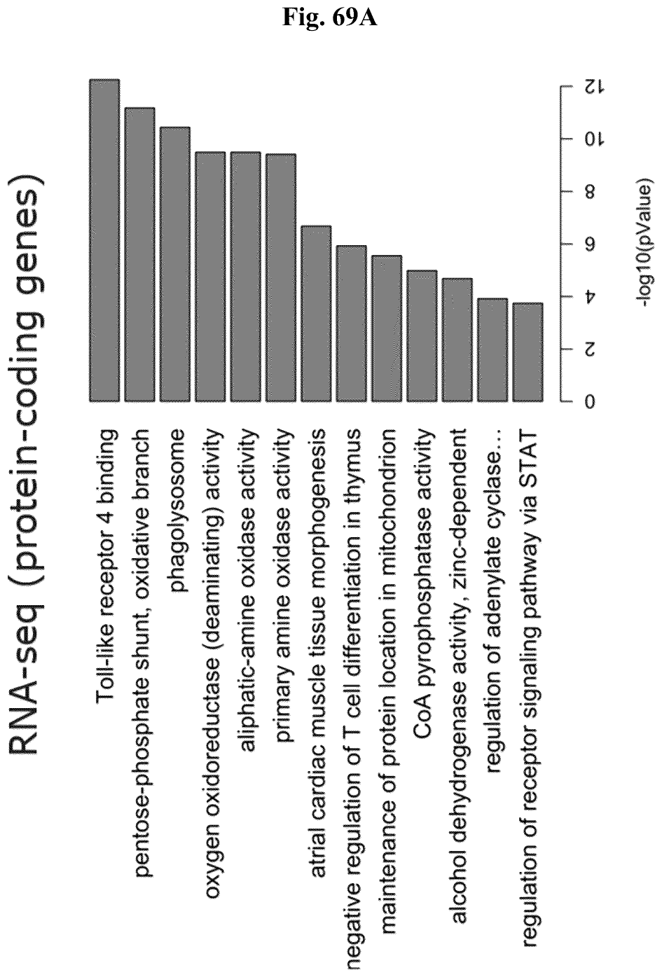

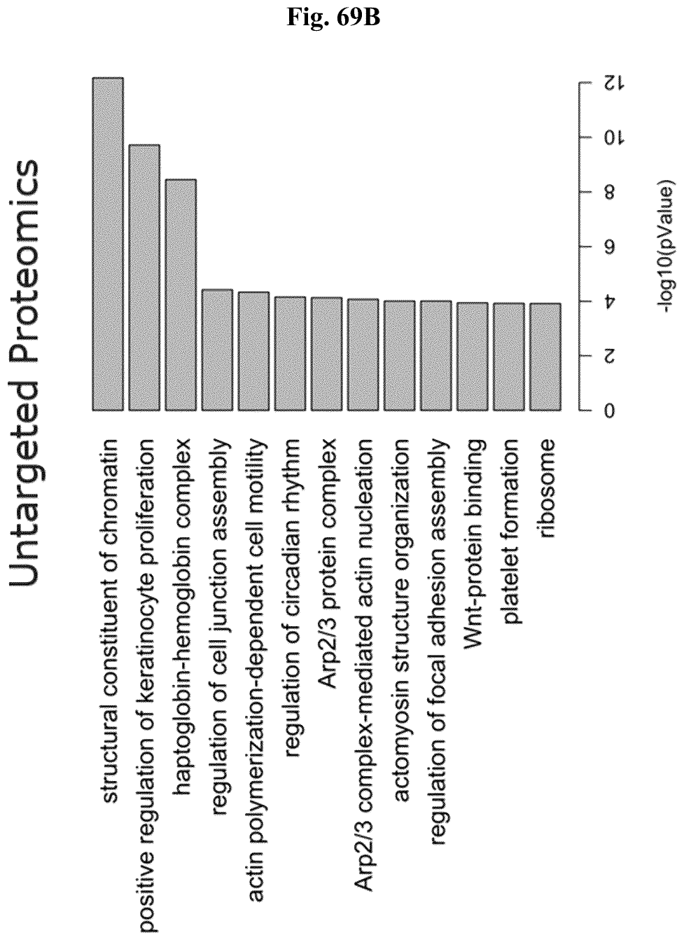

Different biological processes were observed in RNA-seq and untargeted proteomics data. FIG. 69A, for example, illustrates biological processes captured by RNA-seq. Levels of significance observed each biological processes are shown. Toll-like receptor 4 binding was the most statistically significant in the RNA-seq data shown in the figure. FIG. 69B illustrates biological processes captured by untargeted proteomics. Again, levels of significance observed in biological processes shown. Structural constituent of chromatin was found to be the most statistically significant in Untargeted Proteomics. FIGS. 69A-69B show that different molecular assays captured analytes from distinct biological processes.

### Example 10. Multi-Omics Platform Applied to Pancreatic Cancer

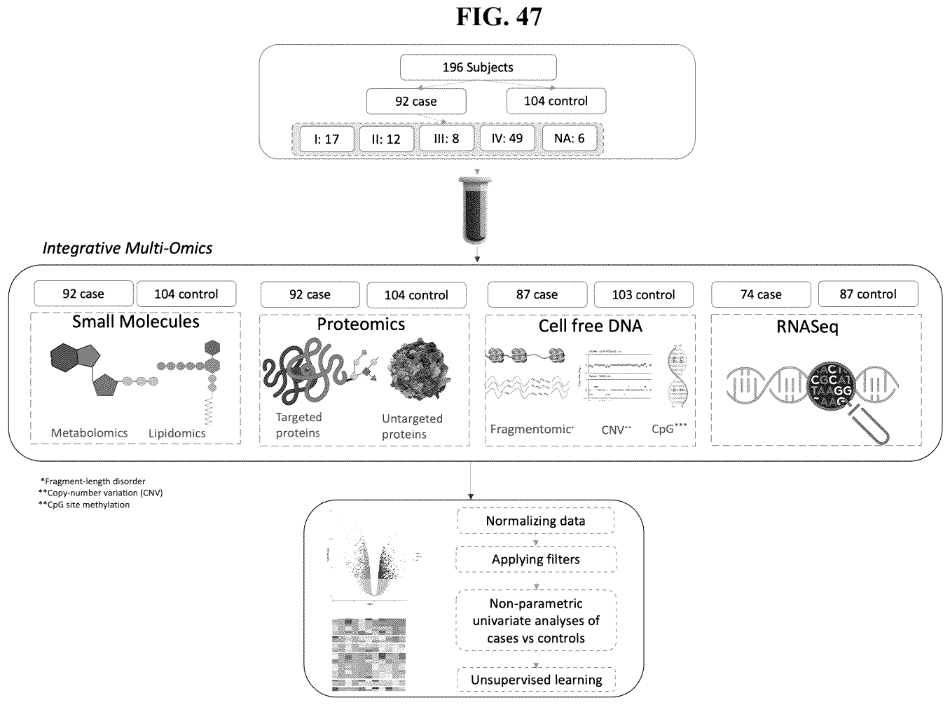

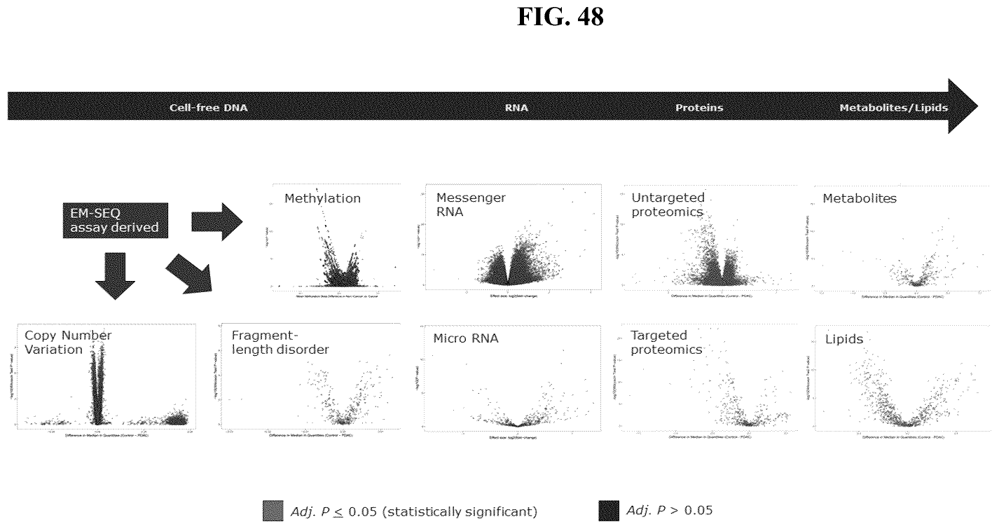

FIG. 47 shows sample and analysis details used in multi-omics experiments for pancreatic cancer. At least some of these samples and study details overlapped with those described in of other examples. The multi-omics cancer biomarkers spanned a genotype-to-phenotype spectrum (FIG. 48).

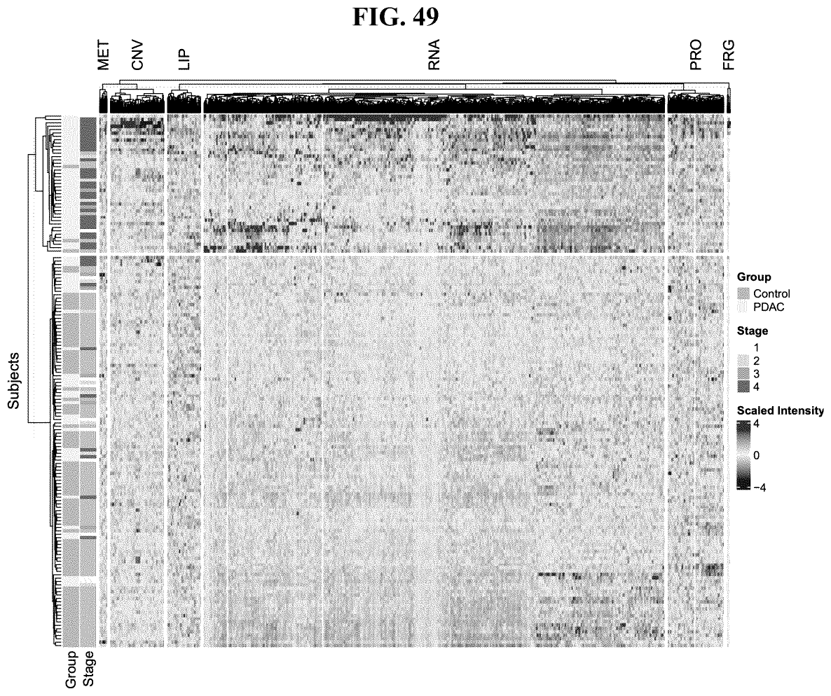

Multi-omics assays captured individual and shared biological signals that aggregately separated cancer from non-cancer. The data in FIG. 49 includes bi-clustering on statistically significant (non-cancer vs. cancer, Adj P<0.05) multi-omics biomarkers.

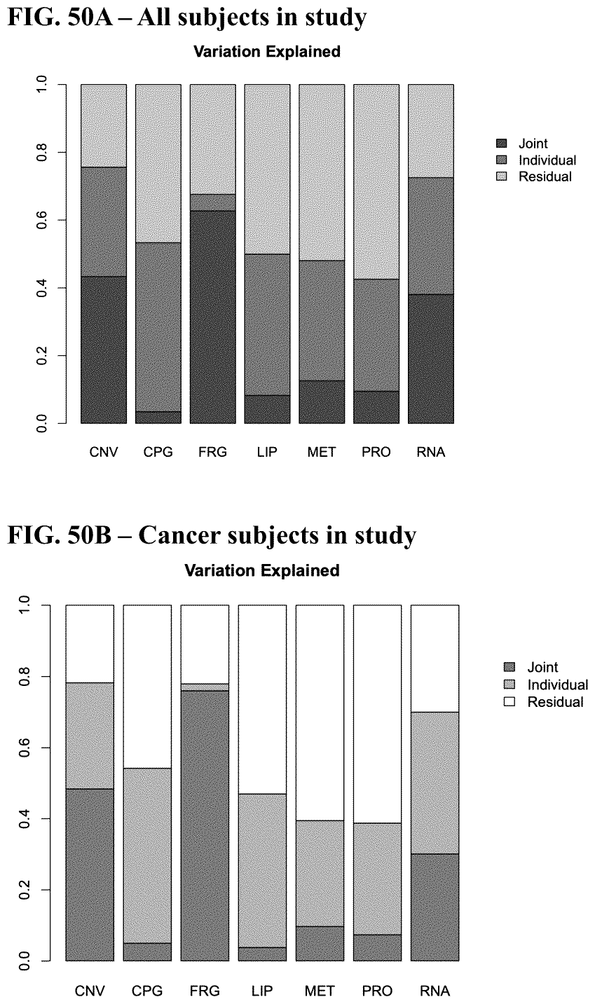

Variance decomposition illustrated that distinct aspects of biology may be uniquely captured by each molecular assay. The data in FIGS. 50A-50B includes unsupervised variance decomposition (JIVE) on statistically significant (Adj P<0.05) biomarkers. Some takeaways were that there was shared biology across the different 'omics (the joint component), and that they could be used as independent lines of evidence to uncover a shared biological signal. An additional takeaway was there was also a lot of biology that was specific to each assay, especially methylation, lipidomics, metabolomics, and proteomics (the individual component).

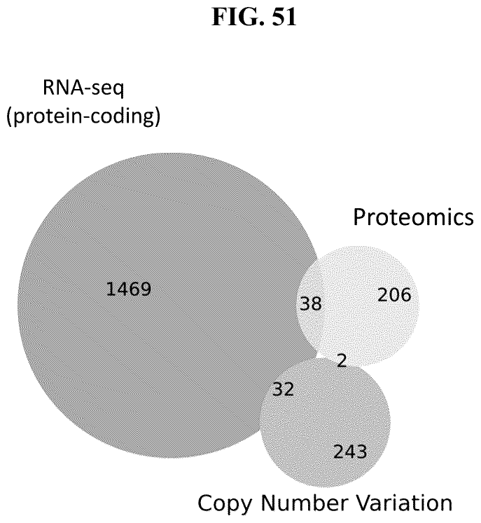

Examining across multi-omics readouts can prioritize biomarkers for further investigation. FIG. 51 includes overlap of statistically significant (Adj P<0.05) biomarkers for RNA-seq (protein-coding genes), proteomics (untargeted+targeted), and copy number change regions. Examining overlaps across multi-omics assays can zero-in on high priority biomarker candidates. The two proteins overlapping with copy number change in FIG. 51 were E and N cadherin and may be relevant for epithelial-to-mesenchymal transition in pancreatic cancer. Additional experiments will be performed to further understand the statistically significant, overlapping gene and copy number as well as gene and protein findings.

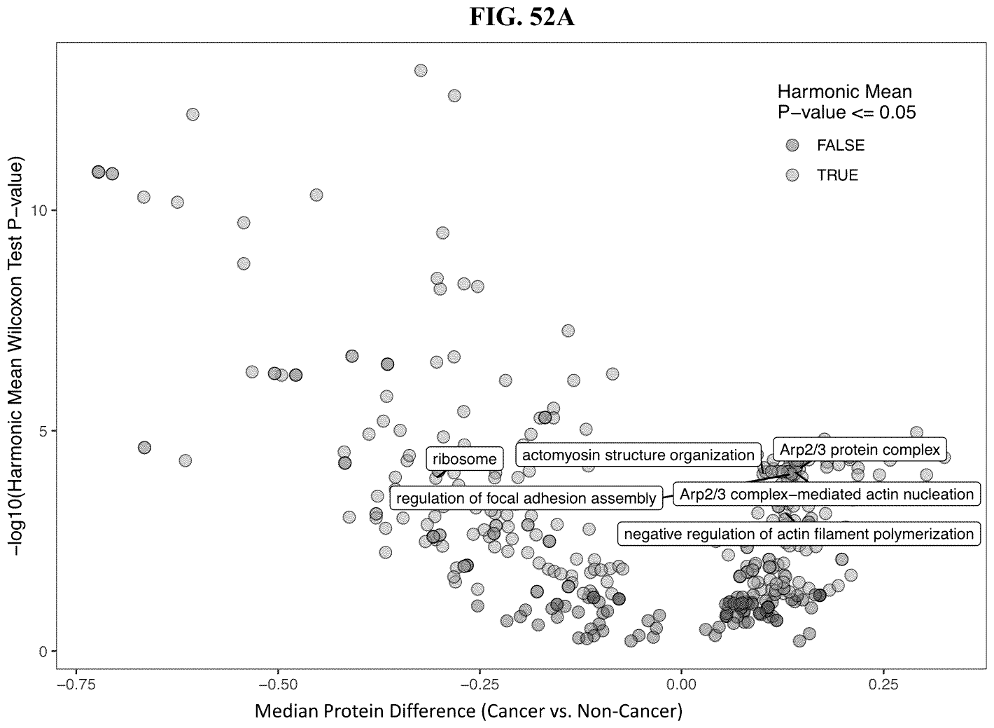

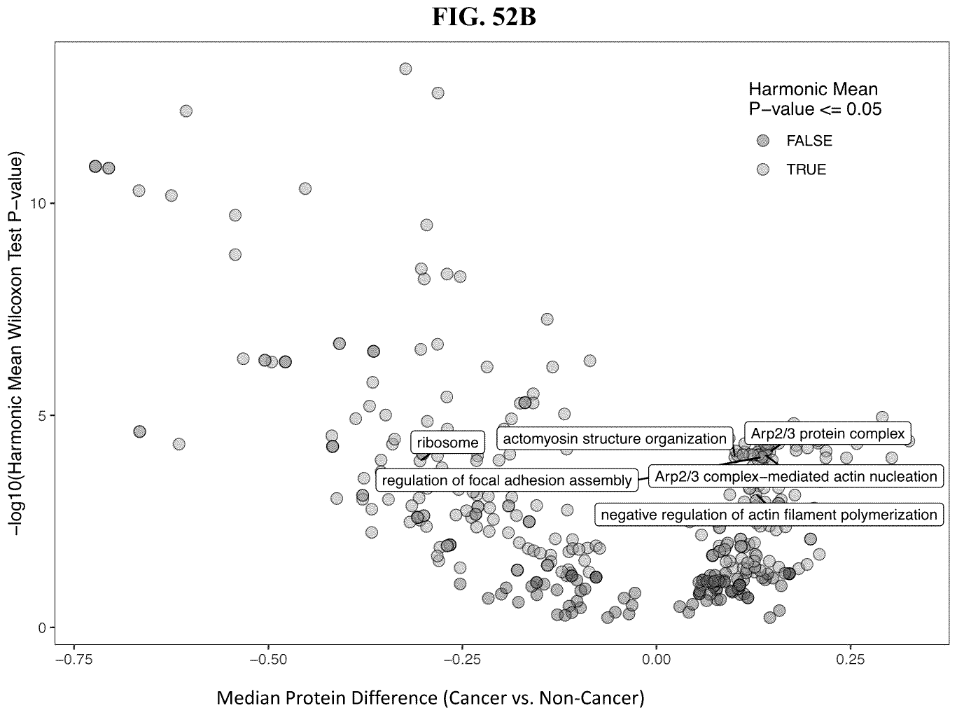

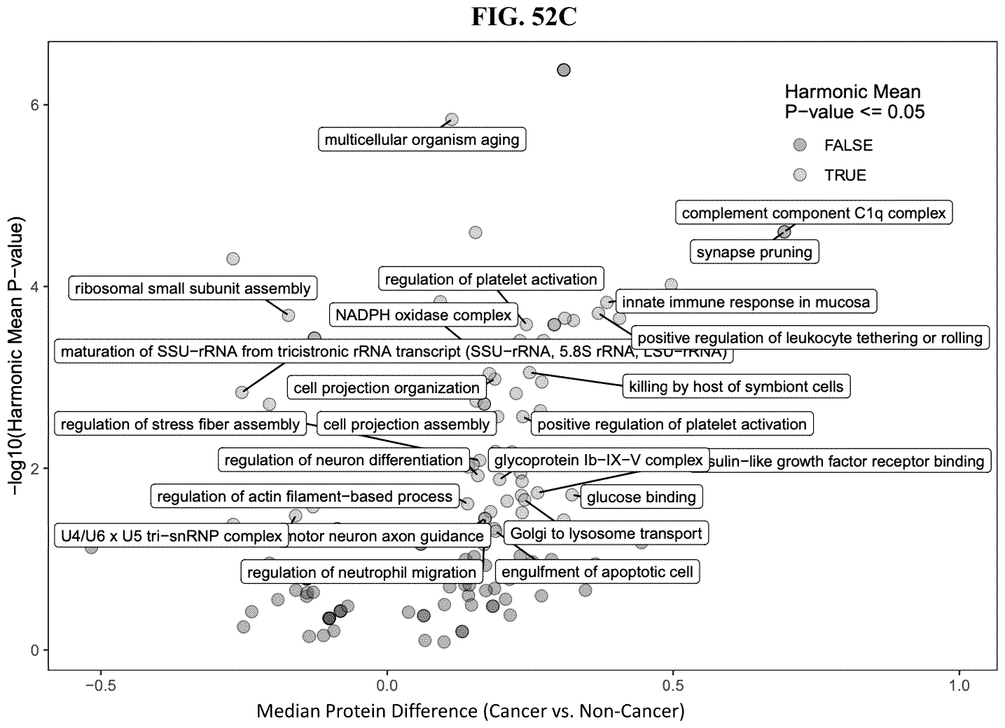

Multi-omics readouts can also be statistically combined to improve the interpretation of biological processes, as shown in FIGS. 52A-52C, which included untargeted proteomics and RNA-seq+untargeted proteomics.

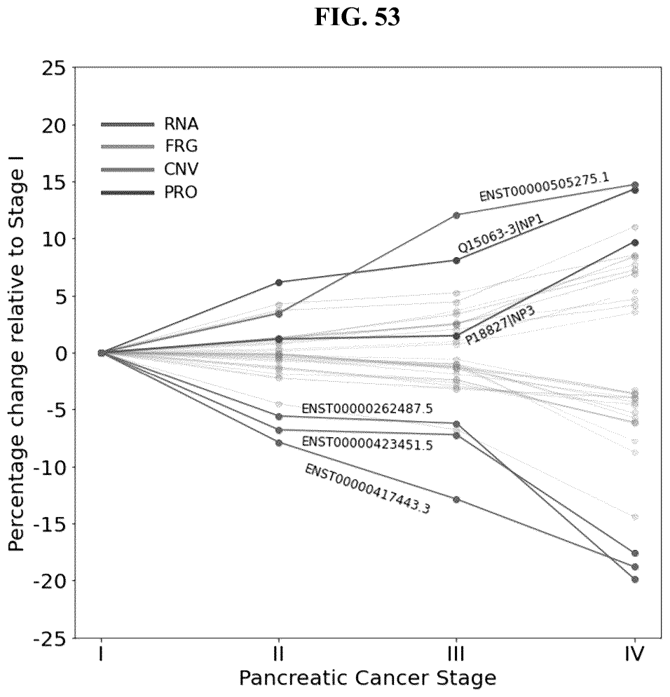

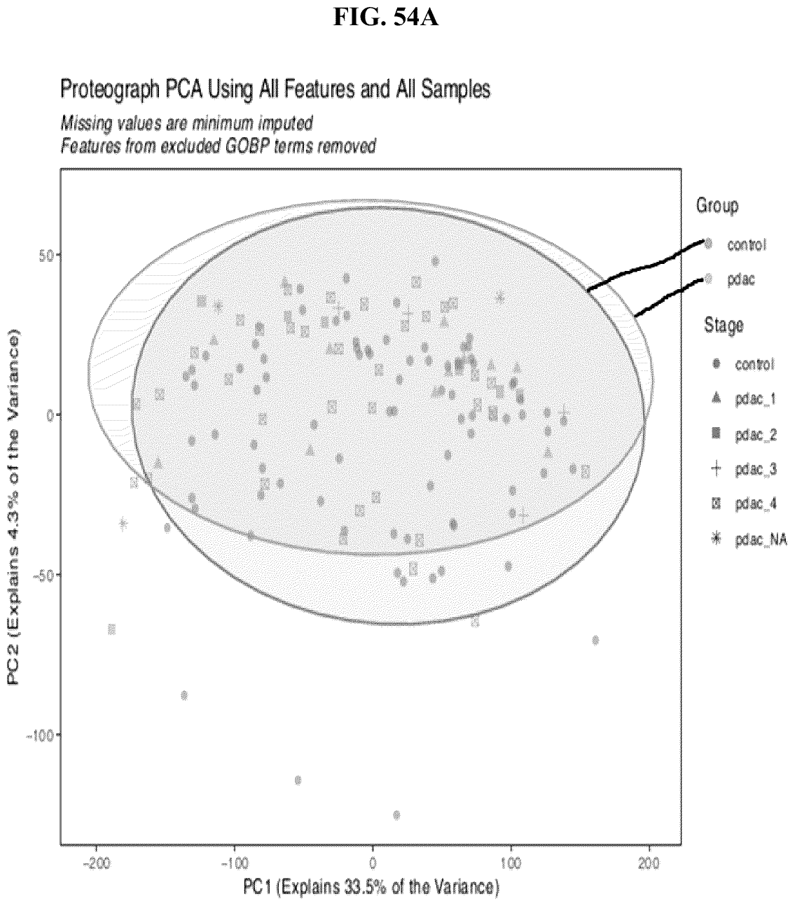

Trend analysis showed correlation of marker abundance with cancer stage (FIG. 53). In the figure, groups of RNA, fragment (FRG), CNV, and protein (PRO) data can be seen. The trend analysis was performed using a one-sided Jonckheere-Tempstra test with Bonferroni procedure for multiple hypothesis correction (Adj P<0.05). Identified markers showed monotonic increases or decreases with cancer stage. As such, a classifier or method herein may be used to distinguish a cancer stage.

Some aspects of FIG. 53 can be further elaborated by reference to Table 14. Any of the biomarkers in this table may be used as biomarkers for pancreatic cancer either alone or in combination.

TABLE 14

| Marker | Gene symbol | Trend |
|---|---|---|
| ENST00000423451.5 | ST6GAL1 | ↓ |
| ENST00000417443.3 | SMIM10L2A | ↓ |
| ENST00000262487.5 | ISM1 | ↓ |
| ENST00000505275.1 | HAUS1P1 | ↑ |
| Q15063-3\|NP1 | POSTN | ↑ |
| P18827\|NP3 | SDC1 | ↑ |

### Example 11. Validated Pancreatic Cancer Classifier Based on a Multi-Omics Panel with Targeted Mass Spectrometry Assay in a Case-Control Study of 146 Subjects

### Summary

Effective and reliable methods to detect pancreatic cancer are urgently needed given the current lack of early detection tools, the often-asymptomatic disease course, and the poor prognosis associated with late-stage diagnosis. This study used acute status-focused, multi-omics analyses to construct and validate a new 20-feature classifier that discriminates pancreatic ductal adenocarcinoma (PDAC) subjects from non-cancer controls across all stages. Features included protein, metabolite, lipid, and RNA data from blood samples collected from 146 age- and sex-matched subjects, and exploratory analyses indicated a number of potentially differentiating signals. Repeated cross-validation (RCV) with a training cohort of 74 subjects was applied to construct individual omics models using all features. The 5 features that contributed most to each model were identified and used as input for a new RCV with a re-shuffling of the training subjects. A final, 20-feature, multi-omics model was constructed, and examination of the model's coefficients indicated that each omics type contributed significantly. This model was applied to another cohort of 72 validation subjects, and it achieved an all-stage classification area under the ROC curve (AUC) of 0.977 with 80.8% sensitivity at 99% specificity. Early-stage (stage I/II) subjects had an AUC of 0.965 with 71.4% sensitivity at 99% specificity. The model comprises a novel combination of analytes both unknown and known to be related to PDAC and demonstrates the value of combining multiple different omics to develop clinically useful tests for early pancreatic cancer detection.

### Introduction

Pancreatic cancer is currently the fourth most common cause of cancer-related mortality in the United States, and demographic trends suggest that it will become the second leading cause by 2030. Pancreatic ductal adenocarcinoma (PDAC) and its variants compose more than 90% of pancreatic malignancies. These are feared diagnoses because the majority of cases are not detected until late stage, with 80-85% of initial presentations representing incurable locally advanced or metastatic unresectable disease. This contributes to the low all-stage 5-year survival rate of approximately 10%. However, as diagnosis at early-stage has a markedly superior 5-year survival rate of over 40%, early detection, potentially with peripheral blood biomarker tests, with the potential to downstage initial diagnosis, hold significant promise in reducing PDAC-associated morbidity and mortality in appropriate screening populations. Multiple clinical and investigational biomarkers are in use, including CA19-95 and those based on proteins and DNA methylation, however, given the limited performance of those markers, the US Preventative Services Task Force currently recommends against routine screening for PDAC.

PDAC is challenging to detect as the onset of clinical symptoms typically coincides with the progression to invasive growth and loss of opportunity to resect. In addition, the unique tumor microenvironment, consisting of PDAC-associated stroma, creates an immune-privileged compartment that has been refractory to recent advances in immuno-oncology-based therapeutics. Given the complexity of PDAC progression, a plurality of signal inputs, such as from different blood analytes, may be necessary to detect PDAC early enough for interventions that improve patient survival. Thus, a multi-omics model utilizing a combination of orthogonal features (e.g., proteins, metabolites, lipids, and RNAs) sampling a wide variety of physiological systems and pathways may demonstrate superior classification performance compared to any individual omics model and may be practical for clinical development. Here, the feasibility of this approach was validated using a case-controlled study of PDAC subjects versus age- and sex-matched non-cancer controls. Using broad, unbiased platforms to collect analyte data for each omics type, individual omics classification models were used to select the most important features and then combined those selected features into a single, multi-omics classifier. The final performance of this model, against all and early-stage PDAC, was demonstrated in a separate validation cohort.

### Study Design and Subject Population

A case-controlled study comprising diagnosis-aware, but treatment-naïve, PDAC subjects and age- and sex-matched non-cancer control subjects was conducted. The subjects came from an ongoing, IRB-approved, observational study collecting a variety of blood sample types (e.g., plasma, serum, Streck and PAXgene tubes) for 5 different cancers as well as selected co-morbidity controls. For this analysis, a subset of 146 subjects comprising 63 PDAC and 83 non-cancer control subjects was selected from 16 different sites over a more than 2-year period. PDAC subjects were enrolled from 14 of the sites and non-cancer controls enrolled from 4 of the sites. The major inclusion/exclusion criteria were based on newly diagnosed, biopsy confirmed PDAC in the absence of any other cancer or cancer history for at least the preceding 5 years. On average, PDAC subjects' blood samples were collected 21 days after the local hospital pathologist reported PDAC histopathology. The control subjects were established as cancer-free based on self-reported medical history but were allowed to include other, non-related comorbidities (e.g., diabetes) to better evaluate classification model generalization to an eventual intent-to-test population. There were no significant differences in the frequency of 9 reported comorbidities between PDAC and control subjects. Gender, age, and race were not significantly different between PDAC and control subjects for each of the training and validation sets, and the proportion of PDAC stages was not significantly different between the training and validation sets. Though equal distribution across PDAC stages was not a protocol requirement, inclusion of early-stage subjects (Stages I and II; n=20) and late-stage subjects (Stage III and IV; n=40) with 3 subjects having incomplete records for staging were included.

### Proteomics Data Acquisition and Primary Data Processing

To maximize the potential for novel signal collection, an unbiased, non-analyte specific methodology for protein data collection was used. Plasma samples were processed through the Proteograph (Seer, Redwood City, CA) plasma sample preparation platform using the standard 5 nanoparticle panel and 3 process controls following the manufacturer's protocol. Eluted peptide concentration was measured using a quantitative fluorometric peptide assay kit (Thermo Fisher, Waltham MA) and dried down in a Centrivap vacuum concentrator (LabConco, Kansas City MO) at room temperature overnight. Prior to use, peptides were equilibrated at room temperature for 30 minutes and then reconstituted on the Proteograph platform in 0.1% formic acid (Thermo Fisher, Waltham, MA) in LCMS-grade water (Honeywell, Charlotte, NC,) spiked with heavy-labeled retention time peptide standards—iRT (Biogynosys, Switzerland) and Pepcal (SciEX, Redwood City, CA) prepared according to manufacturer's instructions. Isolated peptides were reconstituted in solution by shaking for 10 minutes at 1000 rpm at room temperature on an orbital shaker (Bioshake, Germany) and spun down briefly (˜10 seconds) in a centrifuge (Eppendorf, Germany). Reconstituted peptides were loaded onto Evotip separation tips (Evosep, Denmark) and processed following the manufacturer's protocol using a total of 600 ng of nanoparticle 1-4 peptides and 300 ng of nanoparticle 5 peptides. The processed tips were placed on the Evosep One LC system (Evosep, Denmark) and peptides were separated on a reversed-phase 8 cm×150 μM, 1.5 μM, 100 Å column packed with C18 resin (Pepsep, Denmark) using a 60 samples per day Evosep LC gradient method.

Peptides were analyzed on a timsTOF Pro II (Bruker, Germany) using Data Independent Acquisition (DIA) mode with Parallel accumulation-serial fragmentation; source capillary voltage was set to 1700 V and 200° C. Precursors (MS1) across m/z 100-1700 and within an ion mobility window spanning 1/K0 0.84-1.31 V·s/cm2 were fragmented using collision energies following a linear step-function ranging between 20 eV-63 eV. TIMS cell accumulation time was set at 100 milliseconds and the ramp time at 85 milliseconds. Resulting MS/MS fragment spectra between m/z 390-1250 were analyzed using a DIA schema with 57 Da windows (15 mass steps) with no mass/mobility overlap resulting in a cycle time of just under 0.8 seconds. Primary MS data were processed to protein group and peptide IDs with quantification using the Proteograph Analysis Suite (Seer) comprising the DIANN search engine. For all protein omics analyses, a unique nanoparticle-modified peptide sequence was the analyte feature; thus, proteomics analysis occurred at the (potentially modified) peptide level.

### Lipidomics and Metabolomics Data Acquisition and Primary Data Processing

Lipid data were acquired using a multiplexed, targeted liquid chromatography-mass spectrometry (LC-MS) assay, in which the target analytes were not selected based on any known association with pancreatic ductal adenocarcinoma (PDAC). Total lipid content was extracted using single phase organic extraction method. 5 μL of cohort, NIST SRM1950, and pooled human plasmas were placed in 96 well plates and spiked with 20 μL of 1:20 (v/v) Ultimate SPLASH mix (Avanti Polar, Alabaster, AL) working internal standard. To each sample-internal standard mix, 475 μL of 1:1 (v/v) butanol:methanol mixture was added and shaken for 10 minutes at 500 rpm at 4° C. The mixture was incubated for 15 minutes at 4° C. and shaken for 10 minutes at 500 rpm at 4° C. The sample was re-incubated for 15 minutes at 4° C. and finally centrifuged at 3500 rpm for 10 minutes. Approximately 300 μL of extract was transferred into clean collection plates and stored at −20° C. until LC-MS processing. Two chromatographic analytical separation methods were used for the separation of lipid class using a binary gradient flow system. Data were collected in multiple reaction monitoring (MRM) mode equipped with electrospray ionization in positive and negative polarity using a SCIEX 7500 (SCIEX, Redwood City, CA) triple quadrupole mass spectrometer. Positive mode lipids were separated using a SCIEX LC AD (SCIEX, Redwood City, CA) liquid chromatography system and a Waters Acuity UPLC BEH C18 (50×2.1 mm×1.7 μm) (Waters, Waltham, MA) column with gradient elution containing mobile phase A as water:acetonitrile (40:60 v/v) and mobile phase B as isopropanol:acetonitrile (90:10 v/v) at 0.5 mL/minute and 50° C. Lipids in negative mode were separated using a SCIEX LC AD liquid chromatography system and a Luna NH2 (100×2.0 mm×3 μm) (Phenomenex, Torrance, CA) column with gradient elution containing mobile phase A as water:acetonitrile (50:50 v/v) and mobile phase B as dichloromethane:acetonitrile (7:93 v/v) at 0.6 mL/minute and 40° C. For both separation methods, the autosampler temperature was maintained at 4° C. Positive and negative polarity data were processed separately using SCIEX OS Analytics (SCIEX, Redwood City, CA) software with MQ4 algorithm selected. NIST SRM1950 and pooled plasma quality control samples were utilized to optimize peak integration parameters such as intensity thresholds, signal-to-noise ratio, and smoothing parameters. These methods were utilized to process all the samples. The processed data were manually reviewed and curated to ensure accurate peak integration, exported as text files, and utilized for downstream statistical analysis.

Metabolite data were acquired using a multiplexed, targeted LC-MS assay, in which the target analytes were not selected based on any known association with PDAC. Polar metabolites were extracted from 30 μL of human plasma from cohort, NIST SRM1950, and pooled plasma samples utilizing 1:1 (v/v) water:methanol mixture. Briefly, 20 μL of QreSS 1 and 2 (Cambridge, Tewksbury, MA), a working internal standard, was spiked into the 30 μL plasma samples aliquoted into individual wells of a 96 deep-well plate. The metabolites were extracted by dispensing 450 μL of 1:1 (v/v) water:methanol mixture into each plasma sample. The sample-solvent mixture was shaken for 5 min at 1000 rpm maintained at 4° C. The mixture was then incubated for 60 minutes at 4° C. and centrifuged for 15 minutes at 3000 rpm maintained at 4° C. Data were collected in MRM mode equipped with electrospray ionization in positive and negative polarities using a SCIEX7500 triple quadrupole mass spectrometer. The metabolites were separated using a SCIEX LC AD liquid chromatography system with a Kinetics F5 100 Å (150×2.1 mm×2.6 μm) (Phenomenex, Torrance, CA) column and a gradient elution system containing mobile phase A as 2 mM ammonium acetate and 0.1% formic acid in water and mobile phase B as 0.1% formic acid in acetonitrile at 0.2 mL/minute and 40° C. For both separation methods, the autosampler temperature was maintained at 4° C. Positive and negative polarity data were processed separately using SCIEX OS Analytics software with MQ4 algorithm selected. NIST SRM1950 and pooled plasma quality control samples were utilized to optimize peak integration parameters such as intensity thresholds, signal noise ratio, and smoothing parameters. This method was used to process all the samples under study. The processed data were manually reviewed and curated to ensure accurate peak integration, exported as text files, and utilized for downstream statistical analysis.

### Transcriptomics Data Acquisition and Primary Data Processing

RNA-seq was performed on RNA extracted from PAXgene blood tubes using Qiagen PAXgene Total RNA kits as per the manufacturer's protocols. A 100M paired (200M total) read library of strand-specific, 100 bp reads was prepared using TruSeq Stranded Total RNA With Illumina® Ribo-Zero™ Plus rRNA Depletion+Globin Reduction RNA Library Preparation. Quality control was performed on the fastq files using FastQC (v0.11.9). Reads were aligned using the STAR aligner (v2.7.8a) and deduplicated using PicardTools (v2.25.0). Post-alignment quality control was performed using RNA-SeqC (v2.4.2). Transcript quantitation was done using RSEM (v1.3.3).

### CA19-9 Data Acquisition

CA19-9 levels were evaluated using a clinical-grade assay as per the vendor's instructions (Invitrogen Human CA19-9 ELISA Kit [Cat. No. EHCA199]).

### Published RNA-Seq Data Analysis

RNA-Seq of a wide variety of human tumors and normal tissues was performed previously by TCGA1,2 and GTEx3, respectively. These raw data sets were previously combined and co-processed by others. The RSEM expected counts were used to analyze differential expression between 183 pancreatic tumor and 167 normal pancreas samples using the DESeq2 package in R. Prior to DESeq2 analysis, genes with very low expression were filtered by requiring a minimum sum of counts across the 350 samples of 1000 for a gene to be retained. The ashr shrinkage estimator was employed to moderate fold-change estimates. Fold-changes and adjusted P-values for each of the genes highlighted in the text are shown in FIG. 55.

### Avoidance of Model Overfitting

Given the relatively large of number of analytes involved in this multi-omics study and the modest number of subjects in the training data, the risk of overfitting the data in a model is a potential concern. A number of approaches to mitigate and evaluate this risk were implemented. First, a conservative split of the total subject population was selected, with approximately 60% in the training set and 40% in the validation set. By increasing the size of the validation set, the ability to detect overfitting, if it were to occur, is increased, even if the ability to identify important classifier components is reduced. Second, the study design incorporated an intentional differentiation in date of enrollment and site of enrollment for our PDAC and control groups. These steps mitigate the risk of systematic bias between the groups being carried over from training to validation sets. Third, an extensive cross-validation design was employed when optimizing model engine parameters and important feature selection. Ten rounds of 10-fold cross-validation, while computationally intensive for many input features, is a robust approach to avoid overfitting. Finally, the training subject data groups (e.g., PDAC vs control) were randomly permutated to determine if the 2-stage process described here can produce a final, multi-omics model with similar performance in the validation set as observed in the individual omics models. Class permutation was repeated 10 times feeding from the initial individual omics RCVs into the final combined top feature RCV and achieved a validation set ROC AUC of 0.629 (±0.113 standard deviation). This is significantly different than the validation ROC AUC achieved with the correct class assignments (0.977, p=4.393e-06) and confirms that extreme overfitting did not drive the high performance observed, although a statistically significant positive bias was observed compared to random performance (AUC 0.5, p=0.005457).

### Exploratory Data Analysis, Univariate and Multivariate

For the exploratory data analysis (EDA), all 146 subjects were used for the univariate and multivariate comparisons. The R statistical computing language and appropriate packages were used for all analyses with appropriate additional packages. In general, after primary data processing, the data were normalized with appropriate methods for each omics type. In brief, median normalization was performed using predominantly common features for the proteomics and metabolomic data; features present in 90% of the subjects' proteomics data and 95% of the subjects' metabolomics data were considered as the reference set for individual subject's median determination and mean of the subjects' medians normalization factor calculation. For the lipidomics data, the spiked reference standards were used to median scale the samples. For the RNA data, the DESeq2 algorithm was used for normalization.

The features were filtered to those that were present in ≥50% of at least 1 of the classes (PDAC or non-cancer). Where necessary (e.g., principal component analysis [PCA], etc.), missing values were assumed to be missing below the lower limit of detection rather than missing at random and were imputed with the minimum value for that analyte across the sample set. The non-parametric Wilcoxon test with Bonferroni multiple testing correction was used for the univariate comparison between the study groups using only actual, non-imputed values. For the GO Biological Process (GOBP) term enrichment analysis for protein and RNA omics types, Fisher's test was used to evaluate the significance of proportional differences, and the difference was reported as the log of the odds ratio.

### Initial, Individual Omics Machine Learning-Based Classifier Training Using all Available Features for Each Class

For the machine-learning-based classification model training and validation, distinct splits of the 146 samples were created, comprising 74 subjects (n=37 PDAC and n=37 control) for RCV and final model construction and 72 subjects (n=26 PDAC and n=46 control) for validation. The proportionality of PDAC cancer stages was maintained in the splits. To improve the generalization of the results, and to avoid possible confounding factors, the split of training and validation subjects was collection-site and date-of-enrollment stratified. For the training set, PDAC subjects comprised the first 60% of subjects enrolled in the study across 10 sites, and the control subjects were selected from 3 of the 4 control-enrolling sites. For the validation set, PDAC subjects comprised the last 40% of enrolled subjects and control subjects were selected from a unique site. After the training/validation split, features were filtered to those present in ≥50% of at least 1 of the classes.

A robust machine-learning modeling engine was deployed, XGBoost, which is an implementation of a gradient-boosted, ensemble-tree method. Given the modest numbers of subjects available for model testing, a common approach was employed to avoid overfitting and improve the quality of hyperparameter selection and validation performance estimation using 10 repeats of 10-fold cross-validation as described above. Analyte value scaling was the only form of feature engineering other than missing value imputation prior to RCV. Fifty hyperparameter combinations were tested with selection and distribution of the candidate values using a Latin Hypercube design. For computational efficiency, a futility analysis was performed during repeated cross-validation, which, after an initial burn-in period of 5 (for RNA) or 10 (for proteins) cycles, compared parameter combination results by ANOVA and removed those models from further consideration that were not likely to achieve superiority to the current best combination. With the parameters selected, a final model using all the training subjects was created, noting the feature importance assigned by the algorithm for the model. This basic approach was utilized for all four of the omics types.

Multi-Omics Classifier Construction Using Top 5 Features from Each Individual Omics Model

Although each of the individual omics final training models were evaluated in the validation set, the primary purpose of constructing the final models was to assess feature importance and select the top 5 features from each omics type. This approach uses all analytes in each individual omics type in the XGBoost RCVs for feature selection (top 5 for each type), and then uses the combined 20 selected features and a final, re-shuffled RCV with GLMnet to create the final, multi-omics model. Although it may be preferable to have separate sets of subjects for feature selection and model creation, the training subjects were not split and power was not reduced and, instead, the subjects were re-shuffled into new repeats-folds. No data from the held-out validation set were used for feature selection RCV or final model creation RCV. This approach mitigates the chances of overfitting our model on the training data, and multiple rounds of random class permutations were performed for confirmation.

### Evaluation of a Large Number of Features for EDA and Machine Learning

Blood analyte data (e.g., protein, metabolite, lipid, and RNA) from PDAC and non-cancer control subjects was collected for exploratory data analysis (EDA) and machine learning-based model building. Given the large number of original features for each omics type, univariate and multivariate EDA ensured that reasonable pre-processing of the data, irrespective of class (e.g., PDAC or control), may include a large amount of signal with a minimal amount of noise. After processing and filtering, the numbers of features originally derived from the data for each omics type, as well as the numbers of presence-filtered features used for the EDA and the machine-learning RCV analysis are shown in the Table 15.

TABLE 15

| Omics | Feature | Feature |
|---|---|---|
| Type | Group | Count |
| Proteins | Original | 151,461 |
| | EDA | 54,180 |
| | Classification | 21,176 |
| Metabolites | Original | 377 |
| | EDA | 373 |
| | Classification | 372 |
| Lipids | Original | 898 |
| | EDA | 898 |
| | Classification | 879 |
| RNAs | Original | 202,125 |
| | EDA | 110,734 |
| | Classification | 107,631 |

For the protein and RNA omics types, the large drop in the number of features between the original, primary data and the data used for EDA is primarily due to the stochastic nature of low-frequency data detection for these unbiased, untargeted methods. For example, there could be many features detected in very few of the study samples, which are not useful for either EDA or classification, and so were removed. In addition, for both the protein and RNA groups, a further drop in feature count reflects the removal of GOBP acute-response-associated features as well as any proteins present in the top 25% of the 2017 Human Plasma Proteome Project (HPPP) database concentration profile.

### Univariate and Multivariate Comparisons Highlight Differences in Blood Analytes Between PDAC and Control Subjects

To explore the utility of blood analytes to distinguish PDAC from non-cancer samples, EDA of isolated proteins, metabolites, lipids, and RNAs from PDAC and non-cancer control subjects was performed. After feature normalization and class-presence exclusions (e.g., ≥50% presence detection in at least 1 of the classes: PDAC or non-cancer), the Wilcoxon non-parametric comparison tests were performed, and after Bonferroni multiple testing correct of the resulting p-values, the significance (p≤0.05) was scored and tabulated. The analysis was further constrained to those features present in at least 3 samples in each class, leading to the exclusion of 6 peptide features that were present in ≥50% of the subjects from 1 class and less than 3 subjects in the other. None of the other omics types had any such exclusions. For each omics type, there were statistically significant differences in the expression levels of features in PDAC samples compared to controls.

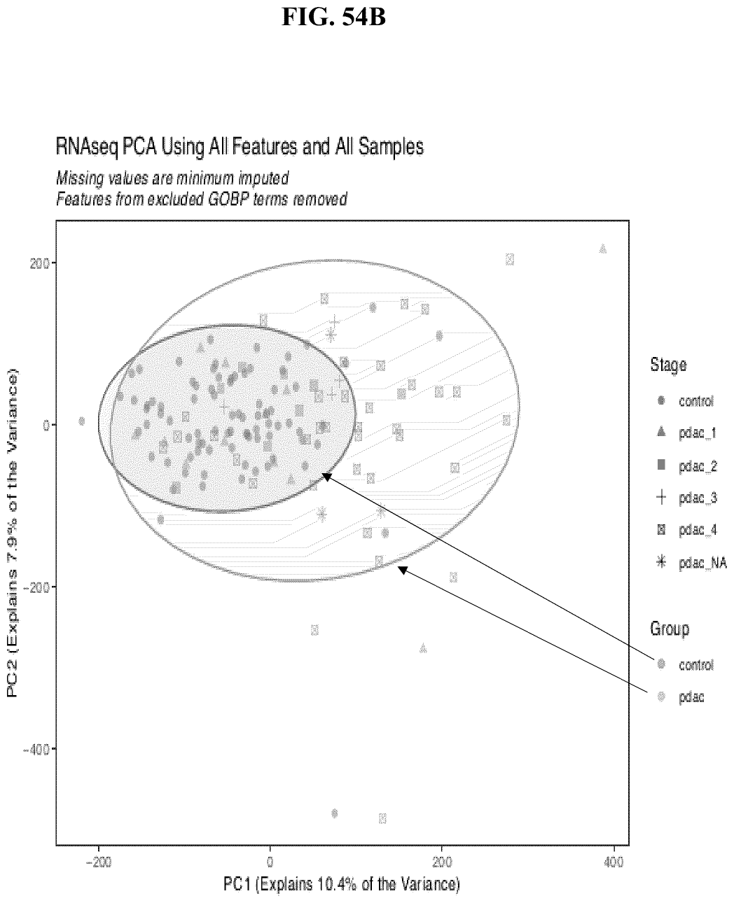

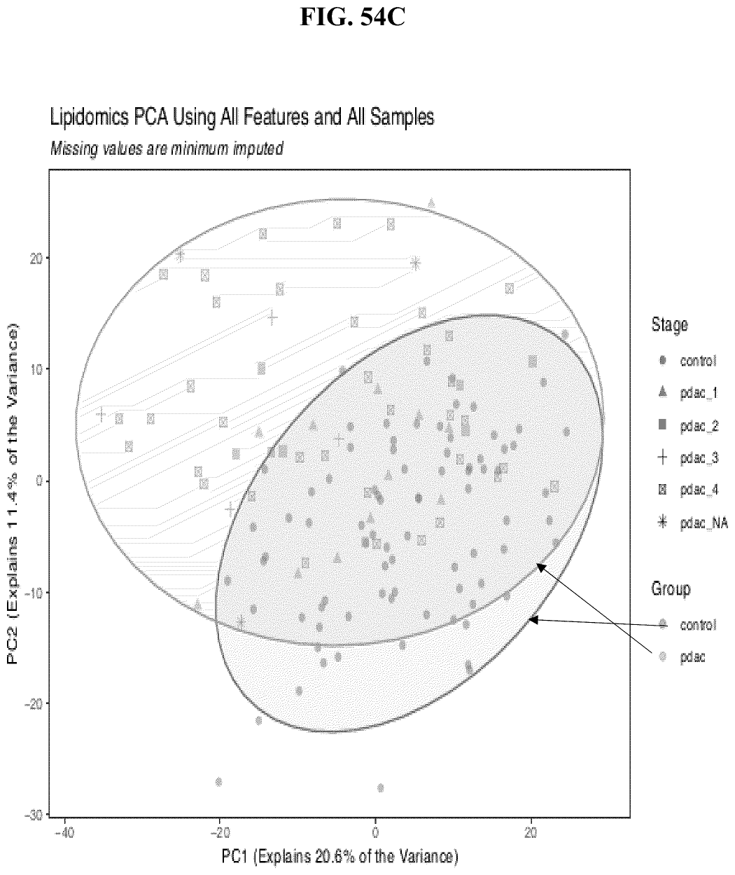

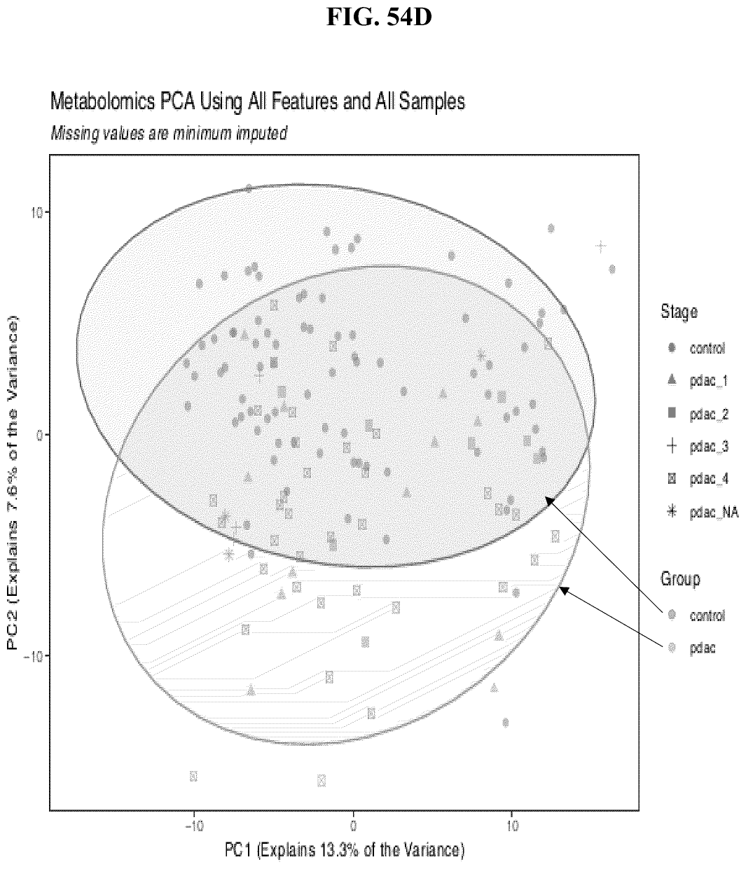

To evaluate the potential for multivariate discrimination between PDAC and control subjects, parametric (e.g., PCA) dimensionality reduction projections were employed to observe group separation (FIGS. 54A-54D). The projections suggest that simple combinations of the underlying features may be insufficient for robust classification and that more sophisticated feature selection and classification model development are necessary.

Mitigation of Potentially Confounding Non-Specific Signals from the Protein and RNA Analytes

To reduce the impact of non-cancer-specific protein/peptides signals, the Wilcoxon comparison hits were used to perform a GOBP term enrichment analysis using Fisher's test and log-odds ratios as measures for the significance and magnitude of potential differences in protein/peptide expression between PDAC and control samples. For each class of hits, increasing in PDAC or decreasing in PDAC, the potential enrichment or depletion of GOBP terms were evaluated (Table 16).

TABLE 16

| | | | PDAC | PDAC |
|---|---|---|---|---|
| Omics Type | All | Significant | Elevated | Reduced |
| Protein | 54,108 | 439 | 397 | 42 |
| Metabolite | 373 | 37 | 13 | 24 |
| Lipid | 898 | 170 | 10 | 160 |
| RNA | 110,734 | 3,058 | 2,127 | 931 |

Although many terms were too sparsely represented in the feature hit list to achieve significance with Bonferroni correction, a number had uncorrected Fisher's proportionality test p-values≤0.05. Even after Bonferroni correction, the GOBP term for acute-phase response remained significantly enriched for proteins with increased values in PDAC, which could be indicative of a non-specific, acute-phase inflammatory response. Therefore, to maximize the potential to identify PDAC-specific individual markers and improve classifier performance, a conservative approach was taken by removing any such non-specific features from the multivariate EDA and machine-learning-based classification. Protein and RNA features filtered from consideration were those with associated GOBP terms including the roots “acute”, inflamma”, and “immun”; 471 protein groups and 6625 RNA ENSTs comprising 2516 associated Uniprot entries were excluded.

Features associated with proteins that were detected in the top 25% by concentration based on the 2017 HPPP were also excluded. Table 15 shows the number of peptide features that remained for consideration after this filtering. With respect to protein groups, each comprised of multiple peptide features, of the original 3851 protein groups that were present in the training subject data, 2686 remained after 50% presence filtering, 2064 remained after top 25% HPPP filtering, and 1794 remained after GOBP filtering. Across the 5 nanoparticles in the Proteograph sample preparation platform, these 1794 protein groups were comprised of 7874 modified peptide sequences with a median of 3 modified sequences per protein group.

### Individual Omics Classifiers Identify Most Important Classification Features for PDAC

In general, the univariate and multivariate results indicated that while a number of statistically significant differences existed between the PDAC and control subject groups, no one omics type and certainly no one feature within an omics type was sufficient to clearly discriminate between the groups. Thus, individual omics classifiers using all features to determine the most important features within each omics type was developed. After 10 repeats of 10-fold XGBoost RCV to evaluate 50 model hyperparameter combinations, the best performing combination for each omics type was identified (Table 17).

TABLE 17

| Omics | Mean | | | | | | | |
|---|---|---|---|---|---|---|---|---|
| Type | AUC | Mtry | Trees | Min_n | Tree_depth | Learn_rate | Loss_reduction | Sample_size |
| Protein | 0.927 | 1076 | 697 | 3 | 9 | 0.0236 | 0.620 | 0.517 |
| Metabolite | 0.910 | 92 | 1442 | 2 | 7 | 0.00000447 | 0.0864 | 0.448 |
| Lipid | 0.889 | 578 | 1461 | 3 | 3 | 0.000190 | 0.00568 | 0.322 |
| RNA | 0.797 | 70483 | 1461 | 3 | 3 | 0.000190 | 0.00568 | 0.322 |

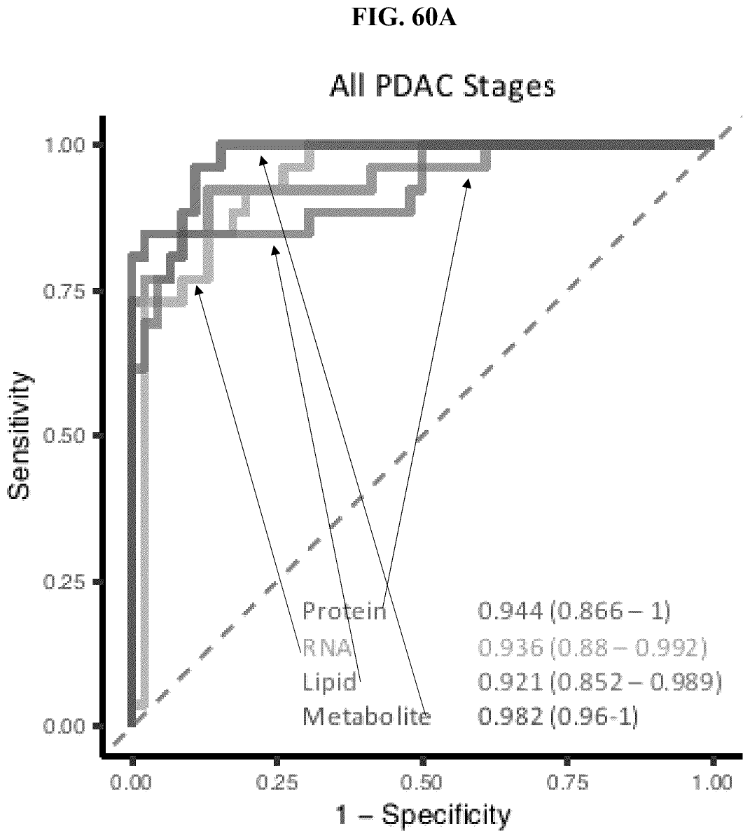

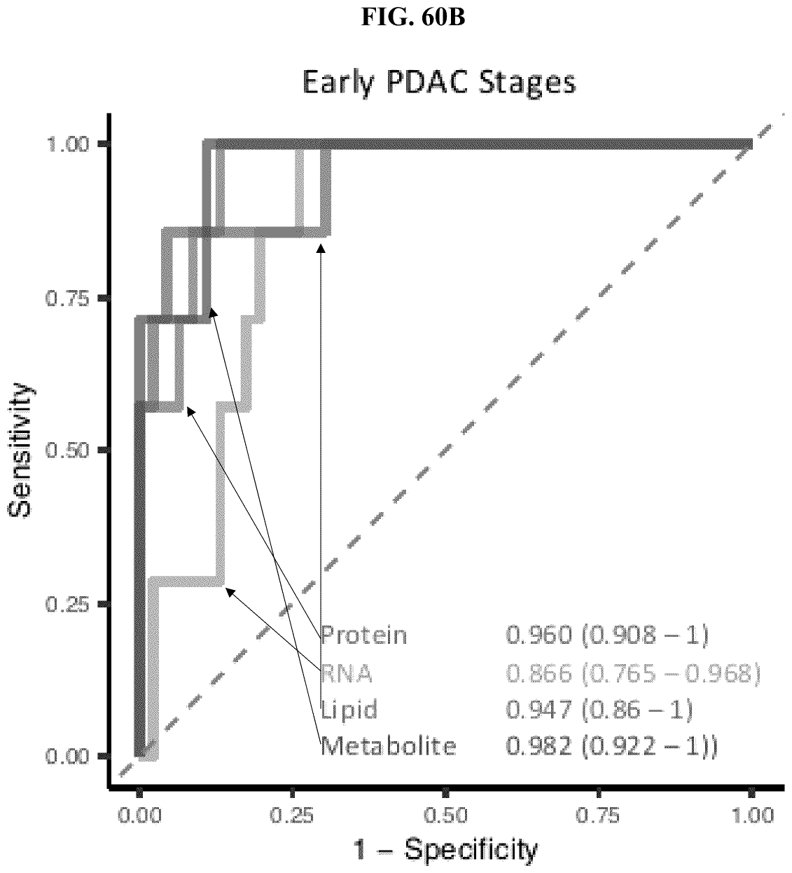

Using the optimized parameters, a final XGBoost model comprising all the training subjects was created individually for each omics type. As is characteristic of such a model evaluated using its own input data, the training set areas under the ROC curve (AUCs) were 1 and do not truly represent the estimated performance in a new set of data and, therefore, is of limited utility in comparing models' performances. The average across the RCV resamples provides a better estimate (Table 17). Notably, the final all-training-data models identify the relative importance of input features for each omics type (FIG. 59), which provides the basis for a combined, top-feature, multi-omics model. Evaluation of the final individual omics models in the validation sample set showed that all omics types achieved high performance with individual validation ROC AUCs of 0.921 for lipids, 0.936 for RNAs, 0.944 for proteins, and 0.982 for metabolites (FIG. 60A).

a Multi-Omics Classification Model Using a Combination of Top Features from Individual Omics Models Achieves High Sensitivity and Specificity for PDAC

An important consideration in collecting multiple omics data types for any classification effort is the potential synergistic effect that this information may impart. Inspection of the individual omics models' class prediction probabilities indicates a change in probability rank order from one model type to another even though the overall performance is not significantly different between models (FIG. 61). Table 18 shows the Kendall rank correlation and its p-value for each omics type pairwise comparison.

TABLE 18

| Omics Type | Kendall Tau | |
|---|---|---|
| Comparison | Correlation | P-value |
| Lipid-RNA | 0.3271 | 4.92334E−05 |
| Protein-RNA | 0.3584 | 8.70228E−06 |
| Protein-Lipid | 0.4617 | 0 |
| Lipid-Metabolite | 0.4851 | 0 |
| Metabolite-RNA | 0.4851 | 0 |
| Protein-Metabolite | 0.5008 | 0 |

The range of these values, between 0.3271 and 0.5008, suggests that these models are neither positively or negatively correlated, and the similarity of their ROC AUCs suggests that the different information available from each omics type may contribute distinct useful information.

To take advantage of possible synergism between omics types, a combined omics model was created using the most important features from each omics type (as determined in the first RCV) in a second 10×10 RCV with new resample groupings. Inspection of the individual omics features suggests that a relatively small number of features may provide most of the discrimination performance (FIGS. 59A-59D)(Tables 19-22). UniMod:4 represents an amino acid modified with an iodoacetamide derivative while UniMod:35 represents an amino acid modified with a methionine sulfoxide.

TABLE 19
Protein Features

| Gene | Protein Marker | SEQ ID |
|---|---|---|
| LTBP2 | GAGGQSMSEAPTGDHAPAPTR | SEQ ID |
| | | NO. 1 |
| CDHR2 | TFVIIPELVLPNR | SEQ ID |
| | | NO. 2 |
| CDHR2 | TFVIIPELVLPNR | SEQ ID |
| | | NO. 2 |
| FGFBP2 | DSC(UniMod: 4)TMRPSSLGQGAGEVWLR | SEQ ID |
| | | NO. 3 |
| THBS2 | DNC(UniMod: 4)PHLPNSGQEDFDK | SEQ ID |
| | | NO. 4 |
| CDHR2 | GLVLGAGWAEGYLR | SEQ ID |
| | | NO. 5 |
| THBS2 | LVFNPDQEDLDGDGRGDIC(UniMod: 4)K | SEQ ID |
| | | NO. 6 |
| ANTXR2 | AFDLYFVLDK | SEQ ID |
| | | NO. 7 |
| CILP2 | VFLVGNVEIR | SEQ ID |
| | | NO. 8 |
| ANTXR2 | RVSPVGETYIHEGLK | SEQ ID |
| | | NO. 9 |
| ANTXR1 | ASEQIYYENR | SEQ ID |
| | | NO. 10 |
| ANTXR1 | VLPGGDTYMHEGFER | SEQ ID |
| | | NO. 11 |
| RPL10A | AVDIPHMDIEALK | SEQ ID |
| | | NO. 12 |
| H2BC18 | AMGIMNSFVNDIFER | SEQ ID |
| | | NO. 13 |
| ANTXR1 | MPEQEYEFPEPR | SEQ ID |
| | | NO. 14 |
| PDCD6 | SGVISDTELQQALSNGTWTPFNPVTVR | SEQ ID |
| | | NO. 15 |
| SLAIN2 | M(UniMod: 35)EDVNSNVNADQEVR | SEQ ID |
| | | NO. 16 |
| VCAN | VGHDYQWIGLNDK | SEQ ID |
| | | NO. 17 |
| THBS2 | HAEC(UniMod: 4)IYLGHFSDPMYK | SEQ ID |
| | | NO. 18 |
| FGL2 | NGIFWGTWPGVSEAHPGGYK | SEQ ID |
| | | NO. 19 |

TABLE 20
RNA Features

| RNA Marker | Associated Gene |
|---|---|
| ENST00000483727.5 | itchy E3 ubiquitin protein ligase |
| ENST00000531734.6 | Adenosine monophosphate deaminase 2 |
| ENST00000437154.6 | ADAM metallopeptidase domain 28 |
| ENST00000531997.1 | Glycine N-acyltransferase-like |
| | protein 1 (GLYATL1) |
| ENST00000424185.7 | NAD(P)HX dehydratase |
| ENST00000652176.1 | BICD cargo adaptor 1 |
| ENST00000392593.9 | Phospholipase D family member 4 |
| ENST00000532853.5 | Solute carrier family 27 member 3 |
| ENST00000429947.1 | Long intergenic non-protein coding RNA 1237 |
| ENST00000580914.1 | Nucleolar protein 11 |
| ENST00000368205.7 | Protein tyrosine phosphatase receptor type K |
| ENST00000531709.6 | Nuclear RNA export factor 1 |
| ENST00000524817.5 | Switching B cell complex subunit SWAP70 |
| ENST00000651281.1 | ERCC excision repair 5, endonuclease |
| ENST00000499685.2 | BTG1 divergent transcript |
| ENST00000311921.8 | Zinc finger protein 507 |
| ENST00000472111.5 | Galactose-1-phosphate uridylyltransferase |
| ENST00000585172.2 | Novel pseudogene |
| ENST00000287713.7 | Nicotinamide nucleotide adenylyltransferase 2 |
| ENST00000547687.2 | Charged multivesicular body protein 1A |

TABLE 21
Lipid Features
Lipid Marker

NEG_PC(18:2_20:5) + AcO
POS_DAG(18:1_20:0) + NH4
NEG_PE(O − 16:0_22:6) − H
NEG_PC(18:2_20:3) + AcO
POS_CER(d18:1/18:0) + H
POS_CE(22:0) + NH4
NEG_PE(14:0_22:5) − H
NEG_PC(20:5_20:5) + AcO
POS_PE(P − 18:0_18:3) + H
NEG_PE(O − 16:0_20:3) − H
POS_CE(18:3) + NH4
NEG_PE(O − 18:0_22:5) − H
NEG_PE(O − 18:0_20:5) − H
POS_PE(P − 20:0_20:3) + H
NEG_PE(O − 16:0_20:2) − H
POS_CER(d18:1/24:0) + H
NEG_PA(20:1_20:3) − H
NEG_PA(20:0_20:5) − H
POS_CE(20:0) + NH4
NEG_PC(16:1_20:3) + AcO

TABLE 22
Metabolite Features
Metabolite Marker

NEG_AICAR
POS_Cystine
NEG_CMP
NEG_Gentisate
POS_Creatine
POS_Imidazoleacetic acid
POS_Inosine
NEG_n-Isovalerylglycine
NEG_Glucose-6-Phosphate
POS_Metanephrine
NEG_N-Acetylglutamate
NEG_5-Thymidilic acid (dTMP)
POS_UMP
NEG_Fructose-6-Phosphate
NEG_Cystine
POS_Ubiquinol
POS_Guanine
NEG_Shikimic Acid
POS_1-Methylimidazole acetate
POS_Flavone 2

5 features from each analyte type (e.g., protein, metabolite, lipid, and RNA) were selected that may seem to include the inflection point of the importance scree plots (Table 23).

TABLE 23

| Omics | | |
|---|---|---|
| Type | Feature ID | Protein (Gene) |
| Proteins | NP4_GAGGQSMSEAPTGDHAPAPTR | Q14767-LTBP2 (LTBP2) |
| | NP3_TFVIIPELVLPNR | Q9BYE9-CDHR2 |
| | | (CDHR2) |
| | NP2_TFVIIPELVLPNR | Q9BYE9-CDHR2 |
| | | (CDHR2) |
| | NP3_DSC(UniMod: 4)TMRPSSLGQGAGEV | Q9BYJ0-FGFBP2 |
| | WLR | (FGFBP2) |
| | NP3_DNC(UniMod: 4)PHLPNSGQEDFDK | P35442-TSP2 (THBS2) |
| Metabolites | NEG_AICAR | |
| | POS_Cystine | |
| | NEG_CMP | |
| | NEG_Gentisate | |
| | POS_Creatine | |
| Lipids | NEG_PC(18: 2_20: 5) + AcO | |
| | POS_DAG(18: 1_20: 0)+NH4 | |
| | NEG_PE(O-16: 0_22: 6) − H | |
| | NEG_PC(18: 2_20: 3) + AcO | |
| | POS_CER(d18: 1/18: 0) + H | |
| RNAs | ENST00000483727.5 | NA (ENSG00000078747) |
| | ENST00000531734.6 | Q01433-AMPD2 |
| | | (AMPD2) |
| | ENST00000437154.6 | Q9UKQ2-ADA28 |
| | | (ADAM28) |
| | ENST00000531997.1 | NA (GLYATL1 |
| | | pseudogene) |
| | ENST00000424185.7 | Q8IW45-NNRD (NAXD) |

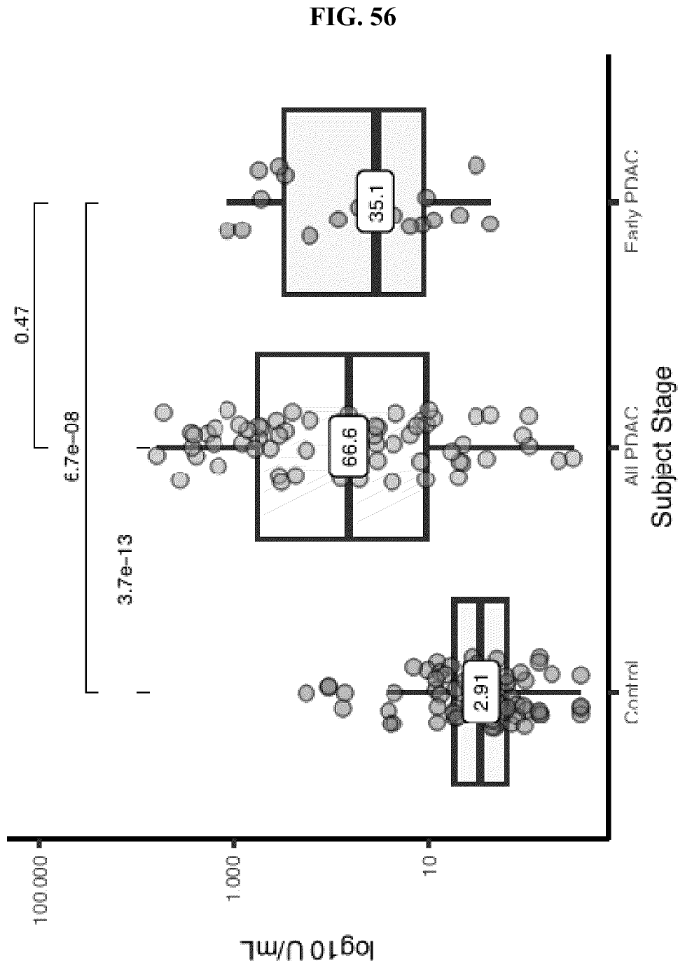

UniMod:4 represents a modification of the amino acid residue to include a iodoacetamide derivative. Two of the top 5 protein features comprise the same peptide, TFVIIPELVLPNR (SEQ ID NO. 2), as detected on 2 different nanoparticles; nanoparticle-peptide combinations were evaluated as unique features in these analyses. By analyzing published RNA-Seq data for the top peptide/protein features, a statistically significant change in their mRNA levels between PDAC tumors and normal pancreas tissue (FIG. 56).

After 10×10 RCV using a grid of 50 hyperparameter combinations, a final GLMnet parameter selection was defined: penalty=0.753 and mixture=0.0917. Using these parameters, a final GLMnet model was created using all the training data. The coefficients for the final regression model are shown in FIG. 55. The model was then applied to the validation set of 72 samples to evaluate performance, achieving ROC AUC values of 0.977 and 0.988 for all and early-stage subjects, respectively. The observed performance at 99% specificity for all-stage and early-stage subjects was 80.8% and 71.4%, respectively.

### Comparison of the Multi-Omics Classifier to CA19-9

A clinical-grade assay to measure CA19-9 for all 146 study subjects was used (FIG. 56). As is demonstrated in the annotated comparisons, CA19-9 was significantly elevated in PDAC subjects as compared to the non-cancer control subjects (66.6 U/mL vs 2.91 U/mL, p<2.22e-16). However, although trending higher, CA19-9 levels were not significantly higher in stage IV PDAC versus stage I PDAC (290 U/mL vs 35.1 U/mL, p=0.18).

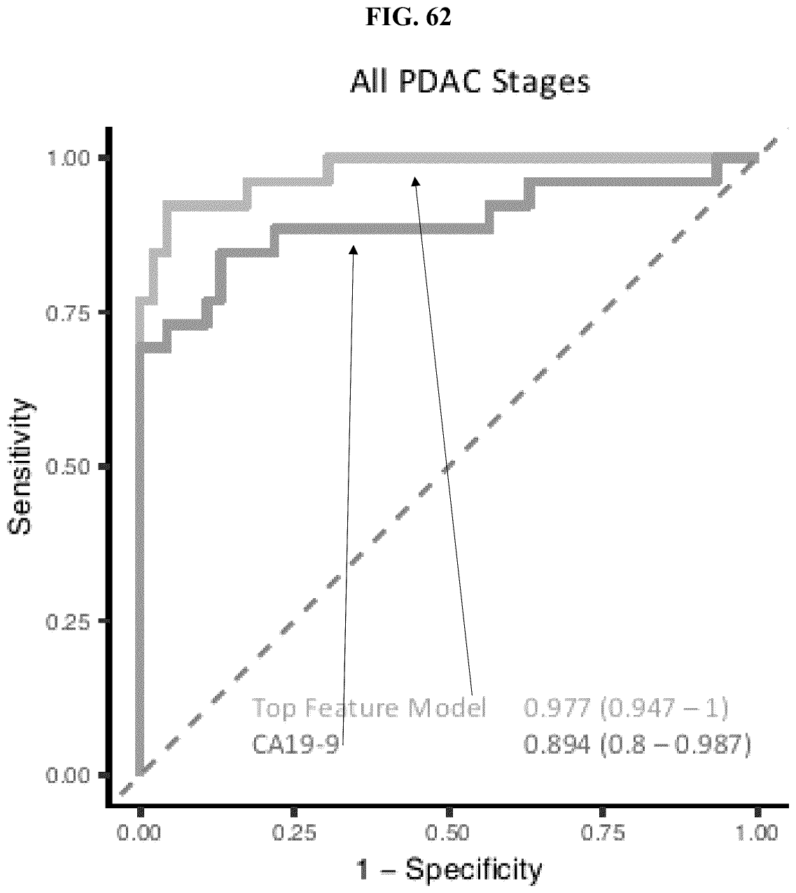

When the training data subjects were used to build a simple regression model of class probabilities, and that model was applied to the validation set, the ROC AUCs for all and early-stage subjects were 0.894 and 0.885, respectively (FIG. 62), with no significant difference in performance, all versus early-stage (p=0.92771). The demonstrated sensitivities at 99% specificity for all and early-stage PDAC were 69.2% and 57.1%, respectively.

### Data Interpretation

This study presents a proof-of-concept study, which leveraged a broad, multi-omics profiling platform to identify novel combinations of known and/or unknown markers into a high-performance biomarker panel for detection and discrimination of PDAC from non-cancer controls. By evaluating proteins, metabolites, lipids, and RNAs present in blood samples with this platform, several physiological spaces were sampled, some potentially directly related to PDAC tumor growth and development and some related to the body's response to that growth. This platform's collection of omics types was characterized as phenotypic in that they collectively report the status of the body's systems as opposed to other common omics profiling types (e.g., single nucleotide polymorphisms, copy number variations, fragmentation, and methylation) that might be characterized as static risk indicators.

Despite widespread efforts over decades to develop pancreatic cancer diagnostics, so far, nothing has been able to improve upon imaging techniques, such as endoscopic ultrasound, that only have clinical utility in targeted, high-risk populations. These attempts have included efforts to identify and implement biomarkers from several biofluids (e.g., blood, saliva, pancreatic bile duct juice, etc.), yet no validated, FDA-cleared marker exists that is suitable for screening use. Recently published reports show the promise as well as the limitations involved in the liquid biopsy approach to pancreatic cancer detection. In the proteomics space, a validation study of a multiplex immunoassay combining an 8-plex biomarker signature and CA19-9 was reported. The report noted a need to exclude Lewis-negative subjects for best sensitivity performance in early and all-stage PDAC classification. Efforts in other individual omics areas, such as DNA methylation, are promising but also challenging, as exemplified in a recent report in which 39.4% sensitivity at 100% specificity was achieved in a validation set of subjects with pancreatic cancer versus subjects without cancer.

This approach of evaluating many potentially contributory analyte features in a modest size study employed a 2-stage system with feature filtering designed to avoid both classifier overfitting and inclusion of confounding, non-specific signals. The panel of 20 analytes, 5 each from the 4 omics types profiled, has the key attributes of excellent early-stage performance from an accessible sample space (e.g., venous blood). The manageable number of easily assayed analytes makes this panel ideal for rapid development, clinical study, and eventual regulatory studies and reviews. Since the final classifier is based on a simple linear model (FIG. 55), it could lead to an assay with individual components that can be discretely measured, which may accelerate the assay's clinical development from a technical and regulatory perspective. The 20-feature classifier demonstrated 80.8% and 71.4% sensitivities at 99% specificity for all-stage and early-stage PDAC, respectively, without any contribution from CA19-9. This demonstrates the usefulness multi-omics approaches to achieve synergistic, superior performance compared to individual omics classification strategies.

### Example 12. Testing Biological Process Coverage with Combined Omic Types

### Summary

An analysis of the pancreatic ductal adenocarcinoma (PDAC) study was conducted to determine the diversity and enrichment of coverage of Gene Ontology-Biological Process (GOBP) terms. A comparison between the protein-derived data (via peptides from Proteograph platform analysis discussed above) and RNA-derived data (via transcripts from RNAseq as discussed above) was performed. Using the 146 subjects comprising the PDAC study and a mix of cancer and non-cancer controls, the analysis was constrained to those features, proteins or RNAs, that were present in at least 25% of the subjects' samples. A total of 12,006 unique GOBP terms were identified and were associated with one or more of the features. Enumeration of the identified GOBP terms determined that the RNA omics type comprised almost all the GOBP terms, with about 50% (5,966) unique to RNA, 50% (5,974) shared between RNA and protein, and less than 1% (66) unique to protein. However, when additional evaluation for enrichment of terms based on differences in the number of underlying, associated features was performed, 40 GOBP terms were identified as significantly enriched in protein or RNA, with 27 enriched in protein and 13 enriched in RNA. Thus, the best depth of coverage for GOBP-defined processes required both RNA and protein and not just RNA.

### Introduction

When choosing traits in order to build a top trait classifier as discussed in the previous example, many different variables may be factored into the equation. While choosing traits that may have the highest predictive power may make logical sense, there may be other variables that motivate a more complex selection criteria. One consideration for how well a classifier, trained on only a sample of the population, will operate when tested against the whole population, is how many different biological processes the traits interrogate. The greater number of biological processes, the greater the likelihood that the classifier will maintain predictive power when applied to the population. An analysis of the coverage of the biological processes of the protein and RNA traits found during the PDAC testing above was conducted.

### Analytical Method

This experiment used protein and RNA data collected from the 146 subjects that comprised the PDAC multi-comics classifier study as discussed above.

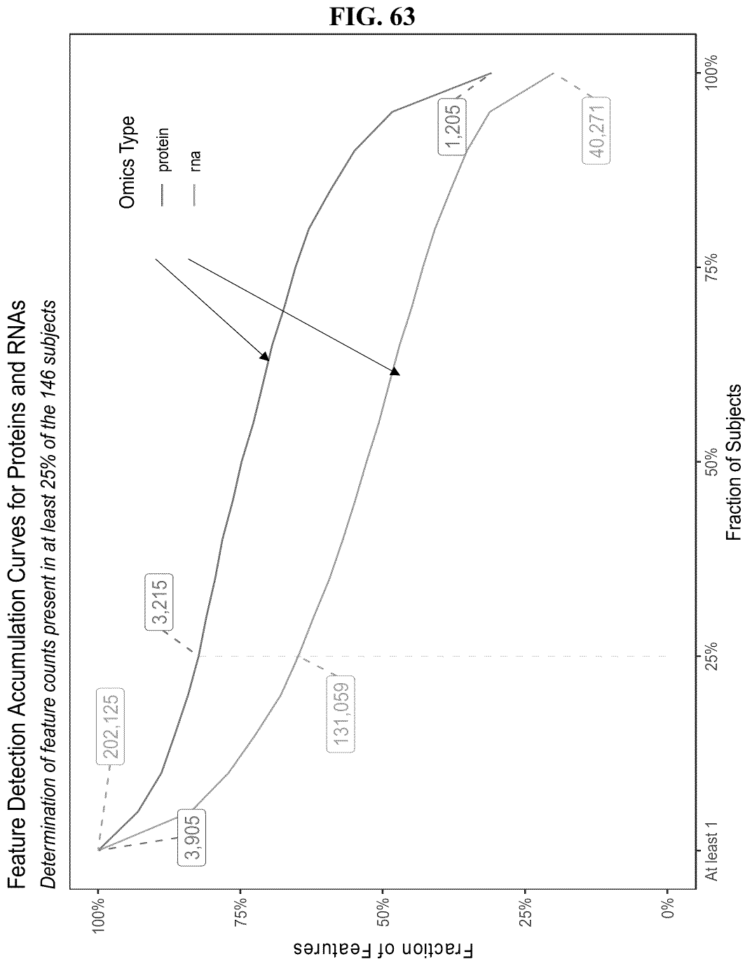

For proteins, the data from Proteograph platform for processing plasma proteins from EDTA tube-based samples were processed as previously described. The proteins associated with the identified peptides were enumerated for each subject's sample, and the frequency of occurrence for each of those proteins across the subjects was determined. That list of proteins was filtered to only those present in at least 25% of the 146 subjects. For RNA, the data from RNAseq analysis was also processed as described, and the detection frequency was similarly calculated. The counts for detection for proteins and RNAs are shown in FIG. 63.

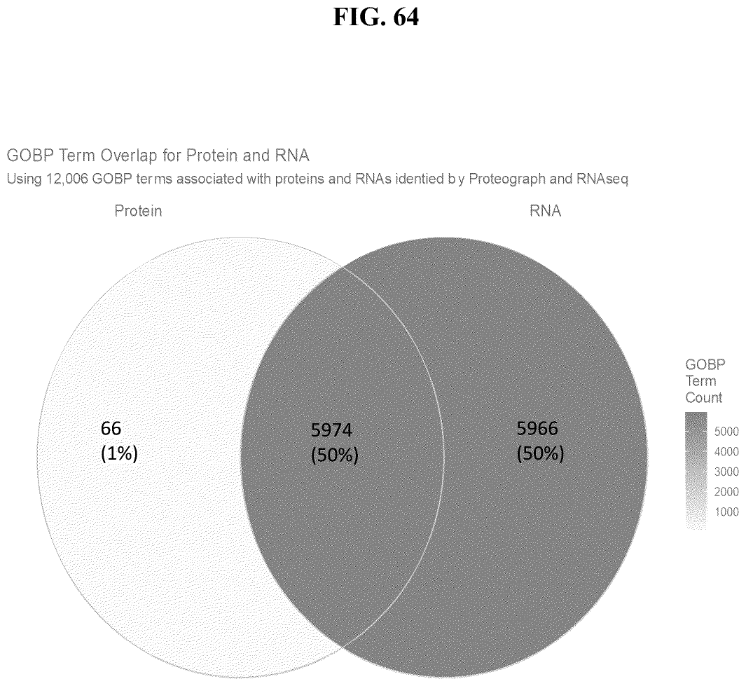

After enumeration of the proteins as Uniprot entries and RNAs as ENST entries, the association of GOBP terms to those entries was made using data downloaded from the UniprotKB online resource (downloaded on Jan. 30, 2023). This downloaded table enumerated annotated associations between Uniprot entries, ENST RNA transcripts, and GOBP terms. Where multiple ENST IDs and GOBP terms were associated with a single Uniprot entry, all possible associations were scored (e.g., if multiple ENSTs and GOBPs existed for an entry, all ENSTs were associated with each of the multiple GOBPs). A total of 12,006 GOBP terms were linked with proteins or RNAs from the study samples, and their listing are shown as a Venn overlap plot (FIG. 64). Proteins detected very few unique GOBP terms, (e.g., 66 or <1%), and almost all protein related GOBP terms were represented on the RNA list. However, this only accounted for tagging of a GOBP term by at least one, and possibly only one feature for a given omics type. It does not reflect enrichment of a GOBP term in one omics class or the other.

Beyond simple tagging of a GOBP term by different numbers of features in a class, an analysis for the enrichment of a given GOBP term in one omics type or the other was used by calculating the ln Odds Ratio (LOR). In this example, the LOR was defined as:

$$LOR = ln ( ( instances for a given GOBP term in protein / total instances of all GoBP terms in protein - instances for the term in protein ) ) / ( ( instances for a given GOBP term in RNA / total instances of all GOBP terms in RNA - instances for the term in RNA ) ) .$$

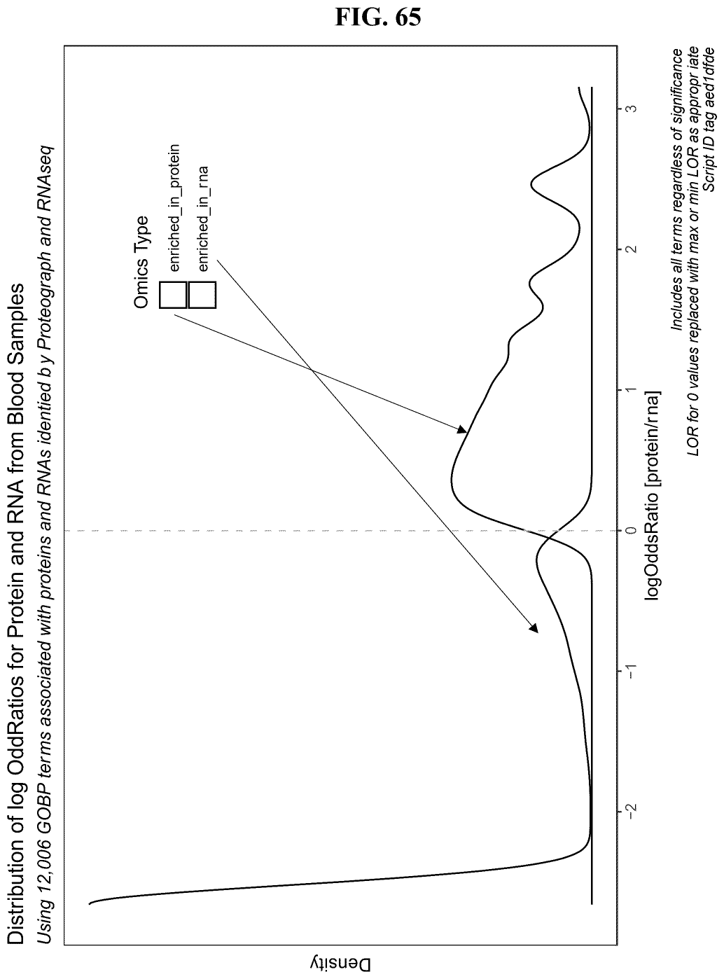

The distribution of these LOR values (FIG. 65) indicated that the majority of enumerated GOBP terms showed a difference in their frequency of representation against the totals. Many of the LOR values were not tightly grouped around 0 as may be the case if their relative proportions in the two omics types were similar. Note that in the case where there was no detection of a GOBP term in one omics type, the LOR value assigned to that term was either a maximum value (for GOBP terms missing in RNA) or a minimum value (for GOBP terms missing in protein). This explains the peak of density in the RNA plot for the large number of GOBP terms not detected at all in protein.

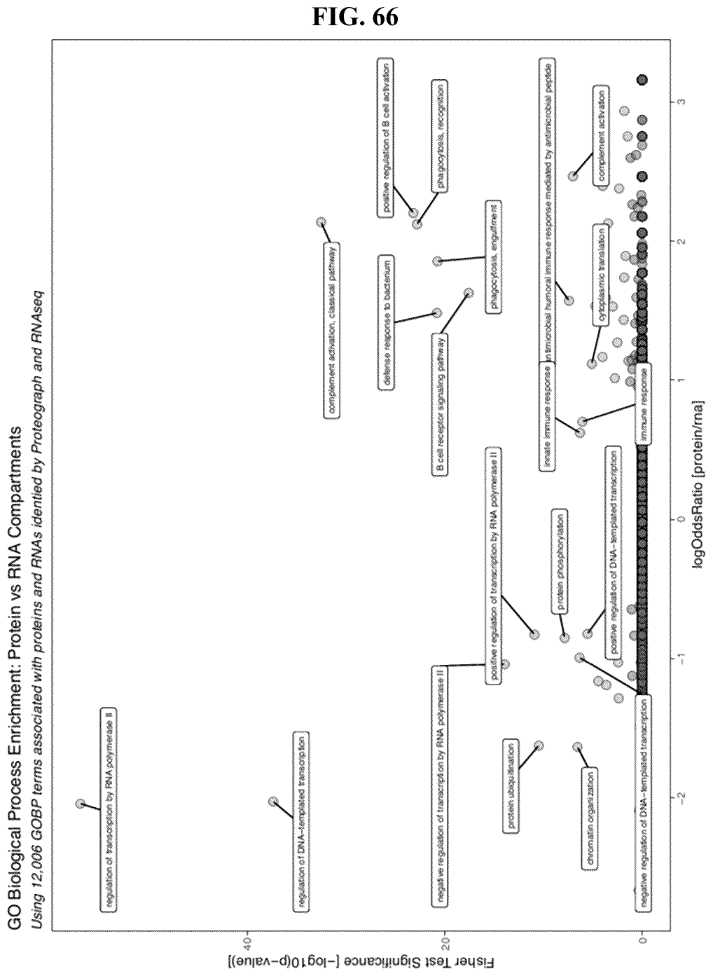

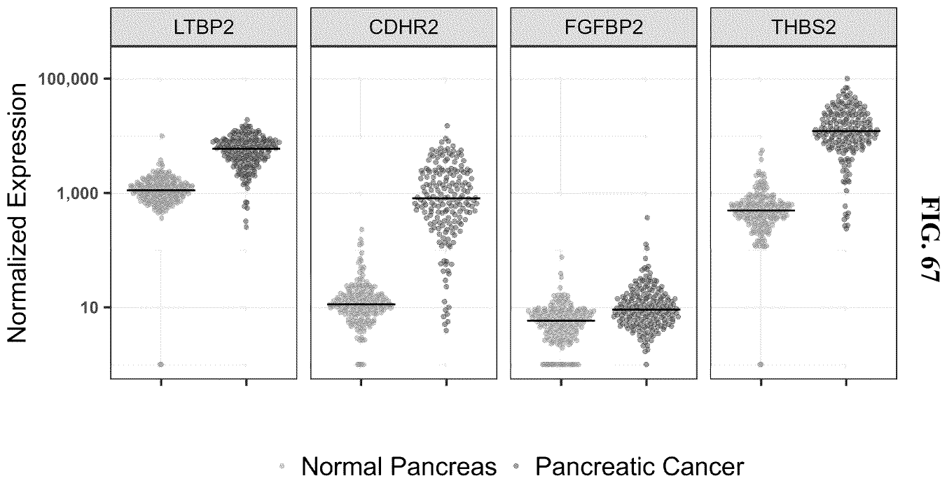

Given the low number of associated-feature counts for many of these GOBP terms in protein (4,027<3 features) and RNA (2,752<3 features), the statistical significance of a given feature's LOR may be uncertain. To address this, a Fisher's test for significance of proportionality differences was used, with Bonferroni correction of the raw p-value to account for multiple testing effects. The results were visualized in a volcano plot of statistical significance of a feature's LOR vs the magnitude of the feature's LOR (FIG. 66). A total of 40 GOBP terms had a statistically significant LOR (light gray circles; FIG. 66), and the names of the top 20 GOBP terms (by significance) are annotated.

The GOBP terms with significant enrichment are listed in Table 24. The features are ranked by the number of underlying features (e.g., proteins) that are associated with that term for proteins (n-protein). Positive LOR values indicate enrichment in protein vs RNA. Bonferroni was used for Fisher test p-value multiple testing correction (Adjusted p-value). The total number of GOBP instances for each omics type are shown in the bottom row. Although the subjects included in this analysis were derived from the PDAC study, this is not a list of terms significantly enriched between cancer and non-cancer. Instead, this is a list of terms significantly enriched between protein and RNA.

### DISCUSSION

This analysis shows the value of interrogating study subjects' samples with more than one omics type. Specifically, in this comparison of biological process exploration as measured by GOBP term coverage between proteins and RNA, there were many differences in GOBP term representation (e.g., wide range of LOR values) some of which achieved significance.

Since this is a blood-based, multi-omics analysis, it is not a given that the overlapping GOBP terms between proteins and RNAs are sampling the same physiological system since the proteins and RNAs could be derived from different cells, tissues, and organs as the blood circulates through the body. While this potential source-distinction might be sufficient in-of-itself to justify collecting both-omics types in a study, this analysis extends that differentiation.

TABLE 24
GOBP terms with significant LOR-based enrichment between protein and RNA

| | n- | | Adjusted p- | | |
|---|---|---|---|---|---|
| GOBP Name | proteins | n-RNAs | value | LOR | Enrichment |
| innate immune | 154 | 976 | 5.03588E−07 | 0.621192405 | protein |
| response | | | | | |
| immune response | 120 | 701 | 8.83006E−07 | 0.702196047 | protein |
| defense response | 89 | 238 | 1.64091E−21 | 1.483956662 | protein |
| to bacterium | | | | | |
| complement | 79 | 110 | 3.0127E−33 | 2.136597499 | protein |
| activation, | | | | | |
| classical pathway | | | | | |
| positive regulation | 77 | 2061 | 1.24493E−11 | −0.8272038 | RNA |
| of transcription by | | | | | |
| RNA polymerase | | | | | |
| II | | | | | |
| B cell receptor | 67 | 155 | 2.67011E−18 | 1.62817367 | protein |
| signaling pathway | | | | | |
| phagocytosis, | 64 | 118 | 1.88135E−21 | 1.855111001 | protein |
| engulfment | | | | | |
| phagocytosis, | 58 | 82 | 1.57962E−23 | 2.120501588 | protein |
| recognition | | | | | |
| protein | 57 | 1564 | 1.42437E−08 | −0.850986528 | RNA |
| phosphorylation | | | | | |
| positive regulation | 56 | 73 | 6.87212E−24 | 2.201613539 | protein |
| of B cell activation | | | | | |
| negative regulation | 54 | 1791 | 1.19507E−14 | −1.041604847 | RNA |
| of transcription by | | | | | |
| RNA polymerase | | | | | |
| II | | | | | |
| positive regulation | 49 | 1306 | 2.91207E−06 | −0.821299133 | RNA |
| of DNA-templated | | | | | |
| transcription | | | | | |
| cytoplasmic | 48 | 185 | 7.90401E−06 | 1.11676598 | protein |
| translation | | | | | |
| extracellular | 41 | 175 | 0.001696639 | 1.014425776 | protein |
| matrix | | | | | |
| organization | | | | | |
| blood coagulation | 39 | 143 | 9.39051E−05 | 1.166389531 | protein |
| antimicrobial | 36 | 88 | 3.95019E−08 | 1.571931041 | protein |
| humoral immune | | | | | |
| response mediated | | | | | |
| by antimicrobial | | | | | |
| peptide | | | | | |
| negative regulation | 34 | 1076 | 4.63512E−07 | −0.992829371 | RNA |
| of DNA-templated | | | | | |
| transcription | | | | | |
| regulation of | 32 | 2865 | 1.2521E−57 | −2.039857304 | RNA |
| transcription by | | | | | |
| RNA polymerase | | | | | |
| II | | | | | |
| negative regulation | 28 | 71 | 1.82927E−05 | 1.534974093 | protein |
| of endopeptidase | | | | | |
| activity | | | | | |
| platelet | 27 | 89 | 0.003040503 | 1.272534433 | protein |
| aggregation | | | | | |
| defense response | 26 | 98 | 0.041051601 | 1.138382399 | protein |
| to Gram-negative | | | | | |
| bacterium | | | | | |
| retina homeostasis | 23 | 55 | 0.000214394 | 1.593444253 | protein |
| regulation of | 22 | 1946 | 4.22804E−38 | −2.024616148 | RNA |
| DNA-templated | | | | | |
| transcription | | | | | |
| acute-phase | 22 | 49 | 0.000158224 | 1.664483 | protein |
| response | | | | | |
| killing of cells of | 22 | 56 | 0.00104966 | 1.530924445 | protein |
| another organism | | | | | |
| antibacterial | 20 | 56 | 0.013439807 | 1.435522954 | protein |
| humoral response | | | | | |
| DNA repair | 19 | 713 | 3.67583E−05 | −1.162498415 | RNA |
| complement | 19 | 19 | 9.64127E−08 | 2.465240283 | protein |
| activation | | | | | |
| cellular response to | 19 | 624 | 0.003718658 | −1.028821183 | RNA |
| DNA damage | | | | | |
| stimulus | | | | | |
| ubiquitin- | 16 | 618 | 0.00023266 | −1.191123141 | RNA |
| dependent protein | | | | | |
| catabolic process | | | | | |
| zymogen | 15 | 31 | 0.015189134 | 1.739074131 | protein |
| activation | | | | | |
| fibrinolysis | 15 | 21 | 0.000352238 | 2.128577697 | protein |
| complement | 14 | 15 | 9.92056E−05 | 2.396034699 | protein |
| activation, | | | | | |
| alternative | | | | | |
| pathway | | | | | |
| protein | 14 | 833 | 3.37705E−11 | −1.624127468 | RNA |
| ubiquitination | | | | | |
| positive regulation | 13 | 23 | 0.020819808 | 1.894406033 | protein |
| of immune | | | | | |
| response | | | | | |
| chromatin | 11 | 467 | 0.004210994 | −1.28529854 | RNA |
| remodeling | | | | | |
| positive regulation | 11 | 12 | 0.004579412 | 2.377890918 | protein |
| of opsonization | | | | | |
| chromatin | 10 | 601 | 2.79585E−07 | −1.633440973 | RNA |
| organization | | | | | |
| complement | 8 | 6 | 0.033405517 | 2.75247075 | protein |
| activation, lectin | | | | | |
| pathway | | | | | |
| opsonization | 8 | 5 | 0.015383821 | 2.934796187 | protein |
| All Terms | 1536 | 21924 | 257760 | | |

### Example 13. Associating Features with Biological Processes Related to PDAC

An analysis was performed on the data from example 11 in order to better understand the association of the features found to be useful in creating a classifier for predicting patients with PDAC. The results for the top features of the top features classifier are displayed in Table 25.

TABLE 25
Connection of features to PDAC or Cancer

| | | | Association | PDAC | Broader |
|---|---|---|---|---|---|
| Omics | | | Previously | Biology | Cancer |
| Type | Feature ID | Protein (Gene) | Reported?¹ | Connection? | Connection? |
| Proteins | NP4_GAGGQSMSEAPTGDHAPAPTR | Q14767-LTBP2 (LTBP2) | Yes | Yes | Yes |
| | NP3_TFVIIPELVLPNR | Q9BYE9-CDHR2 (CDHR2) | | Yes | Yes |
| | NP2_TFVIIPELVLPNR | Q9BYE9-CDHR2 (CDHR2) | | Yes | Yes |
| | NP3_DSC(UniMod: 4)TMRPSSLGQGAGEVWLR | Q9BYJ0-FGFBP2 (FGFBP2) | | | |
| | NP3_DNC(UniMod: 4)PHLPNSGQEDFDK | P35442-TSP2 (THBS2) | Yes | Yes | Yes |
| Metabolites | NEG_AICAR | NA | | | Yes |
| | POS_Cystine | NA | | Yes² | Yes |
| | NEG_CMP | NA | | Yes² | Yes |
| | NEG_Gentisate | NA | | Yes² | Yes |
| | POS_Creatine | NA | | Yes² | Yes |
| Lipids | NEG_PC(18:2_20:5) + AcO | NA | Yes | Yes³ | Yes³ |
| | POS_DAG(18:1_20:0) + NH4 | NA | | | |
| | NEG_PE(O − 16:0_22:6) − H | NA | Yes | | |
| | NEG_PC(18:2_20:3) + AcO | NA | Yes | Yes³ | Yes³ |
| | POS_CER(d18:1/18:0) + H | NA | Yes | | Yes³ |
| RNAS | ENST00000483727.5 | NA (ENSG00000078747) | | | |
| | ENST00000531734.6 | Q01433-AMPD2 (AMPD2) | | | |
| | ENST00000437154.6 | Q9UKQ2-ADA28 (ADAM28) | | | |
| | ENST00000531997.1 | NA (GLYATL1 pseudogene) | | | |
| | ENST00000424185.7 | Q8IW45-NNRD (NAXD) | | | |

¹Blank entries indicate that a previously documented PDAC association was not found
²Connected to PDAC via differential expression (PDAC tumors vs normal pancreatic tissue) of genes that encode enzymes or transporters acting on the metabolite.
³Refers to the particular class of lipids and not necessarily the specific species listed as a feature.

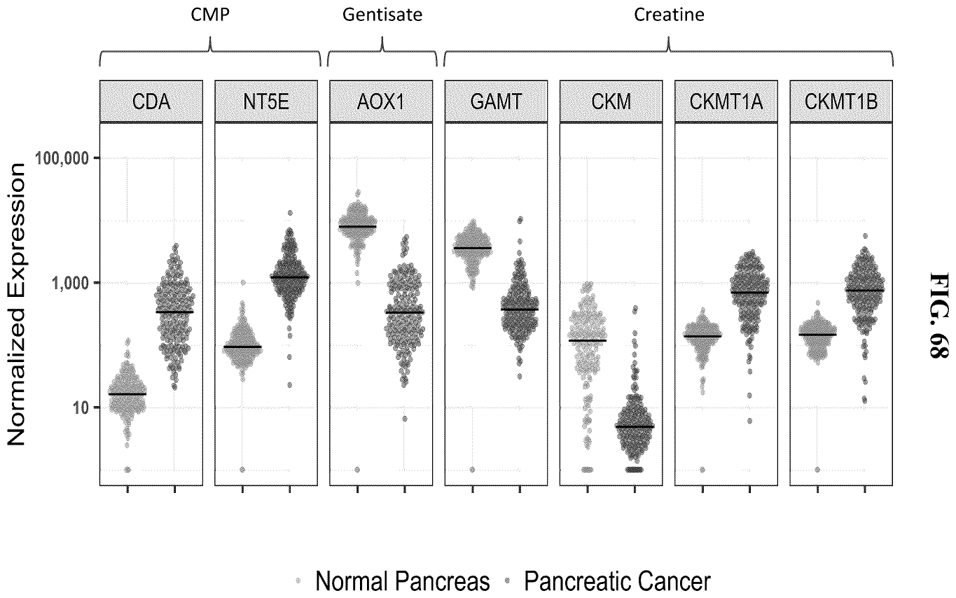

Red entries in Table 25 represent features that were in higher concentration in PDAC blood while blue entries in Table 25 represent features that were in lower concentration in PDAC blood. 3 of 5 proteins represented in the classifier had significantly higher mRNA expression in PDAC tumors than in normal pancreatic tissue (FIG. 67). Two of these proteins (LTBP2 & TSP2) are known to be secreted into the extracellular matrix, where they are thought to act as cellular anti-adhesives. Together, this suggests that the increase of these proteins in the plasma of PDAC subjects may be directly attributable to the excess of these proteins in their tumors. Notably, THBS2 mRNA was more highly expressed in PDAC than in any other of a wide variety of normal and cancer tissue types profiled by the Cancer Genome Atlas and the Genotype Tissue Expression project.

4 of 5 metabolites could be connected to PDAC biology because genes that encode enzymes or transporters acting on them are differentially expressed in PDAC tumors vs normal pancreatic tissue (FIG. 68). The 5th metabolite, AICAR, is an AMP analog that stimulates AMPK, a metabolic tumor suppressor. Reduced AICAR levels in the plasma of PDAC may promote tumor growth by reducing AMPK activity within tumors.

Phosphatidylcholines (PC), as a class, are lower in our PDAC subjects than the controls. Enzymes that metabolize dietary PCs, such as phospholipase A2 group IB, encoded by PLA2G1B, are expressed specifically by the pancreas, and secreted into the intestinal tract. PLA2G1B mRNA is 21-fold lower in PDAC tumors than in normal pancreas and others have found that PLA2G1B is silenced in PDAC samples. Taken together, this suggests that PDAC-driven disturbances to pancreatic PC enzyme secretion into the gut result in lower PC levels in the circulation of PDAC patients.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1.-157. (canceled)

158. A pancreatic cancer evaluation method, the method comprising:
(a) obtaining a data set comprising biomarker measurements of a biofluid sample from a subject, wherein the biomarker measurements correspond to biomarkers comprising AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1, or a combination thereof,
(b) applying a classifier to the data set to qualitatively or quantitatively determine a pancreatic cancer state in the subject, wherein the pancreatic cancer state when determined qualitatively comprises a positive result indicative of the pancreatic cancer in the subject or a negative result indicative of a lack of the pancreatic cancer in the subject, and wherein the pancreatic cancer state when determined quantitatively comprises a degree of risk that the subject has the pancreatic cancer; and
(c) administering a pancreatic cancer treatment to the subject when the pancreatic cancer state is the positive result or when the degree of risk that the subject has the pancreatic cancer is higher than normal; or
(d) observing or treating the subject without administering the pancreatic cancer treatment to the subject when the pancreatic cancer state is the negative result or when the degree of risk is at or below a normal risk that the subject has the pancreatic cancer.

159. The method of claim 158, wherein the classifier is trained to differentiate pancreatic cancer samples from non-pancreatic cancer samples with an area under the curve (AUC) of greater than 0.85.

160. The method of claim 158, wherein the biomarkers comprise two or more of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, APOA1, or CA19-9.

161. The method of claim 160, wherein the biomarkers comprise three or more of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, APOA1, or CA19-9.

162. The method of claim 161, wherein the biomarkers comprise four or more of AACT, A1AT, A2GL, AMPN, LBP, ICAM1, PIGR, CO5, S10A8, CO2, CO9, ITIH3, RET4, FCG3A, TETN, CRP, NOE1, F13B, APOA2, APOA1, or CA19-9.

163. The method of claim 158, wherein the biomarker measurements are obtained by adding an internal standard to the biofluid sample for the biomarkers present in the biofluid sample.

164. The method of claim 163, wherein the internal standard comprises a label.

165. The method of claim 164, wherein the label comprises an isotopic label.

166. The method of claim 158, wherein the biomarker measurements are obtained using mass spectrometry.

167. The method of claim 158, wherein the biomarker measurements are obtained using an immunoassay.

168. The method of claim 158, wherein the biomarker measurements are obtained using molecular probes.

169. The method of claim 158, wherein the biomarker measurements are obtained using chromatography.

170. The method of claim 158, wherein the pancreatic cancer state further comprises a pancreatic cancer stage in the subject.

171. The method of claim 170, wherein the pancreatic cancer is stage I or stage II pancreatic cancer.

172. The method of claim 170, wherein the pancreatic cancer is stage III or stage IV pancreatic cancer.

173. The method of claim 158, wherein the pancreatic cancer comprises pancreatic ductal adenocarcinoma (PDAC).

174. The method of claim 158, wherein the biofluid sample comprises pancreatic cyst fluid, urine, blood, plasma, or serum.

175. The method of claim 158, further comprising performing pancreatic imaging, wherein the pancreatic imaging is performed using an ultrasound or computer tomography.

176. The method of claim 158, wherein the subject is a human.

177. A system, comprising:
one or more processors; and
one or more memories storing machine-executable code that, when executed, cause the one or more processors to:
receive a data set comprising biomarker measurements of a biofluid sample from a subject, wherein the biomarker measurements correspond to biomarkers comprising AACT, A1AT, A2GL, AMPN, LBP, ICAM1, PIGR, CO5, S10A8, CO2, CO9, ITIH3, RET4, FCG3A, TETN, CRP, NOE1, F13B, APOA2, or APOA1, or a combination thereof;
apply a classifier to the data set to qualitatively or quantitatively determine a pancreatic cancer state in the subject, wherein the pancreatic cancer state when determined qualitatively comprises a positive result indicative of the pancreatic cancer in the subject or negative result indicative of a lack of the pancreatic cancer in the subject, and wherein the pancreatic cancer state when determined quantitatively comprises a degree of risk that the subject has the pancreatic cancer; and
provide information to aid in a treatment of the subject, wherein the treatment comprises:
administering a pancreatic cancer treatment to the subject when the pancreatic cancer state is the positive result or when the degree of risk that the subject has the pancreatic cancer is higher than normal; or
observing the subject or treating the subject without administering the pancreatic cancer treatment to the subject when the pancreatic cancer state is the negative result or when the degree of risk is at or below a normal risk that the subject has the pancreatic cancer.


SEQUENCE LISTING (21 sequences)

SEQ ID NO: 1 - protein, 21 aa, Synthetic construct
GAGGQSMSEAPTGDHAPAPTR

SEQ ID NO: 2 - protein, 13 aa, Synthetic construct
TFVIIPELVLPNR

SEQ ID NO: 3 - protein, 20 aa, Synthetic construct
  MOD_RES 3..4: note = Carbamidomethyl
DSCTMRPSSLGQGAGEVWLR

SEQ ID NO: 4 - protein, 16 aa, Synthetic construct
  MOD_RES 3..4: note = Carbamidomethyl
DNCPHLPNSGQEDFDK

SEQ ID NO: 5 - protein, 14 aa, Synthetic construct
GLVLGAGWAEGYLR

SEQ ID NO: 6 - protein, 20 aa, Synthetic construct
  MOD_RES 19..20: note = Carbamidomethyl
LVFNPDQEDLDGDGRGDICK

SEQ ID NO: 7 - protein, 10 aa, Synthetic construct
AFDLYFVLDK

SEQ ID NO: 8 - protein, 10 aa, Synthetic construct
VFLVGNVEIR

SEQ ID NO: 9 - protein, 15 aa, Synthetic construct
RVSPVGETYIHEGLK

SEQ ID NO: 10 - protein, 10 aa, Synthetic construct
ASEQIYYENR

SEQ ID NO: 11 - protein, 15 aa, Synthetic construct
VLPGGDTYMHEGFER

SEQ ID NO: 12 - protein, 13 aa, Synthetic construct
AVDIPHMDIEALK

SEQ ID NO: 13 - protein, 15 aa, Synthetic construct
AMGIMNSFVNDIFER

SEQ ID NO: 14 - protein, 12 aa, Synthetic construct
MPEQEYEFPEPR

SEQ ID NO: 15 - protein, 27 aa, Synthetic construct
SGVISDTELQQALSNGTWTPFNPVTVR

SEQ ID NO: 16 - protein, 15 aa, Synthetic construct
  MOD_RES 1..2: note = Oxidation
MEDVNSNVNADQEVR

SEQ ID NO: 17 - protein, 13 aa, Synthetic construct
VGHDYQWIGLNDK

SEQ ID NO: 18 - protein, 16 aa, Synthetic construct
  MOD_RES 4..5: note = Carbamidomethyl
HAECIYLGHFSDPMYK

SEQ ID NO: 19 - protein, 20 aa, Synthetic construct
NGIFWGTWPGVSEAHPGGYK

SEQ ID NO: 20 - protein, 20 aa, Synthetic construct
DSCTMRPSSLGQGAGEVWLR

SEQ ID NO: 21 - protein, 16 aa, Synthetic construct
DNCPHLPNSGQEDFDK